(19) United States
(12) Patent Application Publication
(10) Pub. No.: US 2023/0192886 A1
(43) Pub. Date: Jun. 22, 2023

(54) ADOPTIVE CELL THERAPY COMBINATION TREATMENT AND COMPOSITIONS THEREOF

(71) Applicants: Immatics Biotechnologies GmbH, Tuebingen (DE); Immatics US, Inc., Houston, TX (US)

(72) Inventors: Harpreet SINGH, Tuebingen (DE); Toni WEINSCHENK, Tuebingen (DE); Steffen WALTER, Houston, TX (US)

(21) Appl. No.: 18/053,705

(22) Filed: Nov. 8, 2022

Related U.S. Application Data
(60) Provisional application No. 63/277,074, filed on Nov. 8, 2021.

(51) Int. Cl. C07K 16/30; A61K 38/17; A61P 35/00

(52) U.S. Cl. CPC C07K 16/3053; A61K 38/1774; A61P 35/00

(57) ABSTRACT

A method of treating a patient who has a recurrent cancer that presents a peptide, including administering to the patient a treatment composition comprising an antigen binding molecule that binds to the peptide, in which the patient has received a prior treatment with a pretreatment composition comprising a second antigen binding molecule that binds to a different peptide.

### CROSS-REFERENCE TO RELATED APPLICATION

This application claims priority to United States Provisional Pat. Application No. 63/277,074, filed 8 Nov. 2021, and entitled “ADOPTIVE CELL THERAPY COMBINATION TREATMENT AND COMPOSITIONS THEREOF”, which is incorporated herein by reference in its entirety.

### REFERENCE TO SEQUENCE LISTING SUBMITTED ELECTRONICALLY

Pursuant to 37 C.F.R. § 1.821-825 (see M.P.E.P. § 2442.03(a)), a Sequence Listing in the form of an ASCII-compliant text file (entitled “3000011-027001_Sequence_listing_ST26.xml” created on 08 Nov. 2022, and 640,390 bytes in size) is submitted concurrently with the instant application. The sequence listing contained in this ASCII-formatted document is part of the specification and is herein incorporated by reference in its entirety. For the avoidance of doubt, if discrepancies exist between the sequences mentioned in the specification and the electronic sequence listing, the sequences in the specification shall be deemed to be the correct ones.

### FIELD

The present disclosure relates to peptides, proteins, nucleic acids and cells for use in immunotherapeutic methods and combination treatment therapies. In particular, the present disclosure relates to the immunotherapy of cancer. The present disclosure furthermore relates to tumor-associated T-cell peptide epitopes, alone or in combination with other tumor-associated peptides that can for example serve as active pharmaceutical ingredients of compositions that stimulate anti-tumor immune responses, or to stimulate T-cells ex vivo and transfer into patients. Peptides bound to molecules of the major histocompatibility complex (MHC), or peptides as such, can also be targets of antibodies, soluble T-cell receptors, and other binding molecules.

The present disclosure further relates to combination of immunotherapies on recurrent cancers presenting multiple targets.

### BACKGROUND

According to the World Health Organization (WHO), cancer ranked among the four major non-communicable deadly diseases worldwide in 2012. For the same year, colorectal cancer, breast cancer and respiratory tract cancers were listed within the top 10 causes of death in high income countries.

Recurrent or refractory advanced cancer remains a major health problem worldwide and, also according to the World Health Organization, ranks second to cardiovascular disease as an overall cause of mortality. Although there has been significant progress over the last few decades, patients with recurrent or refractory advanced solid tumors still have a generally poor prognosis. These patients have a high unmet medical need.

Immunotherapy has significantly changed the standard of care in oncology. See, e.g., Hoos A (2016), Development of immuno-oncology drugs - from CTLA4 to PD1 to the next generations, Nat Rev Drug Discovery 15, 235-247, which is incorporated by reference herein in its entirety. Adoptive cellular therapy is one of the major drivers of this success, including the reinfusion of autologous or allogenic anti-tumor T lymphocytes after ex vivo expansion or genetic engineering with tumor-specific receptors. See, e.g., Rosenberg SA, Restifo NP (2015). Despite of some advances, the targeted patient population has still a high unmet medical need and no remaining standard treatment option and as a consequence a very poor prognosis.

### SUMMARY

Immunotherapy of cancer represents an option of specific targeting of cancer cells while minimizing side effects. Cancer immunotherapy makes use of the existence of tumor associated antigens.

The current classification of tumor associated antigens (TAAs) comprises the following major groups:

a) Cancer-testis antigens: The first TAAs ever identified that can be recognized by T-cells belong to this class, which was originally called cancer-testis (CT) antigens. Since the cells of testis do not express class I and II HLA molecules, these antigens cannot be recognized by T-cells in normal tissues and can therefore be considered as immunologically tumor specific. Well-known examples for CT antigens are the MAGE family members and NY-ESO-1.

b) Differentiation antigens: These TAAs are shared between tumors and the normal tissue from which the tumor arose. Most of the known differentiation antigens are found in melanomas and normal melanocytes. Examples include, but are not limited to, tyrosinase and Melan-A/MART-1 for melanoma or PSA for prostate cancer.

c) Overexpressed TAAs: Genes encoding widely expressed TAAs have been detected in histologically different types of tumors as well as in many normal tissues, generally with lower expression levels. It is possible that many of the epitopes processed and potentially presented by normal tissues are below the threshold level for T-cell recognition, while their overexpression in tumor cells can trigger an anticancer response by breaking previously established tolerance. Prominent examples for this class of TAAs are Her-2/neu, survivin, telomerase, or WT1.

d) Tumor specific antigens: These unique TAAs arise from mutations of normal genes (such as β-catenin, CDK4, etc.). Some of these molecular changes are associated with neoplastic transformation and/or progression. Tumor specific antigens are generally able to induce strong immune responses without bearing the risk for autoimmune reactions against normal tissues. On the other hand, these TAAs are in most cases only relevant to the exact tumor on which they were identified and are usually not shared between many individual tumors. Tumor specificity (or -association) of a peptide may also arise if the peptide originates from a tumor specific (-associated) exon in case of proteins with tumor specific (-associated) isoforms.

e) Oncoviral proteins: These TAAs are viral proteins that may play a critical role in the oncogenic process and, because they are foreign (not of human origin), they can evoke a T-cell response. Examples of such proteins are the human papilloma type 16 virus proteins, E6 and E7, which are expressed in cervical carcinoma.

Human endogenous retroviruses (HERVs) make up a significant portion (~8%) of the human genome. These viral elements integrated into the genome millions of years ago and were since then vertically transmitted through generations. The huge majority of HERVs have lost functional activity through mutation or truncation, yet some endogenous retrovirus, such as the members of the HERV-K clade, still encode functional genes and have been shown to form retrovirus-like particles. Transcription of HERV proviruses is epigenetically controlled and remains silenced under normal physiological conditions. Reactivation and overexpression resulting in active translation of viral proteins has however been described in certain diseases and especially for different types of cancer. This tumor-specific expression of HERV derived proteins can be harnessed for different types of cancer immunotherapy.

f) TAAs arising from abnormal post-translational modifications: Such TAAs may arise from proteins which are neither specific nor overexpressed in tumors but nevertheless become tumor associated by post-translational processes primarily active in tumors. Examples for this class arise from altered glycosylation patterns leading to novel epitopes in tumors as for MUC1 or events like protein splicing during degradation which may or may not be tumor specific.

T-cell-based immunotherapy targets peptide epitopes derived from tumor-associated or tumor specific proteins, which are presented by MHC molecules. The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

There are two classes of MHC molecules, MHC class I and MHC class II. MHC class I molecules are composed of an alpha heavy chain and beta-2-microglobulin, MHC class II molecules of an alpha and a beta chain. Their three-dimensional conformation results in a binding groove, which is used for non-covalent interaction with peptides.

MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation in the literature (Brossart and Bevan, 1997; Rock et al., 1990). MHC class II molecules can be found predominantly on professional antigen presenting cells (APCs), and primarily present peptides of exogenous or transmembrane proteins that are taken up by APCs e.g. during endocytosis and are subsequently processed.

Complexes of peptide and MHC class I are recognized by CD8-positive T-cells bearing the appropriate T-cell receptor (TCR), whereas complexes of peptide and MHC class II molecules are recognized by CD4-positive helper T-cells bearing the appropriate TCR. It is well known that the TCR, the peptide and the MHC are thereby present in a stoichiometric amount of 1:1:1.

CD4-positive helper T-cells play an important role in inducing and sustaining effective responses by CD8-positive cytotoxic T-cells. The identification of CD4-positive T-cell epitopes derived from tumor associated antigens (TAA) is of great importance for the development of pharmaceutical products for triggering anti-tumor immune responses. At the tumor site, T helper cells, support a cytotoxic T-cell (CTL) friendly cytokine milieu and attract effector cells, e.g. CTLs, natural killer (NK) cells, macrophages, and granulocytes.

The present disclosure relates to immunotherapies of recurrent cancers, for example, recurrent sarcoma, including administration of compositions containing antigen binding molecules.

In an aspect, the disclosure provides for methods of treating a patient, including, administering to the patient a treatment composition comprising an antigen binding molecule that binds to a PRAME peptide, wherein the patient has received one or more prior treatments with a pretreatment composition comprising a second antigen binding molecule that binds to a second peptide different from the PRAME peptide.

In another aspect, the disclosure provides for methods of treating a patient who has recurrent cancer, including, administering to the patient a treatment composition comprising an antigen binding molecule that binds to a PRAME peptide, wherein the patient has received one or more prior treatments with a pretreatment composition comprising an antigen binding molecule that binds to a peptide from Table 10.

In another aspect, the disclosure provides for methods of treating a patient who has recurrent cancer, including, administering to the patient a treatment composition comprising an antigen binding molecule that binds to a PRAME peptide, wherein the patient has received one or more prior treatments with a pretreatment composition comprising an antigen binding molecule that binds to a peptide selected from the group consisting of MAG-003, MAGEA1-003, COL6A3-002, and MAGE-A4.

In a preferred aspect, the disclosure provides for methods of treating a patient who has recurrent cancer, including, administering to the patient a treatment composition comprising an antigen binding molecule that binds to SLLQHLIGL (SEQ ID NO: 310), wherein the patient has received one or more prior treatments with a composition comprising an antigen binding molecule that binds to a peptide selected from the group consisting of KVLEHVVRV (SEQ ID NO: 430), KVLEYVIKV (SEQ ID NO: 417), FLLDGSANV (SEQ ID NO: 453), and GVYDGREHTV (SEQ ID NO: 401).

In another aspect, the disclosure provides for methods of treating a patient who has recurrent cancer, including, administering to the patient a treatment composition comprising an antigen binding molecule that binds to a peptide from Table 10, wherein the patient has received one or more prior treatments with a pretreatment composition including an antigen binding molecule that binds to a PRAME peptide.

In yet another aspect, the disclosure provides for methods of treating a patient who has recurrent cancer, including, administering to the patient a treatment composition comprising an antigen binding molecule that binds to a peptide selected from group consisting of KVLEHVVRV (SEQ ID NO: 430), KVLEYVIKV (SEQ ID NO: 417), FLLDGSANV (SEQ ID NO: 453), and GVYDGREHTV (SEQ ID NO: 401), wherein the patient has received one or more prior treatments with a pretreatment composition including an antigen binding molecule that binds to SLLQHLIGL (SEQ ID NO: 310).

In an aspect, the present disclosure provides for methods of treating a patient who has recurrent cancer that presents a peptide other than a PRAME peptide, including administering to the patient a treatment composition containing an antigen binding molecule that binds the peptide other than a PRAME peptide, in which the patient has received a prior treatment with a pretreatment composition containing an antigen binding molecule that binds a PRAME peptide on the cell surface.

In an aspect, SLLQHLIGL (SEQ ID NO: 310) is a PRAME peptide provided herein.

In an aspect, the present disclosure provides for methods of eliciting an immune response in a patient who has a recurrent cancer that presents a peptide other than a PRAME peptide, including administering to the patient a treatment composition containing an antigen binding molecule that binds the peptide other than a PRAME peptide, in which the patient has received a prior treatment with a pretreatment composition containing an antigen binding molecule that binds the PRAME peptide on the cell surface, in which the PRAME peptide optionally contains SLLQHLIGL (SEQ ID NO: 310).

In an aspect, the disclosure provides for methods of treating a patient with cancer including, (1) a first treatment with an antigen binding molecule that binds to a PRAME peptide, such as SLLQHLIGL (SEQ ID NO: 310), and (2) one or more subsequent treatments of the same patient with an antigen binding molecule that binds to a peptide in Table 10.

In another aspect, the disclosure provides for methods of treating a patient with cancer including, (1) a first treatment with an antigen binding molecule that binds to a peptide of Table 10, and (2) one or more subsequent treatments of the same patient with an antigen binding molecule that binds to a PRAME peptide, such as SLLQHLIGL (SEQ ID NO: 310).

In an aspect, the disclosure provides for methods of treating a patient with cancer including, (1) a first treatment with an antigen binding molecule that binds to a PRAME peptide, such as SLLQHLIGL (SEQ ID NO: 310), and (2) one or more subsequent treatments of the same patient with an antigen binding molecule that binds to a peptide selected from the group consisting of MAG-003, MAGEA1-003, COL6A3-002, and MAGE-A4.

In another aspect, the disclosure provides for methods of treating a patient with cancer including, (1) a first treatment with an antigen binding molecule that binds to a peptide selected from the group consisting of MAG-003, MAGEA1-003, COL6A3-002, and MAGE-A4, and (2) one or more subsequent treatments of the same patient with an antigen binding molecule that binds to a PRAME peptide, such as SLLQHLIGL (SEQ ID NO: 310).

In an aspect, the disclosure provides for methods of treating a patient with cancer including, (1) a first treatment with an antigen binding molecule that binds to SLLQHLIGL (SEQ ID NO: 310), and (2) one or more subsequent treatments of the same patient with an antigen binding molecule that binds to a peptide selected from the group consisting of KVLEHVVRV (SEQ ID NO: 430), KVLEYVIKV (SEQ ID NO: 417), FLLDGSANV (SEQ ID NO: 453), and GVYDGREHTV (SEQ ID NO: 401).

In another aspect, the disclosure provides for methods of treating a patient with cancer including, (1) a first treatment with an antigen binding molecule that binds to a peptide selected from the group consisting of KVLEHVVRV (SEQ ID NO: 430), KVLEYVIKV (SEQ ID NO: 417), FLLDGSANV (SEQ ID NO: 453), and GVYDGREHTV (SEQ ID NO: 401), and (2) one or more subsequent treatments of the same patient with an antigen binding molecule that binds to a PRAME peptide, such as SLLQHLIGL (SEQ ID NO: 310).

In an aspect, an antigen binding molecule described herein may include a T cell receptor (TCR) and/or an antibody.

In another aspect, a TCR that binds to PRAME includes
(1) a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
(2) a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
(3) a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
(4) a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
(5) a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
(6) a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
(7) a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
   wherein the T-cell receptor is capable of binding to a peptide consisting of the amino acid sequence of SLLQHLIGL (SEQ ID NO: 310) in a complex with HLA-A*02.

In another aspect, a TCR that binds to PRAME includes
(1) an α chain variable domain comprising SEQ ID NO: 15, and a β chain variable domain comprising SEQ ID NO: 21, or
(2) an α chain variable domain comprising SEQ ID NO: 27, and a β chain variable domain comprising SEQ ID NO: 33, or
(3) an α chain variable domain comprising SEQ ID NO: 39, and a β chain variable domain comprising SEQ ID NO: 45, or
(4) an α chain variable domain comprising SEQ ID NO: 51, and a β chain variable domain comprising SEQ ID NO: 57, or
(5) an α chain variable domain comprising SEQ ID NO: 63, and a β chain variable domain comprising SEQ ID NO: 69, or
(6) an α chain variable domain comprising SEQ ID NO: 75, and a β chain variable domain comprising SEQ ID NO: 81, or
(7) an α chain variable domain comprising SEQ ID NO: 87, and a β chain variable domain comprising SEQ ID NO: 93, or
(8) an α chain variable domain comprising SEQ ID NO: 111, and a β chain variable domain comprising SEQ ID NO: 117,
   wherein the T-cell receptor is capable of binding to a peptide consisting of the amino acid sequence of SLLQHLIGL (SEQ ID NO: 310) in a complex with HLA-A*02.

In another aspect, the antigen binding molecule is expressed in a T cell. In another aspect, the T cell includes CD4+ T cell, CD8+ T cell, CD4+CD8+ T cell, CD4-CD8- T cell, and/or γδ T cell.

In another aspect, the compositions described herein further may include at least one adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23).

In another aspect, antigen binding molecules that bind to PRAME may include a first polypeptide chain and a second polypeptide chain, wherein the first polypeptide chain comprises a first hinge domain and/or a first Fc domain,
wherein said first polypeptide chain comprising 95% identity to any one of SEQ ID NOs 178, 184, 187, 189, 190, 192, 195, 197, 200, 206, 208, 210, 212, 216, 218, 219, 220, 221, 222, 229, 230, 232, 234, 236, 238, 240, 241, 242, 243, 244, 246, 250, 251, 252, 253, 254, 255, 256, 257, 258, 259, 260, 261, 262, 263, 265, 298, 299, 300, 302, or 304 comprises the complementarity determining regions (CDRs) of said sequence;
wherein the second polypeptide chain comprises a second hinge domain and/or a second Fc domain,
wherein said second polypeptide comprising 95% identity to any one of SEQ ID NOs 179, 180, 181, 182, 183, 185, 186, 188, 191, 193, 194, 196, 198, 199, 201, 202, 203, 204, 205, 207, 209, 211, 213, 214, 215, 217, 223, 224, 225, 226, 227, 228, 231, 233, 235, 237, 239, 245, 247, 248, 249, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 301, or 303 comprises the CDRs of said sequence.

In another aspect, said first polypeptide chain is fused to said second polypeptide chain by covalent and/or non-covalent bonds between the first hinge domain and the second hinge domain, and/or between the first Fc domain and the second Fc domain.

In another aspect, said first and second Fc domains may each include at least one Fc effector function silencing mutation.

In another aspect, said first and second Fc domains may each include a CH3 domain comprising at least one mutation that facilitates the formation of heterodimers.

In another aspect, said first and second Fc domains may each include CH2 and CH3 domains comprising at least two additional cysteine residues.

In another aspect, the first antigen binding molecule may include
a) a first polypeptide chain comprising a first variable domain comprising three complementary determining regions (CDRs) CDRa1, CDRa2 and CDRa3, wherein
   the CDRa1 comprises or consists of the amino acid sequence DRGSQS (SEQ ID NO: 135) or an amino acid sequence at least 85% identical to SEQ ID NO: 135),
   the CDRa2 comprises or consists of the amino acid sequence IYQEGD (SEQ ID NO: 138) and
   the CDRa3 comprises or consists of the amino acid sequence CAAVIDNDQGGILTF (SEQ ID NO: 142), and
b) a second polypeptide chain comprising a second variable domain comprising three complementary determining regions (CDRs) CDRb1, and CDRb3, wherein
   the CDRb1 comprises or consists of the amino acid sequence PGHRA (SEQ ID NO: 167) or PGHRS (SEQ ID NO: 168), preferably PGHRA (SEQ ID NO: 167), or an amino acid sequence at least 85% identical to SEQ ID NO: 167) or SEQ ID NO: 168), preferably SEQ ID NO: 167);
   the CDRb2 comprises or consists of the amino acid sequence YVHGEE (SEQ ID NO: 170) or an amino acid sequence at least 85% identical to SEQ ID NO: 170), and
   the CDRb3 comprises or consists of the amino acid sequence CASSPWDSPNEQYF (SEQ ID NO: 172) or CASSPWDSPNVQYF (SEQ ID NO: 173), preferably CASSPWDSPNVQYF (SEQ ID NO: 173), or an amino acid sequence at least 85% identical to SEQ ID NO: 172) or SEQ ID NO: 173), preferably CASSPWDSPNVQYF (SEQ ID NO: 173).

In another aspect, antigen binding molecules described herein include
a) TCR variable domains variable domains that bind the PRAME-004:MHC complex selected from the following pairs:
   V_{A} comprises or consists of the amino acid sequence of SEQ ID NO: 305; and V_{B} comprises or consists of the amino acid sequence of SEQ ID NO: 306;
   V_{A} comprises or consists of the amino acid sequence of SEQ ID NO: 305; and V_{B} comprises or consists of the amino acid sequence of SEQ ID NO: 307;
   V_{A} comprises or consists of the amino acid sequence of SEQ ID NO: 305; and V_{B} comprises or consists of the amino acid sequence of SEQ ID NO: 308;
   V_{A} comprises or consists of the amino acid sequence of SEQ ID NO: 309; and V_{B} comprises or consists of the amino acid sequence of SEQ ID NO: 306;
   V_{A} comprises or consists of the amino acid sequence of SEQ ID NO: 309; and V_{B} comprises or consists of the amino acid sequence of SEQ ID NO: 307; or
   V_{A} comprises or consists of the amino acid sequence of SEQ ID NO: 309; and V_{B} comprises or consists of the amino acid sequence of SEQ ID NO: 306; and
b) antibody V_{H} and V_{L} domains that bind CD3, selected from the following pairs:
   V_{H} comprising or consisting of SEQ ID NO: 193; and a V_{L} comprising or consisting of SEQ ID NO: 192;
   V_{H} comprising or consisting of SEQ ID NO: 196; or SEQ ID NO: 198; (A02) or SEQ ID NO: 199; (D01) or SEQ ID NO: 200; (A02_H90Y) or SEQ ID NO: 201; (D01_H90Y), and a V_{L} comprising or consisting of SEQ ID NO: 197;
   V_{H} comprising or consisting of SEQ ID NO: 202; or SEQ ID NO: 207; (N100D) or SEQ ID NO: 209; (N100E) or SEQ ID NO: 211; (S101A) and a V_{L} comprising or consisting of SEQ ID NO: 204.

In another aspect, the recurrent cancer is selected from the group consisting of adrenocortical carcinoma, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, uterine corpus endometrial carcinoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin’s lymphoma, glioblastoma, cervical carcinoma, cervical squamous cell carcinoma and endocervical adenocarcinoma, cholangiocarcinoma, hepatocellular carcinoma, liver hepatocellular carcinoma, Ewing’s sarcoma, endometrial cancer, epithelial cancer of the larynx, esophageal carcinoma, oral carcinoma, atypical meningioma, papillary thyroid carcinoma, thymoma, brain tumors, salivary duct carcinoma, extranodal T/NK-cell lymphomas, rectal cancer, mouth and throat cancer, and multiple myeloma.

In an aspect, the first peptide may be undetectable in the cancer before the prior treatment.

In an aspect, the patient may have a treatment free interval for more than about three months prior to the initiation of the administering.

In an aspect, the PRAME peptide and the peptide other than the PRAME peptide may each be in a complex with an MHC molecule.

In an aspect, the treatment composition may contain a molecule that blocks an interaction between PD-1 and PD-L1.

In an aspect, the molecule that blocks an interaction between PD-1 and PD-L1 may be a monoclonal antibody.

In an aspect, the molecule that blocks an interaction between PD-1 and PD-L1 may be atezolizumab, pembrolizumab, nivolumab, cemiplimab, or combinations thereof.

In an aspect, the antigen binding molecule that binds the PRAME peptide may be TCR R11P3D3_KE and the peptide other than the PRAME peptide may be derived from MAGE-A4. In an aspect, the antigen binding molecule binds a peptide comprising a sequence GVYDGREHTV (SEQ ID NO: 401).

In an aspect, the treatment composition further comprises at least one adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23).

### BRIEF DESCRIPTION OF THE DRAWINGS

FIG. 1 shows exemplary experimental data showing γδ T-cell expansion using Zoledronate (Zometa) in defined medium, which contains IL-2, IL-15, and Amphotericin B.

FIG. 2A shows exemplary experimental data showing that, as compared with Vγ9δ2 T-cells without viral transduction (Mock), 34.9% of Vγ9δ2 T-cells transducing with αβ-TCR retrovirus and CD8αβ retrovirus αβ-TCR + CD8) stained positive by peptide/MHC-dextramer (TAA/MHC-dex) and anti-CD8 antibody (CD8), indicating the generation of Vγ9δ2 T-cells expressing both αβ-TCR and CD8αβ on cell surface (αβ-TCR +CD8αβ engineered Vg9d2 T-cells).

FIG. 2B shows exemplary experimental data showing that, as compared with Vγ9δ2 T-cells without viral transduction (Mock), 23.1% of Vγ9δ2 T-cells transduced with αβ-TCR retrovirus and CD8αβ retrovirus (αβ-TCR + CD8) incubated with target cells, e.g., A375 cells, stained positive by anti-CD107a antibody, indicating that αβ-TCR +CD8αβ engineered Vg9d2 T-cells are cytolytic by carrying out degranulation, when exposed to A375 cells.

FIG. 2C shows exemplary experimental data showing that, as compared with Vγ9δ2 T-cells without viral transduction (Mock), 19.7% of Vγ9δ2 T-cells transduced with αβ-TCR retrovirus and CD8αβ retrovirus (αβ-TCR + CD8) stained positive by anti-IFN-γ antibody, indicating that αβ-TCR +CD8αβ engineered Vγ9δ2 T-cells are cytolytic by releasing IFN-γ, when exposed to A375 cells. Cytolytic activity were evaluated at 24 hours post-exposure to A375 cells by gating on apoptosis of non-CD3 T-cells, i.e., A375 cells. Apoptosis was assessed by staining the harvested culture with live/dead dye.

FIG. 2D shows exemplary experimental data showing that, as compared with Vγ9δ2 T-cells without viral transduction (Mock), αβ-TCR +CD8αβ engineered Vγ9δ2 T-cells (αβ-TCR + CD8) induced apoptosis in 70% of A375 cells, indicating that αβ-TCR +CD8αβ engineered Vγ9δ2 T-cells are cytolytic by killing A375 cells.

FIG. 2E shows exemplary experimental data showing that, while non-transduced γδ T-cells showed cytotoxic potential due to intrinsic anti-tumor properties of γδ T-cells, αβTCR+CD8αβ transduced γδ T-cells showed similar cytotoxic potential as compared to αβTCR transduced αβ T-cells, indicating that αβTCR+CD8αβ transduced γδ T-cells can be engineered to target and kill tumor cells.

FIG. 3 shows exemplary experimental data showing IFNγ release from CD8+ T-cells electroporated with alpha and beta chain RNA of TCR R11P3D3 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

FIG. 4 shows exemplary experimental data showing IFNγ release from CD8+ T-cells electroporated with alpha and beta chain RNA of TCR R16P1C10 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-046 and IFN-041.

FIG. 5 shows exemplary experimental data showing IFNγ release from CD8+ T-cells electroporated with alpha and beta chain RNA of TCR R16P1E8 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

FIG. 6 shows exemplary experimental data showing IFNγ release from CD8+ T-cells electroporated with alpha and beta chain RNA of TCR R17P1A9 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

FIG. 7 shows exemplary experimental data showing IFNγ release from CD8+ T-cells electroporated with alpha and beta chain RNA of TCR R17P1D7 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

FIG. 8 shows exemplary experimental data showing IFNγ release from CD8+ T-cells electroporated with alpha and beta chain RNA of TCR R17P1 G3 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-046 and IFN-041.

FIG. 9 shows exemplary experimental data showing IFNγ release from CD8+ T-cells electroporated with alpha and beta chain RNA of TCR R17P2B6 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

FIG. 10 shows exemplary experimental data showing IFNγ release from CD8+ T-cells electroporated with alpha and beta chain RNA of TCR R11P3D3 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 11 shows exemplary experimental data showing IFNγ release from CD8+ T-cells electroporated with alpha and beta chain RNA of TCR R16P1C10 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 12 shows exemplary experimental data showing IFNγ release from CD8+ T-cells electroporated with alpha and beta chain RNA of TCR R16P1E8 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 13 shows exemplary experimental data showing IFNγ release from CD8+ T-cells electroporated with alpha and beta chain RNA of TCR R17P1D7 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 14 shows exemplary experimental data showing IFNγ release from CD8+ T-cells electroporated with alpha and beta chain RNA of TCR R17P1 G3 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 15 shows exemplary experimental data showing IFNγ release from CD8+ T-cells electroporated with alpha and beta chain RNA of TCR R17P2B6 (Table 7) after co-incubation with T2 target cells loaded with PRAME-004 peptide (SEQ ID NO: 310) in various peptide loading concentrations from 10 µM to 10pM. IFNγ release data were obtained with CD8+ T-cells derived from two different healthy donors. Different donors were analyzed, TCRA-0003 and TCRA-0017.

FIG. 16 shows exemplary experimental data showing HLA-A*02/PRAME-004 tetramer or HLA-A*02/NYESO1-001 (SEQ ID NO: 311) tetramer staining, respectively, of CD8+ T-cells electroporated with alpha and beta chain RNA of TCR R16P1C10 (Table 7). CD8+ T-cells electroporated with RNA of 1G4 TCR (SEQ ID: 85-96) that specifically binds to the HLA-A*02/NYESO1-001 (SEQ ID NO: 311) complex and mock electroporated CD8+ T-cells served as controls.

FIG. 17 shows exemplary experimental data showing IFNγ release from CD8+ T-cells lentivirally transduced with TCR R11P3D3 (Table 7) (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6 and 7) but unrelated peptides ACPL-001, HSPB3-001, UNC7-001, SCYL2-001, RPS2P8-001, PCNXL3-003, AQP6-001, PCNX-001, AQP6-002 TRGV10-001, NECAP1-001, FBXW2-001 or control peptide NYESO1-001 (SEQ ID NO: 311). IFNγ release data were obtained with CD8+ T-cells derived from two different healthy donors, D103805 and D191451.

FIG. 18 shows exemplary experimental data showing IFNγ release from CD8+ T-cells lentivirally transduced with TCR R11P3D3 (Table 7) after co-incubation with T2 target cells loaded with 100 nM PRAME-004 peptide (SEQ ID NO: 310) or similar (identical to PRAME-004 in positions 3, 5, 6 and 7) but unrelated peptides or control peptide NYESO1-001 (SEQ ID NO: 311). IFNγ release data were obtained with CD8+ T-cells derived from two different healthy donors, TCRA-0087 and TCRA-0088.

FIG. 19 shows exemplary experimental data showing IFNγ release from CD8+ T-cells lentivirally transduced with TCR R11P3D3 (Table 7) (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release data were obtained with CD8+ T-cells derived from two different healthy donors, D103805 and D191451.

FIG. 20 shows exemplary experimental data showing IFNγ release from CD8+ T-cells lentivirally transduced with TCR R11P3D3 (Table 7) after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copies of PRAME-004 per cells. T-cells alone served as controls. IFNγ release data were obtained with CD8+ T-cells derived from two different healthy donors, TCRA-0084 and TCRA-0085.

FIG. 21 shows exemplary experimental data showing IFNγ release from CD8+ T-cells lentivirally transduced with enhanced TCR R11P3D3_KE (Table 7) (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6 and 7) but unrelated peptide ACPL-001, HSPB3-001, UNC7-001, SCYL2-001, RPS2P8-001, PCNXL3-003, AQP6-001, PCNX-001, AQP6-002, TRGV10-001, NECAP1-001, FBXW2-001 or control peptide NYESO1-001 (SEQ ID NO: 311). IFNγ release data were obtained with CD8+ T-cells derived from two different healthy donors, D103805 and D191451.

FIG. 22 shows exemplary experimental data showing IFNγ release from CD8+ T-cells lentivirally transduced with enhanced TCR R11P3D3_KE (Table 7) after co-incubation with T2 target cells loaded with 100 nM PRAME-004 peptide (SEQ ID NO: 310) or similar (identical to PRAME-004 in positions 3, 5, 6 and 7) but unrelated peptides or control peptide NYESO1-001 (SEQ ID NO: 311). IFNγ release data were obtained with CD8+ T-cells derived from two different healthy donors, TCRA-0087 and TCRA-0088.

FIG. 23 shows exemplary experimental data showing IFNγ release from CD8+ T-cells lentivirally transduced with enhanced TCR R11P3D3_KE (Table 7) (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release data were obtained with CD8+ T-cells derived from two different healthy donors, D103805 and D191451.

FIG. 24 shows exemplary experimental data showing IFNγ release from CD8+ T-cells lentivirally transduced with enhanced TCR R11P3D3_KE (Table 7) after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copies of PRAME-004 per cells. T-cells alone served as controls. IFNγ release data were obtained with CD8+ T-cells derived from two different healthy donors, TCRA-0084 and TCRA-0085.

FIG. 25 shows exemplary experimental data showing IFNγ release from CD8+ T-cells lentivirally transduced with TCR R11P3D3 or enhanced TCR R11P3D3_KE (Table 7) or non-transduced cells after co-incubation with tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release of both TCRs correlates with PRAME-004 presentation and R11P3D3_KE induces higher responses compared to R11P3D3.

FIG. 26 shows exemplary experimental data showing the results of an exemplary potency assay evaluating cytolytic activity of lentivirally transduced T-cells expressing TCR R11P3D3 or enhanced TCR R11P3D3_KE against PRAME-004+ tumor cells. Cytotoxic response of R11P3D3 and R11P3D3_KE transduced and non-transduced (NT) T-cells measured against A-375 (PRAME-004 low) or U2OS (PRAME-004 medium) tumor cells. The assays were performed in a 72-hour fluorescence microscopy-based cytotoxicity assay. Results are shown as fold tumor growth over time.

FIG. 27 shows exemplary experimental data showing the results of an exemplary potency assay evaluating cytolytic activity of lentivirally transduced T-cells expressing TCR R11P3D3 or enhanced TCR R11P3D3_KE against PRAME-004+ tumor cells. Cytotoxic response of R11P3D3 and R11P3D3_KE transduced and non-transduced (NT) T-cells measured against A-375 (PRAME-004 low) or U2OS (PRAME-004 medium) tumor cells. The assays were performed in a 72-hour fluorescence microscopy-based cytotoxicity assay. Results are shown as fold tumor growth over time.

FIG. 28 shows exemplary experimental data showing the results of an exemplary LDH-release assay with the bispecific TCR/mAb diabody construct IA_5 targeting tumor-associated peptide PRAME-004 (SEQ ID NO: 310) presented on HLA-A*02. CD8-positive T-cells isolated from a healthy donor were co-incubated with cancer cell lines UACC-257, SW982 and U2OS presenting differing amounts of PRAME-004:HLA-A*02-1 complexes on the cell surface (approx. 1100, approx. 770 and approx. 240 copies per cell, respectively, as determined by M/S analysis) at an effector:target ratio of 5:1 in the presence of increasing concentrations of TCR/mAb diabody molecules. After 48 hours of co-culture target cell lysis was quantified utilizing LDH-release assays according to the manufacturer’s instructions (Promega).

FIG. 29 shows exemplary experimental data showing the results of an exemplary LDH-release assay with the bispecific TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. CD8-positive T-cells isolated from a healthy donor were co-incubated with the cancer cell line U2OS presenting approx. 240 copies per cell of PRAME-004:HLA-A*02-1 complexes or non-loaded T2 cells (effector:target ratio of 5:1) in the presence of increasing concentrations of TCR/mAb diabody molecules. After 48 hours of coculture target cell lysis was quantified utilizing LDH-release assays according to the manufacturer’s instructions (Promega).

FIG. 30 shows exemplary experimental data showing the results of an exemplary heat-stress stability study of the TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. For this, the proteins were formulated in PBS at a concentration of 1 mg/mL and subsequently stored at 40° C. for two weeks. Protein integrity and recovery was assessed utilizing HPLC-SEC. Thereby the amount of high-molecular weight species was determined according to percentage of peak area eluting before the main peak. Recovery of monomeric protein was calculated by comparing main peak areas of unstressed and stressed samples.

FIG. 31 shows exemplary experimental data showing binding kinetics of bispecific molecules comprising different R16P1C10 variants. FAB2G sensors were used for the scTCR-Fab format (20 µg/ml loaded for 120 s), AHC sensors for the diabody-F_{c} formats (10 µg/ml loaded for 120 s for improved variant; 5 µg/ml loaded for 120 s for stabilized variant, LoAff3, CDR6, HiAff1). Analyzed concentrations of HLA-A*02/PRAME-004 are represented in nM. Graphs show curves of measured data and calculated fits.

FIG. 32 shows exemplary experimental data showing lysis of PRAME-positive tumor cell lines induced by bispecific molecules containing CDR6, HiAff1 or LoAff3 TCR variants, respectively, in presence of CD8+ T-cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 33 shows exemplary experimental data showing lysis of PRAME-negative tumor cell lines induced by bispecific molecules containing CDR6, HiAff1 or LoAff3 TCR variants, respectively, in presence of CD8+ T-cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 34 shows exemplary experimental data showing in vivo efficacy. NOG mice bearing HS695T tumors of approximately 50 mm³ were transplanted with human PBMCs and treated with PBS (group 1), 0.5 mg/kg body HiAff1/antiCD3 diabody-Fc (group 2) or 0.5 mg/kg antiHIV/antiCD3 diabody-Fc (group) i.v. twice a week. Tumor volumes were measured with a caliper and calculated by length x width² /2.

FIG. 35 shows exemplary experimental data showing in vitro cytotoxicity of TCER® molecules on target-positive and target-negative tumor cell lines. PBMC from a healthy HLA-A*02-positive donor were incubated with either target-positive tumor cell line Hs695T (•) or target-negative, but HLA-A*02-positive tumor cell line T98G (◯), respectively, at a ratio of 1:10 in the presence of increasing TCER® concentrations. TCER®-induced cytotoxicity was quantified after 48 hours of co-culture by measurement of released LDH. Results for experiments assessing TPP-93 and TPP-79 are shown in the upper and lower panel, respectively.

FIG. 36 shows exemplary experimental data showing in vitro cytotoxicity of TCER® molecule TPP-105 on target-positive and target-negative tumor cell lines. PBMC from a healthy HLA-A*02-positive donor were incubated with either target-positive tumor cell line Hs695T (•) or target-negative, but HLA-A*02-positive tumor cell line T98G (◯), respectively, at a ratio of 1:10 in the presence of increasing concentrations of TPP-105. TCER®-induced cytotoxicity was quantified after 48 hours of co-culture by measurement of released LDH.

FIG. 37 shows a summary of exemplary cytotoxicity data of TCER® Slot III molecules. EC₅₀ values of dose-response curves obtained in LDH-release assays were calculated utilizing non-linear 4-point curve fitting. For each assessed TCER®-molecule calculated EC₅₀ values on target-positive tumor cell lines Hs695T (•), U2OS (o), and target-negative but HLA-A*02-positive tumor cell line T98G (*) are depicted. Thereby, each symbol represents one assay utilizing PBMC derived from various HLA-A*02-positive donors. For TPP-871/T98G, the EC₅₀ is estimated, as T98G was not recognized by TPP-871.

FIGS. 38A-38C shows exemplary experimental data showing in vitro cytotoxicity of TCER® Slot III variants on T2 cells loaded with different concentrations of target peptide. Cytotoxicity was determined by quantifying LDH released into the supernatants. Human PBMC were used as effector cells at an E:T ratio of 5:1. Readout was performed after 48 h.

FIG. 39 shows exemplary experimental data showing normal tissue cell safety analysis for selected TCER® Slot III variants. TCER®-mediated cytotoxicity against 5 different normal tissue cell types expressing HLA-A*02 was assessed in comparison to cytotoxicity directed against PRAME-004-positive Hs695T tumor cells. PBMCs from a healthy HLA-A*02+ donor were co-cultured at a ratio 10:1 with the normal tissue cells or Hs695T tumor cells (in triplicates) in a 1:1 mixture of the respective normal tissue cell medium (4, 10a or 13a) and T-cell medium (LDH-AM) or in T-cell medium alone. After 48 hours, lysis of normal tissue cells and Hs695T-cells was assessed by measuring LDH release (LDH-Glo™ Kit, Promega).

FIG. 40 shows exemplary non-limiting atezolizumab dosing schedules, starting at Day 14 post-treatment or Day 21 post-treatment. M indicates month after treatment and D indicates D after treatment.

FIG. 41A shows baseline and post-treatment measurements of an exemplary tumor. FIG. 41A shows a baseline tumor measurement of 14.0 × 28.1 mm and a post-treatment tumor measurement of 1.6 × 9.2 mm. The tumor is indicated by the white arrow.

FIG. 41B shows baseline and post-treatment measurements of an exemplary tumor. FIG. 41B shows a baseline tumor measurement of 11.2 × 26.2 mm and a post-treatment tumor measurement of 12.3 × 24.0 mm. The tumor is indicated by the white arrow.

FIG. 41C shows baseline and post-treatment measurements of an exemplary tumor. FIG. 41C shows a baseline tumor measurement of 26.1 × 29.7 mm and a post-treatment tumor measurement of 9.1 × 22.4 mm. The tumor is indicated by the white arrow.

FIG. 42 is a graph showing the relative change in diameter of an exemplary target lesion upon IMA203 treatment over time. The patient shows a durable response with an ongoing progression-free survival of more than 16 month and a duration of response of more than 15 months.

### DETAILED DESCRIPTION

Before the present disclosure is described in detail, it is to be understood that this present disclosure is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to an aspect of the present disclosure, a peptide comprising the amino acid sequence of SEQ ID NO: 310 (SLLQHLIGL) or a pharmaceutically acceptable salt thereof is provided, said peptide being for use in the (manufacture of a medicament for the) treatment of a patient (i) being diagnosed for, (ii) suffering from or (iii) being at risk of developing recurrent cancer.

This language is deemed to encompass both the Swiss type claim language accepted ins come countries (in this case, brackets are deemed absent) and EPC2000 language (in this case, brackets and content within the brackets is deemed absent).

Alternatively or in addition, a method of treating a patient (i) being diagnosed for, (ii) suffering from or (iii) being at risk of developing recurrent cancer, is provided.

The method comprises administering to the patient a peptide comprising the amino acid sequence of SEQ ID NO: 310 (SLLQHLIGL) or a pharmaceutically acceptable salt thereof, in one or more therapeutically effective doses.

Alternatively or in addition, a pharmaceutical composition for treating recurrent cancer is provided, comprising a peptide comprising the amino acid sequence of SEQ ID NO: 310 (SLLQHLIGL) or a pharmaceutically acceptable salt as an effective ingredient.

In one embodiment, the recurrent cancer is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

Recurrent cancer can be analyzed whether it displays, on the surface of at least one of its cells, a peptide comprising an amino acid sequence described, for example, in Table 10, or said amino acid bound to a major histocompatibility complex, by different means. In another aspect, the peptide is in the PRAME, MAGE, MAG, COL6A3 family of targets. In yet another aspect, the peptide is in the PRAME-004, MAG-003, MAGEA1-003, COL6A3-002, and MAGE-A4 family of peptides. In yet another aspect, the peptides are SLLQHLIGL (SEQ ID NO: 310), KVLEHVVRV (SEQ ID NO: 430), KVLEYVIKV (SEQ ID NO: 417), FLLDGSANV (SEQ ID NO: 453), and/or GVYDGREHTV (SEQ ID NO: 401).

### Combination Treatment With (i) TCR R11P3D3_KE T Cells, (ii) MAGE-A4-Binding Molecule, (iii) a Checkpoint Inhibitor, or (iv) Any Combination Thereof

Studies using TCR-engineered autologous T cells have shown promising success, including objective tumor responses in a relevant portion of patients with solid tumors (Johnson LA, et al. (2009), Gene therapy with human and mouse T-cell receptors mediates cancer regression and targets normal tissues expressing cognate antigen, Blood 114, 535-546, which is incorporated by reference herein in its entirety; Morgan et al., 2013; Robbins PF et al., 2015).

TCR R11P3D3_KE T cells are an autologous T-cell product engineered to express a PRAME specific TCR. The target and TCR were selected based on comprehensive in vitro experimental data. This data package covered the characterization of the target peptide and its source gene PRAME based on data from a large panel of normal and cancer tissues. The data indicates that 1) PRAME is a highly tumor-associated, naturally presented target virtually absent from relevant normal tissues and 2) there is an apparent absence of unexpected off-target recognition or cross reactivity towards the tested healthy cells and peptides similar to the target. Thus, overall, the risk of on-target or off target toxicities is considered low for TCR R11P3D3_KE T cells.

The comprehensive dataset available for PRAME-004, the targeted antigen, ensures (i) that it is naturally presented in HLA-A*02:01 molecules on the tumor at high peptide copy numbers and is proven to be relevant as a cancer target for immunotherapy; and (ii) that the likelihood of autoimmune toxicities is reduced because the source gene PRAME is not expressed at relevant levels on normal tissues.

R11P3D3_KE is a highly specific and extensively characterized, pairing-optimized TCR. This TCR has shown significant anti-PRAME-004 activity in cells pulsed with physiologic concentrations of peptide as well as in tumor cells expressing the source gene PRAME. No relevant indication of potential cross-reactivity was found in experiments using similar peptides (from the human proteome or immunopeptidome), nor was there any significant recognition of various normal cell lines.

For treatment with TCR R11P3D3_KE T cells, among the range of solid cancer indications, synovial sarcoma, uterine cancer (endometrial cancer and uterine carcinoma), melanoma, and ovarian cancer may be of special interest because PRAME is frequently expressed in these tumors. However, patients with other tumor types that are positive for HLA A*02:01 and PRAME may also be treated with TCR R11P3D3_KE T Cells.

Standard-of-care treatments for solid-tumor patients may include, as non-limiting examples, surgery, radiation therapy, systemic chemotherapy, immunotherapy with checkpoint inhibitors, and/or targeted therapies for patients with tumors harboring oncogenic mutations. For a patient to receive treatment with TCR R11P3D3_KE T Cells, there is no limitation on the number of prior therapies the patient may have received.

The expected prevalence of PRAME for listed indications is outlined in Table 1. Target prevalence is defined as the percentage of tumors in the Cancer Genome Atlas (TCGA; cancergenome.nih.gov/) database expressing target messenger ribonucleic acid (mRNA) above an individually defined fragments per kilobase million threshold for PRAME-004. The threshold for PRAME-004 was defined based on the observation that mRNA expression above this level corresponded to a pronounced likelihood of actual peptide detection for the target.

TABLE 1

| | % Prevalence^{a} determined by XPRESIDENT^{®} |
|---|---|
| Synovial sarcoma | 100 |
| Uterine cancer | 100/98^{b} |
| Skin cutaneous melanoma | 95 |
| Ovarian cancer | 81 |
| Lung cancers | 66 / 27 / 54^{c} |
| Breast carcinoma (Triple-negative subtype) | 61 |
| Testicular germ cell tumor | 57 |
| Uveal melanoma | 51 |
| Thymoma | 48 |
| Kidney cancer | 45 / 23^{d} |
| Malignant peripheral nerve sheath tumor | 40 |
| Cholangiocarcinoma | 33 |
| Breast carcinoma (all) | 26 |
| Head and Neck cancer | 25 |
| Cervical carcinoma | 25 |
| Adrenocortical carcinoma | 24 |
| Esophageal cancer | 20 |
| Liver hepatocellular carcinoma | 19 |
| Bladder cancer | 18 |

^{a} Target prevalence is defined as the percentage of tumors in The Cancer Genome Atlas database and/or in-house RNAseq data expressing target messenger RNA above an individually defined fragments per kilobase million threshold for PRAME-004. The threshold for PRAME-004 was defined based on the observation that mRNA expression above this level corresponded to a pronounced likelihood of actual peptide detection for the target.
^{b} Uterine carcinosarcoma / Uterine corpus endometrial carcinoma
^{c} Lung squamous cell carcinoma / Lung adenocarcinoma / Small cell lung cancer
^{d} Kidney renal papillary cell carcinoma / Kidney renal clear cell carcinoma

As shown in Table 1, patients with several cancer indications are expected to express the source gene PRAME at sufficient levels to present the target peptide PRAME-004 in their HLA molecules. Therefore, patients diagnosed with these or other solid tumors, if positive for PRAME-004 source gene expression, may be treated with TCR R11P3D3_KE T cells. Patients from niche indications not covered by TCGA data may also be treated with TCR R11P3D3_KE T cells, particularly if other data sources suggest a reasonable potential expression of PRAME.

Patients with tumor types that are positive for HLA A*02 and MAGE-A4 may be treated with a MAGE-based program, for example, ADP-A2M4. The ADP-A2M4 program includes genetically engineered autologous specific peptide enhanced affinity receptor (SPEAR) T-cells directed towards the HLA-A2-restricted MAGE-A4230-239 peptide GVYDGREHTV (SEQ ID NO: 401) expressed in the context of HLA-A*02. Patients with tumor types that are positive for MAGE-A4 may be treated with this program or another MAGE-A4-binding molecule. MAGE-A4-binding molecule may be any construct that specifically binds to MAGE-A4. As a non-limiting examples, such constructs may be antibodies, engineered TCR T cells, engineered CAR T cells, or other constructs.

In embodiments, a combination therapy of (i) TCR R11P3D3_KE T cells and (ii) T cells as described in Example 25 or other MAGE-A4 binding molecule is provided. A combination therapy of (i) TCR R11P3D3_KE T cells and (ii) T cells as described in Example 25 or other MAGE-A4 binding molecule may offer outcomes superior to those achieved using TCR R11P3D3_KE T cells or T cells as described in Example 25 as a monotherapy. Combination therapies may be administered in any order. In an aspect, a combination therapy utilizing (1) TCR R11P3D3_KE T cells or another PRAME binding molecule as the first treatment or pretreatment therapy is provided for followed by (2) second treatment with T cells as described in Example 25 or other MAGE-A4 binding molecule. In another aspect, a combination therapy utilizing (1) T cells as described in Example 25 or other MAGE-A4 binding molecule as the first treatment or pretreatment therapy is provided for followed by (2) second treatment with TCR R11P3D3_KE T cells or another PRAME antigen binding molecule.

In embodiments, representative antigen binding molecules that bind MAG-003 are described in US 11,072,645 and US 10,538,573; representative binding molecules that bind MAGEA1-003 are described in US 10,874,731; and representative antigen binding molecules that bind COL6A3 are described in 10,550,182. The contents of each of these patents is hereby incorporated by reference in their entireties.

The increased expression of inhibitory receptors, so-called immune checkpoints, can negatively regulate the function and persistence of transferred T cells by mediating T-cell anergy and exhaustion, which consequently lead to tumor progression. Providing both tumor-specific T cells and removing T-cell inhibitory stimuli through checkpoint inhibition may offer outcomes superior to those achieved with either agent alone. See, e.g., Houot R, et al. (2015), T-cell-based Immunotherapy: Adoptive Cell Transfer and Checkpoint Inhibition, Cancer Immunol Res 3, 1115-1122 and Yoon DH, et al. (2018), Incorporation of Immune Checkpoint Blockade into Chimeric Antigen Receptor T Cells (CAR-Ts): Combination or Built-In CAR-T, Int J Mol Sci 19 (2):340; each of which is incorporated by reference herein in its entirety.

One major immune checkpoint is the programmed death 1 (PD-1) pathway, which may greatly contribute to immunosuppression in the tumor microenvironment and hence may play a role in the lack of clinical responses observed in some patients treated with ACT. Upregulation of PD-ligand 1 (PD-L1) on tumor cells may inhibit T-cell function by binding to PD-1 expressed on T cells. This may be a common mechanism used by tumor cells to escape destruction by the immune system. Blocking the PD–1/PD-L1 interaction by monoclonal antibodies has shown clinical benefit, and several of those antibodies (PD-1/PD-L1 interaction inhibitors) have been approved for treatment in different cancer indications (such as, but not limited to, atezolizumab (such as, but not limited to, Tecentriq®), pembrolizumab (such as, but not limited to, Keytruda®), nivolumab (such as, but not limited to, Imfinzi®), cemiplimab (such as, but not limited to, Libtayo®)). However, some patients do not respond to this therapy, potentially due to the lack of tumor-specific T cells. Administering tumor-specific TCR R11P3D3_KE T cells and/or T cells as described in Example 25 in combination with a blockade of the PD-1/PD-L1 interaction, such as, but not limited to, by administering one or combinations of atezolizumab, pembrolizumab, nivolumab, or cemiplimab, which may remove the inhibition of the transferred T cells within the tumor, may have a synergistic effect. These treatments may be administered in any order or at the same time.

In embodiments, a combination therapy of (i) TCR R11P3D3_KE T cells or T cells as described in Example 25 or other MAGE-A4-binding molecule and (ii) a checkpoint inhibitor such as, but not limited to, PD-L1/ PD-1 checkpoint inhibitors (as non-limiting examples, atezolizumab, pembrolizumab, nivolumab, and/or cemiplimab) is provided. A combination therapy of (i) TCR R11P3D3_KE T cells or T cells as described in Example 25 or other MAGE-A4-binding molecule and (ii) a checkpoint inhibitor such as, but not limited to, PD-L1/ PD-1 checkpoint inhibitors (as non-limiting examples, atezolizumab, pembrolizumab, nivolumab, and/or cemiplimab) may offer outcomes superior to those achieved with any one agent alone. Combination therapies may be administered in any order.

In embodiments, a combination therapy of (i) TCR R11P3D3_KE T cells, (ii) T cells as described in Example 25 or other MAGE-A4-binding molecule, and (iii) a checkpoint inhibitor such as, but not limited to, PD-L1/ PD-1 checkpoint inhibitors (as non-limiting examples, atezolizumab, pembrolizumab, nivolumab, and/or cemiplimab) is provided. A combination therapy of (i) TCR R11P3D3_KE T cells, (ii) T cells as described in Example 25 or other MAGE-A4-binding molecule, and (iii) a checkpoint inhibitor such as, but not limited to, PD-L1/ PD-1 checkpoint inhibitors (as non-limiting examples, atezolizumab, pembrolizumab, nivolumab, and/or cemiplimab) is provided may offer outcomes superior to those achieved with any one agent alone or with TCR R11P3D3_KE T cells and T cells as described in Example 25. Combination therapies may be administered in any order.

In embodiments, a combination therapy described herein is provided after lymphodepletion is performed. An issue in adoptive cellular therapy (ACT) may be the limited persistence of transferred T cells in vivo, which is important because T-cell persistence has been shown to be a marker for clinical effectiveness. See, e.g., Yee C, et al. (2015), Endogenous T-Cell Therapy: Clinical Experience, Cancer J 21, 492-500, which is incorporated by reference herein in its entirety. An approach to address this consistent challenge may be the transient ablation of endogenous lymphocytes by non-myeloablative lymphodepletion chemotherapy prior to the T-cell infusion. Preclinical (see, e.g., Awwad M, North RJ (1988), Cyclophosphamide (Cy)-facilitated adoptive immunotherapy of a Cy-resistant tumour. Evidence that Cy permits the expression of adoptive T-cell mediated immunity by removing suppressor T cells rather than by reducing tumour burden, Immunology 65, 87-92 and Rosenberg SA, et al. (1986), A new approach to the adoptive immunotherapy of cancer with tumor-infiltrating lymphocytes, Science 233, 1318-1321, each of which is incorporated by reference herein in its entirety) as well as clinical data (see, e.g., Dudley ME, et al. (2005), Adoptive cell transfer therapy following non-myeloablative but lymphodepleting chemotherapy for the treatment of patients with refractory metastatic melanoma, J Clin Oncol 23, 2346-2357 and Rosenberg SA, et al. (1994), Treatment of patients with metastatic melanoma with autologous tumor-infiltrating lymphocytes and interleukin 2, J Natl Cancer Inst 86, 1159-1166, each of which is incorporated by reference herein in its entirety) suggest that lymphodepletion (LD) prior to T-cell infusions enhances T-cell engraftment and persistence and ultimately the clinical efficacy of ACT approaches (see, e.g., Yee et al., 2015). Without wanting to be bound by theory, mechanistically, it is presumed that this effect is mainly caused by depletion of inhibitory regulatory T cells and provision of “space” in the T-cell compartment (e.g., excess of availability to trophic cytokines and growth factors because of less competition for those by other T cells). A lymphodepletion regimen (LDR) may be administered to a patient(s) prior to T cell infusion. The LDR may comprise administration of fludarabine (FLU), cyclophosphamide (CY), or combinations thereof.

Interleukin 2 (IL-2) may be administered after the infusion of T cells. Administration of IL-2 after infusion of T-cells may positively influence the activation status of transferred T cells, as well as their persistence. See, e.g., Rosenberg SA (2014), IL-2: the first effective immunotherapy for human cancer, J Immunol 192, 5451-5458, which is incorporated by reference herein in its entirety.

In embodiments, a combination therapy described herein followed by administration of Interleukin 2 (IL-2) is provided. A combination of IL-2 administration after T-cell infusion and lymphodepletion prior to T-cell infusion has been shown to further improve the persistence of engrafted anti-tumor T cells and, moreover, has been associated with durable clinical responses. (see, e.g., Dudley ME, et al. (2008). Adoptive cell therapy for patients with metastatic melanoma: evaluation of intensive myeloablative chemoradiation preparative regimens, J Clin Oncol 26, 5233-5239, which is incorporated by reference herein in its entirety; Robbins et al., 2015; and Wallen H, et al. (2009), Fludarabine may modulate immune response and may extend in vivo survival of adoptively transferred CD8 T cells in patients with metastatic melanoma, PLoS One 4, e4749, which is incorporated by reference herein in its entirety. While high- or low-dose IL-2 regimens have been associated with clinical successes in ACT trials, a clear superiority of one regimen over the other has not been shown so far, especially when IL-2 treatment is combined with lymphodepletion. Lower doses of IL-2 may be associated with fewer or less severe side effects. As a non-limiting example, lower dose of IL-2 during the first approximately 5 days after T-cell infusion may reduce the intensity of cytokine release syndrome (CRS) and may protect patients from unwanted secondary pharmacology associated from too strong activation of the immune-system that could be a risk of the higher dose levels.

### Production of TCR R11P3D3_KE T Cells And Related Products and Processes

In one embodiment, one takes a biopsy of the tumor, and subjects it to immunoprecipitation of peptide MHC complexes, with subsequent analysis of the peptidome thus obtained by means of Mass spectrometry. Respective methods are e.g disclosed in Fritsche, J., Rakitsch, B., Hoffgaard, F., Römer, M., Schuster, H., Kowalewski, D. J., Priemer, M., Stos-Zweifel, V., Hörzer, H., Satelli, A., Sonntag, A., Goldfinger, V., Song, C., Mahr, A., Ott, M., Schoor, O., Weinschenk, T., Translating Immunopeptidomics to Immunotherapy-Decision-Making for Patient and Personalized Target Selection Proteomics 2018, 18, 1700284, the content of which is incorporated herein by reference.

Another possibility is to use a labelled T cell receptor or TCR mimetic antibody specific of the peptide MHC complex comprising the peptide of SEQ ID NO: 310 (SLLQHLIGL). A biopsy of the recurrent cancer is obtained, rated with routine immunological methods (sliced, homogenized, or the like) and then incubated with the T cell receptor of TCR mimectic antibody. See e.g. Høydahl LS, Frick R, Sandlie I, Løset GÅ. Targeting the MHC Ligandome by Use of TCR-Like Antibodies. Antibodies (Basel). 2019;8(2):32. Published 2019 May 9. for methods, the content of which is incorporated herein by reference.

Another possibility is to apply RNA seq techniques to the recurrent cancer. RNA-Seq (named as an abbreviation of “RNA sequencing”) is a sequencing technique which uses next-generation sequencing (NGS) to reveal the presence and quantity of RNA in a biological sample at a given moment, analyzing the continuously changing cellular transcriptome. Specifically, RNA-Seq facilitates the ability to look at alternative gene spliced transcripts, post-transcriptional modifications, gene fusion, mutations/SNPs and changes in gene expression over time, or differences in gene expression in different groups or treatments. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries. Recent advances in RNA-Seq include single cell sequencing, in situ sequencing of fixed tissue, and native RNA molecule sequencing with single-molecule real-time sequencing.

In one embodiment, one may look, in the RNA transcriptome, for the mRNA sequence that is specifically encoding the peptide of SEQ ID NO: 310 (SLLQHLIGL).

The respective HLA status can be determined by routine methods of HLA serotyping and HLA haplotyping, as e.g. disclosed in Zhang GL, Keskin DB, Lin HN, et al. Human leukocyte antigen typing using a knowledge base coupled with a high-throughput oligonucleotide probe array analysis. Front Immunol. 2014;5:597, the content of which is incorporated herein by reference.

HLA-A*02 is a human leukocyte antigen serotype within the HLA-A serotype group. The serotype is determined by the antibody recognition of the α2 domain of the HLA-A α-chain. For A*02, the α chain is encoded by the HLA-A*02 gene and the β chain is encoded by the B2M locus.

HLA-A*02 is one particular class I major histocompatibility complex (MHC) allele group at the HLA-A locus. The A*02 allele group can code for many proteins; as of December 2013 there are 456 different HLA-A*02 proteins. Serotyping can identify as far as HLA-A*02, which is typically enough to prevent transplant rejection (the original motivation for HLA identification). Genes can further be separated by genetic sequencing and analysis. HLAs can be identified with as many as nine numbers and a letter (ex. HLA-A*02:101:01:02N).[2] HLA-A*02 is globally common, but particular variants of the allele can be separated by geographic prominence.

The term “peptide”, as used herein, shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoroacetate) salts. It has to be noted that the salts of the peptides according to the present description differ substantially from the peptides in their state(s) in vivo, as the peptides are not salts in vivo.

As used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral-NH2 group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

SEQ ID NO: 310 (SLLQHLIGL) is a peptide that is related to PRAME, which is a protein encoded by the PRAME gene.

PRAME (Preferentially Expressed Antigen in Melanoma), also known as Opa-interacting protein 4, CT130 and MAPE, is a protein and tumor antigen of the Cancer/Testis antigen group. PRAME has a length of 509 amino acids and a mass of 57,890 Da. PRAME has the Entrez identifier 23532, and the UniProt identifier P78395 (SEQ ID NO: 488).

The nucleotide sequence of PRAME is known and may be found in, for example, GenBank Accession Nos. NM_001291715.2 (SEQ ID NO: 475), NM_001291716.2 (SEQ ID NO: 476), NM_001291717.2 (SEQ ID NO: 477), NM_001291719.2 (SEQ ID NO: 478), NM_001318126.1 (SEQ ID NO: 479), NM_001318127.1 (SEQ ID NO: 480), NM_006115.5 (SEQ ID NO: 481), NM_206956.3 (SEQ ID NO: 482), NM_206955.2 (SEQ ID NO: 483), NM_206954.3 (SEQ ID NO: 484), and NM_206953.2 (SEQ ID NO: 485).

The amino acid sequence of full-length PRAME is known and may be found in, for example, GenBank as Accession Nos. NP_001278646.1 (SEQ ID NO: 486), NP_006106.1 (SEQ ID NO: 487), NP_996837.1 (SEQ ID NO: 488), NP_996836.1 (SEQ ID NO: 489), NP_996839.1 (SEQ ID NO: 490), NP_996838.1 (SEQ ID NO: 491), NP_001278644.1 (SEQ ID NO: 492), NP_001305055.1 (SEQ ID NO: 493), NP_001305056.1 (SEQ ID NO: 494), NP_001278648.1 (SEQ ID NO: 495), and NP_001278645.1 (SEQ ID NO: 496).

The term “PRAME” may include recombinant PRAME or a fragment thereof. The term also encompasses PRAME or a fragment thereof coupled to, for example, histidine tag, mouse or human Fc, or a signal sequence, such as ROR1. In certain embodiments, the term comprises PRAME, or a fragment thereof, in the context of HLA-A2, linked to HLA-A2 or as displayed by HLA-A2. As used herein, the numbering of certain PRAME amino acid residues within the full-length PRAME sequence may be with respect to SEQ ID NO: 488.

PRAME, which is expressed at a high level in a large proportion of tumors, including melanomas, non-small-cell lung carcinomas, ovarian carcinoma renal cell carcinoma (RCC), breast carcinoma, cervix carcinoma, colon carcinoma, sarcoma, neuroblastoma, as well as several types of leukemia. PRAME is the best characterized member of the PRAME family of leucine-rich repeat (LRR) proteins. Mammalian genomes contain multiple members of the PRAME family whereas in other vertebrate genomes only one PRAME-like LRR protein was identified. PRAME is a cancer/testis antigen that is expressed at very low levels in normal adult tissues except testis but at high levels in a variety of cancer cells.

PRAME 004 is a 9 amino acid peptide that is obtained by degradation of PRAME by the ubiquitin-proteasome system (UPS). PRAME 004 is also called PRA425-433, as it comprises AA residues 425-433 of the PRAME protein. PRAME 004 is then presented by major histocompatibility complex (MHC) class I molecules on the cellular surface of the respective cells.

PRAME 004 is displayed, with high selectivity, on MHC class 1 molecules of primary tumors (see, e.g., WO2018172533A2 and US20180273602, the contents which are incorporated by reference in their entireties). As such, PRAME 004 can be used as a target for entities being capable of binding to PRAME 004, for the treatment of different primary tumors.

As used herein, the term “recurrent cancer” shall refer to one which has regrown, either at the initial site or at a distant site, after a response to initial therapy. In some embodiments, the length of time between the completion of initial therapy and the development of recurrent disease is longer than about 3 months, including for example longer than about any of 4, 5, 6, 7, 8, 9, 10, or 11 months. In some embodiments, the length of time between the completion of initial therapy and the development of recurrent disease is longer than about 12 months, including for example, longer than about any of 14, 16, 18, 20, 22, 24, 36, 48 months, or more.

As used herein, the term “recurrent cancer which is PRAME positive” relates to recurrent cancer that comprises cells that express PRAME.

The skilled person has different approaches at his disposal to determine whether or not a cell, or a recurrent cancer, is PRAME positive. Based on the Entrez identifier 23532, and the UniProt identifier P78395, the skilled person can either use immunohistochemical methods (like ELISA, RIA or the like), in which an antibody or binding agent is used that binds to PRAME protein in a suitable tissue sample. As an alternative, the skilled person can detect presence or absence of PRAME mRNA, by means of RT-PCR or other routine methods.

The methods of the present disclosure may be useful for any one or more of the following (and thus in various embodiments can achieve and/or include any one or more of the following): 1) decreasing one or more symptoms resulting from recurrent cancer (such as recurrent sarcoma, for example, recurrent synovial sarcoma); 2) increasing overall response rate of a recurrent cancer (such as recurrent sarcoma for example recurrent synovial sarcoma); 3) increasing partial response rate of a recurrent cancer (such as recurrent sarcoma, for example, recurrent synovial sarcoma); 4) increasing complete response rate of a recurrent cancer (such as recurrent sarcoma, for example, recurrent synovial sarcoma); 5) delaying disease progression of an individual with a recurrent cancer (such as recurrent sarcoma, for example, recurrent synovial sarcoma); 6) increasing the quality of life in an individual with recurrent cancer (such as recurrent sarcoma, for example, recurrent synovial sarcoma); 7) prolonging overall survival of an individual having recurrent cancer (such as recurrent sarcoma, for example, recurrent synovial sarcoma); and 8) prolonging progression-free survival of an individual having recurrent cancer (such as recurrent sarcoma, for example, recurrent synovial sarcoma).

Accordingly, in some embodiments, there is provided a method of decreasing one or more symptoms resulting from a recurrent cancer (such as recurrent sarcoma, for example, recurrent synovial sarcoma) that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310).

In some embodiments, there is provided a method of increasing response rate of recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310).

In some embodiments, there is provided a method of delaying disease progression of an individual with recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310).

In some embodiments, there is provided a method of prolonging survival of an individual having recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310).

In some embodiments, there is provided a method of treating a recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310), wherein the individual may have a partial response to treatment upon completion of less than about any of one, two, three, four, five, six, seven, or eight treatment cycles.

In some embodiments, there is provided a method of treating a recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310), wherein the individual may have a complete response to treatment upon completion of less than about any of one, two, three, four, five, six, seven, or eight treatment cycles. In some embodiments, the treatment cycle is four weeks. In some embodiments, the treatment cycle is three weeks.

In some embodiments, there is provided a method of treating a recurrent cancer (such as recurrent sarcoma, for example, recurrent synovial sarcoma), that present a peptide described herein, as a non-limiting example a PRAME peptide or SLLQHLIGL (SEQ ID NO: 310), on the cell surface, comprising administering to the individual an effective amount of a composition comprising a composition comprising antigen binding molecules that binds to a peptide described herein, as a non-limiting example a PRAME peptide or SLLQHLIGL (SEQ ID NO: 310), wherein the individual may be disease free for at least about any of 0.5, 1, 2, 3, 4, 5, 6, 7, 8, 9, 10, 12, 14, 16, 18, 20, 22, or 24 months upon completion of the treatment.

In some embodiments, there is provided a method of treating a recurrent cancer (such as recurrent sarcoma, for example, recurrent synovial sarcoma), that present a peptide described herein, as a non-limiting example a PRAME peptide or SLLQHLIGL (SEQ ID NO: 310), comprising administering to the individual an effective amount of a composition comprising a composition comprising antigen binding molecules that binds to a peptide described herein, as a non-limiting example a PRAME peptide or SLLQHLIGL (SEQ ID NO: 310), wherein the individual does not show a symptom resulting from the recurrent cancer for at least about any of 0.5, 1, 2, 3, 4, 5, 6, 7, 8, 9, 10, 12, 14, 16, 18, 20, 22, or 24 months upon completion of the treatment.

The amount of composition of the present disclosure administered to an individual (such as a human) may vary with the particular composition, the method of administration, and the particular type of recurrent cancer being treated. The amount should be sufficient to produce a desirable beneficial effect. For example, in some embodiments, the amount of the composition of the present disclosure is effective to result in an objective response (such as a partial response or a complete response). In some embodiments, the amount of the composition is sufficient to result in a complete response in the individual. In some embodiments, the amount of the composition of the present disclosure is sufficient to result in a partial response in the individual. In some embodiments, the amount of the composition of the present disclosure administered (for example when administered alone) is sufficient to produce an overall response rate of more than about any of 40%, 50%, 60%, or 64% among a population of individuals treated with the composition of the present disclosure. Responses of an individual to the treatment of the methods described herein can be determined, for example, based on response evaluation criteria in solid tumors (RECIST). For example, when evaluating target lesions, complete response (CR) may indicate disappearance of all target lesions; partial response (PR) may indicate at least a 30% decrease in the sum of the longest diameter (LD) of target lesions, taking as reference the baseline sum LD; stable disease (SD) may indicate neither sufficient shrinkage to qualify for PR nor sufficient increase to qualify for PD, taking as reference the smallest sum LD since the treatment started; progressive disease (PD) may indicate at least a 20% increase in the sum of the LD of target lesions, taking as reference the smallest sum LD recorded since the treatment started or the appearance of one or more new lesions.

In some embodiments, the amount of the composition of the present disclosure is sufficient to prolong progress-free survival of the individual (for example as measured by RECIST changes). In some embodiments, the amount of the composition of the present disclosure is sufficient to prolong overall survival of the individual. In some embodiments, the amount of the composition of the present disclosure (for example when administered along) is sufficient to produce clinical benefit of more than about any of 50%, 60%, 70%, or 77% among a population of individuals treated with the composition of the present disclosure.

According to one embodiment of the present disclosure, said peptide has the ability to bind to an MHC class I or class II molecule, and/or said peptide, when bound to said MHC, is capable of being recognized by CD4 or CD8 T-cells.

Complexes of peptide and MHC class I are recognized by CD8-positive T-cells bearing the appropriate T-cell receptor (TCR).

According to one embodiment of the present disclosure, the pharmaceutically acceptable salt is a chloride salt or an acetate salt.

According to further embodiments, the peptide may also have an overall length of from 9 to 30 amino acids. Preferably, it has from 9 to 12 amino acids. In one embodiment said peptide comprises 1 to 4 additional amino acids at the C- and/or N-terminus of SEQ ID NO: 310. See table 2 for further details:

TABLE 2

Combinations of the elongations of peptides of the present disclosure

| C-terminus | N-terminus |
|---|---|
| 4 | 0 |
| 3 | 0 or 1 |
| 2 | 0 or 1 or 2 |
| 1 | 0 or 1 or 2 or 3 |
| 0 | 0 or 1 or 2 or 3 or 4 |
| N-terminus | C-terminus |
| 4 | 0 |
| 3 | 0 or 1 |
| 2 | 0 or 1 or 2 |
| 1 | 0 or 1 or 2 or 3 |
| 0 | 0 or 1 or 2 or 3 or 4 |

In one embodiment, the peptide has a length according to the respective peptides in Table 10. In another embodiment, the peptide has a length according to SEQ ID NO: 310. In one embodiment, the peptide consists or consists essentially of the amino acid sequence according to SEQ ID NO: 310.

According to another aspect of the present disclosure, an antibody, or a functional fragment thereof, is provided. The antibody or functional fragment specifically recognizes, or binds to, the peptide according to the above description, or to the peptide according to the above description when bound to an MHC molecule.

The antibody or functional fragment is provided for use in the (manufacture of a medicament for the) treatment of a patient (i) being diagnosed for, (ii) suffering from or (iii) being at risk of developing recurrent cancer.

Alternatively or in addition, a method of treating a patient (i) being diagnosed for, (ii) suffering from or (iii) being at risk of developing recurrent cancer, is provided.

The method comprises administering to the patient an antibody, or a functional fragment thereof, which specifically recognizes, or binds to, the peptide according to the above description, or to the peptide according to the above description when bound to an MHC molecule, in one or more therapeutically effective doses.

Alternatively or in addition, a pharmaceutical composition for treating recurrent cancer is provided, comprising an antibody, or a functional fragment thereof, which specifically recognizes, or binds to, the peptide according to the above description, or to the peptide according to the above description when bound to an MHC molecule as an effective ingredient.

As used herein, the term “antibody” shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.

As used herein, the term “functional fragment” shall refer to fragments of such antibody retaining target binding capacities, e.g.
a CDR (complementarity determining region)
a hypervariable region,
a variable domain (Fv)
an IgG or IgM heavy chain (consisting of VH, CH1, hinge, CH2 and CH3 regions)
an IgG or IgM light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerized constructs comprising CH3+VL+VH, and antibody conjugates (e.g. antibody or fragments or derivatives linked to a toxin, a cytokine, a radioisotope or a label). These types are well described in the literature and can be used by the skilled person on the basis of the present disclosure, without adding further inventive activity.

Methods for the production of a hybridoma cell are disclosed in Köhler & Milstein (1975).

Methods for the production and/or selection of chimeric or humanised mAbs are described. For example, US6331415 by Genentech describes the production of chimeric antibodies, while US6548640 by Medical Research Council describes CDR grafting techniques and US5859205 by Celltech describes the production of humanised antibodies. The contents of each of these patents is hereby incorporated by reference in their entireties.

Methods for the production and/or selection of fully human mAbs are known in the art. These can involve the use of a transgenic animal which is immunized with the respective protein or peptide, or the use of a suitable display technique, like yeast display, phage display, B-cell display or ribosome display, where antibodies from a library are screened against human iRhom2 in a stationary phase.

In vitro antibody libraries are, among others, disclosed in US6300064 by MorphoSys and US6248516 by MRC/Scripps/Stratagene. Phage Display techniques are for example disclosed in US5223409 by Dyax. Transgenic mammal platforms are for example described in EP1480515A2 by TaconicArtemis. The contents of each of these patents is hereby incorporated by reference in their entireties.

IgG, IgM, scFv, Fab and/or F(ab)₂ are antibody formats well known to the skilled person. Related enabling techniques are available from the respective textbooks.

As used herein, the term “Fab” relates to an IgG/IgM fragment comprising the antigen binding region, said fragment being composed of one constant and one variable domain from each heavy and light chain of the antibody

As used herein, the term “F(ab)₂” relates to an IgG/IgM fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like. These types are well described in the literature and can be used by the skilled person on the basis of the present disclosure, with adding further inventive activity.

Antibodies capable of binding a peptide bound to an MHC are sometimes called “TCR mimic antibodies” or “TCR like antibodies”. Generally, such antibodies can be generated with the methods described above. Methods how to generate TCR like antibodies are for example disclosed in He, Q., Liu, Z., Liu, Z. et al. TCR-like antibodies in cancer immunotherapy. J Hematol Oncol 12, 99 (2019), the content of which is incorporated herein by reference on its entirety.

TCR mimic antibodies binding to HLA restricted peptide derived from PRAME are for example disclosed in Chang AY et al, A therapeutic T cell receptor mimic antibody targets tumor-associated PRAME peptide/HLA-I antigens. J Clin Invest. 2017 Jun 30;127(7):2705-2718, the content of which is incorporated herein by reference in its entirety. See, also, US 2018/0148503 (T cell receptor-like antibodies specific for a prame peptide) (Eureka Therapeutics Inc), the content of which is incorporated herein by reference in its entirety.

In one embodiment, the recurrent cancer is positive for a peptide described herein, for example, a peptide in Table 10. In one embodiment, the recurrent cancer displays, on the surface of at least one of its cells, a peptide comprising the amino acid sequence of a peptide in Table 10, or said amino acid bound to a major histocompatibility complex.

In one embodiment, the recurrent cancer is positive for a peptide described herein, for example, a peptide in Table 10. In one embodiment, the recurrent cancer displays, on the surface of at least one of its cells, a peptide comprising the amino acid sequence of a peptide in Table 10, or said amino acid bound to a major histocompatibility complex.

In one embodiment, the recurrent cancer is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

According to another aspect of the present disclosure, a T-cell receptor, or a functional fragment thereof, is provided that is reactive with, or binds to, an MHC ligand, wherein said ligand is the peptide according to the above description, or the peptide according to the above description when bound to an MHC molecule. The T-cell receptor is provided for use in the (manufacture of a medicament for the) treatment of a patient (i) being diagnosed for, (ii) suffering from or (iii) being at risk of developing recurrent cancer.

Alternatively or in addition, a method of treating a patient (i) being diagnosed for, (ii) suffering from or (iii) being at risk of developing recurrent cancer, is provided.

The method comprises administering to the patient a T-cell receptor, or a functional fragment thereof, that is reactive with, or binds to, an MHC ligand, wherein said ligand is the peptide according to the above description, or the peptide according to the above description when bound to an MHC molecule, in one or more therapeutically effective doses.

Alternatively or in addition, a pharmaceutical composition for treating recurrent cancer is provided, comprising a T-cell receptor, or a functional fragment thereof, that is reactive with, or binds to, an MHC ligand, wherein said ligand is the peptide according to the above description, or the peptide according to the above description when bound to an MHC molecule, as an effective ingredient.

In one embodiment, the recurrent cancer is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

According to one embodiment, the T-cell receptor is provided as a soluble molecule.

As used herein, a soluble T-cell receptor refers to heterodimeric truncated variants of native TCRs, which comprise extracellular portions of the TCR α-chain and β-chain, for example linked by a disulfide bond, but which lack the transmembrane and cytosolic domains of the native protein. The terms “soluble T-cell receptor α-chain sequence and soluble T-cell receptor β-chain sequence” refer to TCR α-chain and β-chain sequences that lack the transmembrane and cytosolic domains. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Soluble TCR of the present disclosure in any case retain the binding functionality of their parent molecules.

### PRAMESpecific TCRs

Complexes of peptide and MHC class I are recognized by CD8-positive T-cells bearing the appropriate T-cell receptor (TCR), whereas complexes of peptide and MHC class II molecules are recognized by CD4-positive-helper-T-cells bearing the appropriate TCR. It is recognized that the TCR, the peptide and the MHC are thereby present in a stoichiometric amount of 1:1:1.

This interaction is highly specific, for example, in the MHC class I dependent immune reaction, peptides not only have to be able to bind to certain MHC class I molecules expressed by tumor cells, they subsequently also have to be recognized by T-cells bearing a specific T-cell receptor (TCR). Usually, when targeting peptide-MHC by said specific TCRs (e.g., soluble TCRs) and antibodies according to the present disclosure, the presentation is the determining factor for a successful response.

The present disclosure further relates to T-cell receptors (TCRs), in particular soluble TCR (sTCRs) and cloned TCRs engineered into autologous or allogeneic T-cells, and methods of making these, as well as NK cells or other cells bearing said TCR or cross-reacting with said TCRs.

Structurally, a subgroup of these T-cell receptors (TCRs) comprises an alpha chain and a beta chain (“alpha/beta TCRs”). These TCRs specifically bind to a peptide, e.g., SLLQHLIGL (PRAME-004) (SEQ ID NO: 310), according to the present disclosure when presented by an MHC molecule. The present description also relates to fragments of such TCRs according to the present disclosure that are still capable of specifically binding to a peptide antigen e.g., PRAME-004 (SEQ ID NO: 310), according to the present disclosure when presented by an HLA molecule. This relates to soluble TCR fragments, for example, TCRs missing the transmembrane parts and/or constant regions, single chain TCRs, and fusions thereof to, for example, with immunoglobulin (Ig). For example, TCRs and fragments thereof of the present disclosure may include those disclosed in US 20180273602, US 10800832, and US 20200123221, the contents of which are herein incorporated by reference in their entireties.

The alpha and beta chains of alpha/beta TCR’s and the gamma and delta chains of gamma/delta TCRs, structurally have two “domains,” namely variable and constant domains. The variable domain consists of a concatenation of variable region (V) and joining region (J). The variable domain may also include a leader region (L). Beta and delta chains may also include a diversity region (D). The alpha and beta constant domains may also include C-terminal transmembrane (TM) domains that anchor the alpha and beta chains to the cell membrane.

The majority of available TCR structures are αβ TCRs, which are formed of TCRα and TCRβ chains. A small number of TCRs are γδ TCRs, consisting of TCRy and TCRδ chains. The TCRβ and TCRδ chains are considered to be analogous to antibody heavy chains, while the TCRα and TCRy chains are considered to be analogous to antibody light chains (Rudolph M.G., Stanfield R.L., Wilson I.A. How TCRs bind MHCs, peptides, and coreceptors. Annu. Rev. Immunol. 2006, 24:419-466).

As mentioned above, each TCR chain is characterized by two immunoglobulin domains: a variable domain (V) and a constant (C). Both variable and constant domains have a conserved β-sandwich structure, making it possible to number and compare variable domains from different TCRs (Dunbar J., Deane C.M. ANARCI: antigen receptor numbering and receptor classification. Bioinformatics. 2016, 32:298-300.). The IMGT numbering has been used for structural analysis of TCRs (Glanville J., Huang H., Nau A., Hatton O., Wagar L.E., Rubelt F., Ji X., Han A., Krams S.M., Pettus C. et al. Identifying specificity groups in the T-cell receptor repertoire. Nature. 2017, 547:94-98, Dunbar J., Knapp B., Fuchs A., Shi J., Deane C.M. Examining variable domain orientations in antigen receptors gives insight into TCR-like antibody design. PLOS Comput. Biol. 2014, 10:1-10). On each variable domain, there are three hypervariable loops that have the highest degree of sequence and structural variation, known as the complementary-determining regions (CDR1, CDR2, and CDR3). Flanking the CDRs, the remaining portions of the TCR structure are collectively known as the TCR’s “framework.”

The CDRs may comprise one or more “changes,” such as substitutions, additions or deletions from the given sequence, provided that the TCR retains the capacity to bind a peptide:MHC complex. The change may involve substitution of an amino acid for a similar amino acid, e.g., a conservative substitution. A similar amino acid is one which has a side chain moiety with related properties as grouped together, for example, (i) basic side chains: lysine, arginine, histidine, (ii) acidic side chains: aspartic acid and glutamic acid, (iii) uncharged polar side chains: asparagine, glutamine, serine, threonine and tyrosine, and (iv) non-polar side chains: glycine, alanine, valine, leucine, isoleucine, proline, phenylalanine, methionine, tryptophan and cysteine.

Outside of the variable parts of the TCR, TCR structures are highly conserved, and therefore only a very small part of the chains creates the actual specificity of the TCR repertoire. As mentioned above, TCRs are generated by genomic rearrangement of the germline TCR locus, a process termed V(D)J recombination, that has the potential to generate marked diversity of TCRs (estimated to range from 10¹⁵ to as high as 10⁶¹ possible receptors).

Despite this potential diversity, TCRs from T-cells that recognize the same pMHC epitope often share conserved sequence features. Analyses demonstrate that each epitope-specific repertoire contains a clustered group of receptors that share core sequence similarities, together with a dispersed set of diverse “outlier” sequences. By identifying shared motifs in core sequences, key conserved residues driving essential elements of TCR recognition can be highlighted (Glanville J., et al. Identifying specificity groups in the T-cell receptor repertoire. Nature. 2017, 547:94-98. Dash P, et al. Quantifiable predictive features define epitope-specific T-cell receptor repertoires. Nature.2017 Jul 6,547(7661):89-93, both herewith specifically incorporated by reference). These analyses provide insights into the generalizable, underlying features of epitope-specific repertoires and adaptive immune recognition.

Sequence analysis focusing entirely on high probability contact sites in CDR3 seems to provide a means of clustering TCRs by shared specificity, as the majority of these possible contacts are in the CDR3s, and only short, typically linear stretches of amino acids make contact with antigenic peptide residues (IMGT positions 107-116), whereas the stem positions of CDR3 (IMGT positions 104, 105, 106, 117, and 118) are never within 5 Å of the antigen (Glanville J., et al. Identifying specificity groups in the T-cell receptor repertoire. Nature. 2017, 547:94-98). Whereas there is always at least one CDR3β contact, there are multiple cases, in which no CDR3α contact is made, suggesting that the former is required, although typically both are involved. Therefore, now well-established features of TCR repertoire analysis include length, charge, and hydrophobicity of the CDR3 regions, clonal diversity (within individuals), and amino acid sequence sharing (across individuals). Using, for example, the GLIPH algorithm can organize TCR sequences into distinct groups of shared specificity either within an individual or across a group of individuals.

Therefore, the estimated number of specific T-cell receptors and thus the repertoire of amino acid sequences of the relevant variable regions is rather small, and the availability of even only one antigen-determining receptor sequence can readily enable the person of skill to create and search for other related T-cell receptors sharing the same specificity. Since general methods of making TCRs are known, and the specific interactions between the peptide/MHC and the receptor have been extensively studies, even the knowledge about the peptide/MHC complex should provide the person of skill with sufficient information, to be fully able to produce the herein described specific subset of variable regions for the inventive T-cell receptors (or the described specific fragments thereof), without suffering an undue burden, e.g. because of a lack of specific directions regarding the relevant positions of the receptors.

In one aspect, to obtain T-cells expressing TCRs of the present description, nucleic acids encoding TCR-alpha and/or TCR-beta chains of the present description are cloned into expression vectors, such as gamma retrovirus, lentivirus, or non-viral vectors, e.g., transposons, nanoplasmids, and CRISPR. The recombinant viruses or vectors are generated and then tested for functionality, such as antigen specificity and functional avidity. An aliquot of the final product is then used to transduce the target T-cell population (generally purified from patient PBMCs), which is expanded before infusion into the patient.

In another aspect, to obtain T-cells expressing TCRs of the present description, TCR RNAs are synthesized by techniques known in the art, e.g., in vitro transcription systems. The in vitro-synthesized TCR RNAs are then introduced into primary CD8+ T-cells obtained from healthy donors by electroporation to re-express tumor specific TCR-alpha and/or TCR-beta chains.

In an embodiment, a TCR of the present description having at least one mutation in the alpha chain and/or having at least one mutation in the beta chain has modified glycosylation compared to the unmutated TCR.

Alpha/beta heterodimeric TCRs of the present description may have an introduced disulfide bond between their constant domains. Preferred TCRs of this type include those which have a TRAC constant domain sequence and a TRBC1 or TRBC2 constant domain sequence except that Thr 48 of TRAC and Ser 57 of TRBC1 or TRBC2 are replaced by cysteine residues, the said cysteines forming a disulfide bond between the TRAC constant domain sequence and the TRBC1 or TRBC2 constant domain sequence of the TCR.

With or without the introduced inter-chain bond mentioned above, alpha/beta hetero-dimeric TCRs of the present description may have a TRAC constant domain sequence and a TRBC1 or TRBC2 constant domain sequence, and the TRAC constant domain sequence and the TRBC1 or TRBC2 constant domain sequence of the TCR may be linked by the native disulfide bond between Cys4 of exon 2 of TRAC and Cys2 of exon 2 of TRBC1 or TRBC2.

Therefore, in one additional or alternative embodiment the antigen binding molecule of the present disclosure comprises CDR1, CDR2, CDR2bis and CDR3 sequences in a combination as provided in SEQ ID NOs: 12 - 128, which display the respective variable chain allele together with the CDR3 sequence. Therefore, preferred are antigen binding molecules of the present disclosure which comprise at least one, preferably, all four CDR sequences CDR1, CDR2, CDR2bis and CDR3. Preferably, an antigen binding molecule of the present disclosure comprises the respective CDR1, CDR2bis and CDR3 of one individual herein disclosed TCR variable region of the present disclosure (see SEQ ID NOs: 12 - 128 and the example section).

In an embodiment, the TCR alpha variable domain has at least one mutation relative to a TCR alpha domain shown in SEQ ID NOs: 12 - 128, and/or the TCR beta variable domain has at least one mutation relative to a TCR alpha domain shown in SEQ ID NOs: 12 - 128. In an embodiment, a TCR comprising at least one mutation in the TCR alpha variable domain and/or TCR beta variable domain has a binding affinity for, and/or a binding half-life for, a TAA peptide-HLA molecule complex, which is at least double that of a TCR comprising the unmutated TCR alpha domain and/or unmutated TCR beta variable domain.

The antigen binding molecule of the present disclosure may comprise a TCR α or γ chain, and/or a TCR β or δ chain, wherein the TCR α or γ chain comprises a CDR3 having at least one, at least two, at least three, at least four, or at least five amino acid substitutions of an amino acid sequence selected from SEQ ID NOs: 14, 26, 38, 50, 62, 74, 86, and 110 and/orwherein the TCR β or δ chain comprises a CDR3 having at least one, at least two, at least three, at least four, or at least five amino acid substitutions of an amino acid sequence selected from SEQ ID NOs: 20, 32, 44, 56, 68, 80, 92, and 116.

Most preferably, in some additional embodiments, wherein the disclosure refers to antigen binding molecules comprising any one, two, three or all of the CDR1, CDR2, CDR2bis and CDR3 regions of the herein disclosed TCR chains (see Table 7), such antigen binding molecules may be preferred, which comprise the respective CDR sequence of the present disclosure with not more than three, two, and preferably only one, modified amino acid residues. A modified amino acid residue may be selected from an amino acid insertion, deletion or substitution. Most preferred is that the three, two, preferably only one modified amino acid residue is the first or last amino acid residue of the respective CDR sequence. If the modification is a substitution, then it is preferable in some embodiments that the substitution is a conservative amino acid substitution.

Such conservative substitutions may be, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions are herein defined as exchanges within one of the following five groups: Group 1-small aliphatic, nonpolar or slightly polar residues (Ala, Ser, Thr, Pro, Gly), Group 2-polar, negatively charged residues and their amides (Asp, Asn, Glu, Gln), Group 3-polar, positively charged residues (His, Arg, Lys), Group 4-large, aliphatic, nonpolar residues (Met, Leu, Ile, Val, Cys), and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

If substitutions at more than one position are found to result in an antigen binding molecule of the present disclosure with substantially equivalent or greater antigen binding activity, then combinations of those substitutions will be tested to determine if the combined substitutions result in additive or synergistic effects on the antigen binding activity. For example, no more than four positions, no more than three positions, no more than two positions, or no more than one position within the CR3 region of an antigen binding molecule of the present disclosure would be simultaneously substituted.

If the antigen binding molecule of the present disclosure is composed of at least two amino acid chains, such as a double chain TCR, or antigen binding fragment thereof, the antigen binding molecule may comprises in a first polypeptide chain the amino acid sequence according to SEQ ID NO: 14, and in a second polypeptide chain the amino acid sequence according to SEQ ID NO: 20, or in a first polypeptide chain the amino acid sequence according to SEQ ID NO: 26, and in a second polypeptide chain the amino acid sequence according to SEQ ID NO: 32, or in a first polypeptide chain the amino acid sequence according to SEQ ID NO: 38, and in a second polypeptide chain the amino acid sequence according to SEQ ID NO: 44, or in a first polypeptide chain the amino acid sequence according to SEQ ID NO: 50, and in a second polypeptide chain the amino acid sequence according to SEQ ID NO: 56, or in a first polypeptide chain the amino acid sequence according to SEQ ID NO: 62, and in a second polypeptide chain the amino acid sequence according to SEQ ID NO: 68, or in a first polypeptide chain the amino acid sequence according to SEQ ID NO: 74, and in a second polypeptide chain the amino acid sequence according to SEQ ID NO: 80, or in a first polypeptide chain the amino acid sequence according to SEQ ID NO: 86, and in a second polypeptide chain the amino acid sequence according to SEQ ID NO: 92, or in a first polypeptide chain the amino acid sequence according to SEQ ID NO: 110, and in a second polypeptide chain the amino acid sequence according to SEQ ID NO: 116.

Any one of the aforementioned double chain TCR, or antigen binding fragments thereof, are preferred TCR of the present disclosure. In some embodiments, the CDR3 of the double chain TCR of the present disclosure may be mutated. Mutations of the CDR3 sequences as provided above preferably include a substitution, deletion, addition, or insertion of not more than three, preferably two, and most preferably not more than one amino acid residue. In some embodiments, the first polypeptide chain may be a TCR α or γ chain, and the second polypeptide chain may be a TCR β or δ chain. Preferred is the combination of an αβ or γδ TCR.

The TCR, or the antigen binding fragment thereof, is in some embodiments composed of a TCR α and a TCR β chain, or y and δ chain. Such a double chain TCR comprises within each chain variable regions, and the variable regions each comprise one CDR1, one CDR2, or more preferably one CDR2bis, and one CDR3 sequence. The TCRs comprises the CDR1, CDR2, CDR2bis and CDR3 sequences as comprised in the variable chain amino acid sequence of SEQ ID NOs: 15 and 21, or 27 and 33, or 39 and 45, or 51 and 57, or 63 and 69, or 75 and 81, or 87 and 93, or 111 and 117.

Some embodiments of the present disclosure pertain to a TCR, or a fragment thereof, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to SEQ ID NOs: 15 and 21, or 27 and 33, or 39 and 45, or 51 and 57, or 63 and 69, or 75 and 81, or 87 and 93, or 111 and 117.

In a particularly preferred embodiment, the present disclosure provides an improved TCR, designated as R11P3D3_KE, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to SEQ ID NOs: 113 and 119. This TCR showed a surprisingly improved functionality in terms of tumor cell recognition when compared to its parent receptor, designated herein as R11P3D3.

The inventive TCRs may further comprise a constant region derived from any suitable species, such as any mammal, e.g., human, rat, monkey, rabbit, donkey, or mouse. In an embodiment of the present disclosure, the inventive TCRs further comprise a human constant region. In some preferred embodiments, the constant region of the TCR of the present disclosure may be slightly modified, for example, by the introduction of heterologous sequences, preferably mouse sequences, which may increase TCR expression and stability. In some preferred embodiments, the variable region of the TCR of the intervention may be slightly modified, for example, by the introduction of single point mutations to optimize the TCR stability and/or to enhance TCR chain pairing.

Some embodiments of the present disclosure pertain to a TCR, or a fragment thereof, composed of a TCR α and a TCR β chain, wherein said TCR comprises the constant region having at least 50%, 60%, 70%, 80%, 90%, 95%, 98%, 99%, or preferably 100% sequence identity to an amino acid sequence selected from of the α and β chain according to SEQ ID NOs: 16 and 22, or 28 and 34, or 40 and 46, or 52 and 58, or 64 and 70, or 76 and 82, or 88 and 94, or 112 and 118.

The TCR α or γ chain of the present disclosure may further comprise a CDR1 having at least one, at least two, at least three, at least four, or at least five amino acid substitutions of an amino acid sequence selected from SEQ ID NOs: 12, 24, 36, 48, 60, 72, 84 and 108, and/or a CDR2 having at least one, at least two, at least three, at least four, or at least five amino acid substitutions of an amino acid sequence selected from SEQ ID NOs: 13, 25, 37, 49, 61, 73, 85 and 109, and/or more preferably a CDR2bis having at least one, at least two, at least three, at least four, or at least five amino acid substitutions of an amino acid sequence selected from SEQ ID NOs: 120, 121, 122, 123, 124, 125, 126 and 128

According to the present disclosure the TCR β or δ chain may further comprise a CDR1 having at least one, at least two, at least three, at least four, or at least five amino acid substitutions of an amino acid sequence selected from SEQ ID NOs: 18, 30, 42, 54, 66, 78, 90 and 114, and/or a CDR2 having at least one, at least two, at least three, at least four, or at least five amino acid substitutions of an amino acid sequence selected from SEQ ID NOs: 19, 31, 43, 55, 67, 79, 91 and 115, and/or more preferably a CDR2bis having at least one, at least two, at least three, at least four, or at least five amino acid substitutions of an amino acid sequence selected from SEQ ID NOs: 19, 31, 43, 55, 67, 79, 91 and 115.

The antigen binding molecule may in a further embodiment comprise a binding fragment of a TCR, and wherein said binding fragment comprises in one chain CDR1, CDR2, CDR2bis and CDR3, optionally selected from the CDR1, CDR2, CDR2bis and CDR3 sequences having the amino acid sequences of SEQ ID NOs: 12, 13, 14, 120, 11, 18, 19, 20, or 24, 25, 26, 121, or 30, 31, 32, or 36, 37, 38, 122, or 42, 43, 44, or 48, 49, 50, 123, or 54, 55, 56, or 60, 61, 62, 124, or 66, 67, 68, or 72, 73, 74, 125, or 78, 79, 80, or 84, 85, 86, 126, or 90, 91, 92, or 108, 109, 110, 128, or 114, 115, 116

In further embodiments of the present disclosure the antigen binding molecule as described herein elsewhere is a TCR, or a fragment thereof, composed of at least one TCR α and one TCR β chain sequence, wherein said TCR α chain sequence comprises the CDR1, CDR2, CDR2bis and CDR3 sequences having the amino acid sequences of SEQ ID NOs: 12 to 14 and 120, and said TCR β chain sequence comprises the CDR1 to CDR3 sequences having the amino acid sequences of SEQ ID NOs: 18 to 20, or wherein said TCR α chain sequence comprises the CDR1, CDR2, CDR2bis and CDR3 sequences having the amino acid sequences of SEQ ID NOs: 24 to 26 and 121, and said TCR β chain sequence comprises the CDR1 to CDR3 sequences having the amino acid sequences of SEQ ID NOs: 30 to 32, or wherein said TCR α chain sequence comprises the CDR1, CDR2, CDR2bis and CDR3 sequences having the amino acid sequences of SEQ ID NOs: 36 to 38 and 122 and said TCR β chain sequence comprises the CDR1 to CDR3 sequences having the amino acid sequences of SEQ ID NOs: 42 to 44, or wherein said TCR α chain sequence comprises the CDR1, CDR2, CDR2bis and CDR3 sequences having the amino acid sequences of SEQ ID NOs: 48 to 50 and 123, and said TCR β chain sequence comprises the CDR1 to CDR3 sequences having the amino acid sequences of SEQ ID NOs: 54 to 56, or wherein said TCR α chain sequence comprises the CDR1, CDR2, CDR2bis and CDR3 sequences having the amino acid sequences of SEQ ID NOs: 60 to 62 and 124, and said TCR β chain sequence comprises the CDR1 to CDR3 sequences having the amino acid sequences of SEQ ID NOs: 66 to 68, or wherein said TCR α chain sequence comprises the CDR1, CDR2, CDR2bis and CDR3 sequences having the amino acid sequences of SEQ ID NOs: 72 to 74 and 125, and said TCR β chain sequence comprises the CDR1 to CDR3 sequences having the amino acid sequences of SEQ ID NOs: 78 to 80, or wherein said TCR α chain sequence comprises the CDR1, CDR2, CDR2bis and CDR3 sequences having the amino acid sequences of SEQ ID NOs: 84 to 86 and 126, and said TCR β chain sequence comprises the CDR1 to CDR3 sequences having the amino acid sequences of SEQ ID NOs: 90 to 92, or wherein said TCR α chain sequence comprises the CDR1, CDR2, CDR2bis and CDR3 sequences having the amino acid sequences of SEQ ID NOs: 108 to 110 and 128, and said TCR β chain sequence comprises the CDR1 to CDR3 sequences having the amino acid sequences of SEQ ID NOs: 114 to 116.

In further embodiments of the present disclosure the antigen binding molecule as described herein before is a TCR, or a fragment thereof, comprising at least one TCR α and one TCR β chain sequence, wherein said TCR α chain sequence comprises a variable region sequence having the amino acid sequence of SEQ ID NO: 15, and wherein said TCR β chain sequence comprises a variable region sequence having the amino acid sequence of SEQ ID NO: 21, or wherein said TCR α chain sequence comprises a variable region sequence having the amino acid sequence of SEQ ID NO: 27, and wherein said TCR β chain sequence comprises a variable region sequence having the amino acid sequence of SEQ ID NO: 33, or wherein said TCR α chain sequence comprises a variable region sequence having the amino acid sequence of SEQ ID NO: 39, and wherein said TCR β chain sequence comprises a variable region sequence having the amino acid sequence of SEQ ID NO: 45, or wherein said TCR α chain sequence comprises a variable region sequence having the amino acid sequence of SEQ ID NO: 51, and wherein said TCR β chain sequence comprises a variable region sequence having the amino acid sequence of SEQ ID NO: 57, or wherein said TCR α chain sequence comprises a variable region sequence having the amino acid sequence of SEQ ID NO: 63, and wherein said TCR β chain sequence comprises a variable region sequence having the amino acid sequence of SEQ ID NO: 69, or wherein said TCR α chain sequence comprises a variable region sequence having the amino acid sequence of SEQ ID NO: 75, and wherein said TCR β chain sequence comprises a variable region sequence having the amino acid sequence of SEQ ID NO: 81, or wherein said TCR α chain sequence comprises a variable region sequence having the amino acid sequence of SEQ ID NO: 87, and wherein said TCR β chain sequence comprises a variable region sequence having the amino acid sequence of SEQ ID NO: 93, or wherein said TCR α chain sequence comprises a variable region sequence having the amino acid sequence of SEQ ID NO: 111, and wherein said TCR β chain sequence comprises a variable region sequence having the amino acid sequence of SEQ ID NO: 117.

In further embodiments of the present disclosure the antigen binding molecule as described herein before is a TCR, or a fragment thereof, further comprising a TCR constant region having at least 50%, 60%, 70%, 80%, 90%, 95%, 98%, 99%, or 100% sequence identity to an amino acid sequence selected from SEQ ID NOs: 16, 22, 28, 34, 40, 46, 52, 58, 64, 70, 76, 82, 88, 94, 112 and 118, preferably wherein the TCR is composed of at least one TCR α and one TCR β chain sequence, wherein the TCR α chain sequence comprises a constant region having at least 50%, 60%, 70%, 80%, 90%, 95%, 98%, 99%, or 100% sequence identity to an amino acid sequence selected from SEQ ID NOs: 16, 28, 40, 52, 64, 76, 88 and 112, and wherein the TCR β chain sequence comprises a constant region having at least 50%, 60%, 70%, 80%, 90%, 95%, 98%, 99%, or 100% sequence identity to an amino acid sequence selected from SEQ ID NOs: 22, 34, 46, 58, 70, 82, 94, and 118.

Also disclosed are antigen binding molecules as described herein before comprising a first TCR chain having at least 50%, 60%, 70%, 80%, 90%, 95%, 98%, 99%, or 100% sequence identity to the amino acid sequence of SEQ ID NO: 17, and a second TCR chain having at least 50%, 60%, 70%, 80%, 90%, 95%, 98%, 99%, or 100% sequence identity to the amino acid sequence of SEQ ID NO: 23, The present disclosure also provides TCRs comprising a first TCR chain having at least 50%, 60%, 70%, 80%, 90%, 95%, 98%, 99%, or 100% sequence identity to the amino acid sequence of SEQ ID NO: 29, and a second TCR chain having at least 50%, 60%, 70%, 80%, 90%, 95%, 98%, 99%, or 100% sequence identity to the amino acid sequence of SEQ ID NO: 35, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 41, and a second TCR chain having at least 50%, 60%, 70%, 80%, 90%, 95%, 98%, 99%, or 100% sequence identity to the amino acid sequence of SEQ ID NO: 47, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 53, and a second TCR chain having at least 50%, 60%, 70%, 80%, 90%, 95%, 98%, 99%, or 100% sequence identity to the amino acid sequence of SEQ ID NO: 59, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 65, and a second TCR chain having at least 50%, 60%, 70%, 80%, 90%, 95%, 98%, 99%, or 100% sequence identity to the amino acid sequence of SEQ ID NO: 71, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 77, and a second TCR chain having at least 50%, 60%, 70%, 80%, 90%, 95%, 98%, 99%, or 100% sequence identity to the amino acid sequence of SEQ ID NO: 83, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 89, and a second TCR chain having at least 50%, 60%, 70%, 80%, 90%, 95%, 98%, 99%, or 100% sequence identity to the amino acid sequence of SEQ ID NO: 95, In further embodiments, the present disclosure provides antigen binding molecules which are TCR and comprise a first TCR chain having at least 50%, 60%, 70%, 80%, 90%, 95%, 98%, 99%, or 100% sequence identity to the amino acid sequence of SEQ ID NO: 113, and a second TCR chain having at least 50%, 60%, 70%, 80%, 90%, 95%, 98%, 99%, or 100% sequence identity to the amino acid sequence of SEQ ID NO: 119,

As used herein, the term “murine” or “human,” when referring to an antigen binding molecule, or a TCR, or any component of a TCR described herein (e.g., complementarity determining region (CDR), variable region, constant region, α chain, and/or β chain), means a TCR (or component thereof), which is derived from a mouse or a human unrearranged TCR locus, respectively.

In an embodiment of the present disclosure, chimeric TCR are provided, wherein the TCR chains comprise sequences from multiple species. Preferably, a TCR of the present disclosure may comprise an α chain comprising a human variable region of an α chain and, for example, a murine constant region of a murine TCR α chain.

According to another aspect of the present disclosure, a nucleic acid is provided, which encodes for a peptide according to the above description, or for an antibody or fragment thereof according to the above description, or for a T-cell receptor or fragment thereof according to the above description.

Alternatively or in addition, a method of treating a patient (i) being diagnosed for, (ii) suffering from or (iii) being at risk of developing recurrent cancer,

is provided.

The method comprises administering to the patient a nucleic acid which encodes for a peptide according to the above description, or for an antibody or fragment thereof according to the above description, or for a T-cell receptor or fragment thereof according to the above description, in one or more therapeutically effective doses.

Alternatively or in addition, a pharmaceutical composition for treating recurrent cancer is provided, comprising a nucleic acid which encodes for a peptide according to the above description, or for an antibody or fragment thereof according to the above description, or for a T-cell receptor or fragment thereof according to the above description, as an effective ingredient.

In one embodiment, the recurrent cancer is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

Optionally, said nucleic acid is provided for use in the (manufacture of a medicament for the) treatment of a patient (i) being diagnosed for, (ii) suffering from or (iii) being at risk of developing recurrent cancer.

Such nucleic acid can be an mRNA or a DNA. Such nucleic acid can be delivered as a plasmid or a linear molecule. Such nucleic acid can be delivered by a viral vector, or encapsulated into a liposome. Such mRNA can comprise modified nucleosides, like pseudouridine or 1 methyl pseudouridine, to reduce immunogenic effects. Such mRNA can be G/C codon optimized to have a decreased uridine content.

According to another aspect of the present disclosure, a recombinant host cell comprising the peptide according to the above description, the antibody or fragment thereof to the above description, the T-cell receptor or fragment thereof according to the above description or the nucleic acid according to the above description is provided.

According to another aspect of the present disclosure, a recombinant T lymphocyte is provided which expresses at least one vector encoding a T-cell receptor according to the above description.

The T Lymphocyte is provided for use in the (manufacture of a medicament for the) treatment of a patient (i) being diagnosed for, (ii) suffering from or (iii) being at risk of developing recurrent cancer.

Alternatively or in addition, a method of treating a patient (i) being diagnosed for, (ii) suffering from or (iii) being at risk of developing recurrent cancer, is provided.

The method comprises administering to the patient a recombinant T lymphocyte which expresses at least one vector encoding a T-cell receptor according to the above description, in one or more therapeutically effective doses.

Alternatively or in addition, a pharmaceutical composition for recurrent cancer is provided, comprising a recombinant T lymphocyte which expresses at least one vector encoding a T-cell receptor according to the above description, as an effective ingredient.

In one embodiment, the recombinant T lymphocytes are produced by a method comprising isolating a cell from a subject, transforming the cell with at least one vector encoding the T-cell receptor, to produce a recombinant T lymphocyte, and expanding the recombinant T lymphocyte to produce the population of recombinant T lymphocytes.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

In one embodiment, the recombinant T lymphocyte is a CD8+ (CD8 positive) T Lymphocyte. A CD8+ T Lymphocyte (also called cytotoxic T cell CTL, T-killer cell, cytolytic T cell, or killer T cell) is a T lymphocyte hat kills cancer cells, cells that are infected (particularly with viruses), or cells that are damaged in other ways.

Most cytotoxic T cells express T-cell receptors (TCRs) that can recognize a specific antigen. An antigen is a molecule capable of stimulating an immune response and is often produced by cancer cells or viruses. Antigens inside a cell are bound to class I MHC molecules, and brought to the surface of the cell by the class I MHC molecule, where they can be recognized by the T cell. If the TCR is specific for that antigen, it binds to the complex of the class I MHC molecule and the antigen, and the T cell destroys the cell.

For the TCR to bind to the class I MHC molecule, the former must be accompanied by a glycoprotein called CD8, which binds to the constant portion of the class I MHC molecule. Therefore, these T cells are called CD8+ T cells.

According to several embodiments, the T-cell receptor comprises:
(1) a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
(2) a CDR1a chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
(3) a CDR1a chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
(4) a CDR1a chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
(5) a CDR1a chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
(6) a CDR1a chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
(7) a CDR1a chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
wherein the T-cell receptor is capable of binding to a peptide consisting of the amino acid sequence of SLLQHLIGL (SEQ ID NO: 310) in a complex with HLA-A*02.

According to several embodiments, the T-cell receptor comprises:
(1) an α chain variable domain comprising SEQ ID NO: 15, and a β chain variable domain comprising SEQ ID NO: 21, or
(2) an α chain variable domain comprising SEQ ID NO: 27, and a β chain variable domain comprising SEQ ID NO: 33, or
(3) an α chain variable domain comprising SEQ ID NO: 39, and a β chain variable domain comprising SEQ ID NO: 45, or
(4) an α chain variable domain comprising SEQ ID NO: 51, and a β chain variable domain comprising SEQ ID NO: 57, or
(5) an α chain variable domain comprising SEQ ID NO: 63, and a β chain variable domain comprising SEQ ID NO: 69, or
(6) an α chain variable domain comprising SEQ ID NO: 75, and a β chain variable domain comprising SEQ ID NO: 81, or
(7) an α chain variable domain comprising SEQ ID NO: 87, and a β chain variable domain comprising SEQ ID NO: 93, or
(8) an α chain variable domain comprising SEQ ID NO: 111, and a β chain variable domain comprising SEQ ID NO: 117,
wherein the T-cell receptor is capable of binding to a peptide consisting of the amino acid sequence of SLLQHLIGL (SEQ ID NO: 310) in a complex with HLA-A*02.

According to another aspect of the present disclosure, an in vitro method for producing activated T lymphocytes is provided. The method comprises contacting in vitro T-cells with antigen loaded human class I MHC molecules expressed on the surface of a suitable antigen-presenting cell or an artificial construct mimicking an antigen-presenting cell for a period of time sufficient to activate said T lymphocyte in an antigen specific manner. Said antigen is a peptide according to the above description.

According to another aspect of the present disclosure, an activated T lymphocyte, produced by the method according to the above description is provided, which selectively recognizes a cell which presents a peptide according to the above description.

The T Lymphocyte is provided for use in the (manufacture of a medicament for the) treatment of a patient (i) being diagnosed for, (ii) suffering from or (iii) being at risk of developing recurrent cancer.

Alternatively or in addition, a method of treating a patient (i) being diagnosed for, (ii) suffering from or (iii) being at risk of developing recurrent cancer, is provided.

The method comprises administering to the patient an activated T lymphocyte, produced by the method according to the above description, which selectively recognizes a cell which presents a peptide according to the above description, in one or more therapeutically effective doses.

Alternatively or in addition, a pharmaceutical composition for treating recurrent cancer is provided, comprising an activated T lymphocyte, produced by the method according to the above description, which selectively recognizes a cell which presents a peptide according to the above description, as an effective ingredient.

In one embodiment, the recurrent cancer is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

In one embodiment, the activated T lymphocyte is a CD8+ (CD8 positive) T Lymphocyte.

### Adoptive Cellular Therapy: yδ T-Cell Manufacturing

To isolate γδ T-cells, in an aspect, γδ T-cells may be isolated from a subject or from a complex sample of a subject. In an aspect, a complex sample may be a peripheral blood sample, a cord blood sample, a tumor, a stem cell precursor, a tumor biopsy, a tissue, a lymph, or from epithelial sites of a subject directly contacting the external milieu or derived from stem precursor cells. γδ T-cells may be directly isolated from a complex sample of a subject, for example, by sorting γδ T-cells that express one or more cell surface markers with flow cytometry techniques. Wild-type γδ T-cells may exhibit numerous antigen recognition, antigen-presentation, co-stimulation, and adhesion molecules that can be associated with a γδ T-cells. One or more cell surface markers, such as specific γδ TCRs, antigen recognition, antigen-presentation, ligands, adhesion molecules, or co-stimulatory molecules may be used to isolate wild-type γδ T-cells from a complex sample. Various molecules associated with or expressed by γδ T-cells may be used to isolate γδ T-cells from a complex sample, e.g., isolation of mixed population of Vδ1+, Vδ2+, Vδ3+ cells or any combination thereof.

For example, peripheral blood mononuclear cells can be collected from a subject, for example, with an apheresis machine, including the Ficoll-Paque™ PLUS (GE Healthcare) system, or another suitable device/system. γδ T-cell(s), or a desired subpopulation of γδ T-cell(s), can be purified from the collected sample with, for example, with flow cytometry techniques. Cord blood cells can also be obtained from cord blood during the birth of a subject.

Positive and/or negative selection of cell surface markers expressed on the collected γδ T-cells can be used to directly isolate γδ T-cells, or a population of γδ T-cells expressing similar cell surface markers from a peripheral blood sample, a cord blood sample, a tumor, a tumor biopsy, a tissue, a lymph, or from an epithelial sample of a subject. For instance, γδ T-cells can be isolated from a complex sample based on positive or negative expression of CD2, CD3, CD4, CD8, CD24, CD25, CD44, Kit, TCR α, TCR β, TCR α, TCR δ, NKG2D, CD70, CD27, CD30, CD16, CD337 (NKp30), CD336 (NKp46), OX40, CD46, CCR7, and other suitable cell surface markers.

This process may include collecting or obtaining white blood cells or PBMC from leukapheresis products. Leukapheresis may include collecting whole blood from a donor and separating the components using an apheresis machine. An apheresis machine separates out desired blood components and returns the rest to the donor’s circulation. For instance, white blood cells, plasma, and platelets can be collected using apheresis equipment, and the red blood cells and neutrophils are returned to the donor’s circulation. Commercially available leukapheresis products may be used in this process. Another way to obtain white blood cells is to obtain them from the buffy coat. To isolate the buffy coat, whole anticoagulated blood is obtained from a donor and centrifuged. After centrifugation, the blood is separated into plasma, red blood cells, and buffy coat. The buffy coat is the layer located between the plasma and red blood cell layers. Leukapheresis collections may result in higher purity and considerably increased mononuclear cell content than that achieved by buffy coat collection. The mononuclear cell content possible with leukapheresis may typically be 20 times higher than that obtained from the buffy coat. In order to enrich for mononuclear cells, the use of a Ficoll gradient may be needed for further separation.

To deplete αβ T-cells from PBMC, αβ TCR-expressing cells may be separated from the PBMC by magnetic separation, e.g., using CliniMACS® magnetic beads coated with anti-αβ TCR antibodies, followed by cryopreserving αβ TCR-T-cells depleted PBMC. To manufacture “off-the-shelf” T-cell products, cryopreserved αβ TCR-T-cells depleted PBMC may be thawed and activated in small/mid-scale, e.g., 24 to 4-6 well plates or T75/T175 flasks, or in large scale, e.g., 50 ml-100 liter bags, in the presence of aminobisphosphonate, e.g., zoledronate, and/or isopentenylpyrophosphate (IPP) and/or cytokines, e.g., interleukin 2 (IL-2), interleukin 15 (IL-15), and/or interleukin 18 (IL-18), and/or other activators, e.g., Toll-like receptor 2 (TLR2) ligand, for 1 - 10 days, e.g., 2 - 7 days.

### Engineering yδ T-Cells Expressing αβ-TCR and CD8αβ

γδ T-cells of the disclosure may be engineered for use to treat a subject in need of treatment for a condition. To engineer γδ T-cells that express αβ-TCR, e.g., specifically binding to a PRAME-004/MHC complex, αβ-TCR-expressing γ-retrovirus was generated. Because γδ T-cells may not express CD8, γδ T-cells may need CD8α homodimers or CD8αβ heterodimers in addition to αβ-TCR to recognize PRAME-004/MHC-I complexes presented on cell membrane of target cells, e.g., cancer cells. To that end, αβ-TCR/CD8-expressing γ-retrovirus was generated for transducing isolated γδ T-cells using the methods described herein. The sequences of CD8α or the variant thereof and CD8β or the variant thereof may be selected from SEQ ID NO: 1 - 11.

αβ-TCR-expressing Vγ9δ2 T-cells, in which αβ-TCR specifically binds to peptide/MHC complex, were generated by transducing Vγ9δ2 T-cells with αβ-TCR retrovirus and CD8αβ retrovirus.

### Autologous T-Cell Manufacturing Process

Embodiments of the present disclosure may include an about 7 to about 10-day process leading to the manufacturing of over 10 billion (10 × 10⁹) cells without the loss of potency. In addition, the concentrations of several raw materials may be optimized to reduce the cost of good by 30%.

T-cell manufacturing process of the present disclosure may include thawing PBMC on Day 0, followed by resting without cytokines overnight, e.g., 24 hours, followed by activating the rested PBMC with anti-CD3 and anti-CD28 antibodies immobilized on non-tissue culture treated plates. IL-7 is a homeostatic cytokine that promotes survival of T-cells by preventing apoptosis. IL-7 may be added to PBMC during resting.

T-cell manufacturing process of the present disclosure may include thawing PBMC on Day 1, followed by resting in the presence of IL-7 or in the presence of IL-7 + IL-15 or without cytokine for 4-6 hours, followed by activating the rested PBMC with anti-CD3 and anti-CD28 antibodies immobilized on non-tissue culture treated plates.

T-cell manufacturing process of the present disclosure may include thawing PBMC on Day 1 (without resting and without cytokine), followed by activating the thawed PBMC with anti-CD3 and anti-CD28 antibodies immobilized on tissue culture plates. Cells may be harvested and counted on Day 8-10, followed by activation panel analysis.

T-cell manufacturing process of the present disclosure may include resting PBMC for a period of time of about 4 hours according to one embodiment of the present disclosure. For example, a T-cell manufacturing process may include isolation and cryopreservation of PBMC from leukapheresis, in which sterility may be tested; thaw, rest (e.g., about 4 hours) and activate T-cells; transduction with a viral vector; expansion with cytokines; split/feed cells, in which cell count and immunophenotyping may be tested; harvest and cryopreservation of drug product cells, in which cell count and mycoplasma may be tested, and post-cryopreservation release, in which viability, sterility, endotoxin, immunophenotyping, copy number of integrated vector, and vesicular stomatitis virus glycoprotein G (VSV-g) may be tested.

T-cell manufacturing process of the present disclosure may include resting PBMC overnight (about 16 hours). For example, T-cell manufacturing process may include isolation of PBMC, in which PBMC may be used fresh or stored frozen till ready for use, or may be used as starting materials for T-cell manufacturing and selection of lymphocyte populations (e.g., CD8, CD4, or both) may also be possible; thaw and rest lymphocytes overnight, e.g., about 16 hours, which may allow apoptotic cells to die off and restore T-cell functionality (this step may not be necessary, if fresh materials are used); activation of lymphocytes, which may use anti-CD3 and anti-CD28 antibodies (soluble or surface bound, e.g., magnetic or biodegradable beads); transduction with TCRs or bi-specific molecules, which may use lentiviral or retroviral constructs encoding TCRs or bi-specific molecules or may use non-viral methods; and expansion of lymphocytes, harvest, and cryopreservation, which may be carried out in the presence of cytokine(s), serum (ABS or FBS), and/or cryopreservation media.

Table 3a summarizes characteristics of T-cells manufactured with short rest of about 4 hours according to one embodiment of the present disclosure and that with overnight rest of about 16 hours.

TABLE 3a

| Resting for | Fold Expansion | Harvest Count | Viability ≥ 70% | % Live CD3+ ≥ 80% | % CD8+ of CD3+ | % Dex+ of CD8+ ≥ 10% |
|---|---|---|---|---|---|---|
| 4 hours | 78.7 | 28.0 × 10⁹ | 92.0 | 99.7 | 53.4 | 63.7 |
| 16 hours | 45.0 | 15.7 × 10⁹ | 86.0 | 99.5 | 51.9 | 53.0 |

T-cell manufacturing process of the present disclosure may include using fresh PBMCs, which is not obtained by thawing cryopreserved PBMC, thus, minimizing cell loss due to freezing, thawing, and/or resting PBMCs and maximizing cell numbers at the beginning of manufacturing process. For example, T-cell manufacturing process may include Day 0, isolation of fresh PBMC, activation of fresh lymphocytes using, for example, anti-CD3 and anti-CD28 antibodies (soluble or surface bound, e.g., magnetic or biodegradable beads) in bags, e.g., Saint-Gobain VueLife AC Bags, coated with anti-CD3 and anti-CD28 antibodies; Day 1, transduction with TCRs or bi-specific molecules using, for example, lentiviral or retroviral constructs encoding TCRs or bi-specific molecules or non-viral methods, e.g., liposomes; and Day 2, expansion of lymphocytes, Day 5/6, harvest, and cryopreservation in the presence of cytokine(s), serum (ABS or FBS), and/or cryopreservation media.

### Engineering Αβ T-Cells Expressing αβ-TCR and CD8αβ

Engineered αβ T-cells of the disclosure may be used to treat a subject in need of treatment for a condition. To engineer αβ T-cells that express αβ-TCR, e.g., shown below in the sequence listing, specifically binding to a PRAME-004/MHC complex, αβ-TCR-expressing γ-retrovirus was generated. Expression of exogenous CD8α homodimers or CD8αβ heterodimers in CD8+ and/or CD4 T-cells may improve αβ-TCR to recognize PRAME-004/MHC-I complexes on cell membrane of target cells, e.g., cancer cells. To that end, αβ-TCR/CD8-expressing γ-retrovirus was generated for transducing T-cells using the methods described herein. The sequences of CD8α or the variant thereof and CD8β or the variant thereof may be selected from SEQ ID NO: 1 - 11.

### Methods of Treatment

The present disclosure provides therapeutic compositions comprising the PRAME-binding molecules including TCRs and bi-specific molecules or immune effector cells comprising the PRAME TCRs of the present disclosure. Therapeutic compositions in accordance with the present disclosure may be administered with suitable carriers, excipients, and other agents that are incorporated into formulations to provide improved transfer, delivery, tolerance, and the like. A multitude of appropriate formulations can be found in the formulary known to all pharmaceutical chemists: Remington’s Pharmaceutical Sciences, Mack Publishing Company, Easton, PA. These formulations may include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-31 1.

Depending on the severity of the condition, the frequency and the duration of the treatment can be adjusted.

In certain embodiments, the initial dose may be followed by administration of a second or a plurality of subsequent doses of PRAME TCRs or bi-specific molecules of the present disclosure or immune effector cells comprising the PRAME TCRs or bi-specific molecules of the present disclosure in an amount that can be approximately the same or less than that of the initial dose,

In certain situations, the pharmaceutical composition can be delivered in a controlled release system. In some embodiments, a pump may be used.

Injectable preparations may include dosage forms for intravenous, subcutaneous, intracutaneous, intracranial, intraperitoneal and intramuscular injections, drip infusions, etc. The TCRs, bi-specific molecules, pharmaceutical compositions, and cells described herein can be administered via parenteral administration. The preparations of the present disclosure may be prepared by methods publicly known. For example, the preparations may be prepared, e.g., by dissolving, suspending or emulsifying the antigen-binding protein or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

In some embodiments, TCR-expressing immune effector cells may be formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer’s lactate can be utilized. The infusion medium can be supplemented with human serum albumin.

A treatment-effective number of cells in the composition may be typically greater than 10² cells, and up to 10⁶ up to and including 10⁸ or 10⁹ cells and can be more than 10¹⁰ cells. The number of cells may depend upon the ultimate use for which the composition is intended as will the type of cells included therein.

The cells may be autologous or heterologous to the patient undergoing therapy. If desired, the treatment may also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIPIα, etc.) as described herein to enhance induction of the immune response.

The TCR expressing immune effector cell populations of the present disclosure may be administered either alone, or as a pharmaceutical composition in combination with diluents and/or with other components such as IL-2, IL-7, IL-15, or other cytokines or cell populations. Briefly, pharmaceutical compositions of the present disclosure may comprise a TCR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Compositions containing engineered αβ T-cells (e.g., CD4+ and CD8+ T-cells) and/or γδ T-cells that express recombinant TCRs and/or bi-specific molecules binding to PRAME-004 described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. Engineered αβ T-cells and/or γδ T-cells can also be administered to lessen a likelihood of developing, contracting, or worsening a condition. Effective amounts of a population of engineered αβ T-cells and/or γδ T-cells for therapeutic use can vary based on the severity and course of the disease or condition, previous therapy, the subject’s health status, weight, and/or response to the drugs, and/or the judgment of the treating physician.

The composition of the present disclosure may also include one or more adjuvants. Adjuvants are substances that non-specifically enhance or potentiate the immune response (e.g., immune responses mediated by CD8-positive T-cells and helper-T (TH) cells to an antigen and would thus be considered useful in the medicament of the present disclosure. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila’s QS21 stimulon, which is derived from saponin, mycobacterial extracts and synthetic bacterial cell wall mimics, and other proprietary adjuvants such as Ribi’s Detox, Quil, or Superfos. Adjuvants such as Freund’s or GM-CSF are preferred. Several immunological adjuvants (e.g., MF59) specific for dendritic cells and their preparation have been described previously (Allison and Krummel, 1995). Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (US 5,849,589, incorporated herein by reference in its entirety) and acting as immunoadjuvants (e.g., IL-12, IL-15, IL-23, IL-7, IFN-alpha. IFN-beta).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T-cell help. The TH1 bias induced by TLR9 stimulation is maintained even in the presence of vaccine adjuvants such as alum or incomplete Freund’s adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). US 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present disclosure. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present disclosure can readily be determined by the skilled artisan without undue experimentation.

Preferred adjuvants are anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), virosomes, and/or interleukin (IL)-1, IL-2, IL-4, IL-7, IL-12, IL-13, IL-15, IL-21, and IL-23.

In a preferred embodiment, the pharmaceutical composition according to the present disclosure the adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In a preferred embodiment, the pharmaceutical composition according to the present disclosure the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the present disclosure, the adjuvant is cyclophosphamide, imiquimod or resiquimod. Even more preferred adjuvants are Montanide IMS 1312, Montanide ISA 206, Montanide ISA 50V, Montanide ISA-51, poly-ICLC (Hiltonol®) and anti-CD40 mAB, or combinations thereof.

Engineered αβ T-cells and/or γδ T-cells of the present disclosure can be used to treat a subject in need of treatment for a condition, for example, a cancer described herein.

A method of treating a condition (e.g., ailment) in a subject with engineered αβ T-cells and/or γδ T-cells may include administering to the subject a therapeutically effective amount of engineered αβ T-cells and/or γδ T-cells. Engineered αβ T-cells and/or γδ T-cells of the present disclosure may be administered at various regimens (e.g., timing, concentration, dosage, spacing between treatment, and/or formulation). A subject can also be preconditioned with, for example, chemotherapy, radiation, or a combination of both, prior to receiving engineered αβ T-cells and/or γδ T-cells of the present disclosure. A population of engineered αβ T-cells and/or γδ T-cells may also be frozen or cryopreserved prior to being administered to a subject. A population of engineered αβ T-cells and/or γδ T-cells can include two or more cells that express identical, different, or a combination of identical and different tumor recognition moieties. For instance, a population of engineered αβ T-cells and/or γδ T-cells can include several distinct engineered αβ T-cells and/or γδ T-cells that are designed to recognize different antigens, or different epitopes of the same antigen.

In an aspect, engineered αβ T-cells and/or γδ T-cells of the present disclosure may be used to treat an infectious disease. In another aspect, engineered αβ T-cells and/or γδ T-cells of the present disclosure may be used to treat an infectious disease, an infectious disease may be caused a virus. In yet another aspect, engineered αβ T-cells and/or γδ T-cells of the present disclosure may be used to treat an immune disease, such as an autoimmune disease.

Treatment with αβ T-cells and/or γδ T-cells of the present disclosure may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered αβ T-cells and/or γδ T-cells of the present disclosure.

In another aspect, administration of engineered αβ T-cells and/or γδ T-cells of the present disclosure to a subject may modulate the activity of endogenous lymphocytes in a subject’s body. In another aspect, administration of engineered αβ T-cells and/or γδ T-cells to a subject may provide an antigen to an endogenous T-cell and may boost an immune response. In another aspect, the memory T-cell may be a CD4+ T-cell. In another aspect, the memory T-cell may be a CD8+ T-cell. In another aspect, administration of engineered αβ T-cells and/or γδ T-cells of the present disclosure to a subject may activate the cytotoxicity of another immune cell. In another aspect, the other immune cell may be a CD8+ T-cell. In another aspect, the other immune cell may be a Natural Killer T-cell. In another aspect, administration of engineered αβ T-cells and/or γδ T-cells of the present disclosure to a subject may suppress a regulatory T-cell. In another aspect, the regulatory T-cell may be a FOX3+ Treg cell. In another aspect, the regulatory T-cell may be a FOX3- Treg cell. Nonlimiting examples of cells whose activity can be modulated by engineered αβ T-cells and/or γδ T-cells of the disclosure may include: hematopoietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells.

During most bone marrow transplants, a combination of cyclophosphamide with total body irradiation may be conventionally employed to prevent rejection of the hematopoietic stem cells (HSC) in the transplant by the subject’s immune system. In an aspect, incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of αβ T-cells and/or γδ T-cells into humans may require the co-administration of αβ T-cells and/or γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. In another aspect, the disclosure provides a method for administrating engineered αβ T-cells and/or γδ T-cells to a subject without the co-administration of a native cytokine or modified versions thereof, such as IL-2, IL-15, IL-12, IL-21. In another aspect, engineered αβ T-cells and/or γδ T-cells can be administered to a subject without co-administration with IL-2. In another aspect, engineered αβ T-cells and/or γδ T-cells may be administered to a subject during a procedure, such as a bone marrow transplant without the co-administration of IL-2.

### Therapeutic Uses of PRAME TCRs or Immune Effector Cells Comprising PRAME TCRs or PRAME Bi-Specific Molecules

The anti-tumor immune response induced in a subject by administering TCR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

Thus, the PRAME TCRs and/or PRAME bi-specific molecules of the present disclosure may be useful, inter alia, for the treatment, prevention and/or amelioration of any disease or disorder associated with or mediated by PRAME. For example, the present disclosure provides methods for treating a PRAME-associated disease or disorder, such as a PRAME-associated cancer (e.g., a PRAME-positive cancer) (tumor growth inhibition) by administering a PRAME TCR (or pharmaceutical composition comprising a PRAME TCR or a plurality of cells comprising a PRAME TCR or PRAME bi-specific molecules) as described herein to a patient in need of such treatment, and PRAME TCRs (or pharmaceutical composition comprising a PRAME TCR) for use in the treatment of a PRAME-associated cancer. The antigen-binding proteins of the present disclosure may be useful for the treatment, prevention, and/or amelioration of disease or disorder or condition such as a PRAME-associated cancer and/or for ameliorating at least one symptom associated with such disease, disorder or condition. In the context of the methods of treatment described herein, the PRAME TCR (or pharmaceutical composition or plurality of cells or PRAME bi-specific molecules) may be administered as a monotherapy (e.g., as the only therapeutic agent) or in combination with one or more additional therapeutic agents (examples of which are described elsewhere herein).

Accordingly, the present disclosure provides for methods of treating an individual diagnosed with or suspected of having, or at risk of developing, a PRAME-associated disease or disorder, e.g., a PRAME-associated cancer, comprising administering the individual a therapeutically effective amount of the TCR-expressing immune effector cells as described herein.

In some embodiments, the present disclosure provides a method of treating a subject diagnosed with a PRAME-positive cancer comprising removing immune effector cells from a subject diagnosed with a PRAME-positive cancer, genetically modifying said immune effector cells with a vector comprising a nucleic acid encoding a TCR of the present disclosure, thereby producing a population of modified immune effector cells, and administering the population of modified immune effector cells to the same subject. In some embodiments, the immune effector cells comprise T cells.

The methods for administering the cell compositions described herein may include any method which may be effective to result in reintroduction of ex vivo genetically modified immune effector cells that either directly express a TCR of the present disclosure in the subject or on reintroduction of the genetically modified progenitors of immune effector cells that on introduction into a subject differentiate into mature immune effector cells that express the TCR. One method may include transducing peripheral blood T cells ex vivo with a nucleic acid construct in accordance with the present disclosure and returning the transduced cells into the subject.

In some embodiments of the present disclosure, the compositions described herein may be useful for treating subjects suffering from primary or recurrent cancer, including, but not limited to, PRAME-associated cancer, e.g., PRAME-associated cancer may be a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, or a recurrent non-small cell lung cancer. In some embodiments, the PRAME-associated cancer is an ovarian cancer, a melanoma, a non-small cell lung carcinoma, a hepatocellular carcinoma, a colorectal carcinoma, an esophageal squamous cell carcinoma, an esophageal adenocarcinoma, a stomach cancer, a bladder cancer, a head and neck cancer, a gastric cancer, a synovial sarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a myxoid round cell liposarcoma.

The TCRs may be used to treat early stage or late-stage symptoms of the PRAME- associated cancer. In some embodiments, PRAME TCRs or PRAME bi-specific molecules of the present disclosure may be used to treat advanced or metastatic cancer. The PRAME TCRs or PRAME bi-specific molecules may be useful in reducing or inhibiting or shrinking tumor growth. In certain embodiments, treatment with PRAME TCRs or PRAME bi-specific molecules of the present disclosure may lead to more than 40% regression, more than 50% regression, more than 60% regression, more than 70% regression, more than 80% regression or more than 90% regression of a tumor in a subject. In certain embodiments, the TCRs may be used to prevent relapse of a tumor. In certain embodiments, the PRAME TCRs or PRAME bi-specific molecules may be useful in extending progression-free survival or overall survival in a subject with PRAME-associated cancer. In some embodiments, the PRAME TCRs or PRAME bi-specific molecules may be useful in reducing toxicity due to chemotherapy or radiotherapy while maintaining long term survival in a patient suffering from PRAME-associated cancer.

One or more PRAME TCRs or PRAME bi-specific molecules of the present disclosure may be administered to relieve or prevent or decrease the severity of one or more of the symptoms or conditions of the disease or disorder.

It is also contemplated herein to use one or more PRAME TCRs or PRAME bi-specific molecules of the present disclosure prophylactically to patients at risk for developing a disease or disorder such as PRAME-associated disease or disorder, such as a PRAME-associated cancer.

In further embodiments of the present disclosure, the present PRAME TCRs or PRAME bi-specific molecules may be used for the preparation of a pharmaceutical composition for treating patients suffering from PRAME-associated disease or disorder, such as a PRAME-associated cancer. In some embodiments of the present disclosure, the present PRAME TCRs or PRAME bi-specific molecules may be used as adjunct therapy with any other agent or any other therapy known to those skilled in the art useful for treating PRAME-associated cancer.

Combination therapies may include PRAME TCRs or PRAME bi-specific molecules of the present disclosure, such as immune effector cell comprising PRAME TCRs or PRAME bi-specific molecules of the present disclosure, or a pharmaceutical composition of the present disclosure, and any additional therapeutic agent that may be advantageously combined with PRAME TCRs or PRAME bi-specific molecules of the present disclosure. PRAME TCRs or PRAME bi-specific molecules of the present disclosure may be combined synergistically with one or more anti-cancer drugs or therapy used to treat or inhibit a PRAME-associated disease or disorder, such as PRAME-positive cancer, e.g., a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

It is contemplated herein to use PRAME TCRs or PRAME bi-specific molecules of the present disclosure in combination with immuno stimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more PRAME TCRs or PRAME bi-specific molecules of the present disclosure may be used in combination with a PD-1 inhibitor (e.g., an anti-PD-1 antibody such as nivolumab, pembrolizumab, pidilizumab, BGB-A317 or REGN2810), a PD-L1 inhibitor (e.g., an anti-PD-LI antibody such as avelumab, atezolizumab, durvalumab, MDX-1105, or REGN3504 ), a CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2, 3-dioxygenase (IDO) inhibitor, a vascular endothelial growth factor (VEGF) antagonist, e.g., a “VEGF-Trap” such as aflibercept or other VEGF-inhibiting fusion protein as set forth in US 7,087,411, or an anti -VEGF antibody or antigen-binding fragment thereof (e.g., bevacizumab, or ranibizumab) or a small molecule kinase inhibitor of VEGF receptor (e.g., sunitinib, sorafenib, or pazopanib), an Ang2 inhibitor (e.g., nesvacumab), a transforming growth factor beta (TGFβ) inhibitor, an epidermal growth factor receptor (EGFR) inhibitor (e.g., erlotinib, cetuximab), an NY-ESO-1 inhibitor (e.g., an anti-NY-ESO-1 antibody), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a costimulatory agent, a bispecific antibody (e.g., CD3xCD20 bispecific antibody, a PSMAxCD3 bispecific antibody, or a bispecific antibody that acts as a costimulatory agent, such as a bispecific antibody that binds a tumor antigen and has costimulatory activity), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, surgery, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any other therapy care to treat cancer. In certain embodiments, the TCRs of the present disclosure may be used in combination with cancer vaccines including dendritic cell vaccines, oncolytic viruses, tumor cell vaccines, etc. to augment the anti-tumor response.

Examples of cancer vaccines that can be used in combination with PRAME TCRs or PRAME bi-specific molecules of the present disclosure may include MAGE3 vaccine for melanoma and bladder cancer, MUC1 vaccine for breast cancer, EGFRv3 (e.g., Rindopepimut) for brain cancer (including glioblastoma multiforme), ALVAC-CEA (for CEA+ cancers), and NY-ESO-1 vaccine (e.g., for melanoma).

In certain embodiments, PRAME TCRs or PRAME bi-specific molecules of the present disclosure may be administered in combination with radiation therapy in methods to generate long-term durable anti-tumor responses and/or enhance survival of patients with cancer. In some embodiments, PRAME TCRs or PRAME bi-specific molecules of the present disclosure may be administered prior to, concomitantly or after administering radiation therapy to a cancer patient. For example, radiation therapy may be administered in one or more doses to tumor lesions followed by administration of one or more doses of PRAME TCRs or PRAME bi-specific molecules of the present disclosure. In some embodiments, radiation therapy may be administered locally to a tumor lesion to enhance the local immunogenicity of a patient’s tumor (adjuvinating radiation) and/or to kill tumor cells (ablative radiation) followed by systemic administration of PRAME TCRs or PRAME bi-specific molecules of the present disclosure.

The additional therapeutically active agent(s)/component(s) may be administered prior to, concurrent with, or after the administration of PRAME TCRs or PRAME bi-specific molecules of the present disclosure. For purposes of the present disclosure, such administration regimens may be considered the administration of PRAME TCRs or PRAME bi-specific molecules “in combination with” a second therapeutically active component.

The additional therapeutically active component(s) may be administered to a subject prior to administration of PRAME TCRs or PRAME bi-specific molecules of the present disclosure. In other embodiments, the additional therapeutically active component(s) may be administered to a subject after administration of PRAME TCRs or PRAME bi-specific molecules of the present disclosure. In yet other embodiments, the additional therapeutically active component(s) may be administered to a subject concurrent with administration of PRAME TCRs or PRAME bi-specific molecules of the present disclosure. “Concurrent” administration, for purposes of the present disclosure, may include, e.g., administration of PRAME TCRs or PRAME bi-specific molecules and an additional therapeutically active component to a subject in a single dosage form (e.g., co-formulated), or in separate dosage forms administered to the subject within about 30 minutes or less of each other. If administered in separate dosage forms, each dosage form may be administered via the same route; alternatively, each dosage form may be administered via a different route. In any event, administering the components in a single dosage from, in separate dosage forms by the same route, or in separate dosage forms by different routes are all considered “concurrent administration,” for purposes of the present disclosure. For purposes of the present disclosure, administration of PRAME TCRs or PRAME bi-specific molecules “prior to”, “concurrent with,” or “after” (as those terms are defined herein above) administration of an additional therapeutically active component may be considered administration of PRAME TCRs or PRAME bi-specific molecules “in combination with” an additional therapeutically active component).

### Methods of Administration

One or multiple engineered αβ T-cells and/or γδ T-cells populations may be administered to a subject in any order or simultaneously. If simultaneously, the multiple engineered αβ T-cells and/or γδ T-cells can be provided in a single, unified form, such as an intravenous injection, or in multiple forms, for example, as multiple intravenous infusions, s.c, injections or pills. Engineered γδ T-cells can be packed together or separately, in a single package or in a plurality of packages. One or all of the engineered αβ T-cells and/or γδ T-cells can be given in multiple doses. If not simultaneous, the timing between the multiple doses may vary to as much as about a week, a month, two months, three months, four months, five months, six months, or about a year. In another aspect, engineered αβ T-cells and/or γδ T-cells can expand within a subject’s body, in vivo, after administration to a subject. Engineered αβ T-cells and/or γδ T-cells can be frozen to provide cells for multiple treatments with the same cell preparation. Engineered αβ T-cells and/or γδ T-cells of the present disclosure, and pharmaceutical compositions comprising the same, can be packaged as a kit. A kit may include instructions (e.g., written instructions) on the use of engineered αβ T-cells and/or γδ T-cells and compositions comprising the same.

In another aspect, a method of treating a cancer comprises administering to a subject a therapeutically-effective amount of engineered αβ T-cells and/or γδ T-cells, in which the administration treats the cancer. In another embodiments, the therapeutically-effective amount of engineered αβ T-cells and/or γδ T-cells may be administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. In another aspect, the therapeutically-effective amount of the engineered αβ T-cells and/or γδ T-cells may be administered for at least one week. In another aspect, the therapeutically-effective amount of engineered αβ T-cells and/or γδ T-cells may be administered for at least two weeks.

Engineered αβ T-cells and/or γδ T-cells described herein can be administered before, during, or after the occurrence of a disease or condition, and the timing of administering a pharmaceutical composition containing an engineered αβ T-cells and/or γδ T-cell can vary. For example, engineered αβ T-cells and/or γδ T-cells can be used as a prophylactic and can be administered continuously to subjects with a propensity to conditions or diseases in order to lessen the likelihood of occurrence of the disease or condition. Engineered αβ T-cells and/or γδ T-cells can be administered to a subject during or as soon as possible after the onset of the symptoms. The administration of engineered αβ T-cells and/or γδ T-cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In another aspect, the administration of engineered αβ T-cells and/or γδ T-cells of the present disclosure may be an intravenous administration. One or multiple dosages of engineered αβ T-cells and/or γδ T-cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered αβ T-cells and/or γδ T-cells can be administered years after onset of the cancer and before or after other treatments. In another aspect, engineered αβ T-cells and/or γδ T-cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

### Preservation

In an aspect, αβ T-cells and/or γδ T-cells may be formulated in freezing media and placed in cryogenic storage units such as liquid nitrogen freezers (-196° C.) or ultra-low temperature freezers (-65° C., -80° C., -120° C., or -150° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroxyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved αβ T-cells and/or γδ T-cells can be thawed and further processed by stimulation with antibodies, proteins, peptides, and/or cytokines as described herein. The cryopreserved αβ T-cells and/or γδ T-cells can be thawed and genetically modified with viral vectors (including retroviral, adeno-associated virus (AAV), and lentiviral vectors) or non-viral means (including RNA, DNA, e.g., transposons, and proteins) as described herein. The modified αβ T-cells and/or γδ T-cells can be further cryopreserved to generate cell banks in quantities of at least about 1, 5, 10, 100, 150, 200, 500 vials at about at least 101, 102, 103, 104, 105, 106, 107, 108, 109, or at least about 1010 cells per mL in freeze media. The cryopreserved cell banks may retain their functionality and can be thawed and further stimulated and expanded. In another aspect, thawed cells can be stimulated and expanded in suitable closed vessels, such as cell culture bags and/or bioreactors, to generate quantities of cells as allogeneic cell product. Cryopreserved αβ T-cells and/or γδ T-cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In another aspect, no preservatives may be used in the formulation. Cryopreserved αβ T-cells and/or γδ T-cells can be thawed and infused into multiple patients as allogeneic off-the-shelf cell product.

In an aspect, engineered αβ T-cells and/or γδ T-cell described herein may be present in a composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

In an aspect, methods described herein may be used to produce autologous or allogenic products according to an aspect of the disclosure.

According to one embodiment of the present disclosure, the antibody according to the above description or the T-cell receptor according to the above description further comprises an effector moiety, selected from the group consisting of
a) toxin, or
b) immune modulator.

Immune modulators are known. They are molecules which induce or stimulate an immune response, through direct or indirect activation of the humoural or cellular arm of the immune system, such as by activation of T-cells. Examples include: IL-1, IL-1α, IL-3, IL-4, IL-5, IL-6, IL-7, IL-10, IL-11, IL-12, IL-13, IL-15, IL-21, IL-23, TGF-β, IFN-γ, TNFα, Anti-CD2 antibody, Anti-CD3 antibody, Anti-CD4 antibody, Anti-CD8 antibody, Anti-CD44 antibody, Anti-CD45RA antibody, Anti-CD45RB antibody, Anti-CD45RO antibody, Anti-CD49a antibody, Anti-CD49b antibody, Anti-CD49c antibody,Anti-CD49d antibody,Anti-CD49e antibody, Anti-CD49f antibody, Anti-CD16 antibody, Anti-CD28 antibody, Anti-IL-2R antibodies, Viral proteins and peptides, and Bacterial proteins or peptides. Where the immune modulator polypeptide is an antibody it may specifically bind to an antigen presented by a T-cell and may be an scFv antibody.

In one embodiment, the immune modulator is an anti CD3 antibody.

In one embodiment, the immune modulator binds to CD3γ, CD3δ, or CD3ε.

In one embodiment, the immune modulator is the anti CD3 antibody OKT3.

In one embodiment, the immune modulator is the anti CD3 antibody UCHT-1, or its humanized variant hUCHT-1.

In one embodiment, the immune modulator is the anti CD3 antibody BMA031.

In one embodiment, the immune modulator is the anti CD3 antibody 12F6.

In several embodiments,, fragments, like e.g. the V_{H} and V_{L} domains, of these antibodies can be used. The skilled person is aware of how to derive, from a published antibody, its V_{H} and V_{L} domains.

Humanized antibody hUCHT1 is disclosed in Zhu et al., Identification of heavy chain residues in a humanized anti-CD3 antibody important for efficient antigen binding and T-cell activation. J Immunol, 1995, 155, 1903-1910, the content of which is incorporated herein by reference. In particular V_{H} and V_{L} domains derived from the UCHT1 variants UCHT1-V17, UCHT1-V17opt, UCHT1-V21 or UCHT1-V23 can be used, preferably derived from UCHT1-V17. Further preferred embodiments and variants of this antibody are disclosed elsewhere herein.

Antibody BMA031, which targets the TCRα/β CD3 complex, and humanized versions thereof, is disclosed in Shearman et al., Construction, expression and characterization of humanized antibodies directed against the human alpha/beta T-cell receptor, J Immunol, 1991, 147, 4366-73). In particular V_{H} and V_{L} domains derived from BMA031 variants BMA031(V36) or BMA031(V10), preferably derived from BMA031(V36) can be used. Further preferred embodiments and variants of this antibody are disclosed elsewhere herein.

In further embodiments, the immune modulator binds to a cell surface antigen selected from the group consisting of CD4, CD7, CD8, CD10, CD11b, CD11c, CD14, CD16, CD18, CD22, CD25, CD28, CD32a, CD32b, CD33, CD41, CD41b, and/or CD42a.

Toxins to be used to couple with targeting domain are also known. See, e.g., Storz U. Antibody-drug conjugates: Intellectual property considerations. MAbs. 2015;7(6):989-1009. doi: 10.1080/19420862.2015.1082019, the content of which is incorporated herein by reference.

In one embodiment, the toxin is an Auristatin (MMAE, MMAF).

In one embodiment, the toxin is a Maytansinoid,

In one embodiment, the toxin is an Anthracyclin or derivative thereof.

In one embodiment, the toxin is a Calicheamicin.

In one embodiment, the toxin is a Duocarmycin.

In one embodiment, the toxin is a Taxane.

In one embodiment, the toxin is a Pyrrolobenzodiazepine.

In one embodiment, the toxin is a α-Amanitin.

In one embodiment, the toxin is a ribotoxin or RNase.

In one embodiment, the toxin is a Tubulysin.

In one embodiment, the toxin is a Benzodiazepine derivative

According to one embodiment of the present disclosure, a T-cell receptor according to the description above is provided for use in the (manufacture of a medicament for the) treatment of a patient (i) being diagnosed for, (ii) suffering from or (iii) being at risk of developing recurrent cancer.

The T-cell receptor comprises a first polypeptide chain and a second polypeptide chain, wherein said first polypeptide chain comprising 95% identity to any one of
SEQ ID NOs 178, 184, 187, 189, 190, 192, 195, 197, 200, 206, 208, 210, 212, 216, 218, 219, 220, 221, 222, 229, 230, 232, 234, 236, 238, 240, 241, 242, 243, 244, 246, 250, 251, 252, 253, 254, 255, 256, 257, 258, 259, 260, 261, 262, 263, 265, 298, 299, 300, 302, or 304
comprises the complementarity determining regions (CDRs) of said sequence; wherein the second polypeptide chain comprises a second hinge domain and/or a second Fc domain, wherein said second polypeptide comprising 95% identity to any one of SEQ ID NOs 179, 180, 181, 182, 183, 185, 186, 188, 191, 193, 194, 196, 198, 199, 201, 202, 203, 204, 205, 207, 209, 211, 213, 214, 215, 217, 223, 224, 225, 226, 227, 228, 231, 233, 235, 237, 239, 245, 247, 248, 249, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 301, or 303 comprises the CDRs of said sequence.

Alternatively or in addition, a method of treating a patient (i) being diagnosed for, (ii) suffering from or (iii) being at risk of developing recurrent cancer, is provided.

The method comprises administering to the patient a T-cell receptor comprising a first polypeptide chain and a second polypeptide chain, wherein said first polypeptide chain comprising 95% identity to any one of SEQ ID NOs 178, 184, 187, 189, 190, 192, 195, 197, 200, 206, 208, 210, 212, 216, 218, 219, 220, 221, 222, 229, 230, 232, 234, 236, 238, 240, 241, 242, 243, 244, 246, 250, 251, 252, 253, 254, 255, 256, 257, 258, 259, 260, 261, 262, 263, 265, 298, 299, 300, 302, or 304

In an aspect, methods described herein comprise the complementarity determining regions (CDRs) of said sequence; wherein the second polypeptide chain comprises a second hinge domain and/or a second Fc domain, wherein said second polypeptide comprising 95% identity to any one of SEQ ID NOs 179, 180, 181, 182, 183, 185, 186, 188, 191, 193, 194, 196, 198, 199, 201, 202, 203, 204, 205, 207, 209, 211, 213, 214, 215, 217, 223, 224, 225, 226, 227, 228, 231, 233, 235, 237, 239, 245, 247, 248, 249, 264, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 301, or 303.

The said sequences are T cell receptor variable domains. The CDRs of a T cell receptor variable domain can be determined based on Lefranc, M.-P. et al., Dev. Comp. Immunol., 27, 55-77 (2003), the content of which is incorporated herein by reference. Further disclosure can be found in imgt.org/IMGTScientificChart/Numbering/IMGTIGVLsuperfamily.html

Alternatively or in addition, a pharmaceutical composition for treating recurrent cancer is provided, comprising such T cell receptor as an effective ingredient.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

In one embodiment, said first polypeptide chain is fused to said second polypeptide chain by covalent and/or non-covalent bonds between the first hinge domain and the second hinge domain, and/or between the first Fc domain and the second Fc domain.

In one embodiment, said first polypeptide chain is fused to said second polypeptide chain by covalent and/or non-covalent bonds between the first hinge domain and the second hinge domain, and/or between the first Fc domain and the second Fc domain

In one embodiment, said first and second Fc domains each comprise at least one Fc effector function silencing mutation.

For example, the Fc domain on one or both, preferably both polypeptide chains can comprise one or more alterations that inhibit Fc gamma receptor (FcyR) binding. Such alterations can include L234A, L235A.

In a further embodiment, the Fc domain on one or both, preferably both polypeptide chains can comprise a N297Q mutation to remove the N-glycosylation site within the Fc-part. Such a mutation abrogates the Fc-gamma-receptor interaction.

In one embodiment, said first and second Fc domains each comprise a CH3 domain comprising at least one mutation that facilitates the formation of heterodimers.

Accordingly, in some embodiments, the Fc domain of one of the polypeptides, for example Fc1, comprises the amino acid substitutions S354C and T366W (knob) in its CH3 domain and the Fc domain of the other polypeptide, for example Fc2, comprises the amino acid substitution Y349C, T366S, L368A and Y407V (hole) in its CH3 domain, or vice versa. This set of amino acid substitutions can be further extended by inclusion of the amino acid substitutions K409A on one polypeptide and F405K in the other polypeptide as described by Wei et al. (Structural basis of a novel heterodimeric Fc for bispecific antibody production, Oncotarget. 2017). Accordingly, in some embodiments, the Fe domain of one of the polypeptides, for example Fc1, comprises or further comprises the amino acid substitution K409A in its CH3 domain and the Fc domain of the other polypeptide, for example Fe2, comprises or further the amino acid substitution F405K in its CH3 domain, or vice versa.

Accordingly, in one embodiment, the Fe domain of one of the polypeptides, for example Fc1, comprises or further comprises the charge pair substitutions E356K, E356R, D356R, or D356K and D399K or D399R, and the Fc domain of the other polypeptide, for example Fc2, comprises or further comprises the charge pair substitutions R409D, R409E, K409E, or K409D and N392D, N392E, K392E, or K392D, or vice versa.

In one embodiment, said first and second Fc domains each comprise CH2 and CH3 domains comprising at least two additional cysteine residues.

Such cysteine residues may result into the formation of Cystein bridges, which may improve the stability of the antigen binding proteins, optimally without interfering with the binding characteristics of the antigen binding proteins. Such cysteine bridges can further improve heterodimerization. Further amino acid substitutions, such as charged pair substitutions, have been described in the art, for example in EP2970484 to improve the heterodimerization of the resulting proteins.

Some embodiments of the present disclosure may include methods of treating a recurrent cancer that presents a peptide comprising, consisting essentially of, or consisting of a peptide described herein, for example in Table 10, a PRAME peptide such as SLLQHLIGL (SEQ ID NO: 310), a MAG-003 peptide, a MAGEA1-003 peptide, a COL6A3 peptide, or a peptide from the MAGE peptide class, including, for example: identifying a recurrent cancer and administering a T lymphocyte of the present disclosure or activated T lymphocytes produced by methods described herein to the recurrent cancer, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Some embodiments of the present disclosure may include methods of treating a recurrent cancer that presents a peptide comprising, consisting essentially of, or consisting of a peptide described herein, for example in Table 10, a PRAME peptide such as SLLQHLIGL (SEQ ID NO: 310), a MAG-003 peptide, a MAGEA1-003 peptide, a COL6A3 peptide, or a peptide from the MAGE peptide class, including, for example: identifying a recurrent cancer and treating the recurrent cancer with a population of T lymphocytes that bind to and/or are specific for a peptide comprising, consisting essentially of, or consisting of a peptide described herein, for example in Table 10, a PRAME peptide such as SLLQHLIGL (SEQ ID NO: 310), a MAG-003 peptide, a MAGEA1-003 peptide, a COL6A3 peptide, or a peptide from the MAGE peptide class, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Other embodiments of the present disclosure may include methods of treating a recurrent cancer that presents a peptide comprising, consisting essentially of, or consisting of a peptide described herein, for example in Table 10, a PRAME peptide such as SLLQHLIGL (SEQ ID NO: 310), a MAG-003 peptide, a MAGEA1-003 peptide, a COL6A3 peptide, or a peptide from the MAGE peptide class, including, for example: treating the recurrent cancer with a population of T lymphocytes that bind to and/or are specific for a peptide in Table 10, a PRAME peptide such as SLLQHLIGL (SEQ ID NO: 310), a MAG-003 peptide, a MAGEA1-003 peptide, a COL6A3 peptide, or a peptide from the MAGE peptide class, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Other embodiments of the present disclosure may include methods of treating a recurrent cancer that presents a peptide from, for example Table 10, a PRAME peptide such as SLLQHLIGL (SEQ ID NO: 310), a MAG-003 peptide, a MAGEA1-003 peptide, a COL6A3 peptide, or a peptide from the MAGE peptide class on the cell surface, including, for example: selecting a patient having a recurrent cancer and administering to the patient a composition comprising a T lymphocyte of the present disclosure or the activated T lymphocytes produced by methods described herein, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Some embodiments of the present disclosure may include methods of eliciting an immune response to a recurrent cancer that presents a peptide from, for example Table 10, a PRAME peptide such as SLLQHLIGL (SEQ ID NO: 310), a MAG-003 peptide, a MAGEA1-003 peptide, a COL6A3 peptide, or a peptide from the MAGE peptide class, including, for example: identifying a recurrent cancer and administering a T lymphocyte of the present disclosure or activated T lymphocytes produced by methods described herein in the recurrent cancer, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Some embodiments of the present disclosure may include methods of eliciting an immune response to a recurrent cancer that presents a peptide from, for example Table 10, a PRAME peptide such as SLLQHLIGL (SEQ ID NO: 310), a MAG-003 peptide, a MAGEA1-003 peptide, a COL6A3 peptide, or a peptide from the MAGE peptide class, including, for example: identifying a recurrent cancer and treating the recurrent cancer with a population of T lymphocytes that binds to and/or are specific for a peptide from, for example Table 10, a PRAME peptide such as SLLQHLIGL (SEQ ID NO: 310), a MAG-003 peptide, a MAGEA1-003 peptide, a COL6A3 peptide, or a peptide from the MAGE peptide class, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Other embodiments of the present disclosure may include methods of eliciting an immune response to a recurrent cancer that present a peptide from, for example Table 10, a PRAME peptide such as SLLQHLIGL (SEQ ID NO: 310), a MAG-003 peptide, a MAGEA1-003 peptide, a COL6A3 peptide, or a peptide from the MAGE peptide class on the cell surface, including, for example: selecting a patient having a recurrent cancer and administering to the patient a composition comprising a T lymphocyte of the present disclosure or the activated T lymphocytes produced by methods described herein, wherein the recurrent cancer originates from a cancer selected from the group consisting of non-small cell lung cancer, small cell lung cancer, melanoma, mesothelioma, breast cancer, primary brain cancer, ovarian cancer, uterine carcinoma, head and neck squamous cell carcinomas, colon cancer, gastro-intestinal cancer, renal cell carcinoma, sarcoma, germ cell tumor, lymphoma, testicular cancer, bladder cancers, prostate cancer, oral cavity carcinomas, oral squamous carcinoma, acute myeloid leukemia, H. pylori-induced MALT Non-Hodgkin’s lymphoma, glioblastoma, cervical carcinoma, hepatocellular carcinoma, Ewing’s sarcoma, endometrial cancer, epithelial cancer of the larynx, esophageal carcinoma, oral carcinoma, atypical meningioma, papillary thyroid carcinoma, brain tumors, salivary duct carcinoma, and extranodal T/NK-cell lymphomas.

Some embodiments of the present disclosure may include administering to a patient at least one adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23).

Some embodiments of the present disclosure may include methods of preparing a T cell population comprising: obtaining the T cell population from PBMCs; activating the obtained T cell population, transducing the activated T cell population with the nucleic acid of the present disclosure, expanding the transduced T cell population, and wherein the activating, transducing, and expanding are performed in the presence of IL-21 with or without a histone deacetylase inhibitor (HDACi).

In one embodiment, the present disclosure provide a method for reprogramming antigen- specific effector T cells (T_{EEF} cells) into central memory T cells (T_{CM} cells), the method may include obtaining a starting population of lymphocytes comprising T_{EEF} cells from a subject; optionally preparing a sample enriched in T_{EEF} cells from the starting population of lymphocytes comprising T_{EEF} cells; and culturing the starting population of lymphocytes comprising T_{EEF} cells or the sample enriched in T_{EEF} cells in the presence of a histone deacetylase inhibitor (HDACi) and interleukin-21 (IL-21), each in an amount sufficient to re program the T_{EEF} cells into T_{CM} cells, wherein the re-programming produces a population of lymphocytes enriched for T_{CM} cells as compared to the number of T_{CM} cells in the starting population of lymphocytes comprising T_{EEF} cells obtained from a subject.

In some embodiments, obtaining a starting population of lymphocytes comprising T_{EEF} cells may include taking a sample of tumor infiltrating lymphocytes (TILs) or a sample containing peripheral blood mononuclear cells (PBMCs) from a subject. In some embodiments, the method may further include the step of preparing a sample enriched in T_{EEF} cells from the starting population of lymphocytes comprising T_{EEF} cells. In some embodiments, the step of preparing a sample enriched in T_{EEF} cells from the starting population of lymphocytes comprising T_{EEF} cells may include isolating CD8⁺ T_{EEF} cells from the starting population of lymphocytes containing T_{EEF} cells.

In some embodiments, IL-21, a histone deacetylase inhibitor (HDACi), or combinations thereof may be utilized in the field of cancer treatment, with methods described herein, and/or with ACT processes described herein. In an embodiment, the present disclosure provides methods for re-programming effector T cells to a central memory phenotype comprising culturing the effector T cells with at least one HDACi together with IL-21. Representative HDACi include, for example, trichostatin A, trapoxin B, phenylbutyrate, valproic acid, vorinostat (suberanilohydroxamic acid or SAHA), belinostat, panobinostat, dacinostat, entinostat, tacedinaline, and mocetinostat. In particular aspects, the HDACi may be SAHA. In other aspects, the HDAC may be panobinostat.

### Bi-Specific Molecules Against Peptides Described Herein

In an aspect, molecules of the present disclosure comprise a first polypeptide chain and a second polypeptide chain, wherein the chains jointly provide a variable domain of an antibody specific for an epitope of an immune modulator cell surface antigen, and a variable domain of a TCR that is specific for an MHC-associated peptide epitope, e.g., SLLQHLIGL (PRAME-004) (SEQ ID NO: 310), a peptide from, for example Table 10, a PRAME peptide, a MAG-003 peptide, a MAGEA1-003 peptide, a COL6A3 peptide, or a peptide from the MAGE peptide class. Antibody and TCR-derived variable domains are stabilized by covalent and non-covalent bonds formed between Fc-parts or portions thereof located on both polypeptide chains. The dual specificity polypeptide molecule is then capable of simultaneously binding the cellular receptor and the MHC-associated peptide epitope.

As discussed, a variable domain of an antibody may specifically bind an epitope of an immune modulator cell surface antigen at least one selected from the group consisting of CD3γ, CD3δ, CD3ε, CD3ζ, CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, FcεRI, TCRα/β, TCRγ/δ, and HLA-DR.

In the context of the present disclosure, variable domains are derived from antibodies capable of recruiting human immune modulator cells by specifically binding to a surface antigen of said effector cells. In one particular embodiment, said antibodies specifically bind to epitopes of the TCR-CD3 complex of human T-cells, comprising the peptide chains TCRalpha, TCRbeta, CD3gamma, CD3delta, CD3epsilon, and CD3zeta.

In the context of the present disclosure, the dual affinity polypeptide molecule described herein may bind to SLLQHLIGL peptide (SEQ ID NO: 310) when presented as a peptide-MHC complex. In another aspect, the dual affinity polypeptide molecule described herein may bind to a PRAME peptide, a MAG-003 peptide, a MAGEA1-003 peptide, a COL6A3 peptide, or a peptide from the MAGE peptide class

For example, dual affinity polypeptide molecules of the present disclosure may include those disclosed in US20190016801, US20190016802, US20190016803, and US20190016804, the contents of which are herein incorporated by reference in their entireties.

Preferably, the dual specificity polypeptide molecule according to the present disclosure binds with high specificity to both the immune modulator cell antigen and a specific antigen epitope presented as a peptide-MHC complex, e.g., with a binding affinity (KD) of about 100 nM or less, about 30 nM or less, about 10 nM or less, about 3 nM or less, about 1 nM or less, e.g. measured by Bio-Layer Interferometry or as determined by flow cytometry.

Preferred is a dual specificity polypeptide molecule according to the present disclosure, wherein a knob-into-hole mutation is selected from T366W as knob, and T366′S, L368′A, and Y407′V as hole in the CH3 domain (see, e.g., WO 98/50431). This set of mutations can be further extended by inclusion of the mutations K409A and F405′K as described by Wei et al. (Structural basis of a novel heterodimeric Fc for bispecific antibody production, Oncotarget. 2017). Another knob can be T366Y and the hole is Y407′T.

Engineering was performed to incorporate knob-into-hole mutations into CH3-domains with and without additional interchain disulfide bond stabilization; to remove an N-glycosylation site in CH2 (e.g. N297Q mutation); to introduce Fc-silencing mutations; to introduce additional disulfide bond stabilization into VL and VH, respectively, according to the methods described by Reiter et al. (Stabilization of the Fv Fragments in Recombinant Immunotoxins by Disulfide Bonds Engineered into Conserved Framework Regions. Biochemistry, 1994, 33, 5451 - 5459). An overview of produced bispecific TCR/mAb diabodies, the variants as well as the corresponding sequences are listed in Tables 3b and 7.

Preferred is the dual specificity polypeptide molecule according to the present disclosure, wherein said first and second polypeptide chains further comprise at least one hinge domain and/or an Fc domain or portion thereof. In antibodies, the “hinge” or “hinge region” or “hinge domain” refers to the flexible portion of a heavy chain located between the CH1 domain and the CH2 domain. It is approximately 25 amino acids long, and is divided into an “upper hinge,” a “middle hinge” or “core hinge,” and a “lower hinge.” A “hinge subdomain” refers to the upper hinge, middle (or core) hinge or the lower hinge. The amino acids sequence of the hinges of an IgG1 molecule is IgG1: EPKSCDKTHTCPPCPAPELLG (SEQ ID NO: 129), with E being E216 according to EU (imgt.org/IMGTScientificChart/Numbering/Hu_IGHGnber.html) numbering.

Preferred is a dual specificity polypeptide molecule according to the present disclosure, comprising at least one IgG fragment crystallizable (Fc) domain, i.e., a fragment crystallizable region (Fc region), the tail region of an antibody that interacts with Fc receptors and some proteins of the complement system. Fc regions contain two or three heavy chain constant domains (CH domains 2, 3, and 4) in each polypeptide chain. The Fc regions of IgGs also bear a highly conserved N-glycosylation site. Glycosylation of the Fc fragment is essential for Fc receptor-mediated activity. The small size of bispecific antibody formats such as BiTEs® and DARTs (~50 kD) can lead to fast clearance and a short half-life. Therefore, for improved pharmacokinetic properties, the TCR variable only regions (scTv)-cellular receptor (e.g., CD3) dual specificity polypeptide molecule can be fused to a (human IgG1) Fc domain, thereby increasing the molecular mass. Several mutations located at the interface between the CH2 and CH3 domains, such as T250Q/M428L and M252Y/S254T/T256E + H433K/N434F, have been shown to increase the binding affinity to neonatal Fc receptor (FcRn) and the half-life of IgG1 in vivo. By this the serum half-life of an Fc-containing molecule could be further extended.

In the dual specificity polypeptide molecules of the present disclosure, said Fc domain can comprises a CH2 domain comprising at least one Fc effector function silencing mutation. Preferably, these mutations are introduced into the ELLGGP (SEQ ID NO: 130) sequence of human IgG1 (residues 233-238) or corresponding residues of other isotypes) known to be relevant for effector functions. In principle, one or more mutations corresponding to residues derived from IgG2 and/or IgG4 are introduced into IgG1 Fc. Preferred are: E233P, L234V, L235A and no residue or G in position 236. Another mutation is P331S. EP1075496 discloses a recombinant antibody comprising a chimeric domain which is derived from two or more human immunoglobulin heavy chain CH2 domains, which human immunoglobulins are selected from IgG1, IgG2 and IgG4,and wherein the chimeric domain is a human immunoglobulin heavy chain CH2 domain which has the following blocks of amino acids at the stated positions: 233P, 234V, 235A and no residue or G in position 236 and 327G, 330S and 331S in accordance with the EU numbering system, and is at least 98% identical to a CH2 sequence (residues 231-340) from human IgG1, IgG2 or IgG4 having said modified amino acids.

The inventive dual specificity polypeptide molecules according to the present disclosure are exemplified here by a dual specificity polypeptide molecule comprising a first polypeptide chain comprising SEQ ID NO: 131 and a second polypeptide chain comprising SEQ ID NO: 132, or a dual specificity polypeptide molecule comprising a first polypeptide chain comprising SEQ ID NO: 133 and a second polypeptide chain comprising SEQ ID NO: 134.

In an aspect, the disclosure provides for a polypeptide having at least 50%, at least 60%, at least 70%, at least 80%, at least 90%, at least 95%, at least 96%, at least 97%, at least 98%, or at least 99% sequence identity to the amino acid sequence of SEQ ID NO: 131, 132, 133 or 134.

In another aspect, the polypeptides or dual specific polypeptide molecules as disclosed herein can be modified by the substitution of one or more residues at different, possibly selective, sites within the polypeptide chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

In another aspect of the present disclosure, the above object is solved by providing a nucleic acid(s) encoding for a first polypeptide chain and/or a second polypeptide chain as disclosed herein, or expression vector(s) comprising such nucleic acid.

In another aspect of the present disclosure, the above object is solved by providing a host cell comprising vector(s) as defined herein.

In another aspect of the present disclosure, the above object is solved by providing a method for producing a dual specificity polypeptide molecule according to the present disclosure, comprising suitable expression of said expression vector(s) comprising the nucleic acid(s) as disclosed in a suitable host cell, and suitable purification of the molecule(s) from the cell and/or the medium thereof.

In another aspect of the present disclosure, the above object is solved by providing a pharmaceutical composition comprising the dual specificity polypeptide molecule according to the present disclosure, the nucleic acid or the expression vector(s) according to the present disclosure, or the cell according to the present disclosure, together with one or more pharmaceutically acceptable carriers or excipients.

In another aspect of the present disclosure, the present disclosure relates to the dual specificity polypeptide molecule according to the present disclosure, the nucleic acid(s) or the expression vector(s) according to the present disclosure, the cell according to the present disclosure, or the pharmaceutical composition according to the present disclosure, for use in medicine.

In another aspect of the present disclosure, the present disclosure relates to the dual specificity polypeptide molecule according to the present disclosure, the nucleic acid or the expression vector(s) according to the present disclosure, the cell according to the present disclosure, or the pharmaceutical composition according to the present disclosure, for use in the treatment of a disease or disorder as disclosed herein, in particular selected from cancer and infectious diseases.

In another aspect of the present disclosure, the present disclosure relates to a method for the treatment of a disease or disorder comprising administering a therapeutically effective amount of the dual specificity polypeptide molecule according to the present disclosure, the nucleic acid or the expression vector(s) according to the present disclosure, the cell according to the present disclosure, or the pharmaceutical composition according to the present disclosure.

In another aspect of the present disclosure, the present disclosure relates to a method of eliciting an immune response in a patient or subject comprising administering a therapeutically effective amount of the dual specificity polypeptide molecule according to the present disclosure or the pharmaceutical composition according to the present disclosure.

In another aspect, the present disclosure relates to a method of killing target cells in a patient or subject comprising administering to the patient an effective amount of the dual specificity polypeptide molecule according to the present disclosure.

Examples of such dual specificity molecule are given in Table 3b.

TABLE 3b

| | | | | |
|---|---|---|---|---|
| Molecule | TCR | mAb | SEQ IDs | modifications |
| IA_5 | R16P1C10I | hUCHT1 (Var17) | SEQ ID NO: 131 SEQ ID NO: 132 | IgG1 (K/O, KiH-ds) |
| IA_6 | R16P1C10I#6 | hUCHT1 (Var17) | SEQ ID NO: 133 SEQ ID NO: 134 | IgG1 (K/O, KiH-ds) |

KiH: Knob-into-hole; K/O: Fc-silenced; KiH-ds: Knob-into-hole stabilized with artificial disulfide-bond to connect CH3:CH3’; and VH and VL domains derived from the CD3-specific, humanized antibody hUCHT1 (Var17).

In one embodiment, the first variable domain and the second variable domain as herein defined may comprise an amino acid substitution at position 44 according to the IMGT numbering. In a preferred embodiment, said amino acid at position 44 is substituted with another suitable amino acid, in order to improve pairing. In particular embodiments, in which said antigen binding protein is a TCR, said mutation improves for example the pairing of the chains (i.e. paring of α and β chains or paring of γ and δ). In a preferred embodiment, the amino acid as present at position 44 in the variable domain is substituted by one amino acid selected from the group consisting of Q, R, D, E, K, L, W, and V.

In one embodiment, the first variable domain of the antigen binding proteins of the present disclosure comprises:
a CDRa1 comprising or consisting of the amino acid sequence selected from the group consisting of the amino acid sequences DRGSQS (SEQ ID NO: 135) and DRGSQL (SEQ ID NO: 136), and/or
a CDRa2 comprising or consisting of the amino acid sequence selected from the group consisting of the amino acid sequences IYSNGD (SEQ ID NO: 137) and IYQEGD (SEQ ID NO: 138) and/or
a CDRa3 comprising or consisting of the amino acid sequence selected from the group consisting of the amino acid sequences CAAVINNPSGGMLTF (SEQ ID NO: 139), CAAVIDNSNGGILTF (SEQ ID NO: 140), CAAVIDNPSGGILTF (SEQ ID NO: 141), CAAVIDNDQGGILTF (SEQ ID NO: 142), CAAVIPNPPGGKLTF (SEQ ID NO: 143), CAAVIPNPGGGALTF (SEQ ID NO: 144), CAAVIPNSAGGRLTF (SEQ ID NO: 145), CAAVIPNLEGGSLTF (SEQ ID NO: 146), CAAVIPNRLGGYLTF (SEQ ID NO: 147), CAAVIPNTDGGRLTF (SEQ ID NO: 148), CAAVIPNQRGGALTF (SEQ ID NO: 149), CAAVIPNWGGILTF (SEQ ID NO: 150), CAAVITNIAGGSLTF (SEQ ID NO: 151), CAAVIPNNDGGYLTF (SEQ ID NO: 152)), CAAVIPNGRGGLLTF (SEQ ID NO: 153), CAAVIPNTHGGPLTF (SEQ ID NO: 154), CAAVIPNDVGGSLTF (SEQ ID NO: 155), CAAVIENKPGGPLTF (SEQ ID NO: 156), CAAVIDNPVGGPLTF (SEQ ID NO: 157), CAAVIPNNNGGALTF (SEQ ID NO: 158), CAAVIPNDQGGILTF (SEQ ID NO: 159), CAAVIPNVVGGQLTF (SEQ ID NO: 160), CAAVIPNSYGGLLTF (SEQ ID NO: 161), CAAVIPNDDGGLLTF (SEQ ID NO: 162), CAAVIPNAAGGLLTF (SEQ ID NO: 163), CAAVIPNTIGGLLTF (SEQ ID NO: 164) and CAAVIPNTRGGLLTF (SEQ ID NO: 165), and the
second variable domain comprises:
a CDRb1 comprising or consisting of the amino acid sequence selected from the group consisting of the amino acid sequences SGHRS (SEQ ID NO: 166) and PGHRA (SEQ ID NO: 167) and/or
a CDRb2 comprising or consisting of the amino acid sequence selected from the group consisting of the amino acid sequences YFSETQ (SEQ ID NO: 169), YVHGEE (SEQ ID NO: 170) and YVHGAE (SEQ ID NO: 171) and/or
a CDRb3 comprising or consisting of the amino acid sequence selected from the group consisting of the amino acid sequences CASSPWDSPNEQYF (SEQ ID NO: 172) and CASSPWDSPNVQYF (SEQ ID NO: 173).

The inventors of the present disclosure identified in the examples as herein disclosed, the TCR variant “HiAff1” and “LoAff3” of which the CDR amino acid sequences, when used in the antigen binding proteins of the present disclosure, in particular in bispecific antigen binding proteins, more particularly in a F_{c}- containing bispecific TCR/mAb (anti-CD3) diabody format, increase the binding affinity, the stability and the specificity of the antigen binding proteins comprising those CDRs, in particular, in comparison to a reference protein.

Such a reference protein may be, for example, an antigen binding protein comprising the CDRs of the parental / wild type TCR R16P1C10, which is disclosed in WO2018/172533, for instance, a F_{c}-containing bispecific TCR/mAb (anti-CD3) diabody as herein described comprising the CDRs of said TCR R16P1C10 or the reference protein is an antigen binding protein comprising the CDRs of said TCR R16P1C10 and is in the same format as the antigen binding protein with which it is compared. Such a reference protein may also be, for example, an antigen binding protein comprising the CDRs of “CDR6”, for instance, a F_{c}-containing bispecific TCR/mAb (anti-CD3) diabody as herein described comprising the CDRs of “CDR6” or the reference protein is an antigen binding protein comprising the CDRs of “CDR6” and is in the same format as the antigen binding protein with which it is compared, wherein the CDRs of “CDR6” are disclosed herein above.

The inventors demonstrated furthermore that the antigen binding proteins of the present disclosure comprising the above described CDRs have an improved stability in comparison to an antigen binding protein comprising the CDRs of a reference antigen binding protein called “CDR6”, wherein the antigen binding protein called “CDR6” comprises the following alpha and beta CDRs:

CDRa1 comprising or consisting of the amino acid sequence DRGSQS (SEQ ID NO: 135), and CDRa2 comprising or consisting of the amino acid sequence IYSNGD (SEQ ID NO: 137), and CDRa3 comprising or consisting of the amino acid sequence CAAVIDNDQGGILTF (SEQ ID NO: 142), and CDRb1 comprising or consisting of the amino acid sequence PGHRA (SEQ ID NO: 167), and CDRb2 comprising or consisting of the amino acid sequence YVHGEE (SEQ ID NO: 170), and CDRb3 comprising or consisting of the amino acid sequence CASSPWDSPNVQYF (SEQ ID NO: 173).

In one particular embodiment the present disclosure refers to antigen binding proteins comprising the CDRs of the so-called “HiAff#1” and “LoAff#3” variants and variants thereof. Accordingly, in one preferred embodiment, the antigen binding protein of the present disclosure comprises
a) a first polypeptide chain comprising a first variable domain comprising three complementary determining regions (CDRs) CDRa1, CDRa2 and CDRa3, wherein
   the CDRa1 comprises or consists of the amino acid sequence DRGSQS (SEQ ID NO: 135) or an amino acid sequence at least 85% identical to SEQ ID NO: 135),
   the CDRa2 comprises or consists of the amino acid sequence IYQEGD (SEQ ID NO: 138) and
   the CDRa3 comprises or consists of the amino acid sequence CAAVIDNDQGGILTF (SEQ ID NO: 142), and
b) a second polypeptide chain comprising a second variable domain comprising three complementary determining regions (CDRs) CDRb1, and CDRb3, wherein
   the CDRb1 comprises or consists of the amino acid sequence PGHRA (SEQ ID NO: 167) or PGHRS (SEQ ID NO: 168), preferably PGHRA (SEQ ID NO: 167), or an amino acid sequence at least 85% identical to SEQ ID NO: 167) or SEQ ID NO: 168), preferably SEQ ID NO: 167);
   the CDRb2 comprises or consists of the amino acid sequence YVHGEE (SEQ ID NO: 170) or an amino acid sequence at least 85% identical to SEQ ID NO: 170), and
   the CDRb3 comprises or consists of the amino acid sequence CASSPWDSPNEQYF (SEQ ID NO: 172) or CASSPWDSPNVQYF (SEQ ID NO: 173), preferably CASSPWDSPNVQYF (SEQ ID NO: 173), or an amino acid sequence at least 85% identical to SEQ ID NO: 172) or SEQ ID NO: 173), preferably CASSPWDSPNVQYF (SEQ ID NO: 173).

Table 4 sets forth CDR sequences and binding affinities of wild type and maturated TCRs expressed as scTCR-Fab (based on SEQ ID NOs: 81 and 82) or diabody-F_{c} (based on SEQ ID NOs: 119 and 120).

TABLE 4

| | | | | | | | |
|---|---|---|---|---|---|---|---|
| TCR variant | CDRa1 | CDRa2 | CDRa3 | CDRb1 | CDRb2 | CDRb3 | KD [M] |
| Wild type CDRs and framework | DRGSQS | IYSNGD | CAAVISNFGNEKLTF | SGHRS | YFSETQ | CASSPWDSPNEQYF | Cannot be expressed in CHO as scTCR-Fab or diabody-F_{c} |
| Stabilized ¹ | DRGSQS | IYSNGD | CAAVISNFGNEKLTF | PGHRS | YFSETQ | CASSPWDSPNEQYF | 1.2E-06 |
| Stabilized ² | DRGSQS | IYSNGD | CAAVISNFGNEKLTF | PGHRS | YFSETQ | CASSPWDSPNEQYF | 9.3E-07 |
| Improved ¹ | DRGSQS | IYSNGD | CAAVIDNSNGGILTF | PGHRS | YVHGAE | CASSPWDSPNEQYF | 1.0E-08 |
| Improved ² | DRGSQS | IYSNGD | CAAVIDNSNGGILTF | PGHRS | YVHGAE | CASSPWDSPNEQYF | 8.7E-09 |
| Medium-affinity LoAff3 ² | DRGSQS | IYQEGD | CAAVIDNDQGGILTF | PGHRS | YVHGEE | CASSPWDSPNEQYF | 1.8E-09 |
| High-affinity CDR6 ² | DRGSQS | IYSNGD | CAAVIDNDQGGILTF | PGHRA | YVHGEE | CASSPWDSPNVQYF | 3.9E-10 |
| High-affinity HiAff1 ² | DRGSQS | IYQEGD | CAAVIDNDQGGILTF | PGHRA | YVHGEE | CASSPWDSPNVQYF | 3.8E-10 |

¹expressed as scTCR-Fab
²expressed as diabody-F_{c}

All positions and CDR definitions are according to Kabat numbering scheme.TCRs consisting of Valpha and Vbeta domains were designed, produced and tested in a single-chain (scTCR) format coupled to a Fab-fragment of a humanized UCHT1-antibody. Vectors for the expression of recombinant proteins were designed as mono-cistronic, controlled by HCMV-derived promoter elements, pUC19-derivatives. Plasmid DNA was amplified in E.coli according to standard culture methods and subsequently purified using commercial-available kits (Macherey & Nagel). Purified plasmid DNA was used for transient transfection of CHO cells. Transfected CHO-cells were cultured for 10 - 11 days at 32° C. to 37° C.

TABLE 5

Bispecific molecules

| ID | α-chain SEQ ID NO: | β-chain SEQ ID NO: | ID | α-chain SEQ ID NO: | β-chain SEQ ID NO: | ID | α-chain SEQ ID NO: | β-chain SEQ ID NO: |
|---|---|---|---|---|---|---|---|---|
| TPP-70 | 178 | 179 | TPP-218 | 230 | 231 | TPP-268 | 265 | 286 |
| TPP-71 | 178 | 180 | TPP-219 | 240 | 239 | TPP-269 | 265 | 287 |
| TPP-72 | 178 | 181 | TPP-220 | 242 | 239 | TPP-270 | 265 | 288 |
| TPP-73 | 178 | 182 | TPP-221 | 244 | 239 | TPP-271 | 265 | 289 |
| TPP-74 | 178 | 183 | TPP-222 | 246 | 239 | TPP-272 | 218 | 290 |
| TPP-93 | 184 | 185 | TPP-226 | 222 | 247 | TPP-273 | 250 | 291 |
| TPP-79 | 187 | 186 | TPP-227 | 189 | 249 | TPP-274 | 250 | 292 |
| TPP-105 | 189 | 188 | TPP-228 | 250 | 249 | TPP-275 | 250 | 293 |
| TPP-106 | 190 | 191 | TPP-229 | 251 | 249 | TPP-276 | 250 | 294 |
| TPP-108 | 190 | 185 | TPP-230 | 246 | 249 | TPP-277 | 250 | 295 |
| TPP-109 | 195 | 194 | TPP-235 | 253 | 223 | TPP-279 | 250 | 296 |
| TPP-110 | 195 | 186 | TPP-236 | 254 | 223 | TPP-666 | 298 | 297 |
| TPP-111 | 187 | 194 | TPP-237 | 255 | 223 | TPP-669 | 299 | 297 |
| TPP-112 | 184 | 191 | TPP-238 | 256 | 223 | TPP-871 | 300 | 249 |
| TPP-113 | 184 | 203 | TPP-239 | 257 | 223 | TPP-872 | 300 | 301 |
| TPP-114 | 184 | 205 | TPP-240 | 258 | 223 | TPP-876 | 302 | 225 |
| TPP-115 | 206 | 205 | TPP-241 | 259 | 223 | TPP-879 | 298 | 303 |
| TPP-116 | 208 | 205 | TPP-242 | 260 | 223 | TPP-891 | 304 | 225 |
| TPP-117 | 210 | 205 | TPP-243 | 261 | 223 | TPP-892 | 304 | 297 |
| TPP-118 | 212 | 205 | TPP-244 | 262 | 223 | TPP-894 | 299 | 303 |
| TPP-119 | 184 | 213 | TPP-245 | 263 | 223 | TPP-1292 | 216 | 297 |
| TPP-120 | 184 | 214 | TPP-246 | 265 | 264 | TPP-1293 | 219 | 225 |
| TPP-121 | 206 | 214 | TPP-247 | 265 | 266 | TPP-1294 | 221 | 297 |
| TPP-122 | 208 | 214 | TPP-248 | 265 | 267 | TPP-1295 | 221 | 303 |
| TPP-123 | 210 | 214 | TPP-249 | 265 | 268 | TPP-1296 | 304 | 224 |
| TPP-124 | 212 | 214 | TPP-250 | 265 | 269 | TPP-1297 | 304 | 226 |
| TPP-125 | 184 | 215 | TPP-252 | 265 | 270 | TPP-1298 | 299 | 227 |
| TPP-126 | 206 | 215 | TPP-253 | 265 | 271 | TPP-1300 | 299 | 228 |
| TPP-127 | 208 | 215 | TPP-254 | 265 | 272 | TPP-1301 | 229 | 303 |
| TPP-128 | 210 | 215 | TPP-255 | 265 | 273 | TPP-1302 | 299 | 233 |
| TPP-129 | 212 | 215 | TPP-256 | 265 | 274 | TPP-1303 | 299 | 235 |
| TPP-207 | 187 | 217 | TPP-257 | 265 | 275 | TPP-1304 | 299 | 237 |
| TPP-208 | 218 | 217 | TPP-258 | 265 | 276 | TPP-1305 | 229 | 233 |
| TPP-209 | 220 | 217 | TPP-259 | 265 | 277 | TPP-1306 | 229 | 235 |
| TPP-210 | 222 | 217 | TPP-260 | 265 | 278 | TPP-1307 | 229 | 237 |
| TPP-211 | 187 | 223 | TPP-261 | 265 | 279 | TPP-1308 | 299 | 245 |
| TPP-212 | 218 | 225 | TPP-262 | 265 | 280 | TPP-1309 | 299 | 248 |
| TPP-213 | 220 | 225 | TPP-263 | 265 | 281 | TPP-1332 | 238 | 249 |
| TPP-214 | 230 | 223 | TPP-264 | 265 | 282 | TPP-1333 | 241 | 249 |
| TPP-215 | 232 | 231 | TPP-265 | 265 | 283 | TPP-1334 | 243 | 249 |
| TPP-216 | 234 | 231 | TPP-266 | 265 | 284 | | | |
| TPP-217 | 236 | 231 | TPP-267 | 265 | 285 | | | |

In Table 5, except for TPP-70, TPP-71, TPP-72, TPP-73 and TPP74, the term “α-chain” refers to a polypeptide chain comprising a V_{α}, i.e. a variable domain derived from a TCR α-chain. The term “β-chain” refers to a polypeptide chain comprising a V_{β}, i.e. a variable domain derived from a TCR β-chain. For TPP-70, TPP-71, TPP-72, TPP-73 and TPP74, the “α-chain” does not comprise any TCR derived variable domains, but the “β-chain” comprises two TCR-derived variable domains, one derived from a TCR α-chain and one derived from a TCR β-chain.

Conditioned cell supernatant was cleared by filtration (0.22 µm) utilizing Sartoclear Dynamics® Lab Filter Aid (Sartorius). Bispecific molecules were purified using an Äkta Pure 25 L FPLC system (GE Lifesciences) equipped to perform affinity and size-exclusion chromatography in line. Affinity chromatography was performed on protein L columns (GE Lifesciences) following standard affinity chromatographic protocols. Size exclusion chromatography was performed directly after elution (pH 2.8) from the affinity column to obtain highly pure monomeric protein using Superdex 200 pg 16/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/mL at temperatures of 2-8° C. Final product yield was calculated after completed purification and formulation.

Quality of purified bispecific molecules was determined by HPLC-SEC on MabPac SEC-1 columns (5 µm, 4×300 mm) running in 50 mM sodium-phosphate pH 6.8 containing 300 mM NaCl within a Vanquish uHPLC-System.

Stress stability testing was performed by incubation of the molecules formulated in PBS for up to two weeks at 40° C. Integrity, aggregate-content as well as monomer-recovery was analyzed by HPLC-SEC analyses.

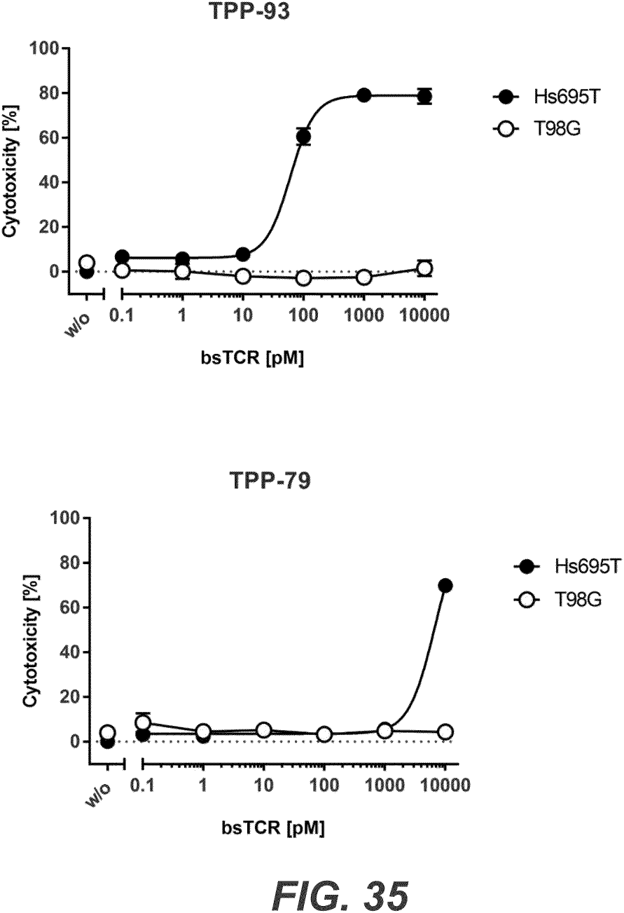

The inventors demonstrate that the antigen binding proteins, in particular TCER® molecules cause cytolysis in T2 cells loaded with target peptide PRAME-004 by LDH release assay (Table 6). The inventors further demonstrate that the antigen binding proteins, in particular TCER® molecules cause cytolysis in a PRAME-positive tumor cell line by LDH release assay while a PRAME-negative tumor cell line was not affected by co-incubation with the TCER® molecules (FIGS. 35 - 37). These in vitro-experiments further evidence the safety of the antigen binding proteins of the present disclosure and document that the cytotoxic effect is highly selective for PRAME-positive tumor tissue. The molecules of the present disclosures, therefore, show beneficial safety profiles.

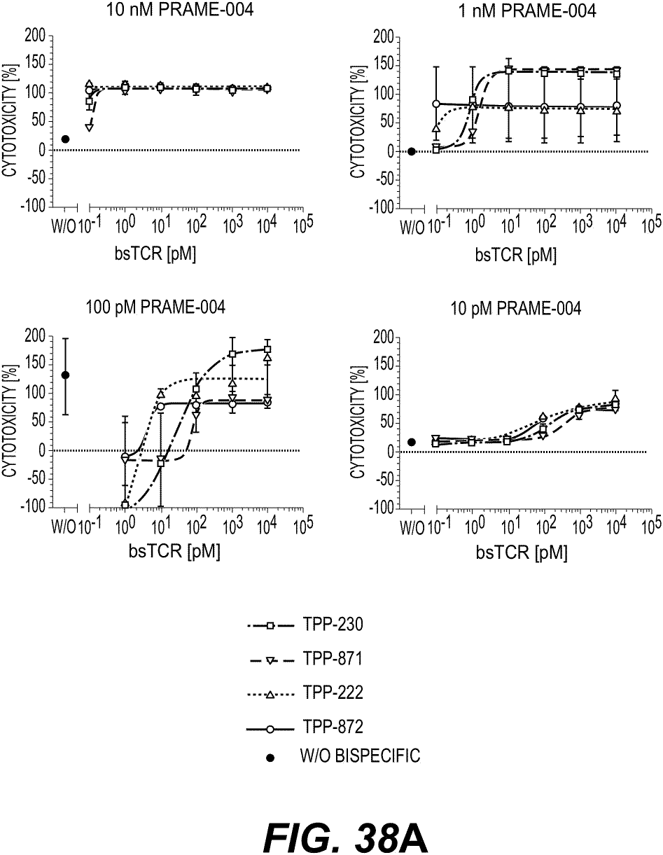

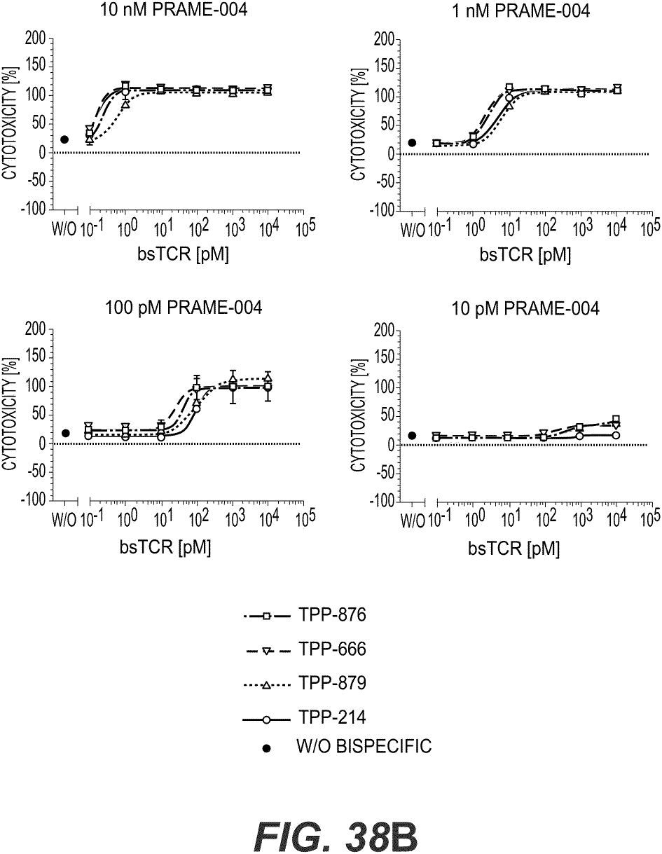

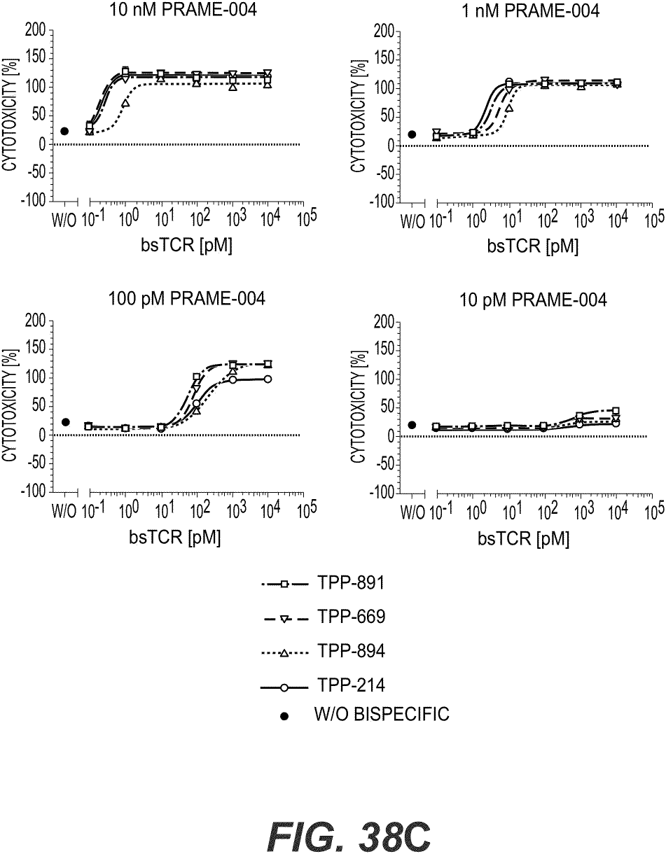

TCER® Slot III variants TPP-214, -222, -230, -666, -669, -871, -872, -876, -879, -891, -894 were additionally characterized for their ability to kill T2 cells loaded with varying levels of target peptide. After loading of the T2 cells with the respective concentrations of PRAME-004 for 2 h, peptide-loaded T2 cells were co-cultured with human PBMCs at an E:T ratio of 5:1 in the presence of increasing concentrations of TCER® variants for 48 h. Levels of LDH released into the supernatant were quantified using CytoTox 96 Non-Radioactive Cytotoxicity Assay Kit (Promega). All TCER® variants showed potent killing of PRAME-004-loaded T2 cells with subpicomolar EC50 values at a peptide loading concentration of 10 nM (FIGS. 38A-C, Table 6). EC50 values increased for decreasing PRAME-004 loading levels. However, even at a very low PRAME-004 loading concentration of 10 pM, killing was induced by all TCER® variants, except for TPP-214.

TABLE 6

In vitro cytotoxicity of TCER® Slot III variants on PRAME-004-loaded T2 cells. T2 cells were co-cultured with human PBMCs at an E:T ratio of 5:1 for 48 h. PRAME-004 loading concentrations are indicated. Ec₅₀ values and cytotoxicity levels in the plateau (Top) were calculated using non-linear 4-point curve fitting.

| TCER® variant | Recruiter | Va, Vb (SEQ ID NO:) | 10 nM PRAME-004 | | 1 nM PRAME-004 | | 100 pM PRAME-004 | | 10 pM PRAME-004 | |
|---|---|---|---|---|---|---|---|---|---|---|
| | | | EC₅₀ [pM] | Top | EC₅₀ [pM] | Top | EC₅₀ [pM] | Top | EC₅₀ [pM] | Top |
| TPP-230 | H2C | 305, 307 | 0.09 | 109 | 0.9 | 139 | 23.2¹ | 179 | 145 | 80 |
| TPP-871 | H2C | 309, 307 | 0.13 | 109 | 1.6 | 143 | 76.5¹ | 90 | 361 | 76 |
| TPP-222 | H2C | 305, 306 | complete killing | 109 | complete killing | 78 | 2.8¹ | 127 | 58 | 90 |
| TPP-872 | H2C | 309, 306 | complete killing | 109 | complete killing | 151 | 4.3¹ | 84 | 49 | 74 |
| TPP-876 | BMA031 (V36)A02 | 309, 306 | 0.16 | 111 | 2.0 | 113 | 24.4 | 100 | 539 | 40 |
| TPP-666 | BMA031 (V36)A02 | 305, 308 | 0.15 | 113 | 2.4 | 113 | 39.8 | 100 | 182 | 35 |
| TPP-879 | BMA031 (V36)A02 | 305, 307 | 0.54 | 106 | 6.2 | 109 | 94.4 | 117 | 1070 | 39 |
| TPP-214 | BMA031 (V36) | 305, 306 | 0.22 | 108 | 5.0 | 109 | 92.8 | 102 | no killing | 20 |
| TPP-891 | BMA031 (V36)D01 | 309, 306 | 0.19 | 120 | 2.2 | 112 | 54.0 | 125 | 611 | 45 |
| TPP-669 | BMA031 (V36)D01 | 305, 308 | 0.22 | 124 | 3.2 | 108 | 84.0 | 126 | 246 | 31 |
| TPP-894 | BMA031 (V36)D01 | 305, 307 | 0.87 | 108 | 9.9 | 115 | 226.0 | 129 | 1084 | 44 |
| TPP-214 | BMA031 (V36) | 305, 306 | 0.26 | 121 | 5.4 | 111 | 105.4 | 99 | no killing | 23 |

¹High variability within replicates do not allow for reliable Ec₅₀ calculation.

According to yet another aspect of the present disclosure, a pharmaceutical composition comprising at least one active agent is provided, the agent selected from the group consisting of at least one of
the peptide according to the above description
the antibody or fragment thereof according to the above description
the T-cell receptor or fragment thereof according to the above description
the nucleic acid or the expression vector according to the above description
the host cell according to the above description,
the recombinant T lymphocyte according to the above description, and/or
the activated T lymphocyte according to the above description
and a pharmaceutically acceptable carrier. The composition is for use in the (manufacture of a medicament for the) treatment of a patient (i) being diagnosed for, (ii) suffering from or (iii) being at risk of developing recurrent cancer.

Alternatively or in addition, a method of treating a patient (i) being diagnosed for, (ii) suffering from or (iii) being at risk of developing recurrent cancer, is provided.

The method comprises administering to the patient at least one active ingredient selected from the group consisting of at least one of
the peptide according to the above description
the antibody or fragment thereof according to the above description
the T-cell receptor or fragment thereof according to the above description
the nucleic acid or the expression vector according to the above description
the host cell according to the above description,
the recombinant T lymphocyte according to the above description, and/or
the activated T lymphocyte according to the above description
and a pharmaceutically acceptable carrier, in one or more therapeutically effective doses.

Alternatively or in addition, a pharmaceutical composition for treating recurrent cancer is provided, comprising such active ingredient as an effective ingredient.

In one embodiment, the recurrent cancer or first cancer being treated is PRAME positive. In one embodiment, the recurrent cancer displays, on the surface of at least one of its cells, a peptide comprising the amino acid sequence of SEQ ID NO: 310 (SLLQHLIGL), or said amino acid bound to a major histocompatibility complex. In another embodiment, the recurrent cancer or first cancer being treated is MAG-003 positive, MAGEA1-003 positive, COL6A3 positive, or MAGE positive.

In one embodiment, the patient is positive for HLA-A*02. This encompasses, inter alia, the haplotypes HLA-A*02:01, HLA-A*02:02, HLA-A*02:03m HLA-A*02:05, HLA-A*02:06, HLA-A*02:07 and HLA-A*02:11. In one embodiment, the patient is positive for HLA-A*02:01.

In different embodiments of the present disclosure, the recurrent cancer is at least one selected from the group consisting of at least one of:
Adrenocortical Carcinoma
Bladder Urothelial Carcinoma
Breast Cancer
Triple-Negative Breast Cancer
Colorectal Cancer
Head And Neck Squamous Cell Carcinoma
Head and Neck Adenocarcinoma
Melanoma
Skin Cutaneous Melanoma
Uveal Melanoma
Lung Cancer
Non-small Cell Lung Cancer
Non-small Cell Lung Squamous Cell Carcinoma
Non-small Cell Lung Adenocarcinoma
Small Cell Lung Cancer
Cholangiocarcinoma
Esophageal Carcinoma
Cervical Squamous Cell Carcinoma and Endocervical Adenocarcinoma
Ovarian Carcinoma
Ovarian Serous Cystadenocarcinoma
Liver Hepatocellular Carcinoma
Renal Cell Carcinoma
Kidney Renal Clear Cell Carcinoma
Kidney Renal Papillary Cell Carcinoma
Sarcoma
Fibrosarcoma
Liposarcoma
Malignant Peripheral Nerve Sheath Tumors
Synovial Sarcoma
Stomach Adenocarcinoma
Testicular Germ Cell Tumors
Thymoma
Uterine Carcinosarcoma
Uterine Corpus Endometrial Carcinoma and/or
Undifferentiated Endometrial Carcinoma

According to further embodiments, the following is provided:

An in vitro method for producing activated T lymphocytes specific for use in the (manufacture of a medicament for the) treatment of a patient (i) being diagnosed for, (ii) suffering from or (iii) being at risk of developing recurrent cancer, the method comprising the steps of providing a synthetic or recombinant peptide consisting in the amino acid sequence of SEQ ID NO: 310, contacting in vitro T cells with antigen loaded human class I major histocompatibility complex (MHC) molecules expressed on the surface of a suitable antigen-presenting cell or an artificial construct mimicking an antigen-presenting cell for a period of time sufficient to activate said T cells in an antigen specific manner, wherein said antigen is a peptide consisting in the amino acid sequence of SEQ ID NO: 310.

A cell line of activated T lymphocytes produced by the method according to item 1, characterized in that said cell line is capable of selectively recognizing recurrent cells which present a peptide consisting of the amino acid sequence of SEQ ID NO: 310.

An in vitro method for producing a soluble T cell receptor, characterized in that the method comprises the steps of:
(i) selecting a specific T cell clone that expresses a T cell receptor which binds to an HLA ligand that consists of a synthetic or recombinant peptide consisting of the amino acid sequence of SEQ ID NO: 310, optionally wherein said peptide is bound to an MHC, optionally wherein said T cell clone been created by immunizing a genetically engineered non- human mammal which is transgenic for the entire human TCR gene loci with a peptide comprising the amino acid sequence of SEQ ID NO: 310 , or with a peptide/MHC complex comprising such peptide, optionally selecting, for example form a library of TCRs or CDR mutants by yeast, phage, or T-cell display, a specific T cell receptor that binds to a synthetic or recombinant peptide comprising the amino acid sequence of SEQ ID NO: 310 , optionally when bound to an MHC; or
(ii) selecting a specific T cell receptor that binds to an HLA ligand that consists of a synthetic or recombinant peptide consisting of the amino acid sequence of SEQ ID NO: 310, optionally wherein said peptide is bound to an MHC from a phage display system, wherein said T cell receptor by virtue of binding to a peptide/MHC complex that comprises a peptide comprising SEQ ID NO: 310 bound to an MHC molecule is capable of reacting with an HLA ligand consisting of a peptide of SEQ ID NO: 310, which is presented recurrent cells.

An in vitro method for producing a recombinant antibody specifically binding to a human major histocompatibility complex (MHC) class I being complexed with a peptide of amino acid sequence of SEQ ID NO: 310, characterized in that the method comprises the steps of
(i) immunizing a genetically engineered non-human mammal which is transgenic for the entire human immunoglobulin gene loci with a peptide comprising the amino acid sequence of SEQ ID NO: 310, or with a peptide/MHC complex comprising such peptide;
(ii) isolating mRNA molecules from antibody producing cells of said non-human mammal;
(iii) producing a phage display library displaying protein molecules encoded by said mRNA molecules; and
(iv) isolating at least one phage from said phage display library, in which the at least one phage contains said antibody that specifically binds to the peptide comprising SEQ ID NO: 310 bound to an MHC class I molecule;
   wherein said antibody by virtue of binding to a peptide/MHC complex that comprises a peptide comprising SEQ ID NO: 310 bound to an MHC class I molecule is capable of specifically recognizing said peptide of SEQ ID NO: 310 when complexed with said MHC molecule,
   wherein said peptide of SEQ ID NO: 310 is expressed in the surface of cells.

A pharmaceutically acceptable salt of the peptide consisting of the amino acid sequence of SEQ ID NO: 310, characterized in that the salt is an acetate, a trifluoro acetate or a chloride.

A pharmaceutical composition comprising the cell line produced according to the method of item 2, the TCR produced according to the in vitro method of item 3, or the antibody produced according to the in vitro method of item 4 and a pharmaceutically acceptable carrier.

### Imaging of Cancer, Such As, Recurrent Cancer

Fluciclovine (¹⁸F) injection, also known as [¹⁸F]-FACBC, FACBC, or anti-1-amino-3-18F-fluorocyclobutane-1-carboxylic acid, is a synthetic amino acid imaging agent which is taken up specifically by amino acid transporters and is used for positron emission tomography (PET). PET may be uniquely suited to evaluate metabolic activity in human tissue for diagnostic imaging purposes. [¹⁸F]-fluoro-2-deoxy-glucose (FDG) is a PET imaging agent for the detection and localization of many forms of cancer. [18F]-FACBC may be used in the imaging of a variety of cancers including primary and recurrent prostate cancer, as it has excellent in vitro uptake and low urinary excretion. PET imaging with [18F]-FACBC better defines tumours compared to other known tracers such as FDG, allowing for better diagnosis and planning of treatment, for example, by directing radiation therapy to the appropriate areas.

The time between acquisition of the first and second PET scan images, e.g., between obtaining a first PET scan image of the subject and obtaining a second PET scan image of the subject, may be as much as one year. In some instances, the time between the first and second PET scans may be about 6 months, 5 months, 4 months, 3 months, 2 months, 1 month or even less than about 1 month. It may be appreciated that administering to the subject a second dose of a detectable amount of [18F]-FACBC and allowing time for [18F]-FACBC to accumulate at one or more areas of interest within the subject and obtaining a second PET scan image of the subject may be repeated as many times as desired and/or necessary in order to obtain multiple scan images which can be used to map the development of a tumour over time.

Once image data has been collected from the second PET scan, the first and second images may be visualized together and used to view the change in extent and location of tumour cells within the subject, allowing for the diagnosis or monitoring of recurrent cancer. For example, if the location of the tumour cells has changed then the subject may be diagnosed with recurrent cancer. In some embodiments, the second scan image can be compared to images of data collected from an earlier PET scan taken before the first PET scan, in addition to comparison with the first PET scan. In addition, any subsequent PET scan images obtained after the second PET scan image can be compared with the first and/or second PET scan images.

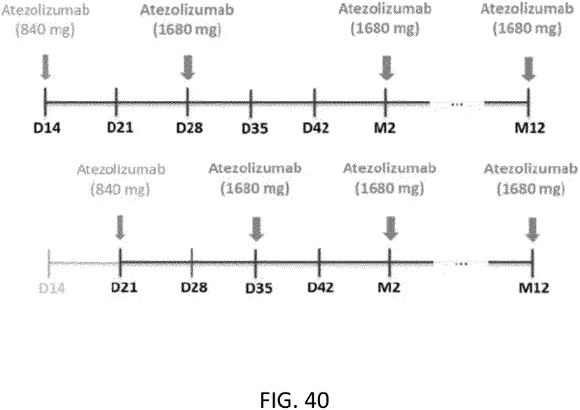

By comparing the images from two or more differing time points, the differences in the tumour uptake of [18F]-FACBC can be analyzed. Comparisons can involve qualitative image comparison (e.g., contrast of tumour uptake from background) and/or quantitative indices derived from the imaging or external radiation detection data (e.g., SUVs). The development, progression or reduction of any tumours can therefore be monitored and diagnosed accordingly. Suitable treatment can then be determined, for example, targeted administration of localized treatment at the site of the tumour. It may be appreciated that the methods described herein can also be used to monitor response to various therapeutic regimens, including immunotherapy. FIGS. 40A, 40B, and 40C show exemplary images before and after treatment for synovial sarcoma. Significant quantitative decrease in fluciclovine (18F) uptake post therapy were seen showing that images taken at different time points can be compared.

The PET scan image obtained in steps b) and d) of the methods described above may be combined with, preceded or followed by anatomical imaging selected from computed tomography (CT) imaging, computerized axial tomography (CAT) imaging, MRI imaging ultrasound, or a combination thereof. For combined imaging, the images can be acquired using a dedicated PET-CT, PET-MRI, PET-ultrasound scanning device or separate PET and CT/MRI/ultrasound scanning devices. If separate PET and CT/MRI/ultrasound imaging devices are used, image analysis techniques can be employed to spatially register the PET images with the anatomical images.

### Description of Figures

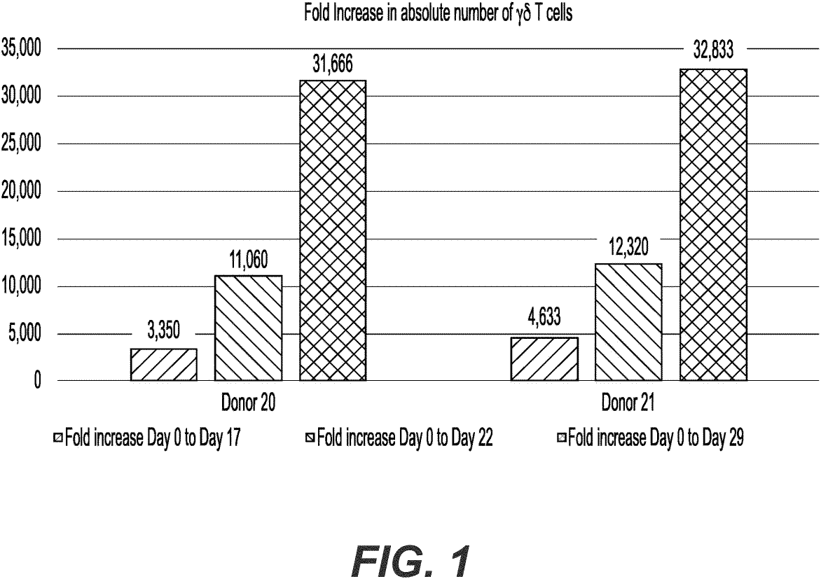

FIG. 1 shows γδ T-cell expansion using Zoledronate (Zometa) in defined medium, which contains IL-2, IL-15, and Amphotericin B. Fold increase in absolute number of γδ T-cells is 3,350-fold, 11,060-fold, and 31,666-fold for Donor 20 from Day 0 to Day 17, from Day 0 to Day 22, and from Day 0 to Day 29, respectively. Similarly, fold increase in absolute number of γδ T-cells is 4,633-fold, 12,320-fold, and 32,833-fold for Donor 21 from Day 0 to Day 17, from Day 0 to Day 22, and from Day 0 to Day 29, respectively. In contrast, as noted above, classic Vγ9δ2 T-cell expansion protocol, at best, could yield only a 100-fold increase in total Vγ9δ2 T-cells within 14 days, thereafter, the expansion rate decreases, which may be caused by an increase of cell death. In an aspect, using the afore-mentioned methods, fold increase in absolute number of γδ T-cells after expansion on Day 29 as compared with that of Day 0 may be from about 1000-fold to about 40,000-fold, from about 3000-fold to about 35,000-fold, from about 5000-fold to about 35,000-fold, from about 6000-fold to about 35,000-fold, from about 7000-fold to about 35,000-fold, from about 8000-fold to 30,000-fold, from about 10,000-fold to about 35,000-fold, from about 15,000-fold to about 35,000-fold, from about 20,000-fold to about 35,000-fold, from about 25,000-fold to about 35,000-fold, from about 30,000-fold to about 35,000-fold, more than about 10,000 fold, more than about 15,000 fold, more than about 20,000 fold, more than about 25,000 fold, more than about 30,000 fold, more than about 40,000 fold, or more than about 40,000 fold.

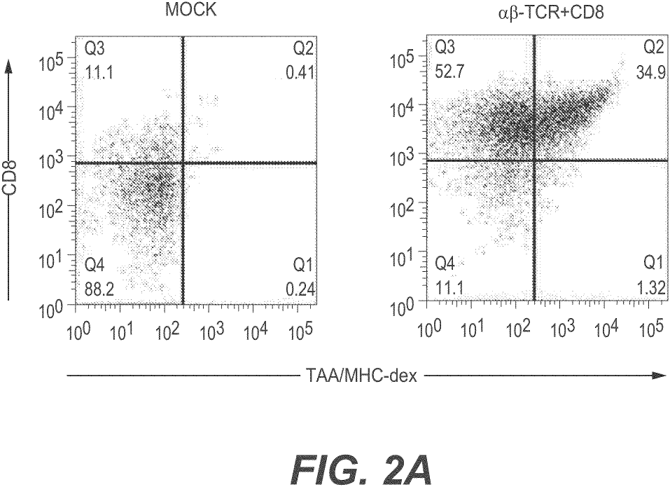

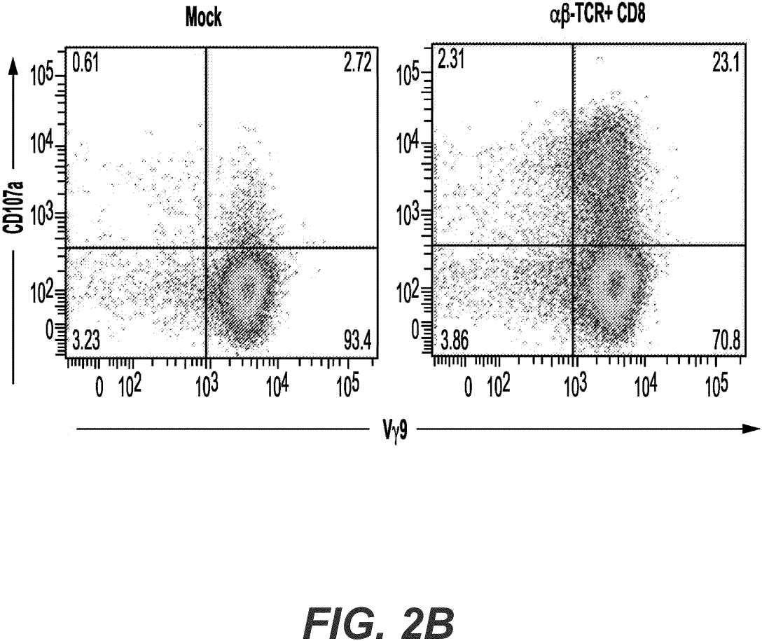

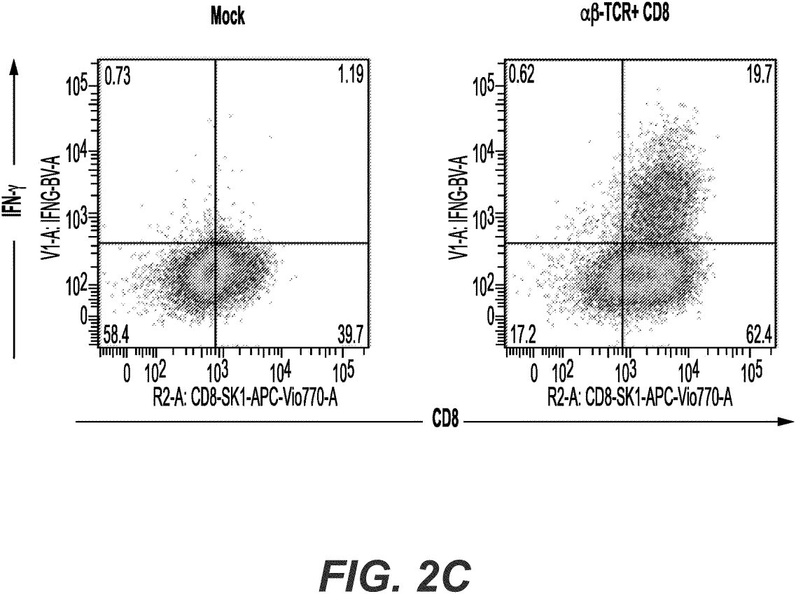

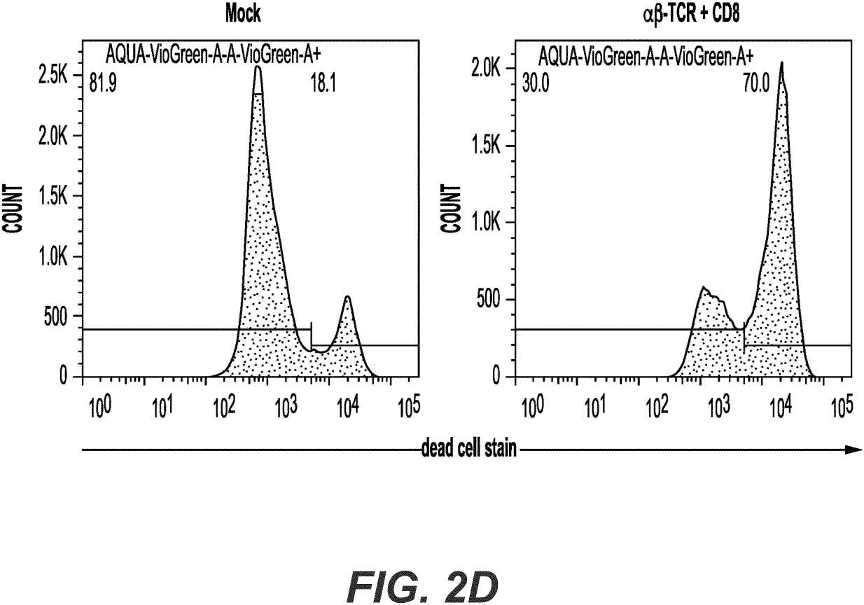

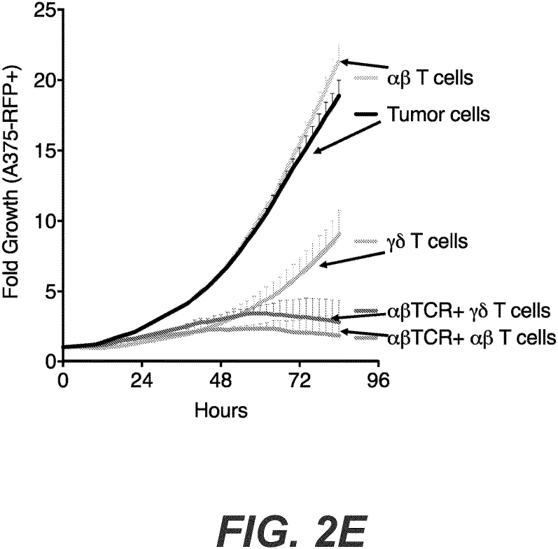

FIG. 2A shows, as compared with Vγ9δ2 T-cells without viral transduction (Mock), 34.9% of Vγ9δ2 T-cells transducing with αβ-TCR retrovirus and CD8αβ retrovirus αβ-TCR + CD8) stained positive by peptide/MHC-dextramer (TAA/MHC-dex) and anti-CD8 antibody (CD8), indicating the generation of Vγ9δ2 T-cells expressing both αβ-TCR and CD8αβ on cell surface (αβ-TCR +CD8αβ engineered Vg9d2 T-cells).

The principle of CD107a degranulation assay is based on killing of target cells via a granule-dependent pathway that utilizes pre-formed lytic granules located within the cytoplasm of cytotoxic cells. The lipid bilayer surrounding these granules contains lysosomal associated membrane glycoproteins (LAMPs), including CD107a (LAMP-1). Rapidly upon recognition of target cells via the T-cell receptor complex, apoptosis-inducing proteins like granzymes and perforin are released into the immunological synapse, a process referred to as degranulation. Thereby, the transmembrane protein CD107a is exposed to the cell surface and can be stained by specific monoclonal antibodies.

FIG. 2B shows, as compared with Vγ9δ2 T-cells without viral transduction (Mock), 23.1% of Vγ9δ2 T-cells transduced with αβ-TCR retrovirus and CD8αβ retrovirus (αβ-TCR + CD8) incubated with target cells, e.g., A375 cells, stained positive by anti-CD107a antibody, indicating that αβ-TCR +CD8αβ engineered Vg9d2 T-cells are cytolytic by carrying out degranulation, when exposed to A375 cells. IFN-γ release assays measure the cell mediated response to antigen-presenting cells, e.g., A375 cells, through the levels of IFN-γ released, when TCR of T-cells specifically binds to peptide/MHC complex of antigen-presenting cells on cell surface.

FIG. 2C shows, as compared with Vγ9δ2 T-cells without viral transduction (Mock), 19.7% of Vγ9δ2 T-cells transduced with αβ-TCR retrovirus and CD8αβ retrovirus (αβ-TCR + CD8) stained positive by anti-IFN-γ antibody, indicating that αβ-TCR +CD8αβ engineered Vγ9δ2 T-cells are cytolytic by releasing IFN-γ, when exposed to A375 cells.

Cytolytic activity were evaluated at 24 hours post-exposure to A375 cells by gating on apoptosis of non-CD3 T-cells, i.e., A375 cells. Apoptosis was assessed by staining the harvested culture with live/dead dye.

FIG. 2D shows, as compared with Vγ9δ2 T-cells without viral transduction (Mock), αβ-TCR +CD8αβ engineered Vγ9δ2 T-cells (αβ-TCR + CD8) induced apoptosis in 70% of A375 cells, indicating that αβ-TCR +CD8αβ engineered Vγ9δ2 T-cells are cytolytic by killing A375 cells. Cytolytic activity was also evaluated in real-time during an 84-hour co-culture assay. Non-transduced and αβTCR+CD8αβ transduced γδ T-cells were co-culture with target positive A375-RFP tumor cells at an effector to target ratio of 3:1. Lysis of target positive A375-RFP tumor cells was assessed in real time by IncuCyte® live cell analysis system (Essen BioScience). Tumor cells alone and non-transduced and αβTCR transduced αβ T-cells were used as negative and positive controls, respectively.

As shown in FIG. 2E, while non-transduced γδ T-cells showed cytotoxic potential due to intrinsic anti-tumor properties of γδ T-cells, αβTCR+CD8αβ transduced γδ T-cells showed similar cytotoxic potential as compared to αβTCR transduced αβ T-cells, indicating that αβTCR+CD8αβ transduced γδ T-cells can be engineered to target and kill tumor cells. These data indicate engineered Vγ9δ2 T-cells produced by the methods of the present disclosure are functional and can be used to kill target cells, e.g., cancer cells, in a peptide-specific manner.

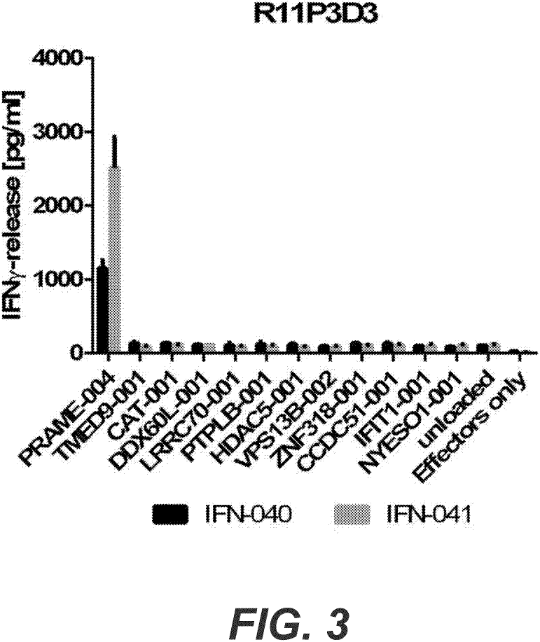

FIG. 3: IFNγ release from CD8+ T-cells electroporated with alpha and beta chain RNA of TCR R11P3D3 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

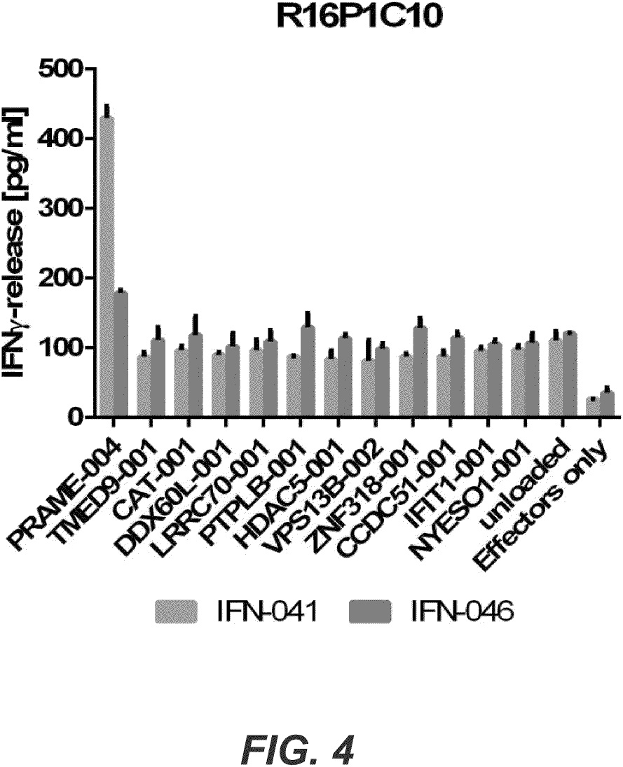

FIG. 4: IFNγ release from CD8+ T-cells electroporated with alpha and beta chain RNA of TCR R16P1C10 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-046 and IFN-041.

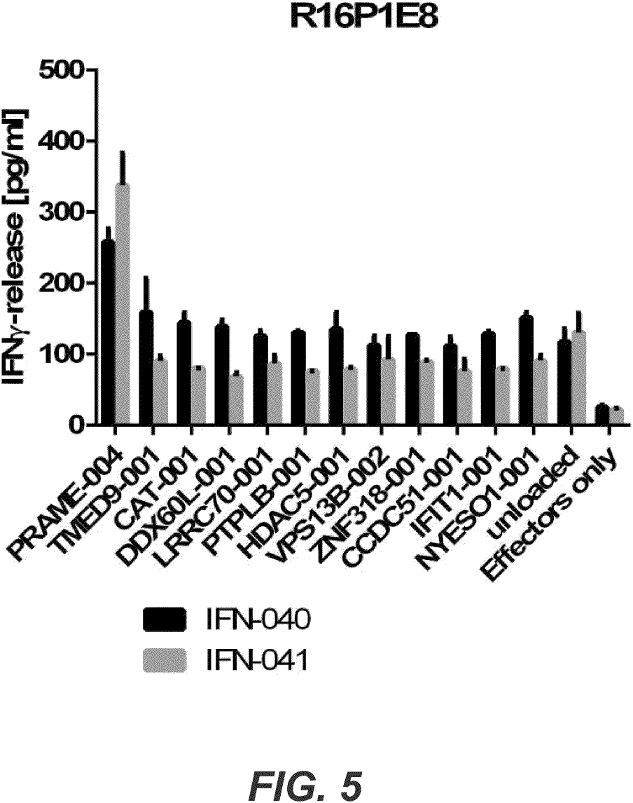

FIG. 5: IFNγ release from CD8+ T-cells electroporated with alpha and beta chain RNA of TCR R16P1E8 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

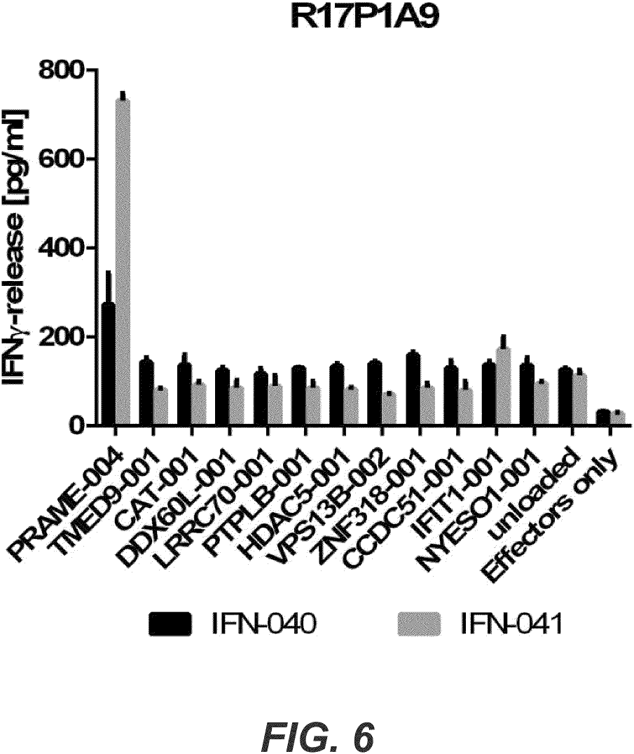

FIG. 6: IFNγ release from CD8+ T-cells electroporated with alpha and beta chain RNA of TCR R17P1A9 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

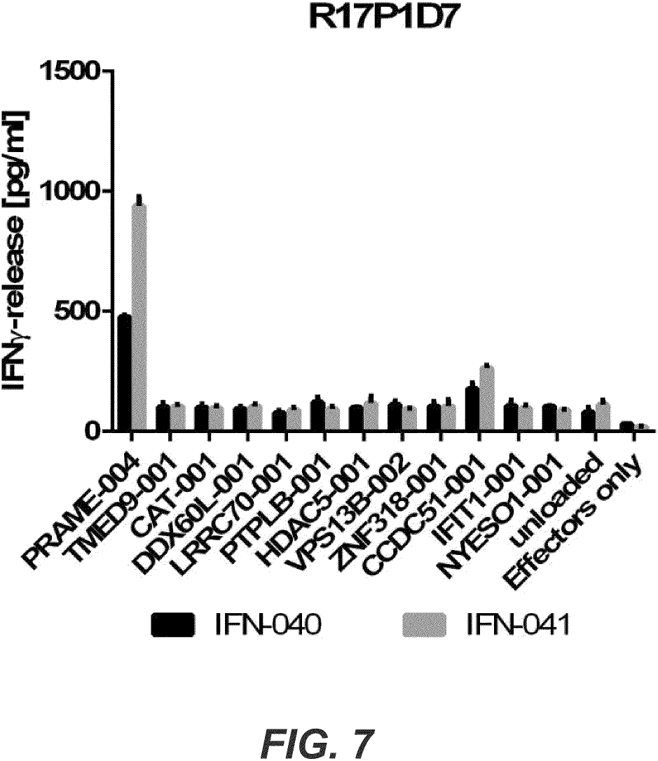

FIG. 7: IFNγ release from CD8+ T-cells electroporated with alpha and beta chain RNA of TCR R17P1D7 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

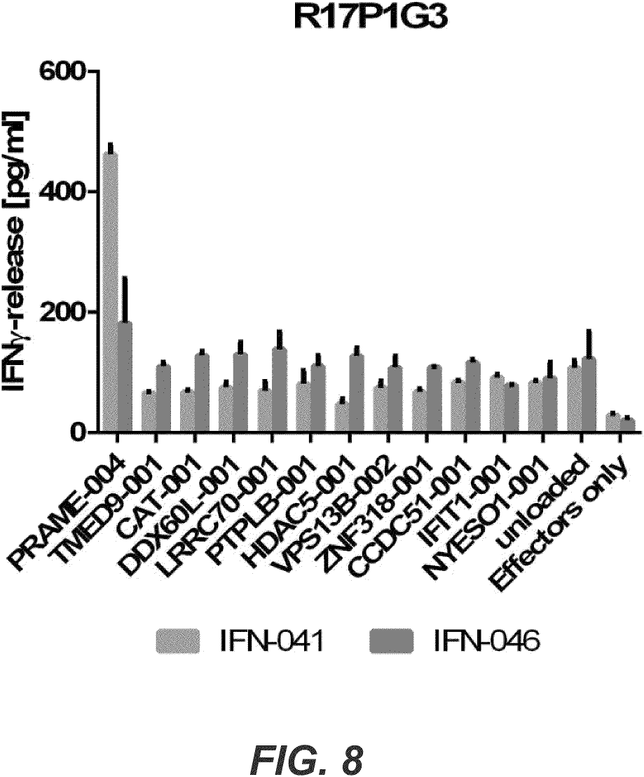

FIG. 8: IFNγ release from CD8+ T-cells electroporated with alpha and beta chain RNA of TCR R17P1G3 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-046 and IFN-041.

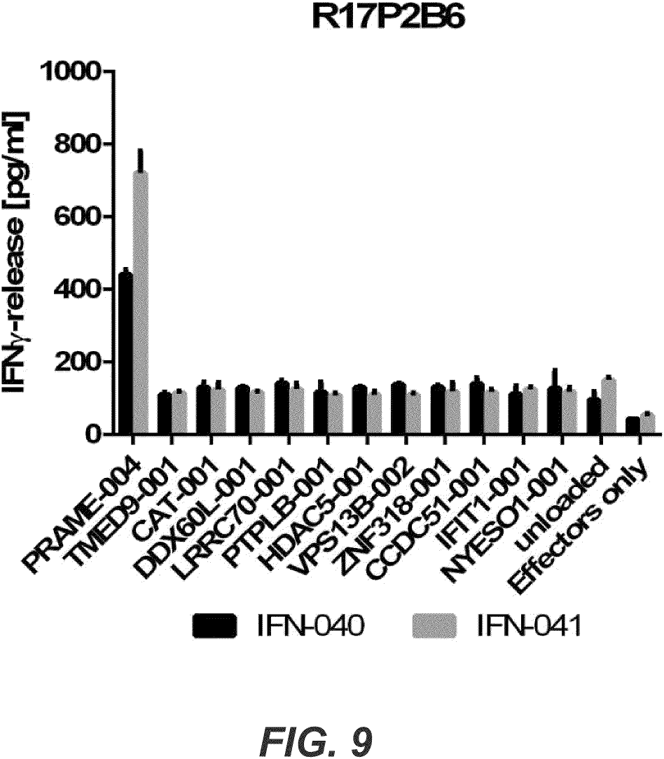

FIG. 9: IFNγ release from CD8+ T-cells electroporated with alpha and beta chain RNA of TCR R17P2B6 (Table 7) after co-incubation with T2 target cells loaded with PRAME-004 peptide (SEQ ID NO: 310) or similar but unrelated peptide TMED9-001, CAT-001, DDX60L-001, LRRC70-001, PTPLB-001, HDAC5-001, VPS13B-002, ZNF318-001, CCDC51-001, IFIT1-001, or control peptide NYESO1-001 (SEQ ID NO: 311). IFNγ release data were obtained with CD8+ T-cells derived from two different healthy donors. RNA electroporated CD8+ T-cells alone or in co-incubation with unloaded target cells served as controls. Different donors were analyzed, IFN-040 and IFN-041.

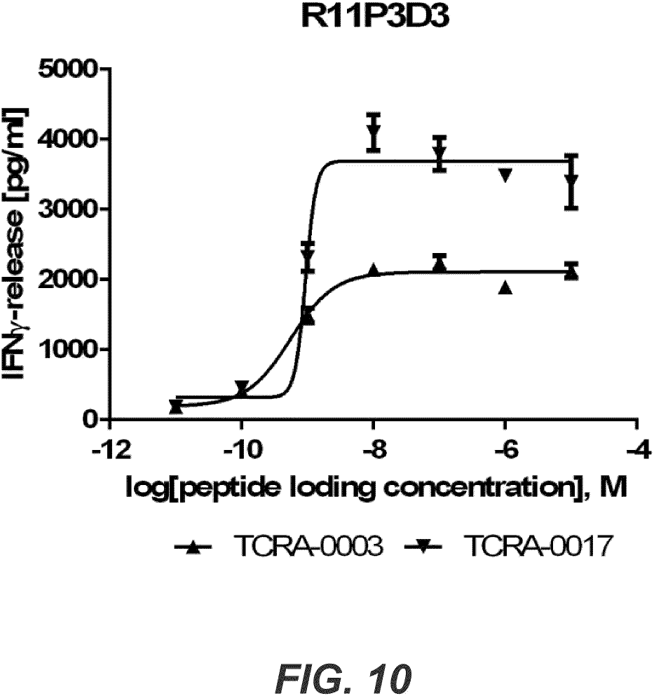

FIG. 10: IFNγ release from CD8+ T-cells electroporated with alpha and beta chain RNA of TCR R11P3D3 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

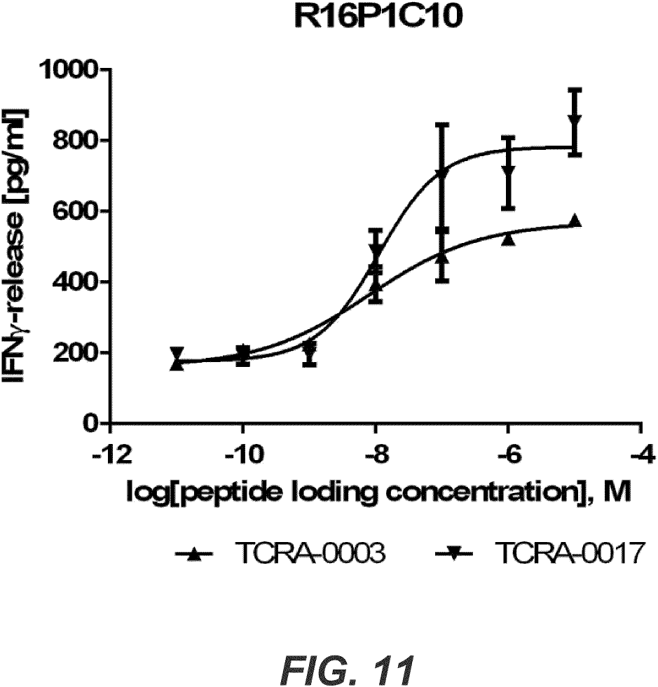

FIG. 11: IFNγ release from CD8+ T-cells electroporated with alpha and beta chain RNA of TCR R16P1C10 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

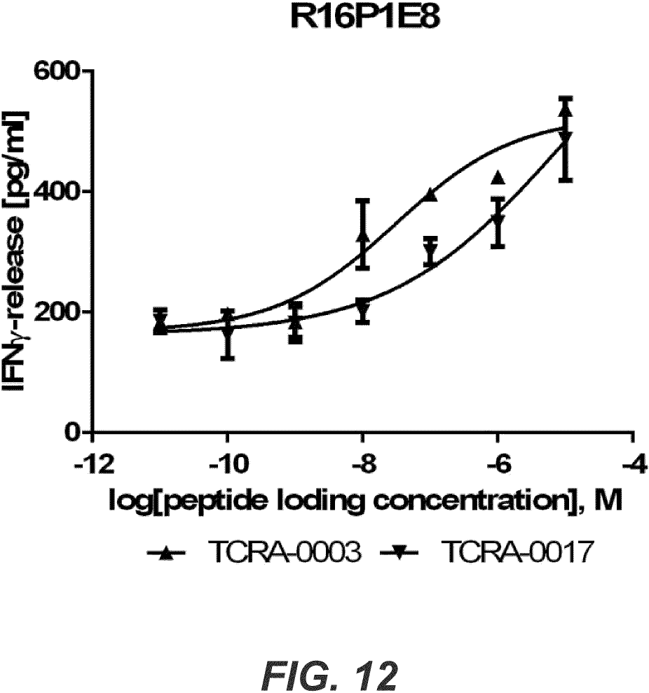

FIG. 12: IFNγ release from CD8+ T-cells electroporated with alpha and beta chain RNA of TCR R16P1E8 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

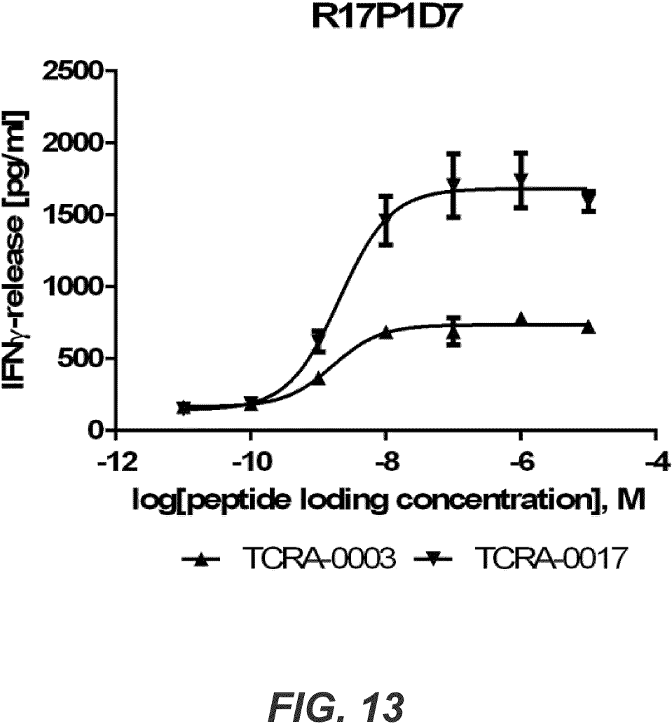

FIG. 13: IFNγ release from CD8+ T-cells electroporated with alpha and beta chain RNA of TCR R17P1D7 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

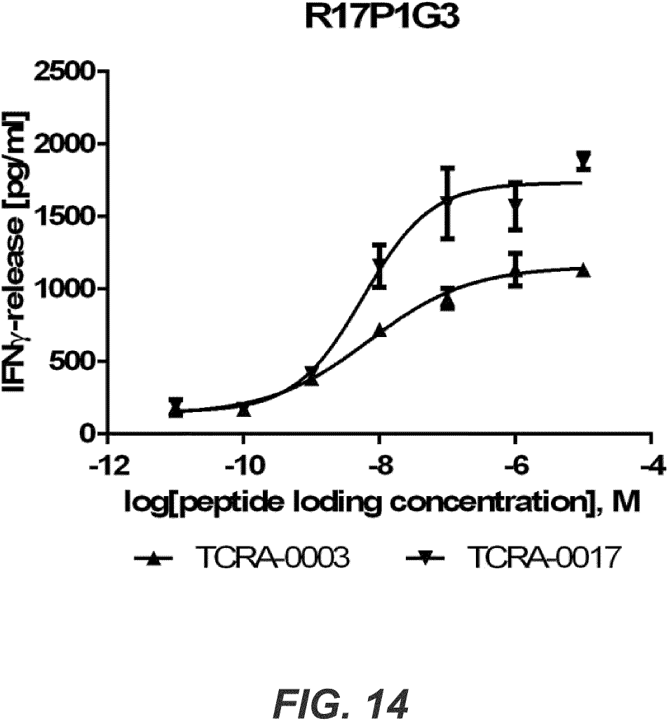

FIG. 14: IFNγ release from CD8+ T-cells electroporated with alpha and beta chain RNA of TCR R17P1G3 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

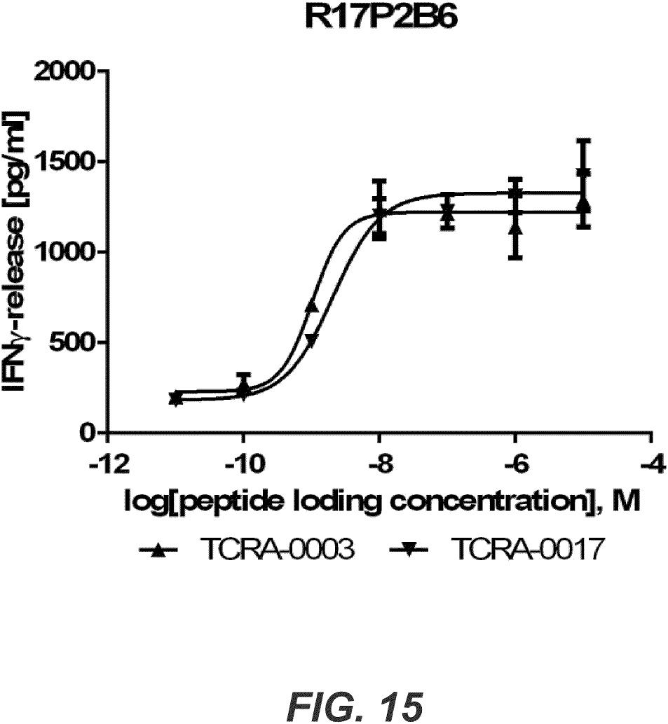

FIG. 15: IFNγ release from CD8+ T-cells electroporated with alpha and beta chain RNA of TCR R17P2B6 (Table 7) after co-incubation with T2 target cells loaded with PRAME-004 peptide (SEQ ID NO: 310) in various peptide loading concentrations from 10 µM to 10 pM. IFNγ release data were obtained with CD8+ T-cells derived from two different healthy donors. Different donors were analyzed, TCRA-0003 and TCRA-0017.

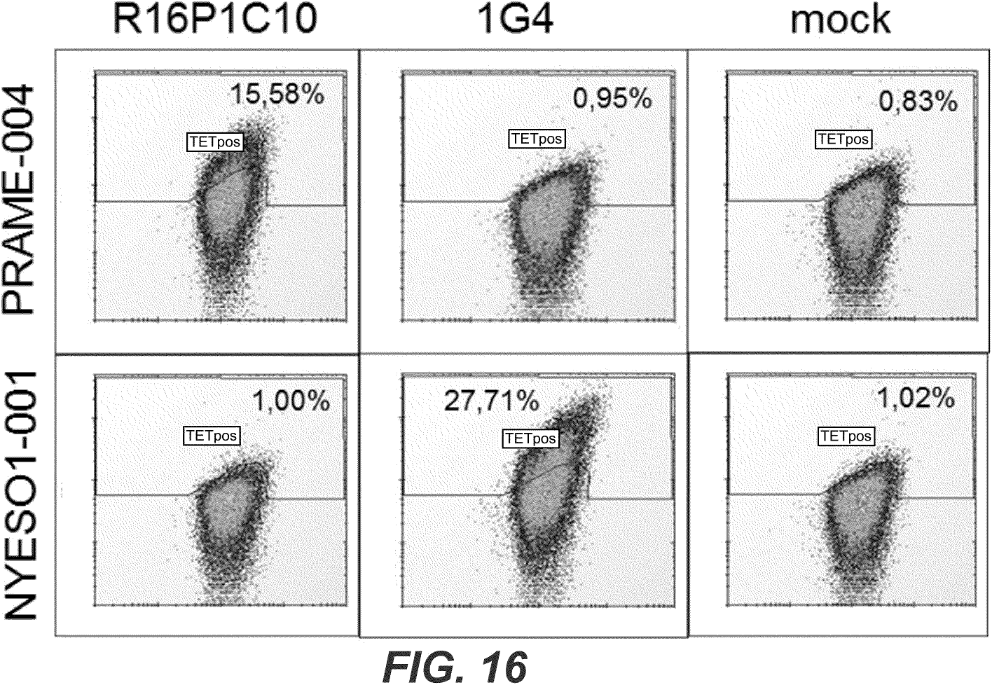

FIG. 16: HLA-A*02/PRAME-004 tetramer or HLA-A*02/NYESO1-001 (SEQ ID NO: 311) tetramer staining, respectively, of CD8+ T-cells electroporated with alpha and beta chain RNA of TCR R16P1C10 (Table 7). CD8+ T-cells electroporated with RNA of 1G4 TCR (SEQ ID: 85-96) that specifically binds to the HLA-A*02/NYESO1-001 (SEQ ID NO: 311) complex and mock electroporated CD8+ T-cells served as controls.

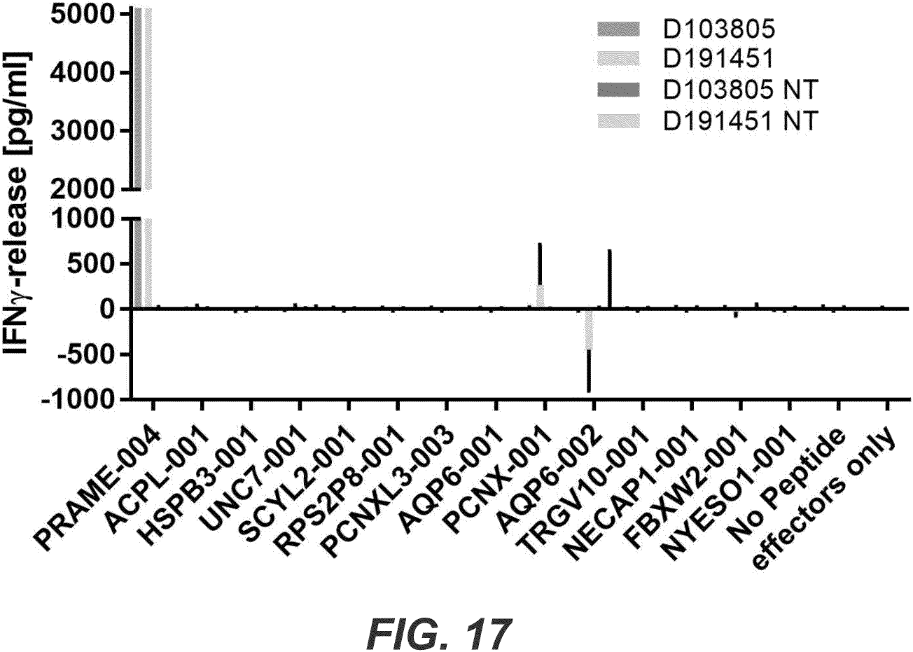

FIG. 17: IFNγ release from CD8+ T-cells lentivirally transduced with TCR R11P3D3 (Table 7) (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6 and 7) but unrelated peptides ACPL-001, HSPB3-001, UNC7-001, SCYL2-001, RPS2P8-001, PCNXL3-003, AQP6-001, PCNX-001, AQP6-002 TRGV10-001, NECAP1-001, FBXW2-001 or control peptide NYESO1-001 (SEQ ID NO: 311). IFNγ release data were obtained with CD8+ T-cells derived from two different healthy donors, D103805 and D191451.

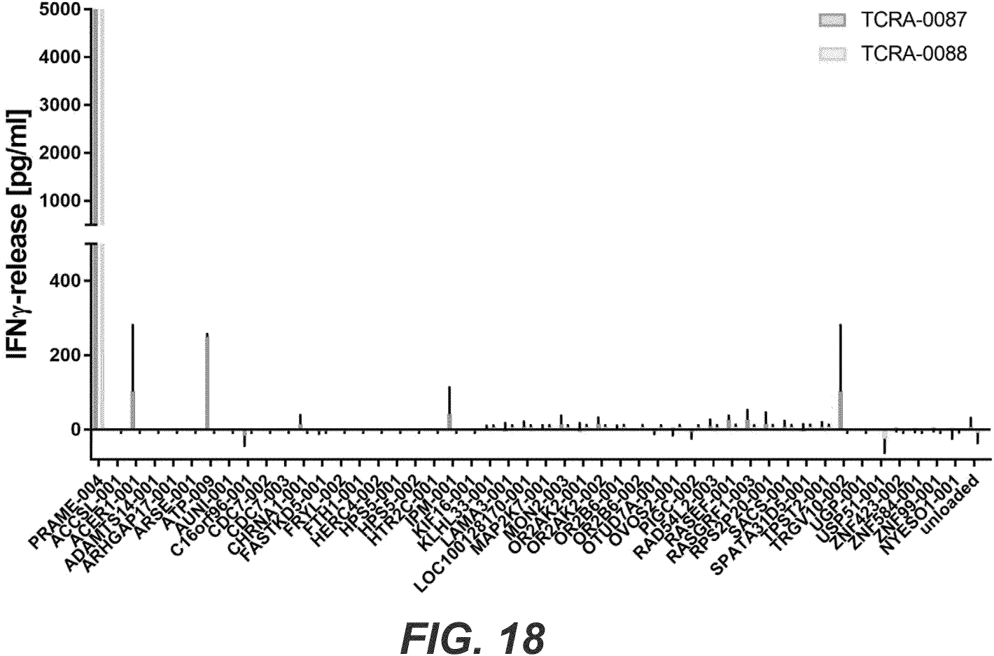

FIG. 18: IFNγ release from CD8+ T-cells lentivirally transduced with TCR R11 P3D3 (Table 7) after co-incubation with T2 target cells loaded with 100 nM PRAME-004 peptide (SEQ ID NO: 310) or similar (identical to PRAME-004 in positions 3, 5, 6 and 7) but unrelated peptides or control peptide NYESO1-001 (SEQ ID NO: 311). IFNγ release data were obtained with CD8+ T-cells derived from two different healthy donors, TCRA-0087 and TCRA-0088.

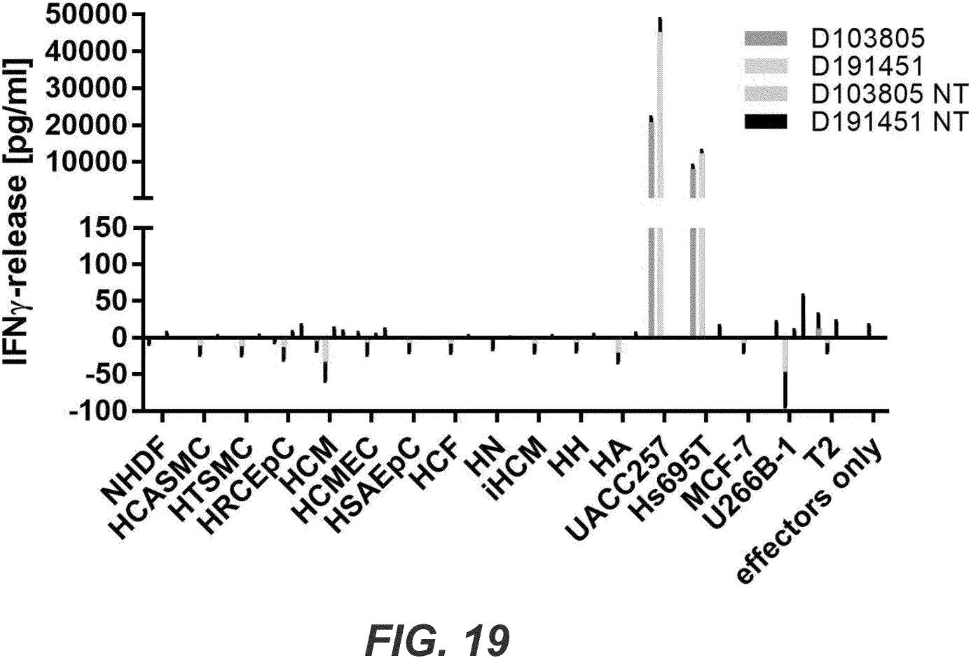

FIG. 19: IFNγ release from CD8+ T-cells lentivirally transduced with TCR R11P3D3 (Table 7) (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release data were obtained with CD8+ T-cells derived from two different healthy donors, D103805 and D191451.

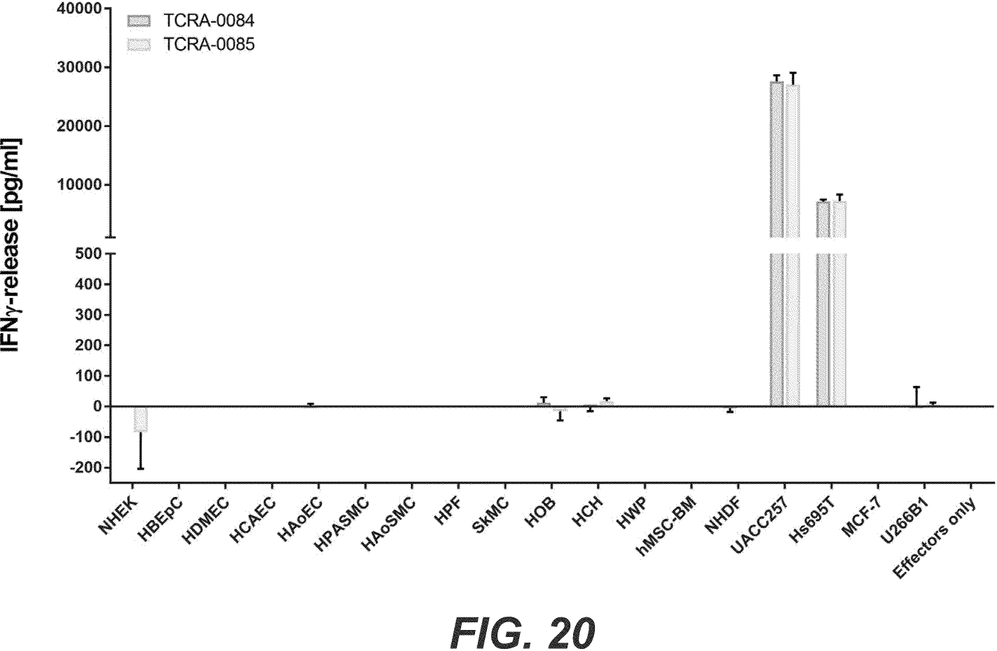

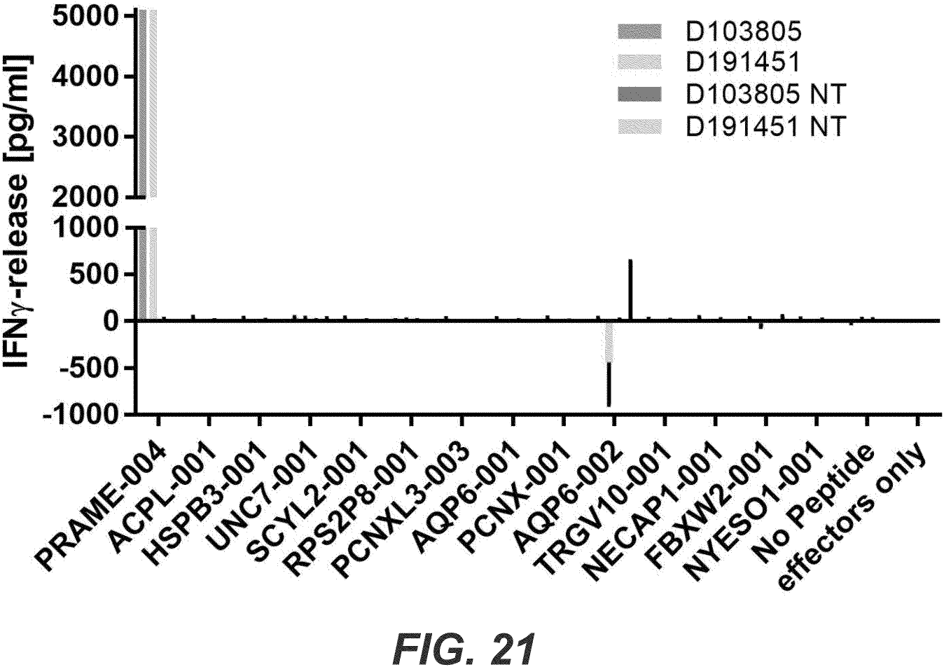

FIG. 20: IFNγ release from CD8+ T-cells lentivirally transduced with TCR R11P3D3 (Table 7) after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copies of PRAME-004 per cells. T-cells alone served as controls. IFNγ release data were obtained with CD8+ T-cells derived from two different healthy donors, TCRA-0084 and TCRA-0085.

FIG. 21: IFNγ release from CD8+ T-cells lentivirally transduced with enhanced TCR R11P3D3_KE (Table 7) (D103805 and D191451) or non-transduced cells (D103805 NT and D191451 NT) after co-incubation with T2 target cells loaded with 100 nM PRAME-004 peptide (SEQ ID NO: 310) or similar (identical to PRAME-004 in positions 3, 5, 6 and 7) but unrelated peptide ACPL-001, HSPB3-001, UNC7-001, SCYL2-001, RPS2P8-001, PCNXL3-003, AQP6-001, PCNX-001, AQP6-002, TRGV10-001, NECAP1-001, FBXW2-001 or control peptide NYESO1-001 (SEQ ID NO: 311). IFNγ release data were obtained with CD8+ T-cells derived from two different healthy donors, D103805 and D191451.

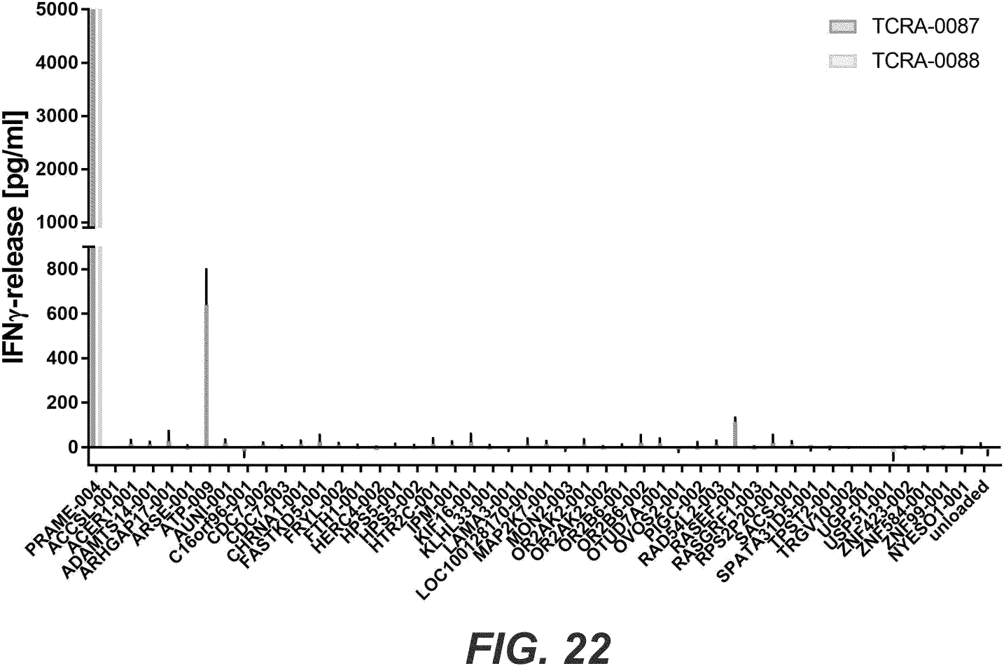

FIG. 22: IFNγ release from CD8+ T-cells lentivirally transduced with enhanced TCR R11P3D3_KE (Table 7) after co-incubation with T2 target cells loaded with 100 nM PRAME-004 peptide (SEQ ID NO: 310) or similar (identical to PRAME-004 in positions 3, 5, 6 and 7) but unrelated peptides or control peptide NYESO1-001 (SEQ ID NO: 311). IFNγ release data were obtained with CD8+ T-cells derived from two different healthy donors, TCRA-0087 and TCRA-0088.

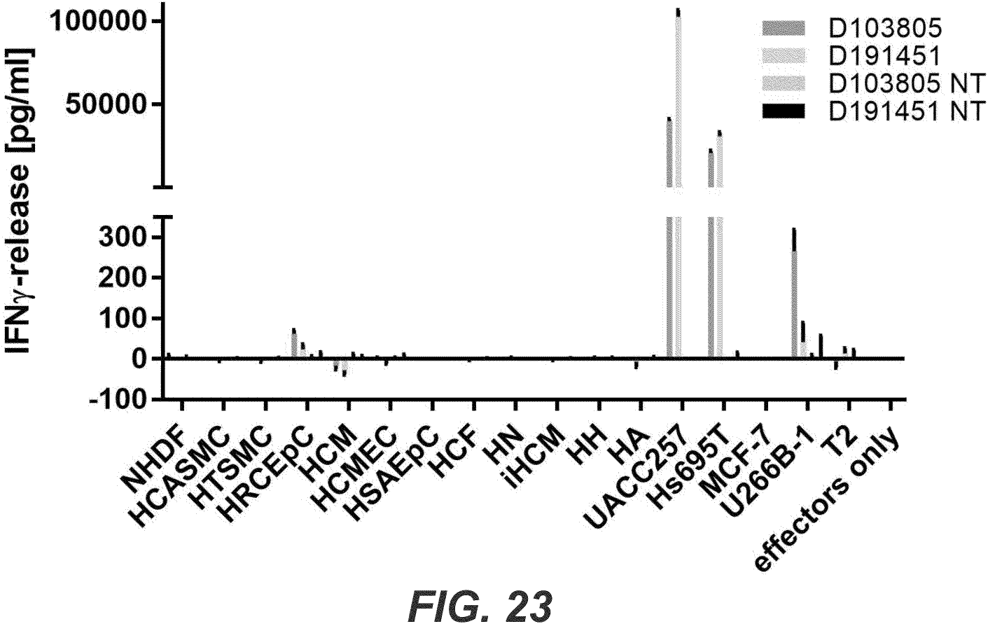

FIG. 23: IFNγ release from CD8+ T-cells lentivirally transduced with enhanced TCR R11P3D3_KE (Table 7) (D103805 and D191451) or non-transduced cells (D103805 NT and D191451 NT) after co-incubation with different primary cells (HCASMC (Coronary artery smooth muscle cells), HTSMC (Tracheal smooth muscle cells), HRCEpC (Renal cortical epithelial cells), HCM (Cardiomyocytes), HCMEC (Cardiac microvascular endothelial cells), HSAEpC (Small airway epithelial cells), HCF (Cardiac fibroblasts)) and iPSC-derived cell types (HN (Neurons), iHCM (Cardiomyocytes), HH (Hepatocytes), HA (astrocytes)). Tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release data were obtained with CD8+ T-cells derived from two different healthy donors, D103805 and D191451.

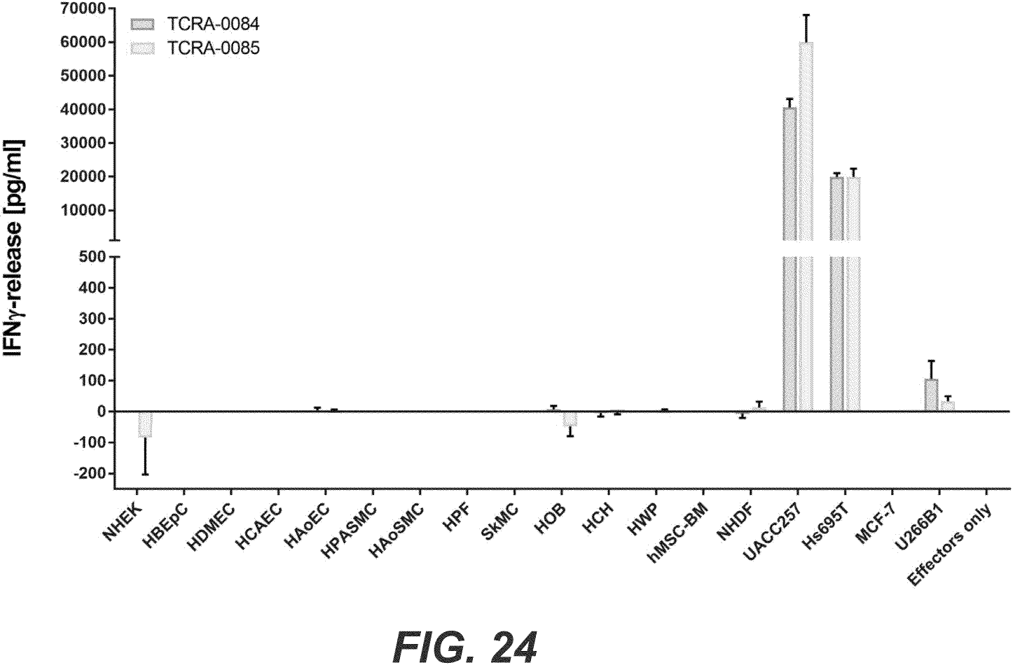

FIG. 24: IFNγ release from CD8+ T-cells lentivirally transduced with enhanced TCR R11P3D3_KE (Table 7) after co-incubation with different primary cells (NHEK (Epidermal keratinocytes), HBEpC (Bronchial epithelial cells), HDMEC (Dermal microvascular endothelial cells), HCAEC (Coronary artery endothelial cells), HAoEC (Aortic endothelial cells), HPASMC (Pulmonary artery smooth muscle cells), HAoSMC (Aortic smooth muscle cells), HPF (Pulmonary fibroblasts), SkMC (Skeletal muscle cells), HOB (osteoblasts), HCH (Chondrocytes), HWP (White preadipocytes), hMSC-BM (Mesenchymal stem cells), NHDF (Dermal fibroblasts). Tumor cell lines UACC-257 (PRAME-004 high), Hs695T (PRAME-004 medium), U266B1 (PRAME-004 very low) and MCF-7 (no PRAME-004) present different copies of PRAME-004 per cells. T-cells alone served as controls. IFNγ release data were obtained with CD8+ T-cells derived from two different healthy donors, TCRA-0084 and TCRA-0085.

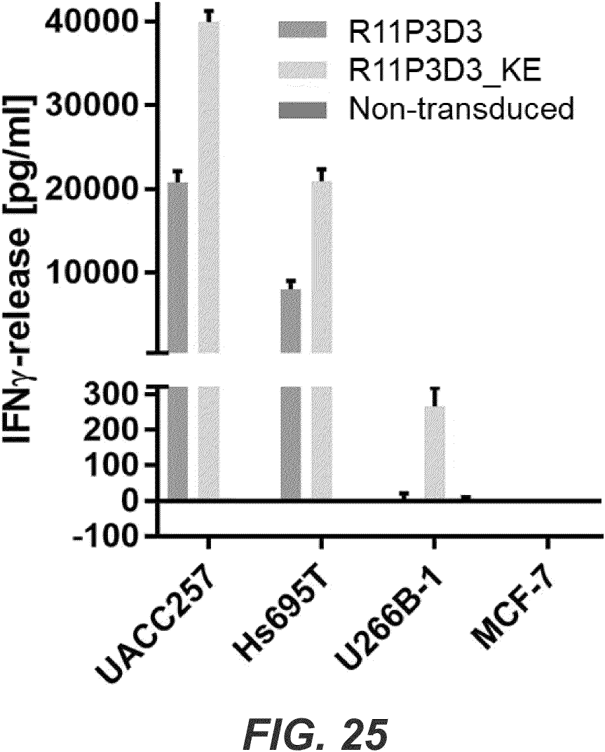

FIG. 25: IFNγ release from CD8+ T-cells lentivirally transduced with TCR R11P3D3 or enhanced TCR R11P3D3_KE (Table 7) or non-transduced cells after co-incubation with tumor cell lines UACC-257 (PRAME-004 high), Hs695T (PRAME-004 medium), U266B1 (PRAME-004 very low) and MCF-7 (no PRAME-004) present different amounts of PRAME-004 per cells. T-cells alone served as controls. IFNγ release of both TCRs correlates with PRAME-004 presentation and R11P3D3_KE induces higher responses compared to R11P3D3.

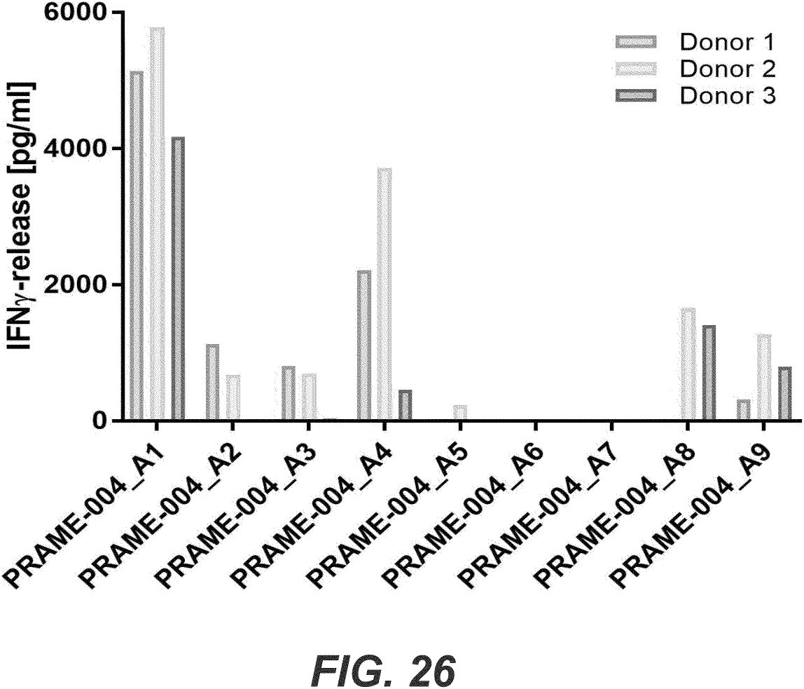

FIG. 26: Potency assay evaluating cytolytic activity of lentivirally transduced T-cells expressing TCR R11 P3D3 or enhanced TCR R11 P3D3_KE against PRAME-004+ tumor cells. Cytotoxic response of R11P3D3 and R11P3D3_KE transduced and non-transduced (NT) T-cells measured against A-375 (PRAME-004 low) or U2OS (PRAME-004 medium) tumor cells. The assays were performed in a 72-hour fluorescence microscopy-based cytotoxicity assay. Results are shown as fold tumor growth over time.

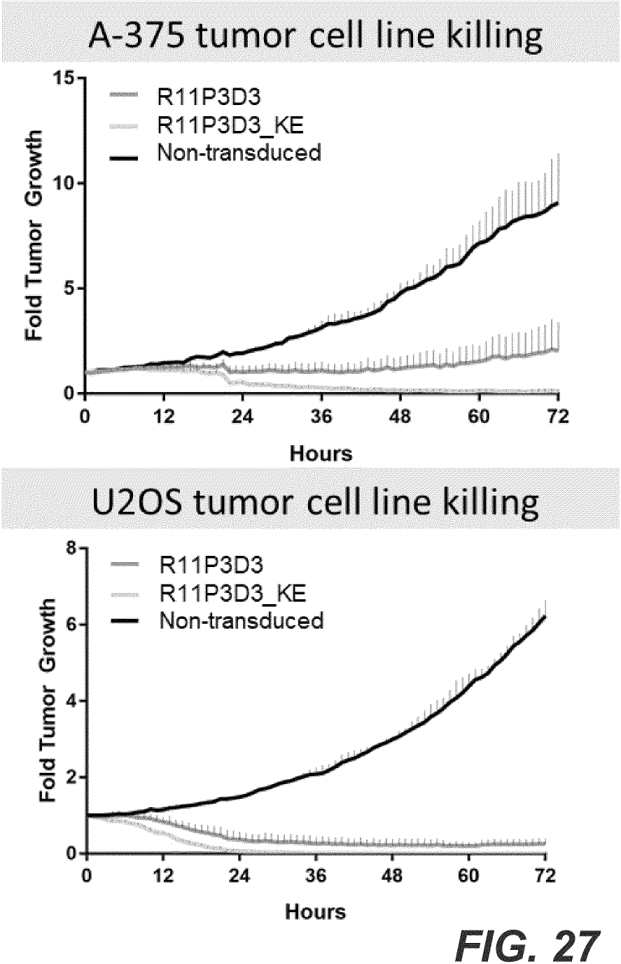

FIG. 27: Potency assay evaluating cytolytic activity of lentivirally transduced T-cells expressing TCR R11 P3D3 or enhanced TCR R11 P3D3_KE against PRAME-004+ tumor cells. Cytotoxic response of R11P3D3 and R11P3D3_KE transduced and non-transduced (NT) T-cells measured against A-375 (PRAME-004 low) or U2OS (PRAME-004 medium) tumor cells. The assays were performed in a 72-hour fluorescence microscopy-based cytotoxicity assay. Results are shown as fold tumor growth over time.

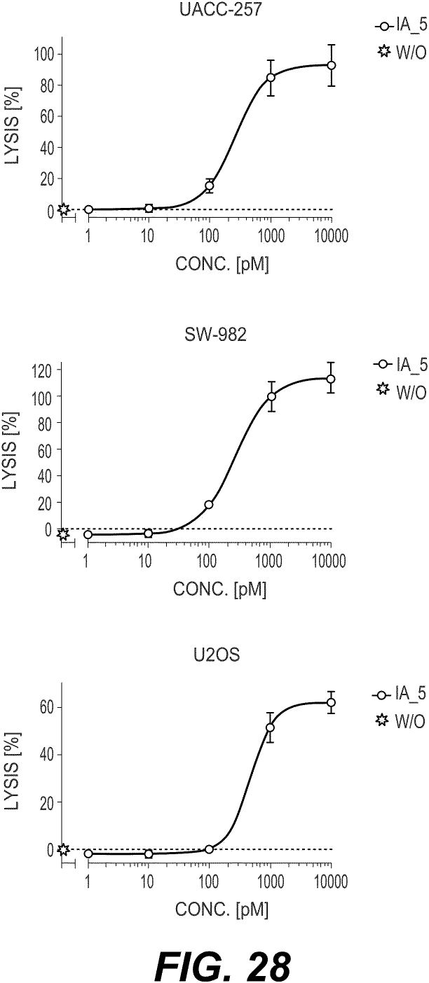

FIG. 28 shows the results of an LDH-release assay with the bispecific TCR/mAb diabody construct IA_5 targeting tumor-associated peptide PRAME-004 (SEQ ID NO: 310) presented on HLA-A*02. CD8-positive T-cells isolated from a healthy donor were co-incubated with cancer cell lines UACC-257, SW982 and U2OS presenting differing amounts of PRAME-004:HLA-A*02-1 complexes on the cell surface (approx. 1100, approx. 770 and approx. 240 copies per cell, respectively, as determined by M/S analysis) at an effector:target ratio of 5:1 in the presence of increasing concentrations of TCR/mAb diabody molecules. After 48 hours of co-culture target cell lysis was quantified utilizing LDH-release assays according to the manufacturer’s instructions (Promega).

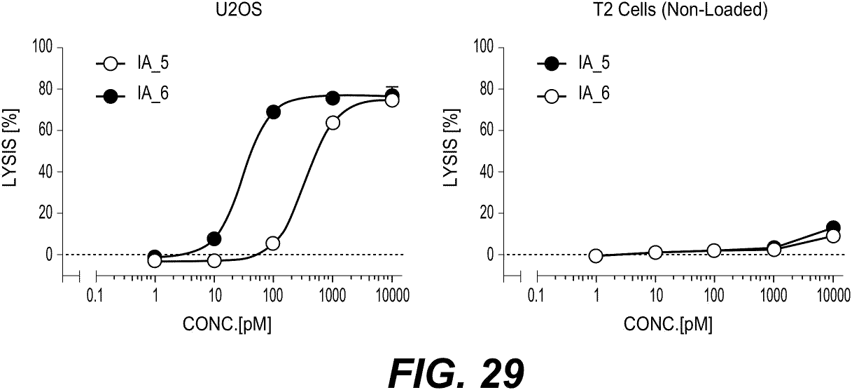

FIG. 29 shows the results of an LDH-release assay with the bispecific TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. CD8-positive T-cells isolated from a healthy donor were co-incubated with the cancer cell line U2OS presenting approx. 240 copies per cell of PRAME-004:HLA-A*02-1 complexes or non-loaded T2 cells (effector:target ratio of 5:1) in the presence of increasing concentrations of TCR/mAb diabody molecules. After 48 hours of coculture target cell lysis was quantified utilizing LDH-release assays according to the manufacturer’s instructions (Promega).

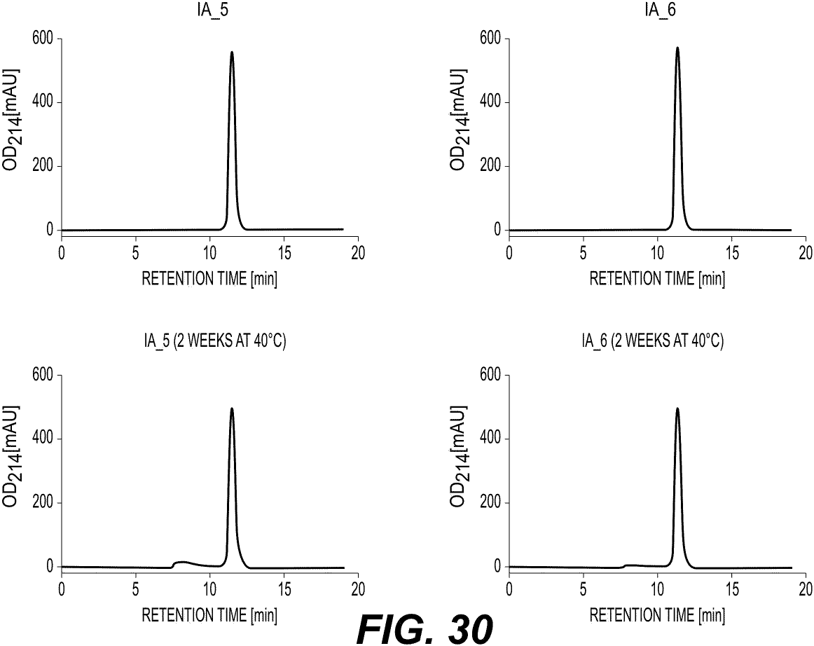

FIG. 30 shows the results of a heat-stress stability study of the TCR/mAb diabody constructs IA_5 and IA_6 utilizing a stability/affinity maturated TCR and an enhanced version thereof, respectively, against the tumor-associated peptide PRAME-004 (SEQ ID NO: 310) presented on HLA-A*02. For this, the proteins were formulated in PBS at a concentration of 1 mg/mL and subsequently stored at 40° C. for two weeks. Protein integrity and recovery was assessed utilizing HPLC-SEC. Thereby the amount of high-molecular weight species was determined according to percentage of peak area eluting before the main peak. Recovery of monomeric protein was calculated by comparing main peak areas of unstressed and stressed samples.

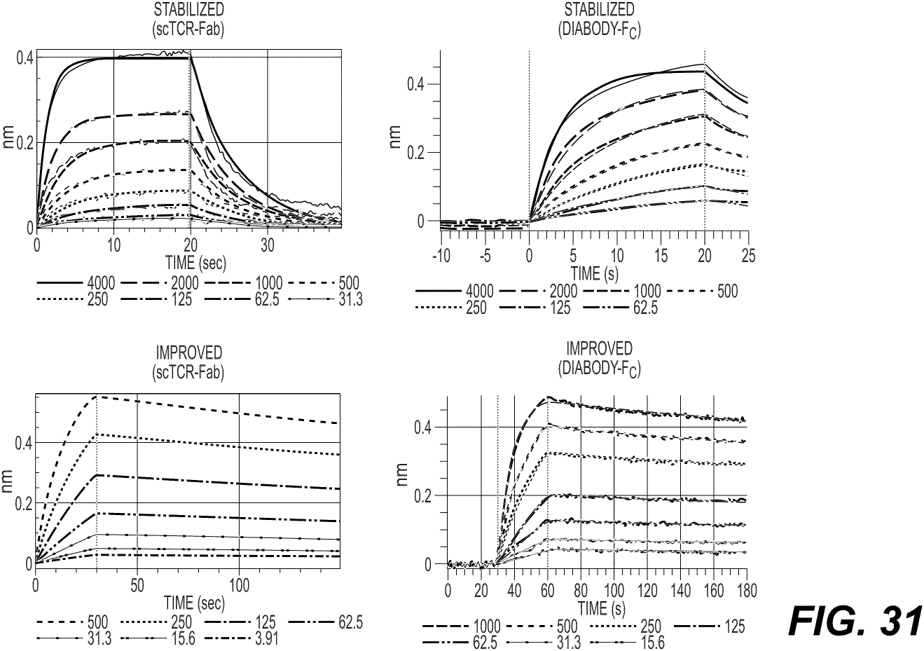

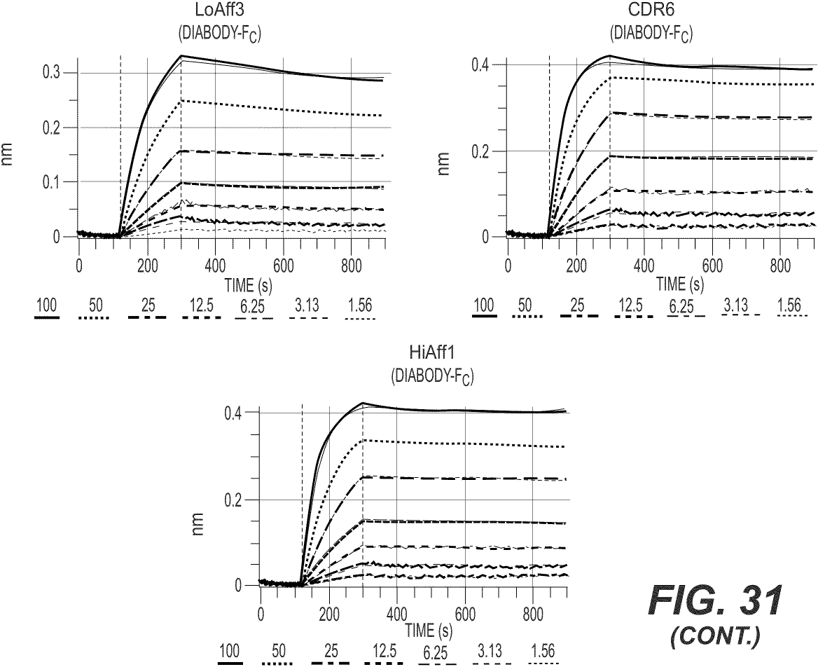

FIG. 31: Binding kinetics of bispecific molecules comprising different R16P1C10 variants. FAB2G sensors were used for the scTCR-Fab format (20 µg/ml loaded for 120 s), AHC sensors for the diabody-F_{c} formats (10 µg/ml loaded for 120 s for improved variant; 5 µg/ml loaded for 120 s for stabilized variant, LoAff3, CDR6, HiAff1). Analyzed concentrations of HLA-A*02/PRAME-004 are represented in nM. Graphs show curves of measured data and calculated fits.

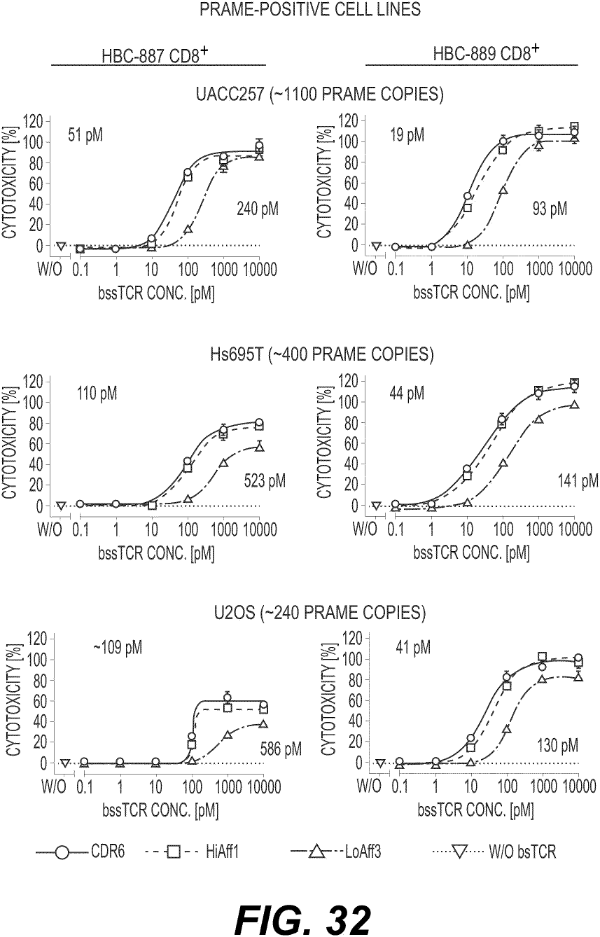

FIG. 32: Lysis of PRAME-positive tumor cell lines induced by bispecific molecules containing CDR6, HiAff1 or LoAff3 TCR variants, respectively, in presence of CD8+ T-cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

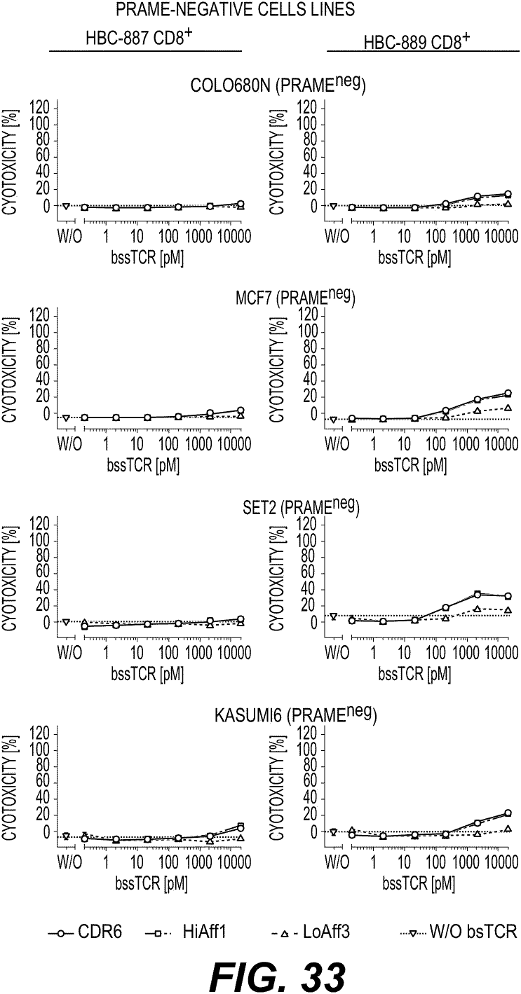

FIG. 33: Lysis of PRAME-negative tumor cell lines induced by bispecific molecules containing CDR6, HiAff1 or LoAff3 TCR variants, respectively, in presence of CD8+ T-cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

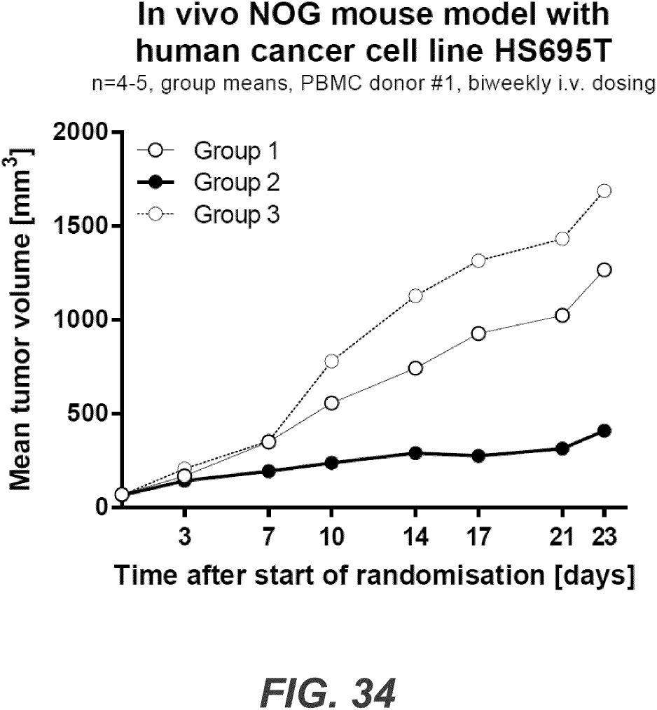

FIG. 34: In vivo efficacy. NOG mice bearing HS695T tumors of approximately 50 mm³ were transplanted with human PBMCs and treated with PBS (group 1), 0.5 mg/kg body HiAff1/antiCD3 diabody-Fc (group 2) or 0.5 mg/kg antiHIV/antiCD3 diabody-Fc (group) i.v. twice a week. Tumor volumes were measured with a caliper and calculated by length × width² /2.

FIG. 35: In vitro cytotoxicity of TCER® molecules on target-positive and target-negative tumor cell lines. PBMC from a healthy HLA-A*02-positive donor were incubated with either target-positive tumor cell line Hs695T (●) or target-negative, but HLA-A*02-positive tumor cell line T98G (◯), respectively, at a ratio of 1:10 in the presence of increasing TCER® concentrations. TCER®-induced cytotoxicity was quantified after 48 hours of co-culture by measurement of released LDH. Results for experiments assessing TPP-93 and TPP-79 are shown in the upper and lower panel, respectively.

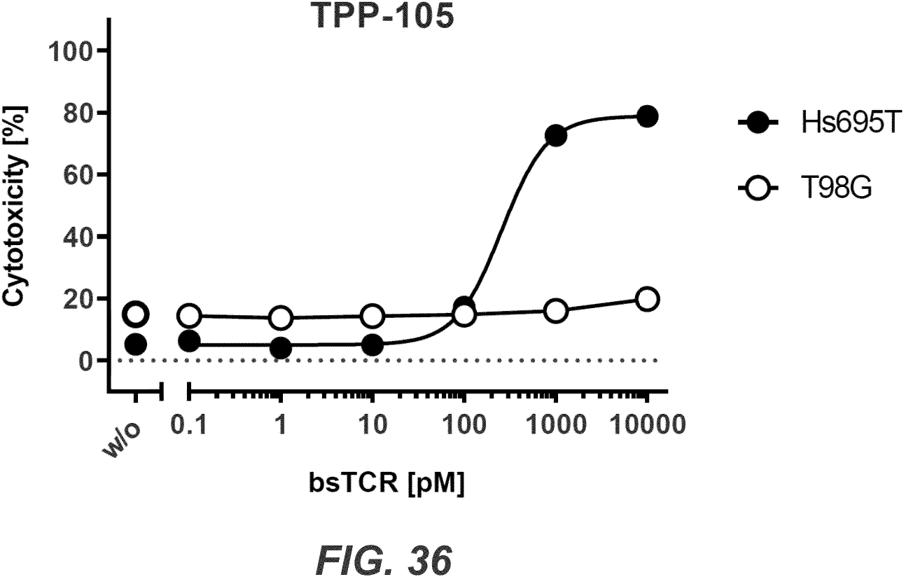

FIG. 36: In vitro cytotoxicity of TCER® molecule TPP-105 on target-positive and target-negative tumor cell lines. PBMC from a healthy HLA-A*02-positive donor were incubated with either target-positive tumor cell line Hs695T (●) or target-negative, but HLA-A*02-positive tumor cell line T98G (◯), respectively, at a ratio of 1:10 in the presence of increasing concentrations of TPP-105. TCER®-induced cytotoxicity was quantified after 48 hours of co-culture by measurement of released LDH.

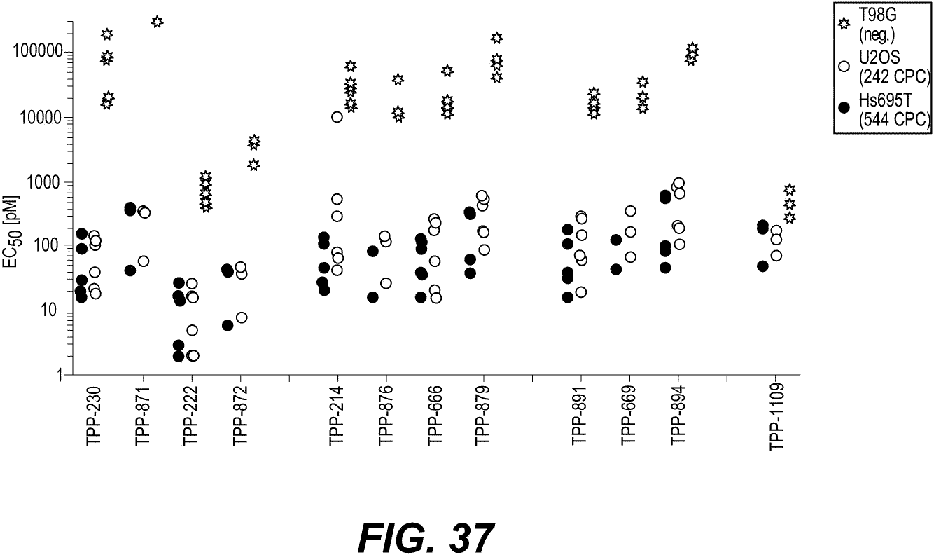

FIG. 37: Summary of cytotoxicity data of TCER® Slot III molecules. Ec₅₀ values of dose-response curves obtained in LDH-release assays were calculated utilizing non-linear 4-point curve fitting. For each assessed TCER®-molecule calculated Ec₅₀ values on target-positive tumor cell lines Hs695T (●), U2OS (o), and target-negative but HLA-A*02-positive tumor cell line T98G (*) are depicted. Thereby, each symbol represents one assay utilizing PBMC derived from various HLA-A*02-positive donors. For TPP-871/T98G, the Ec₅₀ is estimated, as T98G was not recognized by TPP-871.

FIGS. 38A-38C: In vitro cytotoxicity of TCER® Slot III variants on T2 cells loaded with different concentrations of target peptide. Cytotoxicity was determined by quantifying LDH released into the supernatants. Human PBMC were used as effector cells at an E:T ratio of 5:1. Read-out was performed after 48 h.

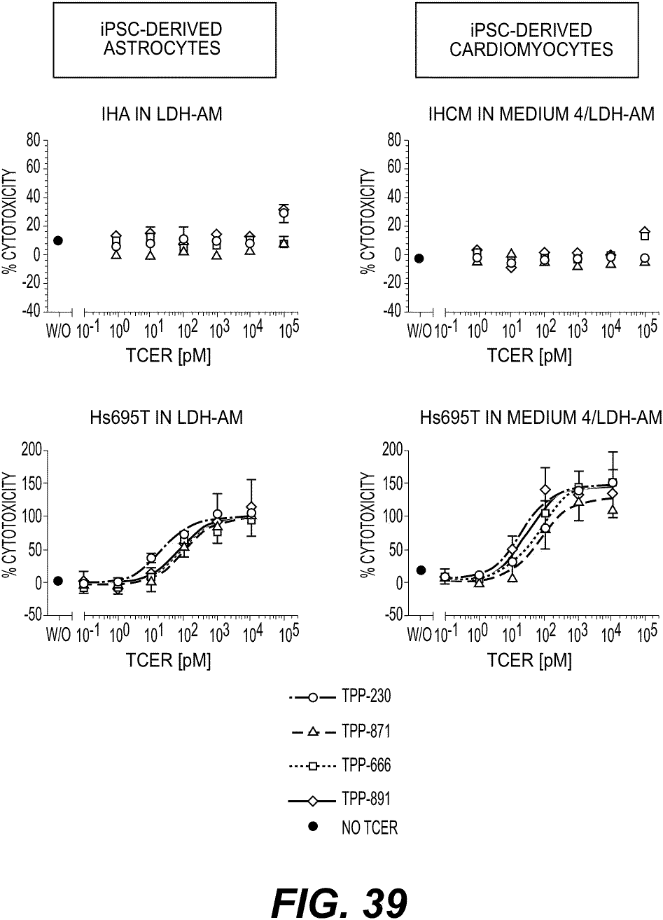

FIG. 39: Normal tissue cell safety analysis for selected TCER® Slot III variants.

TCER®-mediated cytotoxicity against 5 different normal tissue cell types expressing HLA-A*02 was assessed in comparison to cytotoxicity directed against PRAME-004-positive Hs695T tumor cells. PBMCs from a healthy HLA-A*02+ donor were co-cultured at a ratio 10:1 with the normal tissue cells or Hs695T tumor cells (in triplicates) in a 1:1 mixture of the respective normal tissue cell medium (4, 10a or 13a) and T-cell medium (LDH-AM) or in T-cell medium alone. After 48 hours, lysis of normal tissue cells and Hs695T-cells was assessed by measuring LDH release (LDH-Glo™ Kit, Promega).

FIG. 40 shows exemplary non-limiting atezolizumab dosing schedules, starting at Day 14 post-treatment or Day 21 post-treatment. M indicates month after treatment and D indicates D after treatment.

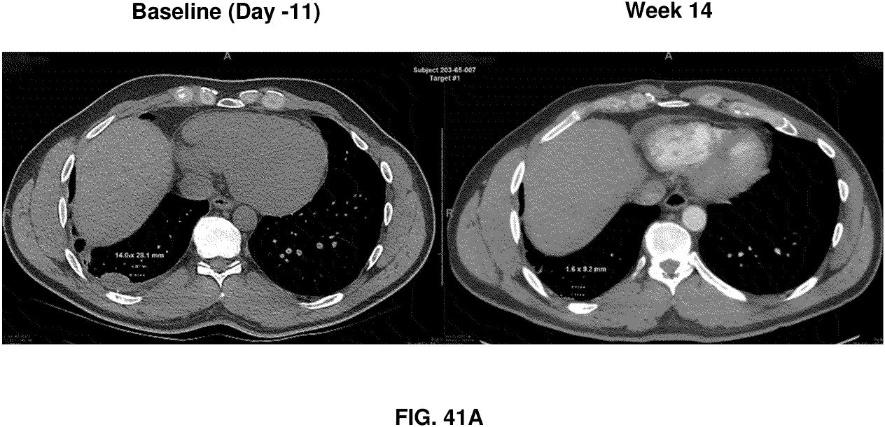

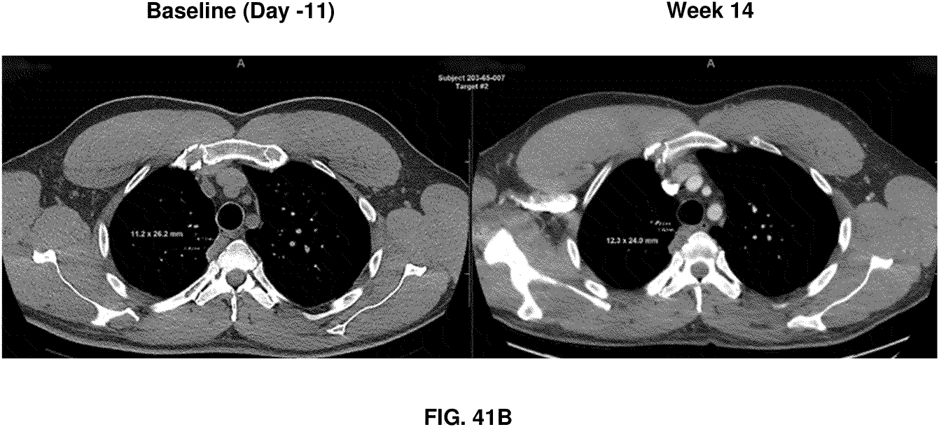

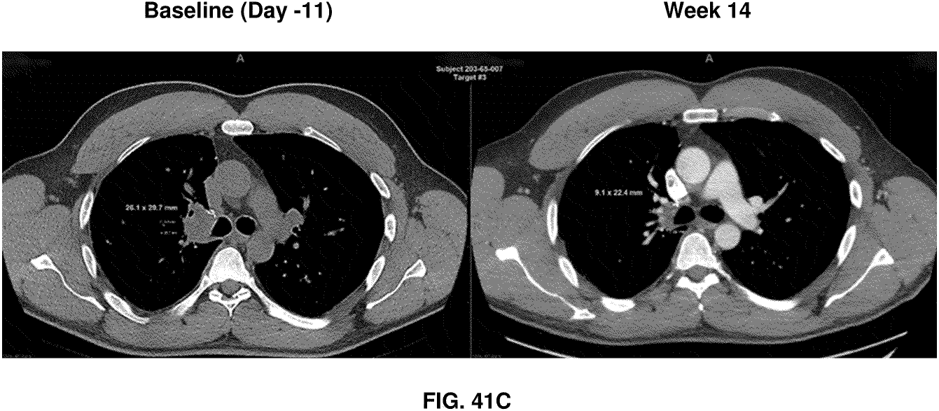

FIG. 41A shows a baseline tumor measurement of 14.0 × 28.1 mm and a post-treatment tumor measurement of 1.6 × 9.2 mm. The tumor is indicated by the white arrow.

FIG. 41B shows a baseline tumor measurement of 11.2 × 26.2 mm and a post-treatment tumor measurement of 12.3 × 24.0 mm. The tumor is indicated by the white arrow.

FIG. 41C shows a baseline tumor measurement of 26.1 × 29.7 mm and a post-treatment tumor measurement of 9.1 × 22.4 mm. The tumor is indicated by the white arrow.

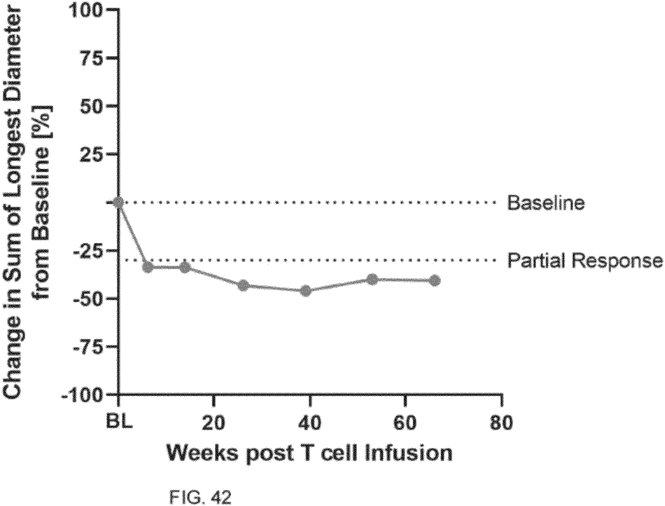

FIG. 42 is a graph showing the relative change in diameter of target lesion upon IMA203 treatment over time. The patient shows a durable response with an ongoing progression-free survival of more than 16 month and a duration of response of more than 15 months.

### EXAMPLES

While the present disclosure has been illustrated and described in detail in the drawings and foregoing description, such illustration and description are to be considered illustrative or exemplary and not restrictive; the present disclosure is not limited to the disclosed embodiments. Other variations to the disclosed embodiments can be understood and effected by those skilled in the art in practicing the claimed present disclosure, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1

### T-Cell Receptor R11P3D3

TCR R11P3D3 (SEQ ID NO: 12 - 23 and 120) is restricted towards HLA-A*02-presented PRAME-004 (SEQ ID NO: 310) (see FIG. 3).

R11P3D3 specifically recognizes PRAME-004, as human primary CD8+ T-cells re-expressing this TCR release IFNγ upon co-incubation with HLA-A*02+ target cells, loaded with PRAME-004 peptide or different peptides showing high degree of sequence similarity to PRAME-004 (FIG. 3). NYESO1-001 (SEQ ID NO: 311) peptide is used as negative control. TCR R11P3D3 has an EC50 of 0.74 nM (FIG. 10) and a binding affinity (K_{D}) of 18 - 26 µM towards HLA-A*02-presented PRAME-004 (SEQ ID NO: 310).

Re-expression of R11P3D3 in human primary CD8+ T-cells leads to selective recognition and killing of HLA-A*02/PRAME-004-presenting tumor cell lines (FIGS. 19, 20, 25 and 27). TCR R11P3D3 does not respond to any of the 25 tested healthy, primary or iPSC-derived cell types (FIGS. 19 and 20) and was tested for cross-reactivity towards further 67 similar peptides (of which 57 were identical to PRAME-004 in positions 3, 5, 6 and 7) but unrelated peptides in the context of HLA-A*02 (FIGS. 3, 17 and 18).

### Example 2

### T-Cell Receptor R16P1C10

TCR R16P1C10 (SEQ ID NOs: 24 - 35 and 121) is restricted towards HLA-A*02-presented PRAME-004 (SEQ ID NO: 310) (see FIG. 4).

R16P1C10 specifically recognizes PRAME-004, as human primary CD8+ T-cells re-expressing this TCR release IFNγ upon co-incubation with HLA-A*02+ target cells and bind HLA-A*02 tetramers (FIG. 16), respectively, loaded either with PRAME-004 peptide or different peptides showing high degree of sequence similarity to PRAME-004 (FIG. 4). NYESO1-001 (SEQ ID NO: 311) peptide is used as negative control. TCR R16P1C10 has an EC50 of 9.6 nM (FIG. 11).

### Example 3

### T-Cell Receptor R16P1E8

TCR R16P1E8 (SEQ ID NOs: 36-47 and 122) is restricted towards HLA-A*02-presented PRAME-004 (SEQ ID NO: 310) (see FIG. 5).

R16P1E8 specifically recognizes PRAME-004, as human primary CD8+ T-cells re-expressing this TCR release IFNγ upon co-incubation with HLA-A*02+ target cells, loaded either with PRAME-004 peptide or alanine or different peptides showing high degree of sequence similarity to PRAME-004 (FIG. 5). NYESO1-001 (SEQ ID NO: 311) peptide (SLLMWITQV, SEQ ID NO: 311) is used as negative control. TCR R16P1E8 has an EC50 of ~1 µM (FIG. 12).

### Example 4

### T-Cell Receptor R17P1A9

TCR R17P1A9 (SEQ ID NOs: 48-59 and 123) is restricted towards HLA-A*02-presented PRAME-004 (SEQ ID NO: 310) (see FIG. 6).

R17P1A9 specifically recognizes PRAME-004, as human primary CD8+ T-cells re-expressing this TCR release IFN_{y} upon co-incubation with HLA-A*02+ target cells, loaded either with PRAME-004 peptide or different peptides showing high degree of sequence similarity to PRAME-004 (FIG. 6). NYESO1-001 (SEQ ID NO: 311) peptide is used as negative control.

### Example 5

### T-Cell Receptor R17P1D7

TCR R17P1D7 (SEQ ID NOs: 60 - 71 and 124) is restricted towards HLA-A*02-presented PRAME-004 (SEQ ID NO: 310) (see FIG. 7).

R17P107 specifically recognizes PRAME-004, as human primary CD8+ T-cells re-expressing this TCR release IFN_{y} upon co-incubation with HLA-A*02+ target cells, loaded either with PRAME-004 peptide or alanine or different peptides showing high degree of sequence similarity to PRAME-004 (FIG. 7). NYESO1-001 (SEQ ID NO: 311) peptide is used as negative control. TCR R17P1D7 has an EC50 of 1.83 nM (FIG. 13).

### Example 6

### T-Cell Receptor R17P1 G3

TCR R17P1G3 (SEQ ID NOS: 72-83 and 125) is restricted towards HLA-A*02-presented PRAME-004 (SEQ ID NO: 310) (see FIG. 8).

R17P1G3 specifically recognizes PRAME-004, as human primary CD8+ T-cells re-expressing this TCR release IFN_{y} upon co-incubation with HLA-A*02+ target cells, loaded either with PRAME-004 peptide or different peptides showing high degree of sequence similarity to PRAME-004 (FIG. 8). NYESO1-001 (SEQ ID NO: 311) peptide is used as negative control. TCR R17P1G3 has an EC50 of 8.63 nM (FIG. 14).

### Example 7

### T-Cell Receptor R17P2B6

TCR R17P2B6 (SEQ ID NOS: 84-95 and 126) is restricted towards HLA-A*02-presented PRAME-004 (SEQ ID NO: 310) (see FIG. 9).

R17P2B6 specifically recognizes PRAME-004, as human primary CD8+ T-cells re-expressing this TCR release IFN_{y} upon co-incubation with HLA-A*02+ target cells, loaded either with PRAME-004 peptide or alanine or different peptides showing high degree of sequence similarity to PRAME-004 (FIG. 9). NYESO1-001 (SEQ ID NO: 311) peptide is used as negative control. TCR R17P2B6 has an EC50 of 2.11 nM (FIG. 15) and a binding affinity (K_{D}) of 13 µM towards HLA-A*02-presented PRAME-004.

### Example 8

### Enhanced T-Cell Receptor R11 P3D3_KE

The mutated “enhanced pairing” TCR R11P3D3_KE is introduced as a variant of R11P3D3, where α and β variable domains, naturally bearing αW44/ βQ44, have been mutated to αK44/ βE44. The double mutation is selected among the list present in PCT/EP2017/081745, herewith specifically incorporated by reference. It is specifically designed to restore an optimal interaction and shape complementarity to the TCR scaffold.

Compared with the parental TCR R11P3D3 the enhanced TCR R11P3D3_KE shows superior sensitivity of PRAME-004 recognition. The response towards PRAME-004-presenting tumor cell lines are stronger with the enhanced TCR R11P3D3_KE compared to the parental TCR R11P3D3 (FIG. 25). Furthermore, the cytolytic activity of R11P3D3_KE is stronger compared to R11P3D3 (FIG. 27). The observed improved functional response of the enhanced TCR R11P3D3_KE is well in line with an increased binding affinity towards PRAME-004, as described in Example 1 (R11P3D3, K_{D} =18-26 µM) and Example 8 (R11P3D3_KE, K_{D} =5.3 µM).

### Example 9

### Generation of Cancer-Targeting Bispecific TCR/mAb Diabody Molecules

To further validate the platform capabilities of bispecific TCR/mAb diabody constructs, the TCR-derived variable domains were exchanged with variable domains of a TCR, which was stability/affinity maturated by yeast display according to a method described previously (Smith et al, 2015, T-cell Receptor Engineering and Analysis Using the Yeast Display Platform. Methods Mol Biol. 1319:95-141). The TCR variable domains specifically binds to the tumor-associated peptide PRAME-004 (SEQ ID NO: 310) bound to HLA-A*02. Furthermore, the variable domains of hUCHT1 (Var17), a humanized version of the UCHT1 antibody, was used to generate the PRAME-004-targeting TCR/mAb diabody molecule IA_5 (comprising SEQ ID NO: 131 and SEQ ID NO: 132). Expression, purification and characterization of this molecule was performed. Purity and integrity of final preparation exceeded 96% according to HPLC-SEC analysis.

Binding affinities of bispecific TCR/mAb diabody constructs towards PRAME-004:HLA-A*02 were determined by biolayer interferometry. Measurements were done on an Octet RED384 system using settings recommended by the manufacturer. Briefly, purified bispecific TCR/mAb diabody molecules were loaded onto biosensors (AHC) prior to analyzing serial dilutions of HLA-A*02/PRAME-004.

The activity of this PRAME-004-targeting TCR/mAb diabody construct with respect to the induction of tumor cell lysis was evaluated by assessing human CD8-positive T-cell-mediated lysis of the human cancer cell lines UACC-257, SW982 and U2OS presenting different copy numbers of PRAME-004 peptide in the context of HLA-A*02 on the tumor cell surface (UACC-257 - about 1100, SW982 - about 770, U2OS - about 240 PRAME-004 copies per cell, as determined by quantitative M/S analysis) as determined by LDH-release assay.

As depicted in FIG. 28, the PRAME-004-targeting TCR/mAb diabody construct IA_5 induced a concentration-dependent lysis of PRAME-004 positive tumor cell lines. Even tumor cells U2OS expressing as little as 240 PRAME-004 copy numbers per tumor cell were efficiently lysed by this TCR/mAb diabody molecule. These results further demonstrate that TCR/mAb diabody format is applicable as molecular platform allowing to introduce variable domains of different TCRs as well as variable domains of different T-cell recruiting antibodies.

### Example 10

### Engineerability of TCR/mAb Diabody Constructs

The variable TCR domains utilized in construct IA_5 were further enhanced regarding affinity towards PRAME-004 and TCR stability, and used for engineering into TCR/mAb diabody scaffold resulting in construct IA_6 (comprising SEQ ID NO: 133 and SEQ ID NO: 134). Expression, purification and characterization of TCR/mAb diabody molecules IA_5 and IA_6 were performed. Purity and integrity of final preparations exceeded 97% according to HPLC-SEC analysis.

Potency of the stability and affinity enhanced TCR/mAb diabody variant IA_6 against PRAME-004 was assessed in cytotoxicity experiments with the tumor cell line U2OS presenting low amounts of PRAME-004:HLA-A*02 or non-loaded T2 cells as target cells and human CD8-positive T-cells as effector cells.

As depicted in FIG. 29, the inventors observed and increased cytotoxic potency of the TCR/Ab diabody molecule IA_6 comprising the variable domains of the stability/affinity enhanced TCR variant when compared to the precursor construct IA_5. For both constructs, IA_5 and IA_6, the PRAME-004-dependent lysis could be confirmed as no cytolysis of target-negative T2 cells was detected.

The protein constructs were further subjected to heat-stress at 40° C. for up to two weeks to analyze stability of the PRAME-004-specific TCR/mAb diabody variants IA_5 and IA_6. HPLC-SEC analyses after heat-stress revealed a significantly improved stability of the variant IA_6 when compared to the precursor construct IA_5 (see FIG. 30). The temperature-induced increase of high-molecular species (i.e., eluting before the main peak) of the constructs was less pronounced for IA_6 than for IA_5. In line with this result, the recovery of intact, monomeric protein after heat-stress was 87% and 92% for IA_5 and IA_6, respectively.

These exemplary engineering data demonstrate that the highly potent and stable of TCR/mAB diabody constructs can further be improved by incorporating stability/affinity enhanced TCR variable domains resulting in therapeutic proteins with superior characteristics.

### Example 11

### Binding Affinities of Maturated TCR Variants

Maturated R16P1C10 TCR variants expressed as soluble bispecific molecules (stabilized, improved: scTCR/antiCD3 Fab format; stabilized, improved, CDR6, HiAff1 and LoAff3: TCR/antiCD3 diabody-F_{c} format) were analyzed for their binding affinity towards HLA-A*02/PRAME-004 monomers via biolayer interferometry. Measurements were performed on an Octet RED384 system using settings recommended by the manufacturer. Briefly, binding kinetics were measured at 30° C. and 1000 rpm shake speed using PBS, 0.05% Tween-20, 0.1% BSA as buffer. Bispecific molecules were loaded onto biosensors (FAB2G or AHC) prior to analyzing serial dilutions of HLA-A*02/PRAME-004. While a stabilized version of R16P1C10 showed an affinity of approximately 1 µM (1.2 µM as scTCR-Fab, 930 nM as diabody-F_{c}), considerably lower K_{D} values were determined for all variants containing maturated CDRs (Tables 4 and 7, FIG. 31). To further validate that the affinity of a TCR variant is influenced by the format only to a minor extent, K_{D} values of an affinity-maturated TCR variant were measured as scTCR-Fab or diabody-F_{c} format. The scTCR-Fab and diabody-F_{c} formats showed K_{D} values of 10 nM and 8.7 nM, respectively, further highlighting good comparability between the different formats (Tables 4 and 7, FIG. 31).

### Example 12

### Killing of Target-Positive and Target-Negative Tumor Cell Lines

Maturated R16P1C10 TCR variants were expressed as soluble bispecific molecules employing a TCR/antiCD3 diabody-F_{c} format. The cytotoxic activity of the bispecific molecules against PRAME-positive and PRAME-negative tumor cell lines, respectively was analyzed by LDH-release assay. Therefore, tumor cell lines presenting variable amounts of HLA-A*02/PRAME-004 on the cell surface were co-incubated with CD8+ T-cells isolated from two healthy donors in presence of increasing concentrations of bispecific molecules. After 48 hours, lysis of target cell lines was measured utilizing CytoTox 96 Non-Radioactive Cytotoxicity Assay Kits (PROMEGA). As shown in FIG. 32, for all tested PRAME-positive cell lines, highly efficient induction of lysis was detectable and clearly depending on concentration of bispecific molecules. In similar experiments utilizing cell lines expressing HLA-A*02 but not presenting the peptide PRAME-004 at detectable levels, FIG. 33 shows no or only marginal lysis of targets was induced by the bispecific molecules indicating the specificity of the TCR domains.

### Example 13

### In Vivo Efficacy

Maturated R16P1C10 TCR variant HiAff1 and a HIV-specific high affinity control TCR were expressed as soluble bispecific molecules employing a TCR/antiCD3 diabody-F_{c} format. A pharmacodynamic study designed to test the ability of the bispecific TCR molecules in recruiting and directing the activity of human cytotoxic CD3+ T-cells against a PRAME-positive tumor cell line HS695T was performed in the hyper immune-deficient NOG mouse strain. The NOG mouse strain hosted the subcutaneously injected human tumor cell line HS695T and intravenously injected human peripheral blood mononuclear cell xenografts. Human peripheral blood mononuclear cells (5×1 0⁶ cells/mouse, intravenous injection) were transplanted within 24 hours when individual tumor volume reached 50 mm³. Treatment was initiated within one hour after transplantation of human blood cells. Four to five female mice per group received intravenous bolus injections (5 mL/kg body weight, twice weekly dosing, up to seven doses, starting one day after randomization) into the tail vein. The injected dose of the PRAME-targeting bispecific TCR molecule was 0.5 mg/kg body weight per injection (group 2), PBS was used in the vehicle control group (group 1) and the HIV-targeting control TCR bispecific molecule (0.5 mg/kg body weight per injection) in the negative control substance group (group 3). At the indicated time points, mean tumor volumes were calculated for every group based on the individual tumor volumes that were measured with a caliper and calculated as length x width² / 2. Treatment with PRAME-targeting bispecific TCR molecule inhibited tumor growth as indicated by reduced increase of tumor volume from basal levels (start of randomization) of 65 to 409 mm³ in comparison to the increase observed in the vehicle control group from basal levels of 69 to 1266 mm³ and the negative control substance group from basal levels of 66 to 1686 mm³ at day 23 (FIG. 34).

### Example 14

### Production and Characterization of Soluble scTCR-Fab Molecules

The variable domains of TCR that bind the PRAME-004:MHC complex may be selected from the following:
V_{A} comprises or consists of the amino acid sequence of SEQ ID NO: 305; and
V_{B} comprises or consists of the amino acid sequence of SEQ ID NO: 306;
V_{A} comprises or consists of the amino acid sequence of SEQ ID NO: 305; and
V_{B} comprises or consists of the amino acid sequence of SEQ ID NO: 307;
V_{A} comprises or consists of the amino acid sequence of SEQ ID NO: 305; and
V_{B} comprises or consists of the amino acid sequence of SEQ ID NO: 308;
V_{A} comprises or consists of the amino acid sequence of SEQ ID NO: 309; and
V_{B} comprises or consists of the amino acid sequence of SEQ ID NO: 306;
V_{A} comprises or consists of the amino acid sequence of SEQ ID NO: 309; and
V_{B} comprises or consists of the amino acid sequence of SEQ ID NO: 307; or
V_{A} comprises or consists of the amino acid sequence of SEQ ID NO: 309; and
V_{B} comprises or consists of the amino acid sequence of SEQ ID NO: 306.

Most preferably, V_{A} comprises or consists of the amino acid sequence of SEQ ID NO: 305; and V_{B} comprises or consists of the amino acid sequence of SEQ ID NO: 306.

For targeting of the TCR-CD3 complex, V_{H} and V_{L} domains derived from the CD3-specific, humanized antibody hUCHT1 (Zhu et al., Identification of heavy chain residues in a humanized anti-CD3 antibody important for efficient antigen binding and T-cell activation. J Immunol, 1995, 155, 1903-1910) can be used, in particular V_{H} and V_{L} domains derived from the UCHT1 variants UCHT1-V17, UCHT1-V17opt, UCHT1-V21 or UCHT1-V23, preferably derived from UCHT1-V17, more preferably a V_{H} comprising or consisting of SEQ ID NO: 193; and a V_{L} comprising or consisting of SEQ ID NO: 192; Alternatively, V_{H} and V_{L} domains derived from the antibody BMA031, which targets the TCRα/β CD3 complex, and humanized versions thereof (Shearman et al., Construction, expression and characterization of humanized antibodies directed against the human alpha/beta T-cell receptor, J Immunol, 1991, 147, 4366-73) may be used, in particular V_{H} and V_{L} domains derived from BMA031 variants BMA031 (V36) or BMA031(V10), preferably derived from BMA031(V36), more preferably a V_{H} comprising or consisting of SEQ ID NO: 196; or SEQ ID NO: 198; (A02) or SEQ ID NO: 199; (D01) or SEQ ID NO: 200; (A02_H90Y) or SEQ ID NO: 201; (D01_H90Y), and a V_{L} comprising or consisting of SEQ ID NO: 197; As another alternative, V_{H} and V_{L} domains derived from the CD3ε-specific antibody H2C (described in EP2 1 55 783) may be used, in particular a V_{H} comprising or consisting of SEQ ID NO: 202; or SEQ ID NO: 207; (N100D) or SEQ ID NO: 209; (N100E) or SEQ ID NO: 211; (S101A) and a V_{L} comprising or consisting of SEQ ID NO: 204.

### Example 15

### Identification and Quantitation of Tumor Associated Peptides Presented on The Cell Surface

### Tissue Samples

Patients’ tissues were obtained from: BiolVT (Detroit, MI, USA & Royston, Herts, UK); Bio-Options Inc. (Brea, CA, USA); BioServe (Beltsville, MD, USA); Capital BioScience Inc. (Rockville, MD, USA); Conversant Bio (Huntsville, AL, USA); Cureline Inc. (Brisbane, CA, USA); DxBiosamples (San Diego, CA, USA); Geneticist Inc. (Glendale, CA, USA); Indivumed GmbH (Hamburg, Germany); Kyoto Prefectural University of Medicine (KPUM) (Kyoto, Japan); Osaka City University (OCU) (Osaka, Japan); ProteoGenex Inc. (Culver City, CA, USA); Tissue Solutions Ltd (Glasgow, UK); Universitat Bonn (Bonn, Germany); Asklepios Clinic St. Georg (Hamburg, Germany); Val d′Hebron University Hospital (Barcelona, Spain); Center for cancer immune therapy (CCIT), Herlev Hospital (Herlev, Denmark); Leiden University Medical Center (LUMC) (Leiden, Netherlands); Istituto Nazionale Tumori “Pascale”, Molecular Biology and Viral Oncology Unit (Naples, Italy); Stanford Cancer Center (Palo Alto, CA, USA); University Hospital Geneva (Geneva, Switzerland); University Hospital Heidelberg (Heidelberg, Germany); University Hospital Munich (Munich, Germany); University Hospital Tuebingen (Tuebingen, Germany).

Written informed consents of all patients had been given before surgery or autopsy. Tissues were shock-frozen immediately after excision and stored until isolation of TUMAPs at -70° C. or below.

### Isolation of HLA Peptides From Tissue Samples

HLA peptide pools from shock-frozen tissue samples were obtained by immune precipitation from solid tissues according to a slightly modified protocol (Falk et al., 1991; Seeger et al., 1999) using the HLA-A*02 specific antibody BB7.2, the HLA-A, -B, -C specific antibody w6/32, the HLA-DR specific antibody L243 and the HLA-DP specific antibody B7/21, CNBr-activated sepharose, acid treatment, and ultrafiltration.

### Mass Spectrometry Analyses

The HLA peptide pools as obtained were separated according to their hydrophobicity by reversed-phase chromatography (nanoAcquity UPLC system, Waters) and the eluting peptides were analyzed in LTQ Velos and Fusion hybrid mass spectrometers (Thermo) equipped with an ESI source. Peptide pools were loaded directly onto the analytical fused-silica micro-capillary column (75 µm i.d. x 250 mm) packed with 1.7 µm C18 reversed-phase material (Waters) applying a flow rate of 400 nL per minute. Subsequently, the peptides were separated using a two-step 180 minute-binary gradient from 10% to 33% B at a flow rate of 300 nL per minute. The gradient was composed of Solvent A (0.1% formic acid in water) and solvent B (0.1% formic acid in acetonitrile). A gold coated glass capillary (PicoTip, New Objective) was used for introduction into the nanoESI source. The LTQ-Orbitrap mass spectrometers were operated in the data-dependent mode using a TOP5 strategy. In brief, a scan cycle was initiated with a full scan of high mass accuracy in the orbitrap (R = 30000), which was followed by MS/MS scans also in the orbitrap (R = 7500) on the 5 most abundant precursor ions with dynamic exclusion of previously selected ions. Tandem mass spectra were interpreted by SEQUEST at a fixed false discovery rate (q≤0.05) and additional manual control. In cases where the identified peptide sequence was uncertain it was additionally validated by comparison of the generated natural peptide fragmentation pattern with the fragmentation pattern of a synthetic sequence-identical reference peptide.

Label-free relative LC-MS quantitation was performed by ion counting i.e., by extraction and analysis of LC-MS features (Mueller et al., 2007). The method assumes that the peptide’s LC-MS signal area correlates with its abundance in the sample. Extracted features were further processed by charge state deconvolution and retention time alignment (Mueller et al., 2008; Sturm et al., 2008). Finally, all LC-MS features were cross-referenced with the sequence identification results to combine quantitative data of different samples and tissues to peptide presentation profiles. The quantitative data were normalized in a two-tier fashion according to central tendency to account for variation within technical and biological replicates. Thus, each identified peptide can be associated with quantitative data allowing relative quantification between samples and tissues. In addition, all quantitative data acquired for peptide candidates was inspected manually to assure data consistency and to verify the accuracy of the automated analysis. A presentation profile was calculated showing the mean sample presentation as well as replicate variations.

### Example 16

### Absolute Quantitation of Tumor Associated Peptides Presented on Cell Surface

The generation of binders, such as antibodies and/or TCRs, is a laborious process, which may be conducted only for a number of selected targets. In the case of tumor associated and specific peptides, selection criteria include, but are not restricted to, exclusiveness of presentation and the density of peptide presented on the cell surface. In addition to the isolation and relative quantitation of peptides as described in the examples, the inventors analyzed absolute peptide copies per cell as described in WO 2016/107740. The quantitation of TUMAP copies per cell in solid tumor samples requires the absolute quantitation of the isolated TUMAP, the efficiency of the TUMAP isolation process, and the cell count of the tissue sample analyzed.

### Peptide Quantitation by Nano LC-MS/MS

For an accurate quantitation of peptides by mass spectrometry, a calibration curve was generated for SEQ ID NO: 310 /PRAME-004, using two different isotope labeled peptide variants (one or two isotope-labeled amino acids are included during TUMAP synthesis). These isotopes labeled variants differ from the tumor-associated peptide only in their mass but show no difference in other physicochemical properties (Anderson et al., 2012). For the peptide calibration curve, a series of nano LC-MS/MS measurements was performed to determine the ration of MS/MS signals of titrated (singly isotope-labeled peptide) to constant (doubly isotope labeled peptide) isotope labeled peptides.

The doubly isotope labeled peptide, also called internal standard, was further spiked to each MS sample and all MS signals were normalized to the MS signal of the internal standard to level out potential technical variances between MS experiments.

The calibration curves were prepared in at least three different matrices, i.e., HLA peptide eluates from natural samples similar to the routine MS samples, and each preparation was measured in duplicate MS runs. For evaluation, MS signals were normalized to the signal of the internal standard and a calibration curve was calculated by logistic regression.

For the quantitation of tumor-associated peptides from tissue samples, the respective samples were also spiked with the internal standard; the MS signals were normalized to the internal standard and quantified using the peptide calibration curve.

### Efficiency of Peptide-MHC Isolation

As for any protein purification process, the isolation of proteins from tissue samples is associated with a certain loss of the protein of interest. To determine the efficiency of TUMAP isolation, peptide-MHC complexes were generated for all TUMAPs selected for absolute quantitation. To be able to discriminate the spiked from the natural peptide-MHC complexes, single-isotope-labelled versions of the TUMAPs were used, i.e., one isotope-labelled amino acid was included in TUMAP synthesis. These complexes were spiked into the freshly prepared tissue lysates, i.e. at the earliest possible point of the TUMAP isolation procedure, and then captured like the natural peptide-MHC complexes in the following affinity purification. Measuring the recovery of the single-labelled TUMAPs therefore allows conclusions regarding the efficiency of isolation of individual natural TUMAPs.

The efficiency of isolation was analyzed in a small set of samples and was comparable among these tissue samples. In contrast, the isolation efficiency differs between individual peptides. This suggests that the isolation efficiency, although determined in only a limited number of tissue samples, may be extrapolated to any other tissue preparation. However, it is necessary to analyze each TUMAP individually as the isolation efficiency may not be extrapolated from one peptide to others.

### Determination of the Cell Count in Solid, Frozen Tissue

In order to determine the cell count of the tissue samples subjected to absolute peptide quantitation, the inventors applied DNA content analysis. This method is applicable to a wide range of samples of different origin and, most importantly, frozen samples (Alcoser et al., 2011; Forsey and Chaudhuri, 2009; Silva et al., 2013). During the peptide isolation protocol, a tissue sample is processed to a homogenous lysate, from which a small lysate aliquot is taken. The aliquot is divided in three parts, from which DNA is isolated (QiaAmp DNA Mini Kit, Qiagen, Hilden, Germany). The total DNA content from each DNA isolation is quantified using a fluorescence-based DNA quantitation assay (Qubit dsDNA HS Assay Kit, Life Technologies, Darmstadt, Germany) in at least two replicates.

In order to calculate the cell number, a DNA standard curve from aliquots of isolated healthy blood cells from several donors, with a range of defined cell numbers, has been generated. The standard curve is used to calculate the total cell content from the total DNA content from each DNA isolation. The mean total cell count of the tissue sample used for peptide isolation is then extrapolated considering the known volume of the lysate aliquots and the total lysate volume.

### Example 17

### Expression Profiling of Genes Encoding the Peptides of the Present Disclosure

Over-presentation or specific presentation of a peptide on tumor cells compared to normal cells is sufficient for its usefulness in immunotherapy, and some peptides are tumor-specific despite their source protein occurring also in normal tissues. Still, mRNA expression profiling adds an additional level of safety in selection of peptide targets for immunotherapies. Especially for therapeutic options with high safety risks, such as affinity-matured TCRs, the ideal target peptide will be derived from a protein that is unique to the tumor and not found on normal tissues.

### RNA Sources and Preparation

Surgically removed tissue specimens were provided as indicated above (see Example 1) after written informed consent had been obtained from each patient. Tumor tissue specimens were snap-frozen immediately after surgery and later homogenized with mortar and pestle under liquid nitrogen. Total RNA was prepared from these samples using TRI Reagent (Ambion, Darmstadt, Germany) followed by a cleanup with RNeasy (QIAGEN, Hilden, Germany); both methods were performed according to the manufacturer’s protocol.

Total RNA from healthy human tissues for RNASeq experiments was obtained from: Asterand (Detroit, MI, USA & Royston, Herts, UK); Bio-Options Inc. (Brea, CA, USA); Geneticist Inc. (Glendale, CA, USA); ProteoGenex Inc. (Culver City, CA, USA); Tissue Solutions Ltd (Glasgow, UK).

Total RNA from tumor tissues for RNASeq experiments was obtained from: Asterand (Detroit, MI, USA & Royston, Herts, UK); BioCat GmbH (Heidelberg, Germany); BioServe (Beltsville, MD, USA); Geneticist Inc. (Glendale, CA, USA); Istituto Nazionale Tumori “Pascale” (Naples, Italy); ProteoGenex Inc. (Culver City, CA, USA); University Hospital Heidelberg (Heidelberg, Germany).

Quality and quantity of all RNA samples were assessed on an Agilent 2100 Bioanalyzer (Agilent, Waldbronn, Germany) using the RNA 6000 Pico LabChip Kit (Agilent).

### RNAseq Experiments

Gene expression analysis of tumor and normal tissue RNA samples was performed by next-generation sequencing (RNAseq) by GENEWIZ Germany GmbH (Leipzig, Germany). Briefly, sequencing libraries were prepared from total RNA using the NEBNext® Ultra™ II Directional RNA Library Prep Kit for Illumina according to the manufacturer’s instructions (New England Biolabs, Ipswich, MA, USA), which includes mRNA selection, RNA fragmentation, cDNA conversion and addition of sequencing adaptors. For sequencing, libraries were multiplexed and loaded onto the Illumina NovaSeq 6000 sequencer (Illumina Inc., San Diego, CA, USA) according to the manufacturer’s instructions, generating a minimum of 80 million 150 bp paired-end raw reads per sample. After quality control, adapter trimming and mapping to the reference genome, RNA reads supporting the peptide were counted and are shown as exemplary expression profiles of peptides of the present disclosure that are highly overexpressed or exclusively expressed in recurrent cancers, e.g., adrenocortical carcinoma, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, uterine corpus endometrial carcinoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin’s lymphoma, glioblastoma, cervical carcinoma, cervical squamous cell carcinoma and endocervical adenocarcinoma, cholangiocarcinoma, hepatocellular carcinoma, liver hepatocellular carcinoma, Ewing’s sarcoma, endometrial cancer, epithelial cancer of the larynx, esophageal carcinoma, oral carcinoma, atypical meningioma, papillary thyroid carcinoma, thymoma, brain tumors, salivary duct carcinoma, extranodal T/NK-cell lymphomas, rectal cancer, mouth and throat cancer, and multiple myeloma.

### Sequences

The following sequences form part of the disclosure of the present application. A WIPO ST 26 compatible electronic sequence listing is provided with this application, too. For the avoidance of doubt, if discrepancies exist between the sequences in the following table and the electronic sequence listing, the sequences in this table shall be deemed to be the correct ones.

Note also that in some embodiments, the respective amino acid sequence has or has not a signal peptide/lead peptide. All embodiments shall be deemed to be disclosed together with the signal peptide/lead peptide and without the signal peptide/lead peptide.

Note also that in some embodiments, the respective amino acid sequence of the toxin shows a deimmunized version thereof. All embodiments shall be deemed to be disclosed with either the wildtype toxin sequence or the deimmunized variant.

TABLE 7

Sequences

| SEQ ID | Identifier | Sequence |
|---|---|---|
| 1 | CD8α1 | MALPVTALLLPLALLLHAARPSQFRVSPLDRTWNLGET VELKCQVLLSNPTSGCSWLFQP RGAAASPTFLLYLSQNKPKAAEGLDTQRFSGKRLGDTF VLTLSDFRRENEGYYFCSALSN |
| | | SIMYFSHFVPVFLPAKPTTTPAPRPPTPAPTIASQPLSLR PEACRPAAGGAVHTRGLDFA CDIYIWAPLAGTCGVLLLSLVITLYCNHRNRRRVCKCPR PWKSGDKPSLSARYV |
| 2 | CD8α2 | MALPVTALLLPLALLLHAARPSQFRVSPLDRTWNLGET VELKCQVLLSNPTSGCSWLFQP RGAAASPTFLLYLSQNKPKAAEGLDTQRFSGKRLGDTF VLTLSDFRRENEGCYFCSALSN SIMYFSHFVPVFLPAKPTTTPAPRPPTPAPTIASQPLSLR PEACRPAAGGAVHTRGLDFA CDIYIWAPLAGTCGVLLLSLVITLYCNHRNRRRVCKCPR PWKSGDKPSLSARYV |
| 3 | m1CD8α | MALPVTALLLPLALLLHAARPSQFRVSPLDRTWNLGET VELKCQVLLSNPTSGCSWLFQP RGAAASPTFLLYLSQNKPKAAEGLDTQRFSGKRLGDTF VLTLSDFRRENEGYYFCSALSN SIMYFSHFVPVFLPASWDFLPTTAQPTKKSTLKKRVCR LPRPETQKGPLCSPIYIWAPL AGTCGVLLLSLVITLYCNHRNRRRVCKCPRPVVKSGDK PSLSARYV |
| 4 | m2CD8α | MALPVTALLLPLALLLHAARPSQFRVSPLDRTWNLGET VELKCQVLLSNPTSGCSWLFQP RGAAASPTFLLYLSQNKPKAAEGLDTQRFSGKRLGDTF VLTLSDFRRENEGCYFCSALSN SIMYFSHFVPVFLPASVVDFLPTTAQPTKKSTLKKRVCR LPRPETQKGPLCSPIYIWAPL AGTCGVLLLSLVITLYCNHRNRRRVCKCPRPVVKSGDK PSLSARYV |
| 5 | CD8β1 | MRPRLWLLLAAQLTVLHGNSVLQQTPAYIKVQTNKMVM LSCEAKISLSNMRIYWLRQRQA PSSDSHHEFLALWDSAKGTIHGEEVEQEKIAVFRDASR FILNLTSVKPEDSGIYFCMIVG SPELTFGKGTQLSWDFLPTTAQPTKKSTLKKRVCRLP RPETQKGPLCSPITLGLLVAGV LVLLVSLGVAIHLCCRRRRARLRFMKQPQGEGISGTFV PQCLHGYYSNTTTSQKLLNPWI LKT |
| 6 | CD8β2 | MRPRLWLLLAAQLTVLHGNSVLQQTPAYIKVQTNKMVM LSCEAKISLSNMRIYWLRQRQA PSSDSHHEFLALWDSAKGTIHGEEVEQEKIAVFRDASR FI LNLTSVKPEDSGIYFCMIVG SPELTFGKGTQLSWDFLPTTAQPTKKSTLKKRVCRLP RPETQKGLKGKVYQEPLSPNAC MDTTAILQPHRSCLTHGS |
| 7 | CD8β3 | LQQTPAYIKVQTNKMVMLSCEAKISLSNMRIYWLRQRQ APSSDSHHEFLALWDSAKGTIH GEEVEQEKIAVFRDASRFILNLTSVKPEDSGIYFCMIVGS PELTFGKGTQLSVVDFLPTT AQPTKKSTLKKRVCRLPRPETQKGPLCSPITLGLLVAGV LVLLVSLGVAIHLCCRRRRAR |
| | | LRFMKQFYK |
| 8 | CD8β4 | LQQTPAYIKVQTNKMVMLSCEAKISLSNMRIYWLRQRQ APSSDSHHEFLALWDSAKGTIH GEEVEQEKIAVFRDASRFILNLTSVKPEDSGIYFCMIVGS PELTFGKGTQLSVVDFLPTT AQPTKKSTLKKRVCRLPRPETQKGPLCSPITLGLLVAGV LVLLVSLGVAIHLCCRRRRAR LRFMKQLRLHPLEKCSRMDY |
| 9 | CD8β5 | LQQTPAYIKVQTNKMVMLSCEAKISLSNMRIYWLRQRQ APSSDSHHEFLALWDSAKGTIH GEEVEQEKIAVFRDASRFILNLTSVKPEDSGIYFCMIVGS PELTFGKGTQLSVVDFLPTT AQPTKKSTLKKRVCRLPRPETQKGPLCSPITLGLLVAGV LVLLVSLGVAIHLCCRRRRAR LRFM KQKFNIVCLKISGFTTCCCFQILQISREYGFGVLLQ KDIGQ |
| 10 | CD8β6 | LQQTPAYIKVQTNKMVMLSCEAKISLSNMRIYWLRQRQ APSSDSHHEFLALWDSAKGTIH GEEVEQEKIAVFRDASRFILNLTSVKPEDSGIYFCMIVGS PELTFGKGTQLSVVDFLPTT AQPTKKSTLKKRVCRLPRPETQKGPLCSPITLGLLVAGV LVLLVSLGVAIHLCCRRRRAR LRFMKQKFNIVCLKISGFTTCCCFQILQISREYGFGVLLQ KDIGQ |
| 11 | CD8β7 | LQQTPAYIKVQTNKMVMLSCEAKISLSNMRIYWLRQRQ APSSDSHHEFLALWDSAKGTIH GEEVEQEKIAVFRDASRFILNLTSVKPEDSGIYFCMIVGS PELTFGKGTQLSVVDFLPTT AQPTKKSTLKKRVCRLPRPETQKGPLCSPITLGLLVAGV LVLLVSLGVAIHLCCRRRRAR LRFMKQPQGEGISGTFVPQCLHGYYSNTTTSQKLLNP WILKT |
| 12 | R11P3D3 alpha CDR1 | SSNFYA |
| 13 | R11P3D3 alpha CDR2 | MTL |
| 14 | R11P3D3 alpha CDR3 | CALYNNNDMRF |
| 15 | R11P3D3 alpha variable domain | MEKNPLAAPLLILWFHLDCVSSILNVEQSPQSLHVQEG DSTNFTCSFPSSNFYALHWYRW ETAKSPEALFVMTLNGDEKKKGRISATLNTKEGYSYLYI KGSQPEDSATYLCALYNNNDM RFGAGTRLTVKP |
| 16 | R11P3D3 alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 17 | R11P3D3 alpha full-length | MEKNPLAAPLLILWFHLDCVSSILNVEQSPQSLHVQEG DSTNFTCSFPSSNFYALHWYRW |
| | | ETAKSPEALFVMTLNGDEKKKGRISATLNTKEGYSYLYI KGSQPEDSATYLCALYNNNDM RFGAGTRLTVKPNIQNPDPAVYQLRDSKSSDKSVCLFT DFDSQTNVSQSKDSDVYITDKT VLDMRSMDFKSNSAVAWSNKSDFACANAFNNSIIPEDT FFPSPESSCDVKLVEKSFETDT NLNFQNLSVIGFRILLLKVAGFNLLMTLRLWSS |
| 18 | R11P3D3 beta CDR1 | SGHNS |
| 19 | R11P3D3 beta CDR2 | FNNNVP |
| 20 | R11P3D3 beta CDR3 | CASSPGSTDTQYF |
| 21 | R11P3D3 beta variable domain | MDSWTFCCVSLCILVAKHTDAGVIQSPRHEVTEMGQEV TLRCKPISGHNSLFWYRQTMMR GLELLIYFNNNVPIDDSGMPEDRFSAKMPNASFSTLKIQ PSEPRDSAVYFCASSPGSTDT QYFGPGTRLTVL |
| 22 | R11P3D3 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTS ESYQQGVLSATI LYE I LLGKATLY AVLVSALVLMAMVKRKDSRG |
| 23 | R11P3D3 beta full-length | MDSWTFCCVSLCILVAKHTDAGVIQSPRHEVTEMGQEV TLRCKPISGHNSLFWYRQTMMR GLELLIYFNNNVPIDDSGMPEDRFSAKMPNASFSTLKIQ PSEPRDSAVYFCASSPGSTDT QYFGPGTRLTVLEDLKNVFPPEVAVFEPSEAEISHTQKA TLVCLATGFYPDHVELSWWVN GKEVHSGVSTDPQPLKEQPALNDSRYCLSSRLRVSATF WQNPRNHFRCQVQFYGLSENDE WTQDRAKPVTQIVSAEAWGRADCGFTSESYQQGVLSA TILYEILLGKATLYAVLVSALVL MAMVKRKDSRG |
| 24 | R16P1C10 alpha CDR1 | DRGSQS |
| 25 | R16P1C10 alpha CDR2 | IY |
| 26 | R16P1C10 alpha CDR3 | CAAVISNFGNEKLTF |
| 27 | R16P1C10 alpha variable domain | MKSLRVLLVILWLQLSWVWSQQKEVEQNSGPLSVPEG AIASLNCTYSDRGSQSFFWYRQY SGKSPELIMFIYSNGDKEDGRFTAQLNKASQYVSLLIRD SQPSDSATYLCAAVISNFGNE KLTFGTGTRLTIIP |
| 28 | R16P1C10 alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF |
| | | RILLLKVAGFNLLMTLRLWSS |
| 29 | R16P1C10 alpha full-length | MKSLRVLLVILWLQLSWVWSQQKEVEQNSGPLSVPEG AIASLNCTYSDRGSQSFFWYRQY SGKSPELIMFIYSNGDKEDGRFTAQLNKASQYVSLLIRD SQPSDSATYLCAAVISNFGNE KLTFGTGTRLTIIPNIQNPDPAVYQLRDSKSSDKSVCLFT DFDSQTNVSQSKDSDVYITD KTVLDMRSMDFKSNSAVAWSNKSDFACANAFNNSIIPE DTFFPSPESSCDVKLVEKSFET DTNLNFQNLSVIGFRILLLKVAGFNLLMTLRLWSS |
| 30 | R16P1C10 beta CDR1 | SGHRS |
| 31 | R16P1C10 beta CDR2 | YFSETQ |
| 32 | R16P1C10 beta CDR3 | CASSPWDSPNEQYF |
| 33 | R16P1C10 beta variable domain | MGSRLLCWVLLCLLGAGPVKAGVTQTPRYLIKTRGQQV TLSCSPISGHRSVSWYQQTPGQ GLQFLFEYFSETQRNKGNFPGRFSGRQFSNSRSEMNV STLELGDSALYLCASSPWDSPNE QYFGPGTRLTVT |
| 34 | R16P1C10 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTS ESYQQGVLSATI LYE I LLGKATLY AVLVSALVLMAMVKRKDSRG |
| 35 | R16P1C10 beta full-length | MGSRLLCWVLLCLLGAGPVKAGVTQTPRYLIKTRGQQV TLSCSPISGHRSVSWYQQTPGQ GLQFLFEYFSETQRNKGNFPGRFSGRQFSNSRSEMNV STLELGDSALYLCASSPWDSPNE QYFGPGTRLTVTEDLKNVFPPEVAVFEPSEAEISHTQK ATLVCLATGFYPDHVELSWWVN GKEVHSGVSTDPQPLKEQPALNDSRYCLSSRLRVSATF WQNPRNHFRCQVQFYGLSENDE WTQDRAKPVTQIVSAEAWGRADCGFTSESYQQGVLSA TILYEILLGKATLYAVLVSALVL MAMVKRKDSRG |
| 36 | R16P1E8 alpha CDR1 | NSAFQY |
| 37 | R16P1E8 alpha CDR2 | TY |
| 38 | R16P1E8 alpha CDR3 | CAMSEAAGNKLTF |
| 39 | R16P1E8 alpha variable domain | MMKSLRVLLVILWLQLSWVWSQQKEVEQDPGPLSVPE GAIVSLNCTYSNSAFQYFMWYRQ YSRKGPELLMYTYSSGNKEDGRFTAQVDKSSKYISLFIR DSQPSDSATYLCAMSEAAGNK LTFGGGTRVLVKP |
| 40 | R16P1E8 alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 41 | R16P1E8 alpha full-length | MMKSLRVLLVI LWLQLSWVWSQQKEVEQDPGPLSVPE GAIVSLNCTYSNSAFQYFMWYRQ YSRKGPELLMYTYSSGNKEDGRFTAQVDKSSKYISLFIR DSQPSDSATYLCAMSEAAGNK LTFGGGTRVLVKPNIQNPDPAVYQLRDSKSSDKSVCLF TDFDSQTNVSQSKDSDVYITDK TVLDMRSMDFKSNSAVAWSNKSDFACANAFNNSIIPED TFFPSPESSCDVKLVEKSFETD TNLNFQNLSVIGFRILLLKVAGFNLLMTLRLWSS |
| 42 | R16P1E8 beta CDR1 | SGHAT |
| 43 | R16P1E8 beta CDR2 | FQNNGV |
| 44 | R16P1E8 beta CDR3 | CASSYTNQGEAFF |
| 45 | R16P1E8 beta variable domain | MGTRLLCWAALCLLGAELTEAGVAQSPRYKIIEKRQSV AFWCNPISGHATLYWYQQILGQ GPKLLIQFQNNGVVDDSQLPKDRFSAERLKGVDSTLKI QPAKLEDSAVYLCASSYTNQGE AFFGQGTRLTVV |
| 46 | R16P1E8 beta constant domain | EDLNKVFPPEVAVFEPSEAEISHTQKATLVCLATGFFPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSVSYQQGVLSATI LYEILLGKATLY AVLVSALVLMAMVKRKDF |
| 47 | R16P1E8 beta full-length | MGTRLLCWAALCLLGAELTEAGVAQSPRYKIIEKRQSV AFWCNPISGHATLYWYQQILGQ GPKLLIQFQNNGVVDDSQLPKDRFSAERLKGVDSTLKI QPAKLEDSAVYLCASSYTNQGE AFFGQGTRLTVVEDLNKVFPPEVAVFEPSEAEISHTQK ATLVCLATGFFPDHVELSWWVN GKEVHSGVSTDPQPLKEQPALNDSRYCLSSRLRVSATF WQNPRNHFRCQVQFYGLSENDE WTQDRAKPVTQIVSAEAWGRADCGFTSVSYQQGVLSA TILYE I LLGKATLYAVLVSALVL MAMVKRKDF |
| 48 | R17P1A9 alpha CDR1 | DRGSQS |
| 49 | R17P1A9 alpha CDR2 | IY |
| 50 | R17P1A9 alpha CDR3 | CAVLNQAGTALIF |
| 51 | R17P1A9 alpha variable domain | MKSLRVLLVILWLQLSWVWSQQKEVEQNSGPLSVPEG AIASLNCTYSDRGSQSFFWYRQY |
| | | SGKSPELIMSIYSNGDKEDGRFTAQLNKASQYVSLLIRD SQPSDSATYLCAVLNQAGTAL IFGKGTTLSVSS |
| 52 | R17P1A9 alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 53 | R17P1A9 alpha full-length | MKSLRVLLVILWLQLSWVWSQQKEVEQNSGPLSVPEG AIASLNCTYSDRGSQSFFWYRQY SGKSPELIMSIYSNGDKEDGRFTAQLNKASQYVSLLIRD SQPSDSATYLCAVLNQAGTAL IFGKGTTLSVSSNIQNPDPAVYQLRDSKSSDKSVCLFTD FDSQTNVSQSKDSDVYITDKT VLDMRSMDFKSNSAVAWSNKSDFACANAFNNSIIPEDT FFPSPESSCDVKLVEKSFETDT NLNFQNLSVIGFRILLLKVAGFNLLMTLRLWSS |
| 54 | R17P1A9 beta CDR1 | SGDLS |
| 55 | R17P1A9 beta CDR2 | YYNGEE |
| 56 | R17P1A9 beta CDR3 | CASSAETGPWLGNEQFF |
| 57 | R17P1A9 beta variable domain | MGFRLLCCVAFCLLGAGPVDSGVTQTPKHLITATGQRV TLRCSPRSGDLSVYWYQQSLDQ GLQFLIQYYNGEERAKGNILERFSAQQFPDLHSELNLSS LELGDSALYFCASSAETGPWL GNEQFFGPGTRLTVL |
| 58 | R17P1A9 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSESYQQGVLSATILYEILLGKATLY AVLVSALVLMAMVKRKDSRG |
| 59 | R17P1A9 beta full-length | MGFRLLCCVAFCLLGAGPVDSGVTQTPKHLITATGQRV TLRCSPRSGDLSVYWYQQSLDQ GLQFLIQYYNGEERAKGNILERFSAQQFPDLHSELNLSS LELGDSALYFCASSAETGPWL GNEQFFGPGTRLTVLEDLKNVFPPEVAVFEPSEAEISHT QKATLVCLATGFYPDHVELSW WVNGKEVHSGVSTDPQPLKEQPALNDSRYCLSSRLRV SATFWQNPRNHFRCQVQFYGLSE NDEWTQDRAKPVTQIVSAEAWGRADCGFTSESYQQG VLSATILYEILLGKATLYAVLVSA LVLMAMVKRKDSRG |
| 60 | R17P1D7 alpha CDR1 | TSESDYY |
| 61 | R17P1D7 alpha CDR2 | QEAY |
| 62 | R17P1D7 alpha CDR3 | CAYRWAQGGSEKLVF |
| 63 | R17P1D7 alpha variable domain | MACPGFLWALVISTCLEFSMAQTVTQSQPEMSVQEAE TVTLSCTYDTSESDYYLFWYKQP PSRQMILVIRQEAYKQQNATENRFSVNFQKAAKSFSLKI SDSQLGDAAMYFCAYRWAQGG SEKLVFGKGTKLTVNP |
| 64 | R17P1D7 alpha constant domain | YIQKPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 65 | R17P1D7 alpha full-length | MACPGFLWALVISTCLEFSMAQTVTQSQPEMSVQEAE TVTLSCTYDTSESDYYLFWYKQP PSRQMILVIRQEAYKQQNATENRFSVNFQKAAKSFSLKI SDSQLGDAAMYFCAYRWAQGG SEKLVFGKGTKLTVNPYIQKPDPAVYQLRDSKSSDKSV CLFTDFDSQTNVSQSKDSDVYI TDKTVLDMRSMDFKSNSAVAWSNKSDFACANAFNNSII PEDTFFPSPESSCDVKLVEKSF ETDTNLNFQNLSVIGFRILLLKVAGFNLLMTLRLWSS |
| 66 | R17P1D7 beta CDR1 | MGHDK |
| 67 | R17P1D7 beta CDR2 | SYGVNS |
| 68 | R17P1D7 beta CDR3 | CATELWSSGGTGELFF |
| 69 | R17P1D7 beta variable domain | MTI RLLCYMGFYFLGAGLM EADIYQTPRYLVIGTGKKITL ECSQTMGHDKMYWYQQDPGM ELHLIHYSYGVNSTEKGDLSSESTVSRIRTEHFPLTLES ARPSHTSQYLCATELWSSGGT GELFFGEGSRLTVL |
| 70 | R17P1D7 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSESYQQGVLSATILYEILLGKATLY AVLVSALVLMAMVKRKDSRG |
| 71 | R17P1D7 beta full-length | MTIRLLCYMGFYFLGAGLMEADIYQTPRYLVIGTGKKITL ECSQTMGHDKMYWYQQDPGM ELHLIHYSYGVNSTEKGDLSSESTVSRIRTEHFPLTLES ARPSHTSQYLCATELWSSGGT GELFFGEGSRLTVLEDLKNVFPPEVAVFEPSEAEISHTQ KATLVCLATGFYPDHVELSWW VNGKEVHSGVSTDPQPLKEQPALNDSRYCLSSRLRVS ATFWQNPRNHFRCQVQFYGLSEN DEWTQDRAKPVTQIVSAEAWGRADCGFTSESYQQGVL SATILYEILLGKATLYAVLVSAL VLMAMVKRKDSRG |
| 72 | R17P1G3 alpha CDR1 | DRGSQS |
| 73 | R17P1G3 alpha CDR2 | IY |
| 74 | R17P1G3 alpha CDR3 | CAVGPSGTYKYIF |
| 75 | R17P1G3 alpha variable domain | MKSLRVLLVILWLQLSWVWSQQKEVEQNSGPLSVPEG AIASLNCTYSDRGSQSFFWYRQY SGKSPELIMSIYSNGDKEDGRFTAQLNKASQYVSLLIRD SQPSDSATYLCAVGPSGTYKY IFGTGTRLKVLA |
| 76 | R17P1G3 alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 77 | R17P1G3 alpha full-length | MKSLRVLLVILWLQLSWVWSQQKEVEQNSGPLSVPEG AIASLNCTYSDRGSQSFFWYRQY SGKSPELIMSIYSNGDKEDGRFTAQLNKASQYVSLLIRD SQPSDSATYLCAVGPSGTYKY IFGTGTRLKVLANIQNPDPAVYQLRDSKSSDKSVCLFTD FDSQTNVSQSKDSDVYITDKT VLDMRSMDFKSNSAVAWSNKSDFACANAFNNSIIPEDT FFPSPESSCDVKLVEKSFETDT NLNFQNLSVIGFRILLLKVAGFNLLMTLRLWSS |
| 78 | R17P1G3 beta CDR1 | MNHEY |
| 79 | R17P1G3 beta CDR2 | SMNVEV |
| 80 | R17P1G3 beta CDR3 | CASSPGGSGNEQFF |
| 81 | R17P1G3 beta variable domain | MGPQLLGYVVLCLLGAGPLEAQVTQNPRYLITVTGKKL TVTCSQNMNHEYMSWYRQDPGL GLRQIYYSMNVEVTDKGDVPEGYKVSRKEKRNFPLILE SPSPNQTSLYFCASSPGGSGNE QFFGPGTRLTVL |
| 82 | R17P1G3 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSESYQQGVLSATILYEILLGKATLY AVLVSALVLMAMVKRKDSRG |
| 83 | R17P1G3 beta full-length | MGPQLLGYVVLCLLGAGPLEAQVTQNPRYLITVTGKKL TVTCSQNMNHEYMSWYRQDPGL GLRQIYYSMNVEVTDKGDVPEGYKVSRKEKRNFPLILE SPSPNQTSLYFCASSPGGSGNE QFFGPGTRLTVLEDLKNVFPPEVAVFEPSEAEISHTQKA TLVCLATGFYPDHVELSWWVN GKEVHSGVSTDPQPLKEQPALNDSRYCLSSRLRVSATF WQNPRNHFRCQVQFYGLSENDE |
| | | WTQDRAKPVTQIVSAEAWGRADCGFTSESYQQGVLSA TILYEILLGKATLYAVLVSALVL MAMVKRKDSRG |
| 84 | R17P2B6 alpha CDR1 | DRGSQS |
| 85 | R17P2B6 alpha CDR2 | IY |
| 86 | R17P2B6 alpha CDR3 | CAVVSGGGADGLTF |
| 87 | R17P2B6 alpha variable domain | MKSLRVLLVILWLQLSWVWSQQKEVEQNSGPLSVPEG AIASLNCTYSDRGSQSFFWYRQY SGKSPELIMFIYSNGDKEDGRFTAQLNKASQYVSLLIRD SQPSDSATYLCAVVSGGGADG LTFGKGTHLIIQP |
| 88 | R17P2B6 alpha constant domain | YIQKPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 89 | R17P2B6 alpha full-length | MKSLRVLLVILWLQLSWVWSQQKEVEQNSGPLSVPEG AIASLNCTYSDRGSQSFFWYRQY SGKSPELIMFIYSNGDKEDGRFTAQLNKASQYVSLLIRD SQPSDSATYLCAVVSGGGADG LTFGKGTHLIIQPYIQKPDPAVYQLRDSKSSDKSVCLFT DFDSQTNVSQSKDSDVYITDK TVLDMRSMDFKSNSAVAWSNKSDFACANAFNNSIIPED TFFPSPESSCDVKLVEKSFETD TNLNFQNLSVIGFRILLLKVAGFNLLMTLRLWSS |
| 90 | R17P2B6 beta CDR1 | PRHDT |
| 91 | R17P2B6 beta CDR2 | FYEKMQ |
| 92 | R17P2B6 beta CDR3 | CASSLGRGGQPQHF |
| 93 | R17P2B6 beta variable domain | MLSPDLPDSAWNTRLLCHVMLCLLGAVSVAAGVIQSPR HLIKEKRETATLKCYPIPRHDT VYWYQQGPGQDPQFLISFYEKMQSDKGSIPDRFSAQQ FSDYHSELNMSSLELGDSALYFC ASSLGRGGQPQHFGDGTRLSIL |
| 94 | R17P2B6 beta constant domain | EDLNKVFPPEVAVFEPSEAEISHTQKATLVCLATGFFPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSVSYQQGVLSATILYEILLGKATLY AVLVSALVLMAMVKRKDF |
| 95 | R17P2B6 beta full-length | MLSPDLPDSAWNTRLLCHVMLCLLGAVSVAAGVIQSPR HLIKEKRETATLKCYPIPRHDT VYWYQQGPGQDPQFLISFYEKMQSDKGSIPDRFSAQQ FSDYHSELNMSSLELGDSALYFC |
| | | ASSLGRGGQPQHFGDGTRLSILEDLNKVFPPEVAVFEP SEAEISHTQKATLVCLATGFFP DHVELSWWVNGKEVHSGVSTDPQPLKEQPALNDSRY CLSSRLRVSATFWQNPRNHFRCQV QFYGLSENDEWTQDRAKPVTQIVSAEAWGRADCGFTS VSYQQGVLSATILYEILLGKATL YAVLVSALVLMAMVKRKDF |
| 96 | 1G4 alpha CDR1 | DSAIYN |
| 97 | 1G4 alpha CDR2 | IQS |
| 98 | 1G4 alpha CDR3 | CAVRPTSGGSYIPTF |
| 99 | 1G4 alpha variable domain | METLLGLLILWLQLQWVSSKQEVTQIPAALSVPEGENLV LNCSFTDSAIYNLQWFRQDPG KGLTSLLLIQSSQREQTSGRLNASLDKSSGRSTLYIAAS QPGDSATYLCAVRPTSGGSYI PTFGRGTSLIVHP |
| 100 | 1G4 alpha constant domain | YIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 101 | 1G4 alpha full-length | METLLGLLILWLQLQWVSSKQEVTQIPAALSVPEGENLV LNCSFTDSAIYNLQWFRQDPG KGLTSLLLIQSSQREQTSGRLNASLDKSSGRSTLYIAAS QPGDSATYLCAVRPTSGGSYI PTFGRGTSLIVHPYIQNPDPAVYQLRDSKSSDKSVCLFT DFDSQTNVSQSKDSDVYITDK TVLDMRSMDFKSNSAVAWSNKSDFACANAFNNSIIPED TFFPSPESSCDVKLVEKSFETD TNLNFQNLSVIGFRILLLKVAGFNLLMTLRLWSS |
| 102 | 1G4 beta CDR1 | MNHEY |
| 103 | 1G4 beta CDR2 | SVGAGI |
| 104 | 1G4 beta CDR3 | CASSYVGNTGELFF |
| 105 | 1G4 beta variable domain | MSIGLLCCAALSLLWAGPVNAGVTQTPKFQVLKTGQSM TLQCAQDMNHEYMSWYRQDPGM GLRLIHYSVGAGITDQGEVPNGYNVSRSTTEDFPLRLLS AAPSQTSVYFCASSYVGNTGE LFFGEGSRLTVL |
| 106 | 1G4 beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTSESYQQGVLSATILYEILLGKATLY AVLVSALVLMAMVKRKDSRG |
| 107 | 1G4 beta full-length | MSIGLLCCAALSLLWAGPVNAGVTQTPKFQVLKTGQSM TLQCAQDMNHEYMSWYRQDPGM GLRLIHYSVGAGITDQGEVPNGYNVSRSTTEDFPLRLLS AAPSQTSVYFCASSYVGNTGE |
| | | LFFGEGSRLTVLEDLKNVFPPEVAVFEPSEAEISHTQKA TLVCLATGFYPDHVELSWWVN GKEVHSGVSTDPQPLKEQPALNDSRYCLSSRLRVSATF WQNPRNHFRCQVQFYGLSENDE WTQDRAKPVTQIVSAEAWGRADCGFTSESYQQGVLSA TILYEILLGKATLYAVLVSALVL MAMVKRKDSRG |
| 108 | R11P3D3_KE alpha CDR1 | SSNFYA |
| 109 | R11P3D3_KE alpha CDR2 | MTL |
| 110 | R11P3D3_KE alpha CDR3 | CALYNNNDMRF |
| 111 | R11P3D3_KE alpha variable domain | MEKNPLAAPLLILWFHLDCVSSILNVEQSPQSLHVQEG DSTNFTCSFPSSNFYALHWYRK ETAKSPEALFVMTLNGDEKKKGRISATLNTKEGYSYLYI KGSQPEDSATYLCALYNNNDM RFGAGTRLTVKP |
| 112 | R11P3D3_KE alpha constant domain | NIQNPDPAVYQLRDSKSSDKSVCLFTDFDSQTNVSQSK DSDVYITDKTVLDMRSMDFKSN SAVAWSNKSDFACANAFNNSIIPEDTFFPSPESSCDVKL VEKSFETDTNLNFQNLSVIGF RILLLKVAGFNLLMTLRLWSS |
| 113 | R11 P3D3_KE alpha full-length | MEKNPLAAPLLILWFHLDCVSSILNVEQSPQSLHVQEG DSTNFTCSFPSSNFYALHWYRK ETAKSPEALFVMTLNGDEKKKGRISATLNTKEGYSYLYI KGSQPEDSATYLCALYNNNDM RFGAGTRLTVKPNIQNPDPAVYQLRDSKSSDKSVCLFT DFDSQTNVSQSKDSDVYITDKT VLDMRSMDFKSNSAVAWSNKSDFACANAFNNSIIPEDT FFPSPESSCDVKLVEKSFETDT NLNFQNLSVIGFRILLLKVAGFNLLMTLRLWSS |
| 114 | R11P3D3_KE beta CDR1 | SGHNS |
| 115 | R11P3D3_KE beta CDR2 | FNNNVP |
| 116 | R11P3D3_KE beta CDR3 | CASSPGSTDTQYF |
| 117 | R11P3D3_KE beta variable domain | MDSWTFCCVSLCILVAKHTDAGVIQSPRHEVTEMGQEV TLRCKPISGHNSLFWYRETMMR GLELLIYFNNNVPIDDSGMPEDRFSAKMPNASFSTLKIQ PSEPRDSAVYFCASSPGSTDT QYFGPGTRLTVL |
| 118 | R11P3D3_KE beta constant domain | EDLKNVFPPEVAVFEPSEAEISHTQKATLVCLATGFYPD HVELSWWVNGKEVHSGVSTDP QPLKEQPALNDSRYCLSSRLRVSATFWQNPRNHFRCQ VQFYGLSENDEWTQDRAKPVTQI VSAEAWGRADCGFTS ESYQQGVLSATILYEILLGKATLY AVLVSALVLMAMVKRKDSRG |
| 119 | R11P3D3_KE beta full-length | MDSWTFCCVSLCILVAKHTDAGVIQSPRHEVTEMGQEV TLRCKPISGHNSLFWYRETMMR GLELLIYFNNNVPIDDSGMPEDRFSAKMPNASFSTLKIQ PSEPRDSAVYFCASSPGSTDT QYFGPGTRLTVLEDLKNVFPPEVAVFEPSEAEISHTQKA TLVCLATGFYPDHVELSWWVN GKEVHSGVSTDPQPLKEQPALNDSRYCLSSRLRVSATF WQNPRNHFRCQVQFYGLSENDE WTQDRAKPVTQIVSAEAWGRADCGFTSESYQQGVLSA TILYEILLGKATLYAVLVSALVL MAMVKRKDSRG |
| 120 | R11P3D3 alpha CDR2bis | MTLNGDE |
| 121 | R16P1C10 alpha CDR2bis | IYSNGD |
| 122 | R16P1E8 alpha CDR2bis | TYSSGN |
| 123 | R17P1A9 alpha CDR2bis | IYSNGD |
| 124 | R17P1D7 alpha CDR2bis | QEAYKQQ |
| 125 | R17P1G3 alpha CDR2bis | IYSNGD |
| 126 | R17P2B6 alpha CDR2bis | IYSNGD |
| 127 | 1G4 alpha CDR2bis | IQSSQRE |
| 128 | R11P3D3_KE alpha CDR2bis | MTLNGDE |
| 129 | hinges of an IgG1 molecule is (EU numbering indicated), staring with E216 | EPKSCDKTHTCPPCPAPELLG |
| 130 | Fc domain can comprise a CH2 domain comprising at least one effector function silencing mutation | ELLGGP |
| 131 | IA_5R16P1C10l hUCHT1 (Var17) | QKEVEQNSGPLSVPEGAIASLNCTYSDRGSQSFFWYR QYSGKSPELIMSIYSNGDKEDGR FTAQLNKASQYFSLLIRDSQPSDSATYLCAAVIDNSNGG ILTFGTGTRLTIIPNIQNGGG SGGGGDIQMTQSPSSLSASVGDRVTITCRASQDIRNYL NWYQQKPGKAPKLLIYYTSRLH |
| | | SGVPSRFSGSGSGTDYTLTISSLQPEDIATYFCQQGQT LPWTFGQGTKVEIKEPKSSDKT HTCPPCPAPPVAGPSVFLFPPKPKDTLMISRTPEVTCV WDVSHEDPEVKFNWYVDGVEV HNAKTKPREEQYQSTYRWSVLTVLHQDWLNGKEYKC KVSNKALPASIEKTISKAKGQPR EPQVYTLPPCRDELTKNQVSLWCLVKGFYPSDIAVEWE SNGQPENNYKTTPPVLDSDGSF FLYSKLTVDKSRWQQGNVFSCSVMHEALHNHYTQKSL SLSP |
| 132 | IA_5R16P1C10l hUCHT1 (Var17) | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGKAGVTQTPRYLIKTRGQQVTLSCSPIP GHRSVSWYQQTPGQGLQFLFEYV HGAERNKGNFPGRFSGRQFSNSSSEMNISNLELGDSA LYLCASSPWDSPNEQYFGPGTRL TVTEDLKNEPKSSDKTHTCPPCPAPPVAGPSVFLFPPK PKDTLMISRTPEVTCWVDVSH EDPEVKFNWYVDGVEVHNAKTKPREEQYQSTYRWSV LTVLHQDWLNGKEYKCKVSNKAL PASIEKTISKAKGQPREPQVCTLPPSRDELTKNQVSLSC AVKGFYPSDIAVEWESNGQPE NNYKTTPPVLDSDGSFFLVSKLTVDKSRWQQGNVFSC SVMHEALHNHYTQKSLSLSP |
| 133 | IA_6R16P1C101#6 hUCHT1 (Var17) | QKEVEQNSGPLSVPEGAIASLNCTYSDRGSQSFFWYR QYSGKSPELIMSIYSNGDKEDGR FTAQLNKASQYVSLLIRDSQPSDSATYLCAAVIDNDQG GILTFGTGTRLTIIPNIQNGGG SGGGGDIQMTQSPSSLSASVGDRVTITCRASQDIRNYL NWYQQKPGKAPKLLIYYTSRLH SGVPSRFSGSGSGTDYTLTISSLQPEDIATYFCQQGQT LPWTFGQGTKVEIKEPKSSDKT HTCPPCPAPPVAGPSVFLFPPKPKDTLMISRTPEVTCV WDVSHEDPEVKFNWYVDGVEV HNAKTKPREEQYQSTYRWSVLTVLHQDWLNGKEYKC KVSNKALPASIEKTISKAKGQPR EPQVYTLPPCRDELTKNQVSLWCLVKGFYPSDIAVEWE SNGQPENNYKTTPPVLDSDGSF FLYSKLTVDKSRWQQGNVFSCSVMHEALHNHYTQKSL SLSPGK |
| 134 | IA_6R16P1C10l# 6hUCHT1 (Var17) | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGKAGVTQTPRYLIKTRGQQVTLSCSPIP GHRAVSWYQQTPGQGLQFLFEYV HGEERNKGNFPGRFSGRQFSNSSSEMNISNLELGDSA LYLCASSPWDSPNVQYFGPGTRL |
| | | TVTEDLKNEPKSSDKTHTCPPCPAPPVAGPSVFLFPPK PKDTLMISRTPEVTCWVDVSH EDPEVKFNWYVDGVEVHNAKTKPREEQYQSTYRWSV LTVLHQDWLNGKEYKCKVSNKAL PASIEKTISKAKGQPREPQVCTLPPSRDELTKNQVSLSC AVKGFYPSDIAVEWESNGQPE NNYKTTPPVLDSDGSFFLVSKLTVDKSRWQQGNVFSC SVMHEALHNHYTQKSLSLSPGK |
| 135 | alpha CDRa1 | DRGSQS |
| 136 | alpha CDRa1 | DRGSQL |
| 137 | alpha CDRa2 | IYSNGD |
| 138 | alpha CDRa2 | IYQEGD |
| 139 | alpha CDRa3 | CAAVINNPSGGMLTF |
| 140 | alpha CDRa3 | CAAVIDNSNGGILTF |
| 141 | alpha CDRa3 | CAAVIDNPSGGILTF |
| 142 | alpha CDRa3 | CAAVIDNDQGGILTF |
| 143 | alpha CDRa3 | CAAVIPNPPGGKLTF |
| 144 | alpha CDRa3 | CAAVIPNPGGGALTF |
| 145 | alpha CDRa3 | CAAVIPNSAGGRLTF |
| 146 | alpha CDRa3 | CAAVIPNLEGGSLTF |
| 147 | alpha CDRa3 | CAAVIPNRLGGYLTF |
| 148 | alpha CDRa3 | CAAVIPNTDGGRLTF |
| 149 | alpha CDRa3 | CAAVIPNQRGGALTF |
| 150 | alpha CDRa3 | CAAVIPNVVGGILTF |
| 151 | alpha CDRa3 | CAAVITNIAGGSLTF |
| 152 | alpha CDRa3 | CAAVIPNNDGGYLTF |
| 153 | alpha CDRa3 | CAAVIPNGRGGLLTF |
| 154 | alpha CDRa3 | CAAVIPNTHGGPLTF |
| 155 | alpha CDRa3 | CAAVIPNDVGGSLTF |
| 156 | alpha CDRa3 | CAAVIENKPGGPLTF |
| 157 | alpha CDRa3 | CAAVIDNPVGGPLTF |
| 158 | alpha CDRa3 | CAAVIPNNNGGALTF |
| 159 | alpha CDRa3 | CAAVIPNDQGGILTF |
| 160 | alpha CDRa3 | CAAVIPNVVGGQLTF |
| 161 | alpha CDRa3 | CAAVIPNSYGGLLTF |
| 162 | alpha CDRa3 | CAAVIPNDDGGLLTF |
| 163 | alpha CDRa3 | CAAVIPNAAGGLLTF |
| 164 | alpha CDRa3 | CAAVIPNTIGGLLTF |
| 165 | alpha CDRa3 | CAAVIPNTRGGLLTF |
| 166 | beta CDRb1 | SGHRS |
| 167 | beta CDRb1 | PGHRA |
| 168 | beta CDRb1 | PGHRS |
| 169 | beta CDRb2 | YFSETQ |
| 170 | beta CDRb2 | YVHGEE |
| 171 | beta CDRb2 | YVHGAE |
| 172 | beta CDRb3 | CASSPWDSPNEQYF |
| 173 | beta CDRb3 | CASSPWDSPNVQYF |
| 174 | scTCR-Fab | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GQKEVEQNSGPLSVPEGAIASLNCTYSDRGSQSFFWY RQYSGKSPELIMSIYQEGDKEDG RFTAQLNKASQYVSLLIRDSQPSDSATYLCAAVIDNDQ GGILTFGTGTRLTIIPNIQNGG GGSGGGGSGGGGSGGGGSGGGGSGSKAGVTQTPRY LIKTRGQQVTLSCSPIPGHRAVSWY QQTPGQGLQFLFEYVHGEERNKGNFPGRFSGRQFSN SSSEMNISNLELGDSALYLCASSP WDSPNVQYFGPGTRLTVTEDLKN |
| 175 | scTCR-Fab | DIQMTQSPSSLSASVGDRVTITCRASQDIRNYLNWYQQ KPGKAPKLLIYYTSRLHSGVPS RFSGSGSGTDYTLTISSLQPEDIATYFCQQGQTLPWTF GQGTKVEIKRTVAAPSVFIFPP SDEQLKSGTASWCLLNNFYPREAKVQWKVDNALQSG NSQESVTEQDSKDSTYSLSSTLT LSKADYEKHKVYACEVTHQGLSSPVTKSFNRGEC |
| 176 | diabody-Fc | QKEVEQNSGPLSVPEGAIASLNCTYSDRGSQSFFWYR QYSGKSPELIMSIYQEGDKEDGR FTAQLNKASQYVSLLIRDSQPSDSATYLCAAVIDNDQG GILTFGTGTRLTIIPNIQNGGG SGGGGDIQMTQSPSSLSASVGDRVTITCRASQDIRNYL NWYQQKPGKAPKLLIYYTSRLH SGVPSRFSGSGSGTDYTLTISSLQPEDIATYFCQQGQT LPWTFGQGTKVEIKEPKSSDKT HTCPPCPAPPVAGPSVFLFPPKPKDTLMISRTPEVTCV WDVSHEDPEVKFNWYVDGVEV HNAKTKPREEQYQSTYRWSVLTVLHQDWLNGKEYKC KVSNKALPASIEKTISKAKGQPR EPQVYTLPPCRDELTKNQVSLWCLVKGFYPSDIAVEWE SNGQPENNYKTTPPVLDSDGSF FLYSKLTVDKSRWQQGNVFSCSVMHEALHNHYTQKSL SLSP |
| 177 | diabody-Fc | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGKAGVTQTPRYLIKTRGQQVTLSCSPIP GHRAVSWYQQTPGQGLQFLFEYV HGEERNKGNFPGRFSGRQFSNSSSEMNISNLELGDSA LYLCASSPWDSPNVQYFGPGTRL TVTEDLKNEPKSSDKTHTCPPCPAPPVAGPSVFLFPPK PKDTLMISRTPEVTCWVDVSH EDPEVKFNWYVDGVEVHNAKTKPREEQYQSTYRWSV LTVLHQDWLNGKEYKCKVSNKAL PASIEKTISKAKGQPREPQVCTLPPSRDELTKNQVSLSC AVKGFYPSDIAVEWESNGQPE NNYKTTPPVLDSDGSFFLVSKLTVDKSRWQQGNVFSC SVMHEALHNHYTQKSLSLSP |
| 178 | α-chain | DIQMTQSPSSLSASVGDRVTITCRASQDIRNYLNWYQQ KPGKAPKLLIYYTSRLHSGVPS RFSGSGSGTDYTLTISSLQPEDIATYFCQQGQTLPWTF GQGTKVEIKRTVAAPSVFIFPP SDEQLKSGTASWCLLNNFYPREAKVQWKVDNALQSG NSQESVTEQDSKDSTYSLSSTLT LSKADYEKHKVYACEVTHQGLSSPVTKSFNRGEC |
| 179 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GILNVEQSPQSLHVQEGDSTNFTCSFPTREFQDLHWY RKETAKSPEFLFYFGPYGVEKKK GRISATLNTKEGYSYLYITDSQPEDSATYLCALYNNNDM RFGAGTRLTVKPGGGGSGGGG SGGGGSGGGGSGGGGSGVIQSPRHLVTEMGQEVTLR CKPISGHNSLFWYRETPMQGLELL IYFQNTAVIDDSGMPEDRFSAKMPNASFSTLKIQPSEPR DSAVYFCASSPGSTDTQYFGP GTRLTVL |
| 180 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV |
| | | SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GILNVEQSPQSLHVQEGDSTNFTCSFPTKEFQDLHWY RKETAKSPEFLFYFGPYGREKKK GRISATLNTKEGYSYLYITDSQPEDSATYLCALYNNNDM RFGAGTRLTVKPGGGGSGGGG SGGGGSGGGGSGGGGSGVIQSPRHLVTEMGQEVTLR CKPISGHNSLFWYRETPMQGLELL IYFQNTAVIDDSGMPEDRFSAKMPNASFSTLKIQPSEPR DSAVYFCASSPGATDTQYFGP GTRLTVL |
| 181 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GILNVEQSPQSLHVQEGDSTNFTCSFPSSNFYNLHWY RKETAKSPEFLFYFGPYGVEKKK GRISATLNTKEGYSYLYITDSQPEDSATYLCALYNNNDM RFGAGTRLTVKPGGGGSGGGG SGGGGSGGGGSGGGGSGVIQSPRHLVTEMGQEVTLR CKPISGHNSLFWYRETPMQGLELL IYFNSETVIDDSGMPEDRFSAKMPNASFSTLKIQPSEPR DSAVYFCASSPGATDTQYFGP GTRLTVL |
| 182 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GILNVEQSPQSLHVQEGDSTNFTCSFPNKEFQDLHWY RKETAKSPEFLFYFGPYGTEKKK GRISATLNTKEGYSYLYITDSQPEDSATYLCALYNNNDM RFGAGTRLTVKPGGGGSGGGG SGGGGSGGGGSGGGGSGVIQSPRHLVTEMGQEVTLR CKPISGHNSLFWYRETPMQGLELL IYFQNTAVIDDSGMPEDRFSAKMPNASFSTLKIQPSEPR DSAVYFCASSPGSTDTQYFGP GTRLTVL |
| 183 | β-chain | EVQLVQSGAEVKKPGASVKVSCKASGYSFTGYTMNWV RQAPGQGLEWMGLI NPYKGVSTY AQKFQDRVTLTVDKSTSTAYMELSSLRSEDTAVYYCAR SGYYGDSDWYFDVWGQGTLVTV |
| | | SSASTKGPSVFPLAPSSKSTSGGTAALGCLVKDYFPEP VTVSWNSGALTSGVHTFPAVLQ SSGLYSLSSWTVPSSSLGTQTYICNVNHKPSNTKVDK KVEPKSCDKTHTSPPSPAPPVA GILNVEQSPQSLHVQEGDSTNFTCSFPVKEFQDLHWY RKETAKSPEFLFYFGPYGKEKKK GRISATLNTKEGYSYLYITDSQPEDSATYLCALYNNNDM RFGAGTRLTVKPGGGGSGGGG SGGGGSGGGGSGGGGSGVIQSPRHLVTEMGQEVTLR CKPISGHNSLFWYRETPMQGLELL IYFQNTAVIDDSGMPEDRFSAKMPNASFSTLKIQPSEPR DSAVYFCASSPGATDTQYFGP GTRLTVL |
| 184 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGDI QMTQSPSSLSASVGDRVTITCRASQDIRNYLNWYQQK PGKAPKLLIYYTSRLHSGVPSRF SGSGSGTDYTLTISSLQPEDIATYFCQQGQTLPWTFGQ GTKVEIKEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASI EKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 185 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHLVTEMGQEVTLRCKPISGH NSLFWYRETPMQGLELLIYFQNT AVIDDSGMPEDRFSAKMPNASFSTLKIQPSEPRDSAVY FCASSPGATDTQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 186 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHLVTEMGQEVTLRCKPISGHNSLFWYRETPMQGLELL IYFQNTAVIDDSGM PEDRFSAK |
| | | MPNASFSTLKIQPSEPRDSAVYFCASSPGATDTQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 187 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 188 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHLVTEMGQEVTLRCKPISGHNSLFWYRETPMQG LELLIYFQNTAVI DDSGMPEDRF SAKMPNASFSTLKIQPSEPRDSAVYFCASSPGATDTQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQG NVFSCSVMHEALHNHYTQKSLSLSP |
| 189 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV |
| | | KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 190 | α-chain | IMNVEQSPQSLHVQEGDSTNFTCSFPVKEFQDLHWYR KETAKSPEFLFYFGPYGKEKKKG RISATLNTKEGYSYLYITDSQPEDSATYLCALYNNNDMR FGAGTRLTVKPGGGSGGGGDI QMTQSPSSLSASVGDRVTITCRASQDIRNYLNWYQQK PGKAPKLLIYYTSRLHSGVPSRF SGSGSGTDYTLTISSLQPEDIATYFCQQGQTLPWTFGQ GTKVEIKEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 191 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHLVTEMGQEVTLRCKPISGH NSLFWYRETPMQGLELLIYFQNT AVIDDSGM PEDRFSAKMPNOSFSTLKIQPSEPRDSAVY FCASSPGATDTQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 192 | α-chain | DIQMTQSPSSLSASVGDRVTITCRASQDIRNYLNWYQQ KPGKAPKLLIYYTSRLHSGVPS RFSGSGSGTDYTLTISSLQPEDIATYFCQQGQTLPWTF GQGTKVEIK |
| 193 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SS |
| 194 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP |
| | | RHLVTEMGQEVTLRCKPISGHNSLFWYRETPMQGLELL IYFQNTAVIDDSGM PEDRFSAK MPNDSFSTLKIQPSEPRDSAVYFCASSPGATDTQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 195 | α-chain | IMNVEQSPQSLHVQEGDSTNFTCSFPVKEFQDLHWYR KETAKSPEFLFYFGPYGKEKKKG RISATLNTKEGYSYLYITDSQPEDSATYLCALYNNNDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 196 | β-chain | EVQLVQSGAEVKKPGASVKVSCKASGYKFTSYVMHWV RQAPGQGLEWMGYINPYNDVTKY AEKFQGRVTLTSDTSTSTAYMELSSLRSEDTAVHYCAR GSYYDYEGFVYWGQGTLVTVSS |
| 197 | α-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIK |
| 198 | β-chain | EVQLVQSGAEVKKPGASVKVSCKASGYKFTRYVMHWV RQAPGQGLEWMGYINPYNDVTKY AEKFQGRVTLTSDTSTSTAYMELSSLRSEDTAVHYCAR GSYYDYEGFVYWGQGTLVTVSS |
| 199 | β-chain | EVQLVQSGAEVKKPGASVKVSCKASGYKFTSYVMHWV RQAPGQGLEWMGYINPRNDVTKY AEKFQGRVTLTSDTSTSTAYMELSSLRSEDTAVHYCAR GSYYDYEGFVYWGQGTLVTVSS |
| 200 | α-chain | EVQLVQSGAEVKKPGASVKVSCKASGYKFTRYVMHWV RQAPGQGLEWMGYINPYNDVTKY AEKFQGRVTLTSDTSTSTAYMELSSLRSEDTAVYYCAR GSYYDYEGFVYWGQGTLVTVSS |
| 201 | β-chain | EVQLVQSGAEVKKPGASVKVSCKASGYKFTSYVMHWV RQAPGQGLEWMGYINPRNDVTKY |
| | | AEKFQGRVTLTSDTSTSTAYMELSSLRSEDTAVYYCAR GSYYDYEGFVYWGQGTLVTVSS |
| 202 | β-chain | EVQLVESGGGLVQPGGSLKLSCAASGFTFNKYAMNWV RQAPGKGLEWVARIRSKYNNYAT YYADSVKDRFTISRDDSKNTAYLQMNNLKTEDTAVYYC VRHGNFGNSYISYWAYWGQGTL VTVSS |
| 203 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHLVTEMGQEVTLRCKPISGH NSLFWYRETPMQGLELLIYFQNT A VI DDSGMPEDRFSAKM PN OS FSTLKIQPSEPRDSAVY FCASSPGATDLQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 204 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVL |
| 205 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHEVTEMGQEVTLRCKPISGH NSLFWYRETPMQGLELLIYFQNT AVIDDSGM PEDRFSAKMPNOSFSTLKIQPSEPRDSAVY FCASSPGATDLQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 206 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGDI QMTQSPSSLSASVGDRVTITCRASQDIRNYLNWYQQK PGKAPKLLIYYTSRLHSGVPSRF |
| | | SGSGSGTDYTLTISSLQPEDIATYFCQQGQTLPWTFGQ GTKVEIKEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 207 | β-chain | EVQLVESGGGLVQPGGSLKLSCAASGFTFNKYAMNWV RQAPGKGLEWVARIRSKYNNYAT YYADSVKDRFTISRDDSKNTAYLQMNNLKTEDTAVYYC VRHGNFGDSYISYWAYWGQGTL VTVSS |
| 208 | α-chain | IMNVEQSPQSLHVQEGDSTNFTCSFPVKEFQDLHWYR KETAKSPEFLFYFGPYGKEKKKG RISATLNTKEGYSYLYITDSQPEDSATYLCALYNNNDMR FGAGTRLTVKPGGGSGGGGDI QMTQSPSSLSASVGDRVTITCRASQDIRNYLNWYQQK PGKAPKLLIYYTSRLHSGVPSRF SGSGSGTDYTLTISSLQPEDIATYFCQQGQTLPWTFGQ GTKVEIKEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 209 | β-chain | EVQLVESGGGLVQPGGSLKLSCAASGFTFNKYAMNWV RQAPGKGLEWVARIRSKYNNYAT YYADSVKDRFTISRDDSKNTAYLQMNNLKTEDTAVYYC VRHGNFGESYISYWAYWGQGTL VTVSS |
| 210 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGDI QMTQSPSSLSASVGDRVTITCRASQDIRNYLNWYQQK PGKAPKLLIYYTSRLHSGVPSRF SGSGSGTDYTLTISSLQPEDIATYFCQQGQTLPWTFGQ GTKVEIKEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 211 | β-chain | EVQLVESGGGLVQPGGSLKLSCAASGFTFNKYAMNWV RQAPGKGLEWVARIRSKYNNYAT YYADSVKDRFTISRDDSKNTAYLQMNNLKTEDTAVYYC VRHGNFGNAYISYWAYWGQGTL VTVSS |
| 212 | α-chain | IMNVEQSPQSLHVQEGDSTNFTCSFPVKEFQDLHWYR KETAKSPEFLFYFGPYGKEKKKG RISATLNTKEGYSYLYITDSQPEDSATYLCALYNNYDMR FGAGTRLTVKPGGGSGGGGDI QMTQSPSSLSASVGDRVTITCRASQDIRNYLNWYQQK PGKAPKLLIYYTSRLHSGVPSRF SGSGSGTDYTLTISSLQPEDIATYFCQQGQTLPWTFGQ GTKVEIKEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASI EKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHN HYTQKSLSLSP |
| 213 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHEVTEMGQEVTLRCKPISGH NSLFWYRETPMQGLELLIYFQNT AVIDDSGM PEDRFSAKMPNOSFSTLKIQPSEPRDSAVY FCASSPGATDKQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 214 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHEVTEMGQEVTLRCKPISGH NSLFWYRETMMQGLELLIYFQNT AVIDDSGMPEDRFSAKMPNDSFSTLKIQPSEPRDSAVY FCASSPGATDLQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP |
| | | PVLDSDGSFFLVSKLTVDKSRWQQGNVFSCSVMHEAL HNHYTQKSLSLSP |
| 215 | β-chain | EVQLVQSGAEVKKPGASVKVSCKASGYSFTGYTMNWV RQAPGQGLEWMGLINPYKGVSTY AQKFQDRVTLTVDKSTSTAYMELSSLRSEDTAVYYCAR SGYYGDSDWYFDVWGQGTLVTV SSGGGSGGGGGVIQSPRHEVTEMGQEVTLRCKPISGH NSLFWYRETMMRGLELLIYFQNT AVIDDSGMPEDRFSAKMPNDSFSTLKIQPSEPRDSAVY FCASSPGATDLQYFGPGTRLTV LEPKSSDKTHTCPPCPAPPVAGPSVFLFPPKPKDTLMIS RTPEVTCWVDVSHEDPEVKF NWYVDGVEVHNAKTKPREEQYQSTYRVVSVLTVLHQD WLNGKEYKCKVSNKALPASIEKT ISKAKGQPREPQVCTLPPSRDELTKNQVSLSCAVKGFY PSDIAVEWESNGQPENNYKTTP PVLDSDGSFFLVSKLTVDKSRWQQGNVFSCSVMHEAL HNHYTQKSLSLSP |
| 216 | α-chain | ILNVEQSPQSLHVQEGDSTKFTCS FPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTRYVMHWVR QAPGQGLEWMGYINPYNDVTKYAE KFQGRVTLTSDTSTSTAYMELSSLRSEDTAVYYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 217 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDLQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 218 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 219 | α-chain | ILNVEQSPQSLHVQEGDSTKFTCS FPVKEFQDLHWYRK ETAKSPEFLFYFGPYGKEKKKG RISATLNTKEGYSYLYITDSQPEDSATYLCALYNNLDMR FGAGTRLTVKPGGGSGGGGEV QLVQSGAEVKKPGASVKVSCKASGYKFTSYVMHWVR QAPGQGLEWMGYINPRNDVTKYAE KFQGRVTLTSDTSTSTAYMELSSLRSEDTAVYYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 220 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 221 | α-chain | ILNVEQSPQSLHVQEGDSTKFTCSFPVKEFQDLHWYRK ETAKSPEFLFYFGPYGKEKKKG |
| | | RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPRNDVTKYAE KFQGRVTLTSDTSTSTAYMELSSLRSEDTAVYYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 222 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 223 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 224 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK |
| | | MPNDSFSTLKIQPSEPRDSAVYFCASSAGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 225 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 226 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGAIDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 227 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSAGSTDAQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL |
| | | PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 228 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSIDAQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 229 | α-chain | ILNVEQSPQSLHVQEGDSTKFTCSFPVKEFQDIHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPRNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 230 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 231 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETMMRGLEL LlYFQNTAVIDDSGMPEDRFSAK MPNDSFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 232 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 233 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSAGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 234 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV |
| | | QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 235 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSTGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 236 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 237 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSPGAIDKQYFGP GTRLTVLEPKSSDKTHTCPPCP |
| | | APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 238 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGDSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 239 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNDSFSTLKIQPSEPRDSAVYFCASSPGATDKQY FGPGTRLTVLEPKSSDKTHTCP PCPAPPVAGPSVFLFPPKPKDTLMISRTPEVTCVWDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 240 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE |
| | | KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 241 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGESYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 242 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 243 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNAYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPR EEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT |
| | | TPPVLDSDGSFFLVSKLTVDKSRWQQGNVFSCSVMHE ALHNHYTQKSLSLSP |
| 244 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 245 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSAGAIDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 246 | α-chain | ILNVEQSPQSLHVQEGDSTKFTCS FPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 247 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 248 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSTGAIDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 249 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNASFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 250 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY |
| | | ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFN WYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 251 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNNDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 252 | α-chain | ILNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRWSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 253 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNADMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP |
| | | KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 254 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNDDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 255 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNEDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 256 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNFDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY |
| | | VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 257 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNHDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 258 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNIDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASI EKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 259 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNLDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK |
| | | AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 260 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNKDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 261 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNQDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 262 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNRDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL |
| | | DSDGSFFLVSKLTVDKSRWQQGNVFSCSVMHEALHNH YTQKSLSLSP |
| 263 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNVDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 264 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNESFSTLKIQPSEPRDSAVYFCASSPGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 265 | α-chain | ILNVEQSPQSLHVQEGDSTNFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASIEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 266 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNRSFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 267 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNKSFSTLKIQPSEPRDSAVYFCASSPGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 268 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNQSFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 269 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK |
| | | MPNNSFSTLKIQPSEPRDSAVYFCASSPGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 270 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNSSFSTLKIQPSEPRDSAVYFCASSPGATDKQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 271 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDRQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 272 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDHQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL |
| | | PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 273 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDEQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 274 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDAQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 275 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDQQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 276 | β-chain | QIQMTQSPSSLSASVGDRVTITCSATSSVSYMHWYQQ KPGKAPKRWIYDTSKLASGVPSR |
| | | FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGATDNQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 277 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDFQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 278 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDYQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 279 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDIQYFGP GTRLTVLEPKSSDKTHTCPPCP |
| | | APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 280 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGATDVQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 281 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGSTDRQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 282 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSTDHQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT |
| | | VDKSRWQQGNVFSCSVMHEALHNHYTQKSLSLSP |
| 283 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSTDEQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 284 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGSTDAQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 285 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGSTDQQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 286 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP |
| | | RHEVTEMGQEVTLRCKPISGHNSLFWYRETPMQGLEL LIYFQNTAVIDDSGMPEDRFSAK MPNDSFSTLKIQPSEPRDSAVYFCASSPGSTDNQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 287 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSTDFQYFGP GTRLTVLEPKSSDKTHTCPPCP APPVAGPSVFLFPPKPKDTLMISRTPEVTCVVVDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 288 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSTDYQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASIEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 289 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LlYFQNTAVIDDSGMPEDRFSAK MPNDSFSTLKIQPSEPRDSAVYFCASSPGSTDIQYFGP GTRLTVLEPKSSDKTHTCPPCP APPVAGPSVFLFPPKPKDTLMISRTPEVTCVVVDVSHE DPEVKFNWYVDGVEVHNAKTKP |
| | | REEQYQSTYRVVSVLTVLHQDWLNGKEYKCKVSNKAL PASlEKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 290 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGSTDVQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASl EKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 291 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNESFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 292 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNRSFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 293 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNKSFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 294 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVI DDSGMPEDRF SAKMPNQSFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 295 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNNSFSTLKIQPSEPRDSAVYFCASSPGATDKQY FGPGTRLTVLEPKSSDKTHTCP PCPAPPVAGPSVFLFPPKPKDTLMISRTPEVTCVWDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 296 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF |
| | | SAKMPNSSFSTLKIQPSEPRDSAVYFCASSPGATDKQY FGPGTRLTVLEPKSSDKTHTCP PCPAPPVAGPSVFLFPPKPKDTLMISRTPEVTCVVVDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 297 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNDSFSTLKIQPSEPRDSAVYFCASSPGSTDAQYFGP GTRLTVLEPKSSDKTHTCPPCP APPVAGPSVFLFPPKPKDTLMISRTPEVTCVVV/DVSHE DPEVKFNWYVDGVEVHNAKTKP REEQYQSTYRVVSVLTVLHQDWLNGKEYKCKVSNKAL PASl EKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 298 | α-chain | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTRYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASlEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 299 | α-chain | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKPGGGSGGGGEV QLVQSGAEVKKPGASVKVSCKASGYKFTSYVMHWVR QAPGQGLEWMGYINPRNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY |
| | | VDGVEVHNAKTKPREEQYQSTYRVVSVLTVLHQDWLN GKEYKCKVSNKALPASlEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 300 | α-chain | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNLDMR FGAGTRLTVKPGGGSGGGGEV QLVESGGGLVQPGGSLKLSCAASGFTFNKYAMNWVR QAPGKGLEWVARIRSKYNNYATYY ADSVKDRFTISRDDSKNTAYLQMNNLKTEDTAVYYCVR HGNFGNSYISYWAYWGQGTLVT VSSEPKSSDKTHTCPPCPAPPVAGPSVFLFPPKPKDTL MISRTPEVTCVWDVSHEDPEV KFNWYVDGVEVHNAKTKPREEQYQSTYRVVSVLTVLH QDWLNGKEYKCKVSNKALPASIE KTISKAKGQPREPQVCTLPPSRDELTKNQVSLSCAVKG FYPSDIAVEWESNGQPENNYKT TPPVLDSDGSFFLVSKLTVDKSRWQQGNVFSCSVMHE ALHNHYTQKSLSLSP |
| 301 | β-chain | QTWTQEPSLTVSPGGTVTLTCGSSTGAVTSGYYPNW VQQKPGQAPRGLIGGTKFLAPGT PARFSGSLLGGKAALTLSGVQPEDEAEYYCALWYSNR WVFGGGTKLTVLGGGSGGGGGVI QSPRHEVTEMGQEVTLRCKPISGHNSLFWYRETPMQG LELLIYFQNTAVIDDSGMPEDRF SAKMPNDSFSTLKIQPSEPRDSAVYFCASSPGATDKQY FGPGTRLTVLEPKSSDKTHTCP PCPAPPVAGPSVFLFPPKPKDTLMISRTPEVTCVWDV SHEDPEVKFNWYVDGVEVHNAK TKPREEQYQSTYRVVSVLTVLHQDWLNGKEYKCKVSN KALPASIEKTISKAKGQPREPQV YTLPPCRDELTKNQVSLWCLVKGFYPSDIAVEWESNG QPENNYKTTPPVLDSDGSFFLYS KLTVDKSRWQQGNVFSCSVMHEALHNHYTQKSLSLSP |
| 302 | α-chain | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNLDMR FGAGTRLTVKPGGGSGGGGEV QLVQSGAEVKKPGASVKVSCKASGYKFTRYVMHWVR QAPGQGLEWMGYINPYNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASlEKTISK AKGQPREPQVCTLPPSRDELTKNQVSLSCAVKGFYPS DIAVEWESNGQPENNYKTTPPVL |
| | | DSDGSFFLVSKLTVDKSRWQQGNVFSCSVMHEALHNH YTQKSLSLSP |
| 303 | β-chain | QIQMTQSPSSLSASVGDRVTITCSATSSVSYMHWYQQ KPGKAPKRWIYDTSKLASGVPSR FSGSGSGTDYTLTISSLQPEDAATYYCQQWSSNPLTFG GGTKVEIKGGGSGGGGGVIQSP RHEVTEMGQEVTLRCKPISGHNSLFWYRETPMQGLEL LIYFQNTAVIDDSGMPEDRFSAK MPNASFSTLKIQPSEPRDSAVYFCASSPGATDKQYFGP GTRLTVLEPKSSDKTHTCPPCP APPVAGPSVFLFPPKPKDTLMISRTPEVTCVWDVSHE DPEVKFNWYVDGVEVHNAKTKP REEQYQSTYRVVSVLTVLHQDWLNGKEYKCKVSNKAL PASl EKTISKAKGQPREPQVYTL PPCRDELTKNQVSLWCLVKGFYPSDIAVEWESNGQPE NNYKTTPPVLDSDGSFFLYSKLT VDKSRWQQGNVFSCSVMHEALHNHYTQKSLSLSP |
| 304 | α-chain | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNLDMR FGAGTRLTVKPGGGSGGGGEV QLVQSGAEVKKPGASVKVSCKASGYKFTSYVMHWVR QAPGQGLEWMGYINPRNDVTKYAE KFQGRVTLTSDTSTSTAYMELSSLRSEDTAVHYCARGS YYDYEGFVYWGQGTLVTVSSEP KSSDKTHTCPPCPAPPVAGPSVFLFPPKPKDTLMISRT PEVTCVWDVSHEDPEVKFNWY VDGVEVHNAKTKPREEQYQSTYRVVSVLTVLHQDWLN GKEYKCKVSNKALPASlEKTISK AKGQPREPQVCTLPPSRDELTKNQVSLSCAVKGFYPS DIAVEWESNGQPENNYKTTPPVL DSDGSFFLVSKLTVDKSRWQQGNVFSCSVMHEALHNH YTQKSLSLSP |
| 305 | Va | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNYDMR FGAGTRLTVKP |
| 306 | Vb | GVIQSPRHEVTEMGQEVTLRCKPISGHNSLFWYRETP MQGLELLIYFQNTAVI DDSGMPE DRFSAKMPNDSFSTLKIQPSEPRDSAVYFCASSPGATD KQYFGPGTRLTVL |
| 307 | Vb | GVIQSPRHEVTEMGQEVTLRCKPISGHNSLFWYRETP MQGLELLIYFQNTAVI DDSGMPE DRFSAKMPNASFSTLKIQPSEPRDSAVYFCASSPGATD KQYFGPGTRLTVL |
| 308 | Vb | GVIQSPRHEVTEMGQEVTLRCKPISGHNSLFWYRETP MQGLELLIYFQNTAVIDDSGMPE DRFSAKMPNDSFSTLKIQPSEPRDSAVYFCASSPGSTD AQYFGPGTRLTVL |
| 309 | Va | lLNVEQSPQSLHVQEGDSTKFTCSFPVKEFQDLHWYRK ETAKSPEFLFYFGPYGKEKKKG RISATLNTKEGYSYLYITDSQPEDSATYLCALYNNLDMR FGAGTRLTVKP |
| 310 | PRAME-004 | SLLQHLIGL |
| 311 | NY-ESO1-001 | SLLMWITQV |
| 312 | KRT5-004 | STASAITPSV |

### Example 18

### General Patient Identification Procedure - TCR R11P3D3_KE T Cells

Blood or cells are obtained from a tumor patient via techniques such as, but not limited to, blood draw or buccal swab. A patient(s) expressing HLA-A*02:01 is identified. An HLA-A*02:01⁺ patient(s) is tested for tumor(s) expressing PRAME. Tumor tissue is obtained via a treatment-specific biopsy or a medically indicated procedure, such as, but not limited to, resection or debulking surgery. Core needle biopsies may be taken; if biopsies are taken, approximately 2 cm of tumor material may be aspirated with an approximately 22G needle. Tumor cell content of the biopsy or tissue may be high, as high normal tissue content may negatively influence assays. The target number of tumor biopsy samples may be approximately 5, approximately 4 of which may be immediately stored in RNA/ater® to test for the expression of PRAME by RT-qPCR. Approximately 1 sample may be prepared as a formalin-fixed paraffin embedded (FFPE) sample for analysis of tumor cell content and further analyses. Tumor tissue may be stored in an RNA-preserving manner, such as, but not limited to storage in an RNA stabilizer, such as RNA/ater® manufactured by Ambion®, Inc. Tumor tissue is tested for the expression of PRAME by reverse transcription real time-quantitative polymerase chain reaction (RT-qPCR), as a non-limiting example, using by the real time-quantitative polymerase chain reaction (RT-qPCR)-based IMADetect® assay. PRAME-004 biomarker testing may be performed using an RT-qPCR based assay. From a biopsy or tissue specimen taken from a patient(s), RNA is isolated, complementary DNA is synthesized, and quantitative expression of the target gene is analyzed using, as a non-limiting example, a Life Technologies 7500 Real-Time PCR System. The assay format may be 1 standardized PCR plate per patient, including primers and fluorescent probes for the target, controls without addition of complementary DNA template, and controls omitting the reverse transcriptase in the complementary DNA synthesis to account for genomic DNA contamination. These plates may be prepared and provided by the manufacturer (Thermo Fisher Scientific, Waltham, MA). Normalization of the data may be performed by measuring levels of 3 different reference genes with pre-tested stable expression across tumors and normal tissues. Target expression values may be calculated relative to the mean expression of the 3 reference genes. Target expression may be called positive if the normalized expression value is above the target-associated pre-defined threshold. An HLA-A*02⁺ patient(s) having a PRAME ⁺ tumor(s) is identified.

### Example 19

### General Patient Identification Procedure - MAGE-A4-Binding Molecules

A patient(s) having a MAGE-A4 positive (MAGE-A4⁺) tumor(s) is identified. Tumor tissue is obtained via a treatment-specific biopsy or a medically indicated procedure, such as, but not limited to, resection or debulking surgery. Core needle biopsies may be taken; if biopsies are taken, approximately 2 cm of tumor material may be aspirated with an approximately 22 G needle. Tumor cell content of the biopsy or tissue may be high, as high normal tissue content may negatively influence assays. The target number of tumor biopsy samples may be approximately 5, approximately 4 of which may be immediately stored in RNAlater to test for the expression of PRAME by RT-qPCR. Approximately 1 sample may be prepared as a FFPE sample for analysis of tumor cell content and further analyses. Tumor tissue may be stored in an RNA-preserving manner, such as, but not limited to storage in an RNA stabilizer, such as RNA/ater®. Tumor tissue is tested for the expression of MAGE-A4

### Example 20

### General Patient Identification Procedure - Genetically Engineered Autologous T Cells Specific For HLA-A2-Restricted MAGE-A4230-239 Peptide GVYDGREHTV (SEQ ID NO: 401) Expressed in the Context of HLA-A*02

Blood or cells are obtained from a tumor patient via techniques such as, but not limited to, blood draw or buccal swab. A patient(s) having HLA-A*02 is identified art-known techniques, such as, but not limited to, PCR-based methods or sequencing methods. An HLA-A*02⁺ patient(s) is tested for tumor(s) expressing MAGE-A4. Tumor tissue is obtained via a treatment-specific biopsy or a medically indicated procedure, such as, but not limited to, resection or debulking surgery. Core needle biopsies may be taken; if biopsies are taken, approximately 2 cm of tumor material may be aspirated with an approximately 22G needle. Tumor cell content of the biopsy or tissue may be high, as high normal tissue content may negatively influence assays. The target number of tumor biopsy samples may be approximately 5, approximately 4 of which may be immediately stored in RNAlater to test for the expression of PRAME by RT-qPCR. Approximately 1 sample may be prepared as a FFPE sample for analysis of tumor cell content and further analyses. Tumor tissue may be stored in an RNA-preserving manner, such as, but not limited to storage in an RNA stabilizer, such as RNA/ater®. Tumor tissue is tested for the expression of MAGE-A4. An HLA-A*02⁺ patient(s) having a MAGE-A4⁺ tumor(s) is identified.

### Example 21

### General Identification Procedure - PD-1/PD-L1 Interaction Inhibitors

A patient(s) having a PD-L1 positive (PD-L1⁺) tumor(s) is identified. Tumor tissue is obtained via a treatment-specific biopsy or a medically indicated procedure, such as, but not limited to, resection or debulking surgery. Core needle biopsies may be taken; if biopsies are taken, approximately 2 cm of tumor material may be aspirated with an approximately 22G needle. Tumor cell content of the biopsy or tissue may be high, as high normal tissue content may negatively influence assays. Tumor tissue may be stored in an RNA-preserving manner, such as, but not limited to storage in an RNA stabilizer, such as RNA/ater®. Tumor tissue is tested for the expression of PD-L1. Tumor mutation burden may also be assessed.

### Example 22

### General Treatment Procedure - Leukapheresis

Leukapheresis is performed according to art-known procedures. The target cell number for collection may be approximately 1 × 10⁹ to approximately 10 × 10¹⁰ mononuclear cells, approximately 5 × 10⁹ to approximately 5 × 10¹⁰ mononuclear cells, or approximately 5 × 10⁹ mononuclear cells. Repeated leukapheresis may be performed if the leukapheresis was insufficient or T-cell product could not be produced from the collected cells.

### Example 23

### General Treatment Procedure - Lymphodepletion

Non-myeloablative chemotherapy for lymphodepletion is performed on a patient(s) prior to infusion treatment(s). Lymphodepletion may be performed, as non-limiting examples, daily for approximately 5 consecutive days prior to infusion of T cells, approximately 4 consecutive days prior to infusion of T cells, approximately 3 consecutive days prior to infusion of T cells, approximately 2 consecutive days prior to infusion of T cells, or approximately 1 day prior to infusion of T cells. Lymphodepletion may be performed, as non-limiting examples, every other day for approximately 11 days prior to infusion of T cells, every other day for approximately 9 days prior to infusion of T cells, every other day for approximately 7 days prior to infusion of T cells, every other day for approximately 5 days prior to infusion of T cells, or every other day for approximately 3 days prior to infusion of T cells. When lymphodepletion is performed, as a non-limiting example, daily for 4 consecutive days, the days may be, as non-limiting examples, about Day -7 to about Day -4, about Day -6 to about Day -3, about Day -5 to about Day -2, or about Day -4 to about Day -1, prior to infusion of T cells. However, T cell infusion may be delayed for up to approximately 7 days post-lymphodepletion (after the last day of lympodepletion) for, as non-limiting examples, management of comorbidity, such as, but not limited to, fever, ongoing infections, or combinations thereof. If T cell infusion may be delayed for longer than approximately 5, approximately 6, or approximately 7 days after the last day of lymphodepletion, a second lymphodepletion may be performed.

Fludarabine (FLU) is a fluorinated nucleotide analog of the antiviral agent vidarabine. Fludarabine phosphate is rapidly dephosphorylated to 2-fluoro-ara-A and then phosphorylated intracellularly by deoxycytidine kinase to the active triphosphate, 2-fluoro-ara-ATP. This metabolite appears to act by inhibiting deoxyribonucleic acid (DNA) polymerase alpha, ribonucleotide reductase, and DNA primase, thus inhibiting DNA synthesis.

Cyclophosphamide (CY) is a cytotoxic drug for the treatment of malignant disease in adults and children. Following IV administration, the elimination half-life of CY may range from approximately 3 to approximately 12 hours.

As non-limiting examples, lymphodepletion regimen (LDR) may comprise administration of drugs such as fludarabine, cyclophosphamide, or combinations thereof. Doses may be calculated, as a non-limiting example, as weight per body surface area (BSA) as defined by the Mosteller formula. Mosteller RD (1987), Simplified calculation of body-surface area. N Engl J Med. 317(17):1098, which is incorporated herein by reference in its entirety.

As non-limiting examples, total doses of CY may be from approximately 500 mg/m² total CY to approximately 3600 mg/m² total CY, 1000 mg/m² total CY to approximately 3000 mg/m² total CY, 1200 mg/m² total CY to approximately 2500 mg/m² total CY, 1500 mg/m² total CY to approximately 2000 mg/m² total CY, approximately 1000 mg/m² total CY, approximately 1600 mg/m² total CY, approximately 1800 mg/m² total CY, approximately 2000 mg/m² total CY, approximately 3000 mg/m² total CY, or approximately 3600 mg/m² total CY.

As non-limiting examples, total doses of FLU may be from approximately 50 mg/m² to approximately 200 mg/m² total FLU, 100 mg/m² to approximately 160 mg/m² total FLU, approximately 80 mg/m² to approximately 160 mg/m² total FLU, approximately 60 mg/m² to approximately 120 mg/m² total FLU, approximately 60 mg/m² total FLU, approximately 80 mg/m² total FLU, approximately 100 mg/m² total FLU, approximately 120 mg/m² total FLU, approximately 140 mg/m² total FLU, or approximately 150 mg/m² total FLU, approximately 160 mg/m² total FLU, approximately 170 mg/m² total FLU, or approximately 200 mg/m² total FLU.

Total doses may be given over one or more days, such as, but not limited to, over 4 days, and may be varied from day to day, or may be the same from day to day.

Doses may be varied, as non-limiting examples, to maintain a high level of wanted primary pharmacology, to reduce potential unwanted secondary pharmacology from too strong activation of immune-cells through the IL-6 axis, to decrease the risk of prolonged cytopenias, or combinations thereof. Doses also may be varied (increased or decreased) to account for patient(s) health status, tumor type, tumor status, other considerations, or combinations thereof.

As non-limiting examples, the LDR(s) for patient(s) with solid tumors and or with hepatocellular carcinoma (HCC) tumors, with adequate renal function and adequate bone marrow reserve may be as outlined in in Table 8, dose regimen 1. As non-limiting examples, the LDRs may be adapted depending on renal impairment, reduced bone marrow reserve, or other increased risks for adverse events from FLU and CY (dose regimens 2, 3), as depicted in Table 8. Doses in Table 8 are given as per day doses, not total doses. Patients who have both conditions, renal impairment and reduced bone marrow reserve, may be considered to be ineligible for lymphodepletion, ineligible for treatment, or both.

TABLE 8

Exemplary FLU and CY Regimens

| Dose | FLU (mg/m²) per day | CY (mg/m²) per day | Days |
|---|---|---|---|
| Dose regimen 1: Patients with adequate renal function^{a} and with adequate bone marrow reserve | | | |
| Solid tumors except HCC | about 30 | about 500 | about 4 days |
| HCC patients | about 25 | about 400 | about 4 days |
| Dose regimen 2: Patients with moderate renal impairment^{b} and with adequate bone marrow reserve | | | |
| Solid tumors except HCC | about 25 | about 500 | about 4 days |
| HCC | about 20 | about 400 | about 4 days |
| Dose regimen 3: Patients with reduced bone marrow reserve^{C} and with adequate renal function^{a} | | | |
| Solid tumors except HCC | about 25 | about 400 | about 4 days |
| HCC | about 20 | about 300 | about 4 days |

^{a} creatinine clearance ≥ 70 mL/min/1.73 m²
^{b} creatinine clearance < 70 mL/min/1.73 m² and ≥ 50 mL/min/1.73 m²
^{c} patients aged >70 years and/or with heavy pre-treatments or other conditions impacting bone marrow reserve
CY = cyclophosphamide; FLU = fludarabine; HCC = hepatocellular carcinoma

Standard Practice Policy guidelines and instructions according to the prescribing information of FLU and CY may be followed. Hydration according to local hospital standard may be administered, may avoid or lessen renal damage, and may start, as a non-limiting example, about 2 hours prior to administration of CY. Hydration using a balanced crystalloid may be employed. (See, e.g., Hoorn EJ (2017), Intravenous fluids: balancing solutions, J Nephrol 30, 485-492, which is incorporated herein in its entirety. Mesna may be administered with CY, and may avoid or lessen bladder and/or renal damage. As a non-limiting example, mesna may be administered according to institutional standards. As a non-limiting example, 2 doses of mesna may be administered daily between from immediately prior to administration of CY to the final day of lymphodepletion. As a non-limiting example, mesna may be administered intravenously at 250 mg/m² over 30 minutes starting immediately prior to CY administration and may be repeated 4 hours post CY administration. As a non-limiting example, anti-emetics may be administered according to institutional standards.

### Example 24

### General Production Procedure - TCR R11P3D3_KE T Cells

Peripheral blood mononuclear cells (PBMC) will be isolated from patient(s) leukapheresis samples after the removal of red blood cells, activated using anti-cluster of differentiation (CD)3 and anti-CD28 antibodies, and then transduced ex vivo with a lentiviral vector containing genes encoding the PRAME-004 specific TCR (TCR R11 P3D3_KE). Transduced T cells will be further expanded ex vivo until sufficient T cells are produced.

### Example 25

### General Production Procedure - Genetically Engineered Autologous T Cells Specific For HLA-A2-Restricted MAGE-A4230-239 Peptide GVYDGREHTV (SEQ ID NO: 401) Expressed in the Context of HLA-A*02

Peripheral blood mononuclear cells (PBMC) isolated from patient(s) leukapheresis samples after the removal of red blood cells may be activated using anti-cluster of differentiation (CD)3 and anti-CD28 antibodies or via other methods, and then transduced ex vivo with a vector containing gene(s) encoding the genetically engineered specific peptide enhanced affinity receptor of ADP-A2M4. The peptide recognized by ADP-A2M4 cells is HLA-A2-restricted MAGE-A4230-239 peptide GVYDGREHTV (SEQ ID NO: 401) expressed in the context of HLA-A*02. Transduced T cells will be further expanded ex vivo until sufficient T cells are produced.

### Example 26

### General Treatment Procedure - TCR R11P3D3_KE T Cells

A patient(s) identified using the selection procedure for PRAME described in Example 18 is selected for treatment. A patient(s) having PRAME⁺ tumor(s) is selected for treatment.

A patient(s) undergoes leukapheresis to obtain autologous T cells for transduction with TCR R11P3D3_KE, an antigen-specific TCR that is highly specific for a human leukocyte antigen (HLA)-A*02:01-presented targeted peptide sequence (PRAME-004) derived from the PRAME protein, as described in Example 22. Autologous T cells are transduced with TCR R11P3D3_KE to produce autologous TCR R11P3D3_KE T cells, as described in Example 24.

Baseline tumor images may be obtained for a patient(s). lmages may be taken using, as non-limiting examples, using CT scanning, MRI scanning, PET scanning, x-ray imaging, or ultrasound. Baseline information from blood sample(s), tissue sample(s), urine sample(s), stool or gut sample(s), or other samples may be obtained. Information obtained may include, but is not limited to, information set forth below in this example.

A patient may undergo lymphodepletion, as described in Example 23 prior to infusion with TCR R11P3D3_KE T cells. Lymphodepletion may be performed, for example, daily for 4 consecutive days (Day -6 to Day -3) prior to infusion.

A patient(s) receives an intravenous (IV) infusion of autologous TCR R11P3D3_KE T cells on Day 0. The cell dose may be based, as a non-limiting example, on the number of viable cluster of differentiation (CD)³⁺ CD⁸⁺ HLA dextramer⁺ cells (which may represent the best available correlate to the number of active, transduced T cells). As non-limiting examples, the cell dose may be total cells (cells) or the cell dose may be measured, as a non-limiting example, per body surface area (BSA) as defined by the Mosteller formula (cells/m²). Mosteller RD, Simplified calculation of body-surface area. N Engl J Med. 1987 Oct 22;317(17):1098, which is incorporated herein by reference in its entirety.

A patient(s) may receive approximately 5 × 10⁷ to approximately 20 × 10¹⁰ cells, approximately 1 × 10⁸ to approximately 10 × 10¹⁰ cells, approximately 1 × 10⁹ to approximately 5 × 10¹⁰ cells, approximately 2 × 10⁹ to approximately 1 × 10¹⁰ cells, approximately 1 × 10⁹ to approximately 9 × 10⁹ cells, approximately 1 × 10⁹ to approximately 2 × 10¹⁰ cells, approximately 3 × 10⁹ to approximately 5 × 10⁹ cells, approximately 0.5 × 10⁹ to approximately 1.2 × 10⁹ cells, approximately 1.2 × 10⁹ to approximately 6 × 10⁹ cells, approximately 4.49 to approximately 9.98 × 10⁹ cells, approximately 8 × 10⁷ to approximately 0.12 × 10⁹ cells, approximately 5 × 10⁸ to approximately 1.2 × 10⁹ cells, approximately 41 × 10⁷ to approximately 9.98 × 10⁹ cells, approximately 41 × 10⁷ cells, approximately 0.08 × 10⁹ cells, approximately 0.1 × 10⁹ cells, approximately 0.12 × 10⁹ cells, approximately 0.15 × 10⁹ cells, approximately 0.5 × 10⁹ cells, approximately 1 × 10⁹ cells, approximately 1.2 × 10⁹ cells, approximately 5 × 10⁹ cells, approximately 6 × 10⁹ cells, approximately 7 × 10⁹ cells, approximately 8 × 10⁹ cells, approximately 9 × 10⁹ cells, approximately 9.8 × 10⁹ cells, approximately 10 × 10⁹ cells, no more than approximately 2 × 10¹⁰ cells, no more than approximately 5 × 10¹⁰ cells or no more than approximately 10 × 10¹⁰ cells,.

A patient(s) may receive approximately 1 × 10⁶ to approximately 18 × 10⁶ cells/m², approximately 12 × 10⁶ to approximately 18 × 10⁶ cells/m², approximately 40 × 10⁶ to approximately 60 × 10⁶ cells/m², approximately 120 × 10⁶ to approximately 180 × 10⁶ cells/m², approximately 240 × 10⁶ to approximately 480 × 10⁶ cells/m², approximately 200 × 10⁶ to approximately 480 × 10⁶ cells/m², approximately 200 × 10⁶ to approximately 500 × 10⁶ cells/m², approximately 200 × 10⁶ to approximately 1200 × 10⁶ cells/m², approximately 12 × 10⁶ to approximately 18 × 10⁶ cells/m², approximately 12 × 10⁶ to approximately 1200 × 10⁶ cells/m², approximately 1 × 10⁷ to approximately 14 × 10⁸ cells/m², approximately 41 × 10⁷ to approximately 2 × 10⁸ cells/m², approximately 1 × 10⁵ to approximately 15 × 10¹⁰ cells/m², approximately 12 × 10⁶ cells/m², approximately 15 × 10⁶ cells/m², approximately 18 × 10⁶ cells/m², approximately 40 × 10⁶ cells/m², approximately 50 × 10⁶ cells/m², approximately 60 × 10⁶ cells/m², approximately 120 × 10⁶ cells/m², approximately 150 × 10⁶ cells/m², approximately 180 × 10⁶ cells/m², approximately 200 × 10⁶ cells/m², approximately 340 × 10⁶ cells/m², approximately 480 × 10⁶ cells/m², approximately 40 × 10⁶ cells/m², approximately 600 × 10⁶ cells/m², approximately 700 × 10⁶ cells/m², approximately 1200 × 10⁶ cells/m², approximately 5.0 × 10¹⁰ or fewer cells/m², approximately 10 × 10¹⁰ or fewer cells/m², or approximately 15 × 10¹⁰ or fewer cells/m².

Intravenous infusion of a first bag of TCR R11 P3D3_KE T cells may be started at a slow rate (about 1 to about 2 mL/minute). The maximum infusion speed may be limited to approximately 5 mL/minute for any remaining bags if not further limited. An exact minimum infusion time may be calculated to ensure the endotoxin limit of < 5 EU/kg/hour is not exceeded. Infusion speed may be further reduced based on patient tolerance; however, a maximum allowable infusion time per bag may be approximately 30 minutes. Regardless of how many bags are used, this infusion may be considered to be a single dose infusion.

A patient(s) may be hospitalized for approximately 3 weeks (starting with the first day of lymphodepletion (Day -6), if lymphodepletion is performed. Patient(s) may be discharged from the hospital when clinically stable at the discretion of the clinician.

A patient(s) may receive prophylaxis for infections, as described in Example 37. A patient(s) may receive prophylaxis for allergic reactions, as described in Example 38. A patient(s) may be administered low-dose subcutaneous (SC) interleukin 2 (IL-2), as described in Example 36.

Patient(s) may additionally be monitored for approximately 2 years or approximately 3 years or more after discharge from the hospital; visits may occur approximately quarterly.

Patient(s) may be evaluated one or more times (pre- and/or post- treatment) for changes in health status, vital signs, and physical examinations, as non-limiting examples. Blood may be drawn from patient(s) one or more times (pre- and/or post-treatment). Blood may be drawn from patient(s) for, as non-limiting examples, health monitoring, analysis, or combinations thereof. PBMCs may be isolated, and characteristics such as T cell persistence (e.g., frequency of TCR engineered T cells as a fraction of blood T cells) (may be measured using techniques such as, but not limited to, standardized qPCR methods, cellular immune monitoring assays, or combinations thereof), functionality of T cells, phenotype of persisting T cells, and/or T-cell longevity may be measured or otherwise assessed, and other analysis may be performed. Peripheral blood mononuclear cells may be isolated from sodium heparin blood samples (approximately 20 mL or approximately 80 mL, as non-limiting examples) at selected time points pre- and post-infusion. Isolated PBMC may be cryopreserved until further analysis. Among other uses, PBMC may be used to assess T-cell persistence in vivo (such as, but not limited to, by qPCR on a unique sequence that is introduced with the lentiviral vector, by multimer staining, by other suitable methods, or combinations thereof). Among other uses, PBMC may be used to address the ex vivo functionality and phenotype of the infused T cells (e.g., by intracellular cytokine analysis or cytotoxicity assays).

The gut microbiome of patient(s) may be sampled, such as, but not limited to, via stool sample(s), and measured or otherwise assessed one or more times (pre-and/or post- treatment).

Gut microbiome composition may affect anti-tumor immunity. It has been reported that differential bacterial signatures exist in responders versus non-responders to therapy (with responders having higher diversity of the gut microbiome and differential composition compared to non-responders). Differences in the gut microbiome were associated with differential immune signatures in the tumor microenvironment. See, e.g., Gopalakrishnan V, et al. (2018), Gut microbiome modulates response to anti-PD-1 immunotherapy in melanoma patients, Science 359, 97-103; Routy B, et al. (2018), Gut microbiome influences efficacy of PD-1-based immunotherapy against epithelial tumors, Science 359, 91-97; Vetizou M, et al. (2015), Anticancer immunotherapy by CTLA-4 blockade relies on the gut microbiota, Science 350, 1079-1084; each of which is incorporated by reference herein in its entirety.

Cellular biomarkers (such as, but not limited to, circulating tumor cells (CTCs)) in sample(s) such as, but not limited to, blood or tissue sample(s), may be measured or otherwise assessed. Non-cellular biomarkers (such as, but not limited to, serum IL-2 concentrations and concentrations of other immune-related biomarkers, such as, but not limited to, cytokines, such as, but not limited to, IL-6, IFN-y, or combinations thereof) may be measured or otherwise assessed. Biomarkers may include, as non-limiting examples, biomarkers potentially associated with safety, biological activity, efficacy, T cell characteristics, or prognosis.

Biopsies of patient(s) tumor(s) may be taken one or more times (pre- and/or post- treatment) and/or tumor material may otherwise be collected (pre- and/or post-treatment), such as during surgery. Core needle biopsies may be taken; if biopsies are taken, approximately 2 cm of tumor material may be aspirated with an approximately 22G needle. Tumor cell content of the biopsy or tissue may be high, as high normal tissue content may negatively influence assays. Tumor tissue from archived formalin-fixed, paraffin-embedded tissues or fresh frozen tissues may also be measured or otherwise assessed. Immune cell, such as, but not limited to, T cell infiltration may be measured or otherwise assessed. Tumor tissue biomarkers and other biomarkers (from, as non-limiting examples, pre- and/or post-treatment biopsies and/or tumor material) may be measured or otherwise assessed. Biomarkers may include, as non-limiting examples, biomarkers potentially associated with safety, biological activity, efficacy, T cell characteristics, or prognosis.

Other measurements and/or assessments that may be performed on patient(s) samples include, as non-limiting examples: presence and functional status of immune cell populations (such as, but not limited to, regulatory T cells, myeloid-derived suppressor cells); serum cytokine levels (such as, but not limited to, IFN-y, IL-6); gene expression analysis (e.g., of immune inhibitory molecules such as PD-L1); immune cell infiltration; tumor mutational burden; gene expression of cancer-specific antigens on CTC; presence of anti-drug antibodies (ADA); gut microbiome composition; PD-L1 status and tumor mutation burden; or combinations thereof.

Patient(s) tumor(s) may be imaged one or more times (pre- and/or post-treatment). lmages may be taken using, as non-limiting examples, using computed tomography (CT) scanning, magnetic resonance imaging (MRI), positron emission tomography (PET) scanning, x-ray imaging, ultrasound analysis, plain film imaging, or combinations thereof. Bone scan(s) may also be performed. Patient(s) tumor(s) may be measured or otherwise assessed one or more times (pre- and/or post- treatment) using imaging, as a non-limiting example.

The status of patient(s) tumor(s) and/or clinical outcome and/or and progression-free survival (PFS) may be measured or otherwise assessed one or more times (pre- and/or post- treatment) with tumor assessment/response-related endpoints, using, as a non-limiting example, the RECIST guidelines, such as RECIST version 1.1 (RECIST 1.1) (see, e.g., Eisenhauer EA, et al. (2009) New response evaluation criteria in solid tumours: revised RECIST guideline (version 1.1). Eur J Cancer. 45(2):228-47; Schwartz LH, et al. (2016), RECIST 1.1-Update and clarification: From the RECIST committee, EurJ Cancer62, 132-137; Schwartz LH, et al. (2016), RECIST 1.1 - Standardisation and disease-specific adaptations: Perspectives from the RECIST Working Group, Eur J Cancer62 , 138-145; each of which is incorporated herein in its entirety), immune related RECIST (irRECIST) (see, e.g., Bohnsack O, et al. (2014), Adaptation and modification of the immune related response criteria (lRRC): IrRECIST, Journal of Clinical Oncology 32 , e22121-e22121; Nishino M, et al., (2014), Optimizing immune-related tumor response assessment: does reducing the number of lesions impact response assessment in melanoma patients treated with ipilimumab?, J Immunother Cancer2, 17; Nishino M, et al. (2015), Cancer immunotherapy and immune-related response assessment: The role of radiologists in the new arena of cancer treatment, Eur J Radiol 84, 1259-1268; each of which is incorporated herein in its entirety), or combinations thereof. Other clinical parameters (such as, but not limited to, C-reactive protein (CRP)) may be measured or otherwise assessed.

Measurable disease may be defined as the presence of at least 1 measurable lesion. A measurable lesion may be a lesion that can be accurately measured in at least 1 dimension (longest diameter in the plane of measurement is to be recorded) with a minimum size of about 10 mm by CT/MRI scan. Measurability of lesions on CT/MRI scan may be defined based on the assumption that CT/MRI slice thickness is about 5 mm or less. Where CT/MRI scans have slice thickness greater than about 5 mm, the minimum size for a measurable lesion may be twice the slice thickness. Non-measurable lesions may be all other lesions, including small lesions (longest diameter less than about 10 mm or pathological lymph nodes with less than or equal to about 10 to less than 15 mm short axis) as well as truly non-measurable lesions. Lesions considered truly non-measurable include, but are not limited to: leptomeningeal disease, ascites, pleural or pericardial effusion, inflammatory breast disease, lymphangitic involvement of skin or lung, or abdominal masses/abdominal organomegaly identified by physical examination that is not measurable by reproducible imaging techniques

To be considered pathologically enlarged and measurable, a lymph node may be ≥ about 15 mm in short axis when assessed by CT/MRI scan (CT/MRI scan slice thickness may be no greater than about 5 mm). Lytic bone lesions or mixed lytic-blastic lesions, with identifiable soft tissue components, that can be evaluated by cross sectional imaging techniques such as CT or MRI may be considered as measurable lesions if the soft tissue component meets the definition of measurability described above. Blastic bone lesions may be non-measurable. Bone scan, PET scan, or plain films are not considered adequate imaging techniques to measure bone lesions. However, these techniques may be used to confirm the presence or disappearance of bone lesions. Lesions that meet the criteria for radiographically defined simple cysts may not be considered as malignant lesions, neither measurable nor non-measurable, because they are, by definition, simple cysts. “Cystic lesions” thought to represent cystic metastases may be considered as measurable lesions if they meet the definition of measurability described above. Lesions that are situated in a previously irradiated area, or in an area subjected to other loco regional therapy, may be considered non-measurable lesions.

Duration of response may be analyzed for patient(s) who reach at least partial response (PR). The duration of response may be analyzed using Kaplan-Meier methods. A patient who experiences any form of tumor progression may be evaluated as a patient with event. The time point of event may be the diagnosis of progression according to RECIST. Duration of response may be calculated, as non-limiting examples, according to RECIST, according to irRECIST, or combinations thereof.

A single dose (single dose infusion) of TCR R11P3D3_KE T cells may be administered. A second or additional doses of TCR R11P3D3_KE T cells may be administered. A second dose of TCR R11P3D3_KE T cells may be desirable, as non-limiting examples, where a patient(s) responded (confirmed PR or CR according to RECIST1.1) to a first infusion of TCR R11P3D3_KE T cells, where a patient(s) developed progressive disease (PD) after partial response (PR) or complete response (CR) to a first infusion of TCR R11P3D3_KE T cells, where the presence/persistence of the PRAME can be re-confirmed in a fresh biopsy sample taken after progressive disease (PD), where a clinician may deem it is in the best interest of a patient(s), where patient(s) did not experience a severe toxicity. Severe toxicity may include, as non-limiting examples: Grade 3 or higher non-hematological adverse event (AE) (with the exceptions of transient nausea, vomiting, and diarrhea, responding to supportive care) that is at least possibly related to the TCR R11P3D3_KE T cells, Grade 2 or higher bronchospasm requiring discontinuation of T-cell infusion, Grade 2 or higher hypersensitivity reactions related to treatment with TCR R11P3D3_KE T cells, Grade 2 or higher autoimmune reaction (CRS is not considered an autoimmune reaction for the purposes of severe toxicity), any AE that leads to a discontinuation of T-cell infusion, CRS, tumor lysis syndrome, T-cell-related encephalopathy syndrome, suspected off-target toxicities related to T-cell infusion and/or target-independent T-cell toxicities, hematological abnormalities lasting more than about 28 days, suspected cardiac toxicities, or combinations thereof. However, even where a patient(s) experienced severe toxicity, a second or additional infusions may be administered if, in the opinion of a clinician(s), such administration(s) is in the best interest of a patient(s).

A second administration, or additional administration, of TCR R11 P3D3_KE T cells may be at any dose level, including, but not limited to, dose levels higher or lower than a first-or additionally administered dose. Prior a second infusion, or additional infusion, of TCR R11P3D3_KE T cells, a lymphodepletion (LD) may be performed, including, but not limited to, administration of the same or different drug(s) at dose levels approximately the same as, higher than, or lower than a performance of a first or additional LD. A second or additional infusion may be administered, as non-limiting example, at least about 2 months, at least about 3 months, or at least about 4 months have passed since the first day of the previous LD.

### Example 27

### General Treatment Procedure - MAGE-A4-Binding Molecules

A patient(s) identified using the selection procedure for MAGE-A4 described in Example 19 or Example 20 is selected for treatment. A patient(s) having MAGE-A4⁺ tumor(s) is selected for treatment. Patient(s) is treated with a MAGE-A4-binding molecule. Treatment, including any pre-treatment, may be carried out in accordance with appropriate art-known techniques and/or according to manufacturer guidelines for the applicable product. Patient(s) may be tested or monitored, measured, or otherwise assessed as set forth in Example 26.

### Example 28

### General Treatment Procedure - Genetically Engineered Autologous T Cells Specific For HLA-A2-Restricted MAGE-A4230-239 Peptide GVYDGREHTV (SEQ ID NO: 401) Expressed in the Context of HLA-A*02

A patient(s) identified using the selection procedure described in Example 20 was selected for treatment. A patient(s) having MAGE-A4⁺ tumor(s) is selected for treatment. A patient(s) was treated with genetically engineered autologous T Cells specific for HLA-A2-restricted MAGE-A4230-239 peptide GVYDGREHTV (SEQ ID NO: 401) expressed in the context of HLA-A*02. (See Example 25). For this treatment , patients positive for HLA-A*02:05 in either allele; having HLA-A*02 alleles having the same protein sequence as HLA-A*02:05 in the peptide binding domains (P groups); positive for HLA-A*02:07 in either allele; or having HLA-A*02 alleles having the same protein sequence as HLA-A*02:07 in the peptide binding domains (P groups) may be excluded from treatment.

A patient(s) undergoes leukapheresis to obtain autologous T cells for transduction with the construct, as described in Example 25. Autologous T cells are transduced with the described construct to produce a MAGE-A4-binding molecule construct T cells, as described in Example 25.

Baseline tumor images may be obtained for a patient(s). lmages may be taken using, as non-limiting examples, using CT scanning, MRI scanning, PET scanning, x-ray imaging, or ultrasound. Baseline information from blood sample(s), tissue sample(s), urine sample(s), stool or gut sample(s), or other samples may be obtained. Information obtained may include, but is not limited to, information set forth below in this example.

A patient may undergo lymphodepletion, as described in Example 23 prior to infusion with the described T cells. Lymphodepletion may be performed, for example, daily for 4 consecutive days (Day -6 to Day -3) prior to infusion.

A patient(s) receives an intravenous (IV) infusion of autologous T cells, as described in Example 25, on Day 0. The cell dose may be based, as a non-limiting example, on the number of viable cluster of differentiation (CD)³⁺ CD⁸⁺ HLA dextramer⁺ cells (which may represent the best available correlate to the number of active, transduced T cells). As non-limiting examples, the cell dose may be total cells (cells) or the cell dose may be measured, as a non-limiting example, per body surface area (BSA) as defined by the Mosteller formula (cells/m²). Mosteller RD, Simplified calculation of body-surface area. N Engl J Med. 1987 Oct 22;317(17):1098, which is incorporated herein by reference in its entirety.

A patient(s) may receive approximately 5 × 10⁷ to approximately 20 × 10¹⁰ cells, approximately 1 × 10⁸ to approximately 10 × 10¹⁰ cells, approximately 1 × 10⁹ to approximately 5 × 10¹⁰ cells, approximately 2 × 10⁹ to approximately 1 × 10¹⁰ cells, approximately 1 × 10⁹ to approximately 9 × 10⁹ cells, approximately 3 × 10⁹ to approximately 5 × 10⁹ cells, approximately 0.5 × 10⁹ to approximately 1.2 × 10⁹ cells, approximately 1.2 × 10⁹ to approximately 6 × 10⁹ cells, approximately 4.49 to approximately 9.98 × 10⁹ cells, approximately 0.08 × 10⁹ to approximately 0.12 × 10⁹ cells, approximately 0.5 × 10⁹ to approximately 1.2 × 10⁹ cells, approximately 4.9 × 10⁹ to approximately 9.98 × 10⁹ cells, approximately 0.08 × 10⁹ cells, approximately 0.1 × 10⁹ cells, approximately 0.12 × 10⁹ cells, approximately 0.15 × 10⁹ cells, approximately 0.5 × 10⁹ cells, approximately 1 × 10⁹ cells, approximately 1.2 × 10⁹ cells, approximately 4.49 × 10⁹ cells, approximately 5 × 10⁹ cells, approximately 6 × 10⁹ cells, approximately 7 × 10⁹ cells, approximately 8 × 10⁹ cells, approximately 9 × 10⁹ cells, approximately 9.8 × 10⁹ cells, or approximately 10 × 10⁹ cells.

A patient(s) may receive approximately 1 × 10⁶ to approximately 18 × 10⁶ cells/m², approximately 12 × 10⁶ to approximately 18 × 10⁶ cells/m², approximately 40 × 10⁶ to approximately 60 × 10⁶ cells/m², approximately 120 × 10⁶ to approximately 180 × 10⁶ cells/m², approximately 240 × 10⁶ to approximately 480 × 10⁶ cells/m², approximately 200 × 10⁶ to approximately 480 × 10⁶ cells/m², approximately 200 × 10⁶ to approximately 500 × 10⁶ cells/m², approximately 200 × 10⁶ to approximately 1200 × 10⁶ cells/m², approximately 12 × 10⁶ to approximately 18 × 10⁶ cells/m², approximately 12 × 10⁶ to approximately 1200 × 10⁶ cells/m², approximately 1 × 10⁷ to approximately 14 × 10⁸ cells/m², approximately 41 × 10⁷ to approximately 2 ×10 ⁸ cells/m², approximately 1 × 10⁵ to approximately 15 × 10¹⁰ cells/m², approximately 12 × 10⁶ cells/m², approximately 15 × 10⁶ cells/m², approximately 18 × 10⁶ cells/m², approximately 40 × 10⁶ cells/m², approximately 50 × 10⁶ cells/m², approximately 60 × 10⁶ cells/m², approximately 120 × 10⁶ cells/m², approximately 150 × 10⁶ cells/m², approximately 180 × 10⁶ cells/m², approximately 200 × 10⁶ cells/m², approximately 340 × 10⁶ cells/m², approximately 480 × 10⁶ cells/m², approximately 40 × 10⁶ cells/m², approximately 600 × 10⁶ cells/m², approximately 700 × 10⁶ cells/m², approximately 1200 × 10⁶ cells/m², approximately 5.0 × 10¹⁰ or fewer cells/m², approximately 10 × 10¹⁰ or fewer cells/m², or approximately 15 × 10¹⁰ or fewer cells/m².

Intravenous infusion of a first bag of described T cells may be started at a slow rate (about 1 to about 2 mL/minute). The maximum infusion speed may be limited to approximately 5 mL/minute for any remaining bags if not further limited. An exact minimum infusion time may be calculated to ensure the endotoxin limit of < 5 EU/kg/hour is not exceeded. Infusion speed may be further reduced based on patient tolerance; however, a maximum allowable infusion time per bag may be approximately 30 minutes. Regardless of how many bags are used, this infusion may be considered to be a single dose infusion.

A patient(s) may be hospitalized for approximately 3 weeks (starting with the first day of lymphodepletion (Day -6), if lymphodepletion is performed. Patient(s) may be discharged from the hospital when clinically stable at the discretion of the clinician.

A patient(s) may receive prophylaxis for infections, as described in Example 37. A patient(s) may receive prophylaxis for allergic reactions, as described in Example 38. A patient(s) may be administered low-dose subcutaneous (SC) interleukin 2 (IL-2), as described in Example 36.

Patient(s) may additionally be monitored for approximately 2 years or approximately 3 years or more after discharge from the hospital; visits may occur approximately quarterly.

Patient(s) may be tested or monitored, measured, otherwise assessed, or combinations thereof, as set forth in Example 26. Patient(s) may administered a second or additional dose(s) of T cells as described in Example 25, using a procedure such as set forth in Example 26.

### Example 29

### General Treatment Procedure - TCR R11 P3D3_KE T Cells Following T Cells as Described in Example 25 or Other MAGE-4A-Binding Molecule

A patient(s) having received treatment with T cells as described in Example 25, as described Example 28, or other MAGE-4A-binding molecule, as described Example 27, is then treated with TCR R11 P3D3_KE T cells, as described Example 26. Combination treatment with R11 P3D3_KE T cells following treatment with T cells as described in Example 25 or other MAGE-4A-binding molecule may be administered, as non-limiting examples, where a patient(s) tumor(s) progresses after treatment with T cells as described in Example 25 or other MAGE-4A-binding molecule, where a patient(s) tumor(s) expresses PRAME after treatment with T cells as described in Example 25 or other MAGE-4A-binding molecule (PRAME may also be expressed on the tumor(s) before treatment with T cells as described in Example 25 or other MAGE-4A-binding molecule), or combinations thereof.

### Example 31

### General Treatment Procedure - T Cells as Described in Example 25 or Other MAGE-4A-Binding Molecule Following TCR R11 P3D3_KE T Cells

A patient(s) having received treatment with TCR R11P3D3_KE T cells as described Example 26 is then treated with T cells as described in Example 25, as described Example 28, or other MAGE-4A-binding molecule, as described Example 27. Combination treatment with T cells as described in Example 25 or other MAGE-4A-binding molecule following treatment with R11P3D3_KE T cells may be administered, as non-limiting examples, where a patient(s) tumor(s) progresses after treatment with R11 P3D3_KE T cells, where a patient(s) tumor(s) expresses MAGE-A4 after treatment with R11P3D3_KE T cells (MAGE-A4 may also be expressed on the tumor(s) before treatment with R11P3D3_KE T cells), or combinations thereof.

### Example 32

### General Treatment Procedure - Atezolizumab

A patient(s) having received treatment with TCR R11P3D3_KE T cells as described Example 26; with T cells as described in Example 25, as described Example 28, or other MAGE-4A-binding molecule, as described Example 27; or with both, as described in Example 29 and Example 30; or a patient(s) scheduled for treatment with TCR R11P3D3_KE T cells, with T cells as described in Example 25 or other MAGE-4A-binding molecule, or with both, is treated with atezolizumab. Atezolizumab is a PD-L1 blocking antibody.

Atezolizumab may be administered intravenously at a dose of approximately 840 mg over approximately 30 to approximately 60 minutes at approximately Day 14 (± approximately 5 days) post-treatment or approximately Day 21 (± approximately 3 days) post-treatment. Approximately two weeks after the first infusion of atezolizumab, patients may receive a dose of approximately 1680 mg atezolizumab intravenously over approximately 30 minutes to approximately 60 minutes. Thereafter patients may receive atezolizumab at a dose of approximately 1680 mg intravenously over approximately 30 minutes to approximately 60 minutes approximately every 4 weeks for up to approximately 1 year. FIG. 40, in which M indicates month after treatment and D indicates D after treatment, shows exemplary non-limiting atezolizumab dosing schedules, starting at Day 14 post-treatment or Day 21 post-treatment.

For patients who respond to atezolizumab therapy, atezolizumab may be continued for another approximately 6 months up to approximately 1 year or longer. Atezolizumab may be discontinued if, as non-limiting examples, patient(s) begins a new anti-cancer therapy, patient(s) shows disease progression, patient(s) shows unacceptable toxicity, physician feels that it is in the best interest of the patient to discontinue treatment, or combinations thereof.

Atezolizumab administration may be delayed until a patient(s) may have achieved hematologic recovery from prior lymphodepletion(s), may have achieved hematologic recovery from prior treatment(s), may have recovered from any infection(s), or combinations thereof. Hematologic recovery may, as a non-limiting example, be defied as a patient(s) having platelets > approximately 50,000 /µL, hemoglobin > approximately 8.0 g/dL, absolute neutrophil count > approximately 1,000 /µL, or combinations thereof.

### Example 33

### General Treatment Procedure - Pembrolizumab

A patient(s) having received treatment with TCR R11P3D3_KE T cells as described Example 26; with T cells as described in Example 25, as described Example 28, or other MAGE-4A-binding molecule, as described Example 27; or with both, as described in Example 29 and Example 30; or a patient(s) scheduled for treatment with TCR R11P3D3_KE T cells, with T cells as described in Example 25 or other MAGE-4A-binding molecule, or with both is treated with pembrolizumab. Pembrolizumab is a PD-L1 blocking antibody. Treatment may be carried out, as non-limiting examples, in accordance with appropriate art-known techniques and/or according to manufacturer guidelines for the applicable product.

For patients who respond to pembrolizumab therapy, pembrolizumab may be continued for another approximately 6 months up to approximately 1 year or longer. Pembrolizumab may be discontinued if, as non-limiting examples, patient(s) begins a new anti-cancer therapy, patient(s) shows disease progression, patient(s) shows unacceptable toxicity, physician feels that it is in the best interest of the patient to discontinue treatment, or combinations thereof.

Pembrolizumab administration may be delayed until a patient(s) may have achieved hematologic recovery from prior lymphodepletion(s), may have achieved hematologic recovery from prior treatment(s), may have recovered from any infection(s), or combinations thereof. Hematologic recovery may, as a non-limiting example, be defied as a patient(s) having platelets > approximately 50,000 /µL, hemoglobin > approximately 8.0 g/dL, absolute neutrophil count > approximately 1,000 /µL, or combinations thereof.

### Example 34

### General Treatment Procedure - Nivolumab

A patient(s) having received treatment with TCR R11 P3D3_KE T cells as described Example 26; with T cells as described in Example 25, as described Example 28, or other MAGE-4A-binding molecule, as described Example 27; or with both, as described in Example 29 and Example 30; or a patient(s) scheduled for treatment with TCR R11 P3D3_KE T cells, with T cells as described in Example 25 or other MAGE-4A-binding molecule, or with both is treated with nivolumab. Nivolumab is a is a PD-1 blocking antibody. Treatment may be carried out, as non-limiting examples, in accordance with appropriate art-known techniques and/or according to manufacturer guidelines for the applicable product.

For patients who respond to nivolumab therapy, nivolumab may be continued for another approximately 6 months up to approximately 1 year or longer. Nivolumab may be discontinued if, as non-limiting examples, patient(s) begins a new anti-cancer therapy, patient(s) shows disease progression, patient(s) shows unacceptable toxicity, physician feels that it is in the best interest of the patient to discontinue treatment, or combinations thereof.

Nivolumab administration may be delayed until a patient(s) may have achieved hematologic recovery from prior lymphodepletion(s), may have achieved hematologic recovery from prior treatment(s), may have recovered from any infection(s), or combinations thereof. Hematologic recovery may, as a non-limiting example, be defied as a patient(s) having platelets > approximately 50,000 /µL, hemoglobin > approximately 8.0 g/dL, absolute neutrophil count > approximately 1,000 /µL, or combinations thereof.

### Example 35

### General Treatment Procedure - Cemiplimab

A patient(s) having received treatment with TCR R11 P3D3_KE T cells as described Example 26; with T cells as described in Example 25, as described Example 28, or other MAGE-4A-binding molecule, as described Example 27; or with both, as described in Example 29 and Example 30; or a patient(s) scheduled for treatment with TCR R11 P3D3_KE T cells, with T cells as described in Example 25 or other MAGE-4A-binding molecule, or with both is treated with cemiplimab. Cemiplimab is a PD-L1 blocking antibody. Treatment may be carried out, as non-limiting examples, in accordance with appropriate art-known techniques and/or according to manufacturer guidelines for the applicable product.

For patients who respond to cemiplimab therapy, cemiplimab may be continued for another approximately 6 months up to approximately 1 year or longer. Cemiplimab may be discontinued if, as non-limiting examples, patient(s) begins a new anti-cancer therapy, patient(s) shows disease progression, patient(s) shows unacceptable toxicity, physician feels that it is in the best interest of the patient to discontinue treatment, or combinations thereof.

Cemiplimab administration may be delayed until a patient(s) may have achieved hematologic recovery from prior lymphodepletion(s), may have achieved hematologic recovery from prior treatment(s), may have recovered from any infection(s), or combinations thereof. Hematologic recovery may, as a non-limiting example, be defied as a patient(s) having platelets > approximately 50,000 /µL, hemoglobin > approximately 8.0 g/dL, absolute neutrophil count > approximately 1,000 /µL, or combinations thereof.

### Example 36

### General Treatment Procedure - Interleukin 2

A patient(s) treated with a therapy described herein or combinations thereof, may be treated with Interleukin 2 (IL-2), such as but not limited to, Aldesleukin. As a non-limiting example, IL-2 may be administered starting approximately 1 day after treatment. IL-2 may be administered subcutaneously (SC), as a non-limiting example. IL-2 may be administered starting approximately 24 hours after treatment, as a non-limiting example. A dose of 1 million IU (approximately 550,000 lU/m²) (or other low-dose) IL-2 may be administered, as non-limiting example. IL-2 may be administered approximately once daily (approximately every 24 hours) for approximately 5 days (approximately 5 doses) followed by twice daily (approximately every 12 hours) for approximately 5 days (approximately 10 doses). Other numbers of doses, such as, but not limited to, approximately 12 doses to approximately 28 doses, approximately 16 doses, approximately 20 doses, approximately 24 doses, or approximately 28 doses may be administered.

Administration of IL-2 may be paused, delayed, or discontinued, as non-limiting examples, if ≥ Grade 2 CRS is suspected, if it is decided to administer tocilizumab to counteract CRS, if ≥ Grade 2 neurotoxicity is suspected or neurotoxicity is confirmed, or combinations thereof, as non-limiting examples. If paused and resumed, or from the start, IL-2 dose may be adapted to any lower dose for safety reasons.

### Example 37

### General Treatment Procedure - Prophylaxis for Infections

A patient(s) treated with lymphodepletion, a therapy described herein, or combinations thereof, may be treated with prophylaxis for infections, such as, but not limited to bacterial, viral, fungal infections, neutropenic fever/sepsis, or combinations thereof. Prophylaxis against infections may be started, as non-limiting examples, before the start of lymphodepletion, before the start of treatment, at the start of lymhpdepletion or at the start of treatment. As non-limiting examples, any or combinations of the following may be administered: anti-bacterial (such as, but not limited to, bactrim double strength (trimethoprim approximately 160 mg and sulfamethoxazole approximately 800 mg) orally approximately 3 times per week or as medically indicated according to hospital/local guideline/recommendation, Levaquin approximately 500 mg orally daily or as medically indicated according to hospital/local guideline/recommendation, or combinations thereof) for approximately 1 month or until patient(s) has achieved hematologic recovery; Herpes zoster virus prophylaxis (with, as a non-limiting example, valacyclovir approximately 500 mg orally daily) for approximately 2 months, or as medically indicated according to hospital/local guideline/recommendation, until patient(s) has achieved hematologic recovery; antifungal (such as, but not limited to, fluconazole approximately 200 mg orally daily or as medically indicated according to hospital/local guideline/recommendation) for approximately 1 month or until patient(s) has achieved hematologic recovery.

Hematologic recovery may, as a non-limiting example, be defied as a patient(s) having platelets > approximately 50,000 /µL,hemoglobin > approximately 8.0 g/dL, absolute neutrophil count > approximately 1,000 /µL, or combinations thereof.

### Example 38

### General Treatment Procedure - Prophylaxis for Allergic Reaction

A patient(s) treated with lymphodepletion, a therapy described herein, or combinations thereof, may be treated with prophylaxis for allergic reaction(s). Prophylaxis against allergic reactions may be started, as non-limiting examples, before the start of lymphodepletion, before the start of treatment, at the start of lymhpdepletion or at the start of treatment. As non-limiting examples, any or combinations of the following may be administered: acetaminophen (paracetamol) approximately 500 mg to approximately 650 mg, diphenhydramine hydrochloride approximately 25 to approximately 50 mg orally or intravenously, or combinations thereof.

### Example 39

### Tumor Regression After Treatment With a MAGEA4-004 Program and TCR R11P3D3_KE T Cells

A patient was selected for treatment with TCR R11P3D3_KE T cells, as described in Example 18. The patient, a 49-year-old white male patient with synovial sarcoma (first diagnosed in September 2011) had previously been treated with 4 surgeries between 2012 and 2017, with radiation therapy in 2012, and with multi-targeted receptor tyrosine kinase inhibitor pazopanib (from May 2018 to September 2019).

In 2019, patient was treated with ADP-A2M4, which are engineered T cells expressing exogenous TCR binding to MAGEA-003 (KVLEYVIKV) (SEQ lD NO: 417). The patient’s tumor regressed, but later progressed. qPCR (quantitative - polymerase chain reaction) analysis on the tissue sample(s) was performed on a tumor biopsy from the indicated patient in January 2021. Briefly, RNA was extracted from patient’s sample(s) and reverse transcribed to cDNA (complementary DNA). The cDNA was used for qPCR reaction to detect cancer-specific antigens (IMADETECT® assay) using Applied Biosystems 7500 real-time PCR instrument. The results indicate that the tumor was positive for MAG-003 (MAGEA4/MAGEA8) and PRAME. The patient underwent leukapheresis for TCR R11 P3D3_KE T cell production (see Example 22) and received non myeloablative chemotherapy for lymphodepletion (FLU: 40 mg/ml² for each of 4 days and CY: 500 mg/ml² for each of 4 days) (see Example 23), then received treatment with autologous TCR R11 P3D3_KE T cells, which are engineered T cells expressing exogenous TCR binding to PRAME-004 (SLLQHLIGL) (SEQ ID NO: 310) in April 2021 (Day 0). The patient was infused with 0.41×10⁹ transduced autologous T cells (total CD3⁺ CD8⁺ HLA dextramer⁺) on Day 0. Patient also received subcutaneous injections of low dose IL-2 post-T cell infusion, starting 6 hours after administration of TCR R11 P3D3_KE T cells and repeated every 12 hours, for a total of 16 doses. Each dose was 1 million IU.

TABLE 9

| Tumor assessment | Assessment date | Sum of longest diameter (mm) | Relative change from baseline (%) | Disease Response (RECIST1.1) |
|---|---|---|---|---|
| Baseline | Day -11 | 84.0 | 0.0 | NA |
| Tumor Asmt -(1) | 6 weeks and one day | 55.8 | -33.6 | PR |
| Tumor Asmt -(2) | 14 weeks | 55.6 | -33.8 | PR |
| Tumor Asmt -(3) | 26 weeks and one day | 47.7 | -43.2 | PR |

Patient’s tumor was imaged using CT scanning on Day -11 (11 days prior to infusion with TCR R11P3D3_KE T cells) (baseline denotes pre-TCR R11P3D3_KE T cells treatment), at 6 weeks and one day after treatment, at 14 weeks after treatment, and at 26 weeks and one day after treatment. Results of imaging are tabulated in Table 9. Baseline images and images taken at 14 weeks after treatment are set forth in FIGS. 41A, 41B, and 41C. Baseline and 14-week images of three target lesions are shown in FIGS. 41A, 41B, and 41C.. FIG. 41A shows a baseline tumor measurement of 14.0 ×28.1 mm and a post-treatment tumor measurement of 1.6 ×9.2 mm. FIG. 41B shows a baseline tumor measurement of 11.2 × 26.2 mm and a post-treatment tumor measurement of 12.3 × 24.0 mm. FIG. 41C shows a baseline tumor measurement of 26.1 × 29.7 mm and a post-treatment tumor measurement of 9.1 × 22.4 mm.

At each post-TCR R11P3D3_KE T cells-treatment imaging, patient’s tumor showed a Partial Response (PR) using the RECIST version 1.1 guidelines.

### Example 40

Representative T AA’s that may be targeted in a pre-treatment, first treatment, second or successive treatment are described below in Table 10. TAA that are capable of being recognized by antigen binding molecules described herein may include at least one amino acid sequence of SEQ ID NO: 313 to SEQ ID NO: 474. (Table 10). Engineered T cells can selectively recognize cells which present a TAA peptide described in the amino acid sequences of SEQ ID NO: 313-474 or any of the patents or applications described herein, for example, those TAA peptides described in U.S. Pat. Application Publication Nos. 2016/0187351; 2017/0165335; 2017/0035807; 2016/0280759; 2016/0287687; 2016/0346371; 2016/0368965; 2017/0022251; 2017/0002055; 2017/0029486; 2017/0037089; 2017/0136108; 2017/0101473; 2017/0096461; 2017/0165337; 2017/0189505; 2017/0173132; 2017/0296640; 2017/0253633; 2017/0260249; 2018/0051080, and 2018/0164315.

TABLE 10

Exemplary List of Tumor Associated Antigens (TAAs)

| SEQ ID NO: | Amino Acid Sequence | SEQ ID NO: | Amino Acid Sequence | SEQ ID NO: | Amino Acid Sequence |
|---|---|---|---|---|---|
| 313 | YLYDSETKNA | 366 | LLWGHPRVALA | 418 | VLLNEILEQV |
| 314 | HLMDQPLSV | 367 | VLDGKVAVV | 419 | SLLNQPKAV |
| 315 | GLLKKINSV | 368 | GLLGKVTSV | 420 | KMSELQTYV |
| 316 | FLVDGSSAL | 369 | KMISAIPTL | 421 | ALLEQTGDMSL |
| 317 | FLFDGSANLV | 370 | GLLETTGLLAT | 422 | VllKGLEElTV |
| 318 | FLYKIIDEL | 371 | TLNTLDINL | 423 | KQFEGTVEI |
| 319 | FILDSAETTTL | 372 | VIIKGLEEI | 424 | KLQEElPVL |
| 320 | SVDVSPPKV | 373 | YLEDGFAYV | 425 | GLAEFQENV |
| 321 | VADKIHSV | 374 | KIWEELSVLEV | 426 | NVAEIVIHI |
| 322 | IVDDLTINL | 375 | LLlPFTlFM | 427 | ALAGIVTNV |
| 323 | GLLEELVTV | 376 | ISLDEVAVSL | 428 | NLLIDDKGTIKL |
| 324 | TLDGAAVNQV | 377 | KISDFGLATV | 429 | VLMQDSRLYL |
| 325 | SVLEKEIYSI | 378 | KLIGNIHGNEV | 430 | KVLEHWRV |
| 326 | LLDPKTIFL | 379 | ILLSVLHQL | 431 | LLWGNLPEI |
| 327 | YTFSGDVQL | 380 | LDSEALLTL | 432 | SLMEKNQSL |
| 328 | YLMDDFSSL | 381 | VLQENSSDYQSNL | 433 | KLLAVIHEL |
| 329 | KVWSDVTPL | 382 | HLLGEGAFAQV | 434 | ALGDKFLLRV |
| 330 | LLWGHPRVALA | 383 | SLVENIHVL | 435 | FLMKNSDLYGA |
| 331 | KlWEELSVlEV | 384 | YTFSGDVQL | 436 | KLIDHQGLYL |
| 332 | LLlPFTlFM | 385 | SLSEKSPEV | 437 | GPGIFPPPPPQP |
| 333 | FLIENLLAA | 386 | AMFPDTIPRV | 438 | ALNESLVEC |
| 334 | LLWGHPRVALA | 387 | FLIENLLAA | 439 | GLAALAVHL |
| 335 | FLLEREQLL | 388 | FTAEFLEKV | 440 | LLLEAVWHL |
| 336 | SLAETIFIV | 389 | ALYGNVQQV | 441 | SIIEYLPTL |
| 337 | TLLEGISRA | 390 | LFQSRIAGV | 442 | TLHDQVHLL |
| 338 | ILQDGQFLV | 391 | ILAEEPIYIRV | 443 | SLLMWITQC |
| 339 | VIFEGEPMYL | 392 | FLLEREQLL | 444 | FLLDKPQDLSI |
| 340 | SLFESLEYL | 393 | LLLPLELSLA | 445 | YLLDMPLWYL |
| 341 | SLLNQPKAV | 394 | SLAETIFIV | 446 | GLLDCPl FL |
| 342 | GLAEFQENV | 395 | AlLNVDEKNQV | 447 | VLIEYNFSI |
| 343 | KLLAVIHEL | 396 | RLFEEVLGV | 448 | TLYNPERTITV |
| 344 | TLHDQVHLL | 397 | YLDEVAFML | 449 | AVPPPPSSV |
| 345 | TLYNPERTITV | 398 | KLIDEDEPLFL | 450 | KLQEELNKV |
| 346 | KLQEKIQEL | 399 | KLFEKSTGL | 451 | KLMDPGSLPPL |
| 347 | SVLEKEIYSI | 400 | SLLEVNEASSV | 452 | ALIVSLPYL |
| 348 | RVlDDSLVV/GV | 401 | GVYDGREHTV | 453 | FLLDGSANV |
| 349 | VLFGELPAL | 402 | GLYPVTLVGV | 454 | ALDPSGNQLI |
| 350 | GLVDIMVHL | 403 | ALLSSVAEA | 455 | ILIKHLVKV |
| 351 | FLNAIETAL | 404 | TLLEGISRA | 456 | VLLDTILQL |
| 352 | ALLQALMEL | 405 | SLIEESEEL | 457 | HLIAEIHTA |
| 353 | ALSSSQAEV | 406 | ALYVQAPTV | 458 | SMNGGVFAV |
| 354 | SLITGQDLLSV | 407 | KLIYKDLVSV | 459 | MLAEKLLQA |
| 355 | QLIEKNWLL | 408 | ILQDGQFLV | 460 | YMLDIFHEV |
| 356 | LLDPKTIFL | 409 | SLLDYEVSI | 461 | ALWLPTDSATV |
| 357 | RLHDENILL | 410 | LLGDSSFFL | 462 | GLASRILDA |
| 358 | YTFSGDVQL | 411 | VIFEGEPMYL | 463 | ALSVLRLAL |
| 359 | GLPSATTTV | 412 | ALSYILPYL | 464 | SYVKVLHHL |
| 360 | GLLPSAESIKL | 413 | FLFVDPELV | 465 | VYLPKl PSW |
| 361 | KTASINQNV | 414 | SEWGSPHAAVP | 466 | NYEDHFPLL |
| 362 | SLLQHLIGL | 415 | ALSELERVL | 467 | VYIAELEKI |
| 363 | YLMDDFSSL | 416 | SLFESLEYL | 468 | VHFEDTGKTLLF |
| 364 | LMYPYIYHV | 417 | KVLEYVl KV | 469 | VLSPFILTL |
| 365 | KVWSDVTPL | | | 470 | HLLEGSVGV |
| | | | | 471 | ALREEEEGV |
| | | | | 472 | KEADPTGHSY |
| | | | | 473 | TLDEKVAEL |
| | | | | 474 | KIQEILTQV |

### Example 41

FIG. 42 shows the relative change in diameter of target lesion upon IMA203 treatment over time. The patient shows a durable response with an ongoing progression-free survival of more than 16 month and a duration of response of more than 15 months.

1. A method of treating a patient who has recurrent cancer that presents a PRAME peptide, comprising
administering to the patient a treatment composition comprising an antigen binding molecule that binds to a PRAME peptide,
wherein the patient has received at least one prior treatment with a pretreatment composition comprising an antigen binding molecule that binds to a second peptide different from the PRAME peptide,
and wherein the PRAME peptide optionally comprises SLLQHLIGL (SEQ ID NO: 310).

2. The method of claim 1, wherein the second peptide is selected from Table 10.

3. The method of claim 1, wherein the treatment composition comprising an antigen binding molecule that binds to a PRAME peptide comprises a T cell receptor (TCR) and/or an antibody.

4. The method of claim 3, wherein the TCR comprises
a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
wherein the T-cell receptor is capable of binding to a peptide consisting of the amino acid sequence of SLLQHLIGL (SEQ ID NO: 310) in a complex with HLA-A*02.

5. The method of claim 3, wherein the TCR comprises
an α chain variable domain comprising SEQ ID NO: 15, and a β chain variable domain comprising SEQ ID NO: 21, or
an α chain variable domain comprising SEQ ID NO: 27, and a β chain variable domain comprising SEQ ID NO: 33, or
an α chain variable domain comprising SEQ ID NO: 39, and a β chain variable domain comprising SEQ ID NO: 45, or
an α chain variable domain comprising SEQ ID NO: 51, and a β chain variable domain comprising SEQ ID NO: 57, or
an α chain variable domain comprising SEQ ID NO: 63, and a β chain variable domain comprising SEQ ID NO: 69, or
an α chain variable domain comprising SEQ ID NO: 75, and a β chain variable domain comprising SEQ ID NO: 81, or
an α chain variable domain comprising SEQ ID NO: 87, and a β chain variable domain comprising SEQ ID NO: 93, or
an α chain variable domain comprising SEQ ID NO: 111, and a β chain variable domain comprising SEQ ID NO: 117,
wherein the T-cell receptor is capable of binding to a peptide consisting of the amino acid sequence of SLLQHLIGL (SEQ ID NO: 310) in a complex with HLA-A*02.

6-15. (canceled)

16. The method of claim 1, wherein the recurrent cancer is selected from the group consisting of adrenocortical carcinoma, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, uterine corpus endometrial carcinoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin’s lymphoma, glioblastoma, cervical carcinoma, cervical squamous cell carcinoma and endocervical adenocarcinoma, cholangiocarcinoma, hepatocellular carcinoma, liver hepatocellular carcinoma, Ewing’s sarcoma, endometrial cancer, epithelial cancer of the larynx, esophageal carcinoma, oral carcinoma, atypical meningioma, papillary thyroid carcinoma, thymoma, brain tumors, salivary duct carcinoma, extranodal T/NK-cell lymphomas, rectal cancer, mouth and throat cancer, and multiple myeloma.

17-18. (canceled)

19. The method of claim 1, wherein the treatment composition comprises an antigen binding molecule specific for SLLQHLIGL (SEQ ID NO: 310) and wherein the one or more pretreatment compositions comprises a second antigen binding molecule specific for one or more of MAG-003, MAGEA1-003 peptide, and/or COL6A3-002.

20. The method of claim 19, wherein the second antigen binding molecule is a monoclonal antibody or a TCR.

21. The method claim 20, wherein the second antigen binding molecule binds to a peptide comprising KVLEHWRV (SEQ ID NO: 430), KVLEYVIKV (SEQ ID NO: 417), or FLLDGSANV (SEQ ID NO: 453).

22-26. (canceled)

27. A method of treating a patient who has a recurrent cancer, comprising
administering to the patient a treatment composition comprising an antigen binding molecule that binds to a peptide other than PRAME,
wherein the patient has received one or more prior treatments with a pretreatment composition comprising an antigen binding molecule that binds to a PRAME peptide on the cell surface,
wherein the PRAME peptide optionally comprises SLLQHLIGL (SEQ ID NO: 310).

28. The method of claim 27, wherein the treatment composition comprising an antigen binding molecule that binds to a peptide other than PRAME is specific for a peptide selected from Table 10.

29. The method of claim 27, wherein the antigen binding molecule that binds to a PRAME peptide comprises a T cell receptor (TCR) and/or an antibody.

30. The method of claim 29, wherein the TCR comprises
a CDR1α chain comprising the amino acid sequence of SEQ ID NO: 12, a CDR2α chain comprising the amino acid sequence of SEQ ID NO: 13, a CDR3α chain comprising the amino acid sequence of SEQ ID NO: 14, a CDR1β chain comprising the amino acid sequences of SEQ ID NO: 18, a CDR2β chain comprising the amino acid sequence of SEQ ID NO: 19, and a CDR3β chain comprising the amino acid sequence of SEQ ID NO: 20, or
a CDR1α chain comprising the amino acid sequence of SEQ ID NO: 24, a CDR2α chain comprising the amino acid sequence of SEQ ID NO: 25, a CDR3α chain comprising the amino acid sequence of SEQ ID NO: 26, a CDR1β chain comprising the amino acid sequences of SEQ ID NO: 30, a CDR2β chain comprising the amino acid sequence of SEQ ID NO: 31, and a CDR3β chain comprising the amino acid sequence of SEQ ID NO: 32, or
a CDR1α chain comprising the amino acid sequence of SEQ ID NO: 36, a CDR2α chain comprising the amino acid sequence of SEQ ID NO: 37, a CDR3α chain comprising the amino acid sequence of SEQ ID NO: 38, a CDR1β chain comprising the amino acid sequences of SEQ ID NO: 42, a CDR2β chain comprising the amino acid sequence of SEQ ID NO: 43, and a CDR3β chain comprising the amino acid sequence of SEQ ID NO: 44, or
a CDR1α chain comprising the amino acid sequence of SEQ ID NO: 48, a CDR2α chain comprising the amino acid sequence of SEQ ID NO: 49, a CDR3α chain comprising the amino acid sequence of SEQ ID NO: 50, a CDR1β chain comprising the amino acid sequences of SEQ ID NO: 54, a CDR2β chain comprising the amino acid sequence of SEQ ID NO: 55, and a CDR3β chain comprising the amino acid sequence of SEQ ID NO: 56,
a CDR1α chain comprising the amino acid sequence of SEQ ID NO: 60, a CDR2α chain comprising the amino acid sequence of SEQ ID NO: 61, a CDR3α chain comprising the amino acid sequence of SEQ ID NO: 62, a CDR1β chain comprising the amino acid sequences of SEQ ID NO: 66, a CDR2β chain comprising the amino acid sequence of SEQ ID NO: 67, and a CDR3β chain comprising the amino acid sequence of SEQ ID NO: 68,
a CDR1α chain comprising the amino acid sequence of SEQ ID NO: 72, a CDR2α chain comprising the amino acid sequence of SEQ ID NO: 73, a CDR3α chain comprising the amino acid sequence of SEQ ID NO: 74, a CDR1β chain comprising the amino acid sequences of SEQ ID NO: 78, a CDR2β chain comprising the amino acid sequence of SEQ ID NO: 79, and a CDR3β chain comprising the amino acid sequence of SEQ ID NO: 80
a CDR1α chain comprising the amino acid sequence of SEQ ID NO: 84, a CDR2α chain comprising the amino acid sequence of SEQ ID NO: 85, a CDR3α chain comprising the amino acid sequence of SEQ ID NO: 86, a CDR1β chain comprising the amino acid sequences of SEQ ID NO: 90, a CDR2β chain comprising the amino acid sequence of SEQ ID NO: 91, and a CDR3β chain comprising the amino acid sequence of SEQ ID NO: 92,
wherein the T-cell receptor is capable of binding to a peptide consisting of the amino acid sequence of SLLQHLIGL (SEQ ID NO: 310) in a complex with HLA-A*02.

31-41. (canceled)

42. The method of claim 27, wherein the recurrent cancer is selected from the group consisting of adrenocortical carcinoma, non-small cell lung cancer, non-small cell lung adenocarcinoma, non-small cell lung squamous cell carcinoma, small cell lung cancer, melanoma, skin cutaneous melanoma, uveal melanoma, mesothelioma, breast cancer, breast carcinoma, triple-negative breast cancer, primary brain cancer, ovarian cancer, ovarian serous cystadenocarcinoma, uterine carcinoma, uterine carcinosarcoma, uterine corpus endometrial carcinoma, head and neck squamous cell carcinomas, head and neck adenocarcinoma, colon cancer, gastro-intestinal cancer, stomach adenocarcinoma, renal cell carcinoma, kidney renal clear cell carcinoma, kidney renal papillary cell carcinoma, sarcoma, fibrosarcoma, liposarcoma, malignant peripheral nerve sheath tumors, synovial sarcoma, germ cell tumor, lymphoma, testicular cancer, testicular germ cell tumors, bladder cancers, bladder urothelial carcinoma, prostate cancer, oral cavity carcinomas, oral squamous carcinoma, acute myeloid leukemia, H. pylori-induced MALT Non-Hodgkin’s lymphoma, glioblastoma, cervical carcinoma, cervical squamous cell carcinoma and endocervical adenocarcinoma, cholangiocarcinoma, hepatocellular carcinoma, liver hepatocellular carcinoma, Ewing’s sarcoma, endometrial cancer, epithelial cancer of the larynx, esophageal carcinoma, oral carcinoma, atypical meningioma, papillary thyroid carcinoma, thymoma, brain tumors, salivary duct carcinoma, extranodal T/NK-cell lymphomas, rectal cancer, mouth and throat cancer, and multiple myeloma.

43-44. (canceled)

45. The method of claim 27, wherein the treatment composition comprises an antigen binding molecule specific for one or more of MAG-003, MAGEA1-003 peptide, and/or COL6A3-002 and the pretreatment composition comprises an antigen binding molecule specific for SLLQHLIGL (SEQ ID NO: 310).

46. The method of claim 45, wherein the treatment or pre-treatment compositions a monoclonal antibody or a TCR.

47. The method of claim 46, wherein the treatment composition comprises an antigen binding molecule that binds to KVLEHVVRV (SEQ ID NO: 430), KVLEYVIKV (SEQ ID NO: 417), or FLLDGSANV (SEQ ID NO: 453) and the pretreatment composition comprises an antigen binding molecule that binds to GVYDGREHTV peptide (SEQ ID NO: 401).

48-52. (canceled)

53. A method of eliciting an immune response in a patient who has a recurrent cancer that presents a PRAME peptide, comprising
administering to the patient a treatment composition comprising an antigen binding molecule that binds to a PRAME peptide,
wherein the patient has received a prior treatment with one or more pretreatment compositions comprising a second antigen binding molecule that binds a second peptide, and
wherein the PRAME peptide optionally comprises SLLQHLIGL (SEQ ID NO: 310).

54. The method of claim 53, wherein the second peptide is selected from Table 10.

55. The method of claim 53, wherein the antigen binding molecule that binds to a PRAME peptide comprises a T cell receptor (TCR) and/or an antibody.

56-75. (canceled)


SEQUENCE LISTING (496 sequences)

SEQ ID NO: 1 - protein, 235 aa, synthetic construct
  REGION 1..235: note = antibody or TCR sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 235 aa, synthetic construct
  REGION 1..235: note = antibody or TCR sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - protein, 226 aa, synthetic construct
  REGION 1..226: note = antibody or TCR sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 4 - protein, 226 aa, synthetic construct
  REGION 1..226: note = antibody or TCR sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 5 - protein, 243 aa, synthetic construct
  REGION 1..243: note = antibody or TCR sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 6 - protein, 198 aa, synthetic construct
  REGION 1..198: note = antibody or TCR sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 7 - protein, 189 aa, synthetic construct
  REGION 1..189: note = antibody or TCR sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 8 - protein, 200 aa, synthetic construct
  REGION 1..200: note = antibody or TCR sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 9 - protein, 225 aa, synthetic construct
  REGION 1..225: note = antibody or TCR sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 10 - protein, 225 aa, synthetic construct; identical to SEQ ID NO: 9
  REGION 1..225: note = antibody or TCR sequence

SEQ ID NO: 11 - protein, 222 aa, synthetic construct
  REGION 1..222: note = antibody or TCR sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 12 - protein, 6 aa, synthetic construct
  REGION 1..6: note = antibody or TCR sequence
SSNFYA

SEQ ID NO: 13 - skipped

SEQ ID NO: 14 - protein, 11 aa, synthetic construct
  REGION 1..11: note = antibody or TCR sequence
CALYNNNDMRF

SEQ ID NO: 15 - protein, 132 aa, synthetic construct
  REGION 1..132: note = antibody or TCR sequence
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKP

SEQ ID NO: 16 - protein, 141 aa, synthetic construct
  REGION 1..141: note = antibody or TCR sequence
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 273 aa, synthetic construct
  REGION 1..273: note = antibody or TCR sequence
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 5 aa, synthetic construct
  REGION 1..5: note = antibody or TCR sequence
SGHNS

SEQ ID NO: 19 - protein, 6 aa, synthetic construct
  REGION 1..6: note = antibody or TCR sequence
FNNNVP

SEQ ID NO: 20 - protein, 13 aa, synthetic construct
  REGION 1..13: note = antibody or TCR sequence
CASSPGSTDTQYF

SEQ ID NO: 21 - protein, 132 aa, synthetic construct
  REGION 1..132: note = antibody or TCR sequence
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVL

SEQ ID NO: 22 - protein, 179 aa, synthetic construct
  REGION 1..179: note = antibody or TCR sequence
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 311 aa, synthetic construct
  REGION 1..311: note = antibody or TCR sequence
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 6 aa, synthetic construct
  REGION 1..6: note = antibody or TCR sequence
DRGSQS

SEQ ID NO: 25 - skipped

SEQ ID NO: 26 - protein, 15 aa, synthetic construct
  REGION 1..15: note = antibody or TCR sequence
CAAVISNFGNEKLTF

SEQ ID NO: 27 - protein, 134 aa, synthetic construct
  REGION 1..134: note = antibody or TCR sequence
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIP

SEQ ID NO: 28 - protein, 141 aa, synthetic construct; identical to SEQ ID NO: 16
  REGION 1..141: note = antibody or TCR sequence

SEQ ID NO: 29 - protein, 275 aa, synthetic construct
  REGION 1..275: note = antibody or TCR sequence
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 5 aa, synthetic construct
  REGION 1..5: note = antibody or TCR sequence
SGHRS

SEQ ID NO: 31 - protein, 6 aa, synthetic construct
  REGION 1..6: note = antibody or TCR sequence
YFSETQ

SEQ ID NO: 32 - protein, 14 aa, synthetic construct
  REGION 1..14: note = antibody or TCR sequence
CASSPWDSPNEQYF

SEQ ID NO: 33 - protein, 132 aa, synthetic construct
  REGION 1..132: note = antibody or TCR sequence
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVT

SEQ ID NO: 34 - protein, 179 aa, synthetic construct; identical to SEQ ID NO: 22
  REGION 1..179: note = antibody or TCR sequence

SEQ ID NO: 35 - protein, 311 aa, synthetic construct
  REGION 1..311: note = antibody or TCR sequence
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - protein, 6 aa, synthetic construct
  REGION 1..6: note = antibody or TCR sequence
NSAFQY

SEQ ID NO: 37 - skipped

SEQ ID NO: 38 - protein, 13 aa, synthetic construct
  REGION 1..13: note = antibody or TCR sequence
CAMSEAAGNKLTF

SEQ ID NO: 39 - protein, 133 aa, synthetic construct
  REGION 1..133: note = antibody or TCR sequence
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKP

SEQ ID NO: 40 - protein, 141 aa, synthetic construct; identical to SEQ ID NO: 16
  REGION 1..141: note = antibody or TCR sequence

SEQ ID NO: 41 - protein, 274 aa, synthetic construct
  REGION 1..274: note = antibody or TCR sequence
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 5 aa, synthetic construct
  REGION 1..5: note = antibody or TCR sequence
SGHAT

SEQ ID NO: 43 - protein, 6 aa, synthetic construct
  REGION 1..6: note = antibody or TCR sequence
FQNNGV

SEQ ID NO: 44 - protein, 13 aa, synthetic construct
  REGION 1..13: note = antibody or TCR sequence
CASSYTNQGEAFF

SEQ ID NO: 45 - protein, 132 aa, synthetic construct
  REGION 1..132: note = antibody or TCR sequence
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVV

SEQ ID NO: 46 - protein, 177 aa, synthetic construct
  REGION 1..177: note = antibody or TCR sequence
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 309 aa, synthetic construct
  REGION 1..309: note = antibody or TCR sequence
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 48 - protein, 6 aa, synthetic construct
  REGION 1..6: note = antibody or TCR sequence
DRGSQS

SEQ ID NO: 49 - skipped

SEQ ID NO: 50 - protein, 13 aa, synthetic construct
  REGION 1..13: note = antibody or TCR sequence
CAVLNQAGTALIF

SEQ ID NO: 51 - protein, 132 aa, synthetic construct
  REGION 1..132: note = antibody or TCR sequence
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSS

SEQ ID NO: 52 - protein, 141 aa, synthetic construct; identical to SEQ ID NO: 16
  REGION 1..141: note = antibody or TCR sequence

SEQ ID NO: 53 - protein, 273 aa, synthetic construct
  REGION 1..273: note = antibody or TCR sequence
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 5 aa, synthetic construct
  REGION 1..5: note = antibody or TCR sequence
SGDLS

SEQ ID NO: 55 - protein, 6 aa, synthetic construct
  REGION 1..6: note = antibody or TCR sequence
YYNGEE

SEQ ID NO: 56 - protein, 17 aa, synthetic construct
  REGION 1..17: note = antibody or TCR sequence
CASSAETGPWLGNEQFF

SEQ ID NO: 57 - protein, 135 aa, synthetic construct
  REGION 1..135: note = antibody or TCR sequence
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVL

SEQ ID NO: 58 - protein, 179 aa, synthetic construct; identical to SEQ ID NO: 22
  REGION 1..179: note = antibody or TCR sequence

SEQ ID NO: 59 - protein, 314 aa, synthetic construct
  REGION 1..314: note = antibody or TCR sequence
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 60 - protein, 7 aa, synthetic construct
  REGION 1..7: note = antibody or TCR sequence
TSESDYY

SEQ ID NO: 61 - protein, 4 aa, synthetic construct
  REGION 1..4: note = antibody or TCR sequence
QEAY

SEQ ID NO: 62 - protein, 15 aa, synthetic construct
  REGION 1..15: note = antibody or TCR sequence
CAYRWAQGGSEKLVF

SEQ ID NO: 63 - protein, 136 aa, synthetic construct
  REGION 1..136: note = antibody or TCR sequence
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNP

SEQ ID NO: 64 - protein, 141 aa, synthetic construct
  REGION 1..141: note = antibody or TCR sequence
YIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - protein, 277 aa, synthetic construct
  REGION 1..277: note = antibody or TCR sequence
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 5 aa, synthetic construct
  REGION 1..5: note = antibody or TCR sequence
MGHDK

SEQ ID NO: 67 - protein, 6 aa, synthetic construct
  REGION 1..6: note = antibody or TCR sequence
SYGVNS

SEQ ID NO: 68 - protein, 16 aa, synthetic construct
  REGION 1..16: note = antibody or TCR sequence
CATELWSSGGTGELFF

SEQ ID NO: 69 - protein, 134 aa, synthetic construct
  REGION 1..134: note = antibody or TCR sequence
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVL

SEQ ID NO: 70 - protein, 179 aa, synthetic construct; identical to SEQ ID NO: 22
  REGION 1..179: note = antibody or TCR sequence

SEQ ID NO: 71 - protein, 313 aa, synthetic construct
  REGION 1..313: note = antibody or TCR sequence
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 6 aa, synthetic construct
  REGION 1..6: note = antibody or TCR sequence
DRGSQS

SEQ ID NO: 73 - skipped

SEQ ID NO: 74 - protein, 13 aa, synthetic construct
  REGION 1..13: note = antibody or TCR sequence
CAVGPSGTYKYIF

SEQ ID NO: 75 - protein, 132 aa, synthetic construct
  REGION 1..132: note = antibody or TCR sequence
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLA

SEQ ID NO: 76 - protein, 141 aa, synthetic construct; identical to SEQ ID NO: 16
  REGION 1..141: note = antibody or TCR sequence

SEQ ID NO: 77 - protein, 273 aa, synthetic construct
  REGION 1..273: note = antibody or TCR sequence
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 5 aa, synthetic construct
  REGION 1..5: note = antibody or TCR sequence
MNHEY

SEQ ID NO: 79 - protein, 6 aa, synthetic construct
  REGION 1..6: note = antibody or TCR sequence
SMNVEV

SEQ ID NO: 80 - protein, 14 aa, synthetic construct
  REGION 1..14: note = antibody or TCR sequence
CASSPGGSGNEQFF

SEQ ID NO: 81 - protein, 132 aa, synthetic construct
  REGION 1..132: note = antibody or TCR sequence
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVL

SEQ ID NO: 82 - protein, 179 aa, synthetic construct; identical to SEQ ID NO: 22
  REGION 1..179: note = antibody or TCR sequence

SEQ ID NO: 83 - protein, 311 aa, synthetic construct
  REGION 1..311: note = antibody or TCR sequence
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - protein, 6 aa, synthetic construct
  REGION 1..6: note = antibody or TCR sequence
DRGSQS

SEQ ID NO: 85 - skipped

SEQ ID NO: 86 - protein, 14 aa, synthetic construct
  REGION 1..14: note = antibody or TCR sequence
CAVVSGGGADGLTF

SEQ ID NO: 87 - protein, 133 aa, synthetic construct
  REGION 1..133: note = antibody or TCR sequence
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQP

SEQ ID NO: 88 - protein, 141 aa, synthetic construct; identical to SEQ ID NO: 64
  REGION 1..141: note = antibody or TCR sequence

SEQ ID NO: 89 - protein, 274 aa, synthetic construct
  REGION 1..274: note = antibody or TCR sequence
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 5 aa, synthetic construct
  REGION 1..5: note = antibody or TCR sequence
PRHDT

SEQ ID NO: 91 - protein, 6 aa, synthetic construct
  REGION 1..6: note = antibody or TCR sequence
FYEKMQ

SEQ ID NO: 92 - protein, 14 aa, synthetic construct
  REGION 1..14: note = antibody or TCR sequence
CASSLGRGGQPQHF

SEQ ID NO: 93 - protein, 142 aa, synthetic construct
  REGION 1..142: note = antibody or TCR sequence
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSIL

SEQ ID NO: 94 - protein, 177 aa, synthetic construct; identical to SEQ ID NO: 46
  REGION 1..177: note = antibody or TCR sequence

SEQ ID NO: 95 - protein, 319 aa, synthetic construct
  REGION 1..319: note = antibody or TCR sequence
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 96 - protein, 6 aa, synthetic construct
  REGION 1..6: note = antibody or TCR sequence
DSAIYN

SEQ ID NO: 97 - skipped

SEQ ID NO: 98 - protein, 15 aa, synthetic construct
  REGION 1..15: note = antibody or TCR sequence
CAVRPTSGGSYIPTF

SEQ ID NO: 99 - protein, 133 aa, synthetic construct
  REGION 1..133: note = antibody or TCR sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 100 - protein, 141 aa, synthetic construct
  REGION 1..141: note = antibody or TCR sequence
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 101 - protein, 274 aa, synthetic construct
  REGION 1..274: note = antibody or TCR sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 5 aa, synthetic construct
  REGION 1..5: note = antibody or TCR sequence
MNHEY

SEQ ID NO: 103 - protein, 6 aa, synthetic construct
  REGION 1..6: note = antibody or TCR sequence
SVGAGI

SEQ ID NO: 104 - protein, 14 aa, synthetic construct
  REGION 1..14: note = antibody or TCR sequence
CASSYVGNTGELFF

SEQ ID NO: 105 - protein, 132 aa, synthetic construct
  REGION 1..132: note = antibody or TCR sequence
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 106 - protein, 179 aa, synthetic construct; identical to SEQ ID NO: 22
  REGION 1..179: note = antibody or TCR sequence

SEQ ID NO: 107 - protein, 311 aa, synthetic construct
  REGION 1..311: note = antibody or TCR sequence
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 108 - protein, 6 aa, synthetic construct
  REGION 1..6: note = antibody or TCR sequence
SSNFYA

SEQ ID NO: 109 - skipped

SEQ ID NO: 110 - protein, 11 aa, synthetic construct
  REGION 1..11: note = antibody or TCR sequence
CALYNNNDMRF

SEQ ID NO: 111 - protein, 132 aa, synthetic construct
  REGION 1..132: note = antibody or TCR sequence
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKP

SEQ ID NO: 112 - protein, 141 aa, synthetic construct; identical to SEQ ID NO: 16
  REGION 1..141: note = antibody or TCR sequence

SEQ ID NO: 113 - protein, 273 aa, synthetic construct
  REGION 1..273: note = antibody or TCR sequence
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 114 - protein, 5 aa, synthetic construct
  REGION 1..5: note = antibody or TCR sequence
SGHNS

SEQ ID NO: 115 - protein, 6 aa, synthetic construct
  REGION 1..6: note = antibody or TCR sequence
FNNNVP

SEQ ID NO: 116 - protein, 13 aa, synthetic construct
  REGION 1..13: note = antibody or TCR sequence
CASSPGSTDTQYF

SEQ ID NO: 117 - protein, 132 aa, synthetic construct
  REGION 1..132: note = antibody or TCR sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVL

SEQ ID NO: 118 - protein, 179 aa, synthetic construct; identical to SEQ ID NO: 22
  REGION 1..179: note = antibody or TCR sequence

SEQ ID NO: 119 - protein, 311 aa, synthetic construct
  REGION 1..311: note = antibody or TCR sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 120 - protein, 7 aa, synthetic construct
  REGION 1..7: note = antibody or TCR sequence
MTLNGDE

SEQ ID NO: 121 - protein, 6 aa, synthetic construct
  REGION 1..6: note = antibody or TCR sequence
IYSNGD

SEQ ID NO: 122 - protein, 6 aa, synthetic construct
  REGION 1..6: note = antibody or TCR sequence
TYSSGN

SEQ ID NO: 123 - protein, 6 aa, synthetic construct
  REGION 1..6: note = antibody or TCR sequence
IYSNGD

SEQ ID NO: 124 - protein, 7 aa, synthetic construct
  REGION 1..7: note = antibody or TCR sequence
QEAYKQQ

SEQ ID NO: 125 - protein, 6 aa, synthetic construct
  REGION 1..6: note = antibody or TCR sequence
IYSNGD

SEQ ID NO: 126 - protein, 6 aa, synthetic construct
  REGION 1..6: note = antibody or TCR sequence
IYSNGD

SEQ ID NO: 127 - protein, 7 aa, synthetic construct
  REGION 1..7: note = antibody or TCR sequence
IQSSQRE

SEQ ID NO: 128 - protein, 7 aa, synthetic construct
  REGION 1..7: note = antibody or TCR sequence
MTLNGDE

SEQ ID NO: 129 - protein, 21 aa, synthetic construct
  REGION 1..21: note = antibody or TCR sequence
EPKSCDKTHTCPPCPAPELLG

SEQ ID NO: 130 - protein, 6 aa, synthetic construct
  REGION 1..6: note = antibody or TCR sequence
ELLGGP

SEQ ID NO: 131 - protein, 461 aa, synthetic construct
  REGION 1..461: note = antibody or TCR sequence
QKEVEQNSGPLSVPEGAIASLNCTYSDRGSQSFFWYRQYSGKSPELIMSIYSNGDKEDGRFTAQLNKASQYFSLLIRDSQPSDSATYLCAAVIDNSNGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 132 - protein, 477 aa, synthetic construct
  REGION 1..477: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRSVSWYQQTPGQGLQFLFEYVHGAERNKGNFPGRFSGRQFSNSSSEMNISNLELGDSALYLCASSPWDSPNEQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 133 - protein, 463 aa, synthetic construct
  REGION 1..463: note = antibody or TCR sequence
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 479 aa, synthetic construct
  REGION 1..479: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - protein, 6 aa, synthetic construct
  REGION 1..6: note = antibody or TCR sequence
DRGSQS

SEQ ID NO: 136 - protein, 6 aa, synthetic construct
  REGION 1..6: note = antibody or TCR sequence
DRGSQL

SEQ ID NO: 137 - protein, 6 aa, synthetic construct
  REGION 1..6: note = antibody or TCR sequence
IYSNGD

SEQ ID NO: 138 - protein, 6 aa, synthetic construct
  REGION 1..6: note = antibody or TCR sequence
IYQEGD

SEQ ID NO: 139 - protein, 15 aa, synthetic construct
  REGION 1..15: note = antibody or TCR sequence
CAAVINNPSGGMLTF

SEQ ID NO: 140 - protein, 15 aa, synthetic construct
  REGION 1..15: note = antibody or TCR sequence
CAAVIDNSNGGILTF

SEQ ID NO: 141 - protein, 15 aa, synthetic construct
  REGION 1..15: note = antibody or TCR sequence
CAAVIDNPSGGILTF

SEQ ID NO: 142 - protein, 15 aa, synthetic construct
  REGION 1..15: note = antibody or TCR sequence
CAAVIDNDQGGILTF

SEQ ID NO: 143 - protein, 15 aa, synthetic construct
  REGION 1..15: note = antibody or TCR sequence
CAAVIPNPPGGKLTF

SEQ ID NO: 144 - protein, 15 aa, synthetic construct
  REGION 1..15: note = antibody or TCR sequence
CAAVIPNPGGGALTF

SEQ ID NO: 145 - protein, 15 aa, synthetic construct
  REGION 1..15: note = antibody or TCR sequence
CAAVIPNSAGGRLTF

SEQ ID NO: 146 - protein, 15 aa, synthetic construct
  REGION 1..15: note = antibody or TCR sequence
CAAVIPNLEGGSLTF

SEQ ID NO: 147 - protein, 15 aa, synthetic construct
  REGION 1..15: note = antibody or TCR sequence
CAAVIPNRLGGYLTF

SEQ ID NO: 148 - protein, 15 aa, synthetic construct
  REGION 1..15: note = antibody or TCR sequence
CAAVIPNTDGGRLTF

SEQ ID NO: 149 - protein, 15 aa, synthetic construct
  REGION 1..15: note = antibody or TCR sequence
CAAVIPNQRGGALTF

SEQ ID NO: 150 - protein, 15 aa, synthetic construct
  REGION 1..15: note = antibody or TCR sequence
CAAVIPNVVGGILTF

SEQ ID NO: 151 - protein, 15 aa, synthetic construct
  REGION 1..15: note = antibody or TCR sequence
CAAVITNIAGGSLTF

SEQ ID NO: 152 - protein, 15 aa, synthetic construct
  REGION 1..15: note = antibody or TCR sequence
CAAVIPNNDGGYLTF

SEQ ID NO: 153 - protein, 15 aa, synthetic construct
  REGION 1..15: note = antibody or TCR sequence
CAAVIPNGRGGLLTF

SEQ ID NO: 154 - protein, 15 aa, synthetic construct
  REGION 1..15: note = antibody or TCR sequence
CAAVIPNTHGGPLTF

SEQ ID NO: 155 - protein, 15 aa, synthetic construct
  REGION 1..15: note = antibody or TCR sequence
CAAVIPNDVGGSLTF

SEQ ID NO: 156 - protein, 15 aa, synthetic construct
  REGION 1..15: note = antibody or TCR sequence
CAAVIENKPGGPLTF

SEQ ID NO: 157 - protein, 15 aa, synthetic construct
  REGION 1..15: note = antibody or TCR sequence
CAAVIDNPVGGPLTF

SEQ ID NO: 158 - protein, 15 aa, synthetic construct
  REGION 1..15: note = antibody or TCR sequence
CAAVIPNNNGGALTF

SEQ ID NO: 159 - protein, 15 aa, synthetic construct
  REGION 1..15: note = antibody or TCR sequence
CAAVIPNDQGGILTF

SEQ ID NO: 160 - protein, 15 aa, synthetic construct
  REGION 1..15: note = antibody or TCR sequence
CAAVIPNVVGGQLTF

SEQ ID NO: 161 - protein, 15 aa, synthetic construct
  REGION 1..15: note = antibody or TCR sequence
CAAVIPNSYGGLLTF

SEQ ID NO: 162 - protein, 15 aa, synthetic construct
  REGION 1..15: note = antibody or TCR sequence
CAAVIPNDDGGLLTF

SEQ ID NO: 163 - protein, 15 aa, synthetic construct
  REGION 1..15: note = antibody or TCR sequence
CAAVIPNAAGGLLTF

SEQ ID NO: 164 - protein, 15 aa, synthetic construct
  REGION 1..15: note = antibody or TCR sequence
CAAVIPNTIGGLLTF

SEQ ID NO: 165 - protein, 15 aa, synthetic construct
  REGION 1..15: note = antibody or TCR sequence
CAAVIPNTRGGLLTF

SEQ ID NO: 166 - protein, 5 aa, synthetic construct
  REGION 1..5: note = antibody or TCR sequence
SGHRS

SEQ ID NO: 167 - protein, 5 aa, synthetic construct
  REGION 1..5: note = antibody or TCR sequence
PGHRA

SEQ ID NO: 168 - protein, 5 aa, synthetic construct
  REGION 1..5: note = antibody or TCR sequence
PGHRS

SEQ ID NO: 169 - protein, 6 aa, synthetic construct
  REGION 1..6: note = antibody or TCR sequence
YFSETQ

SEQ ID NO: 170 - protein, 6 aa, synthetic construct
  REGION 1..6: note = antibody or TCR sequence
YVHGEE

SEQ ID NO: 171 - protein, 6 aa, synthetic construct
  REGION 1..6: note = antibody or TCR sequence
YVHGAE

SEQ ID NO: 172 - protein, 14 aa, synthetic construct
  REGION 1..14: note = antibody or TCR sequence
CASSPWDSPNEQYF

SEQ ID NO: 173 - protein, 14 aa, synthetic construct
  REGION 1..14: note = antibody or TCR sequence
CASSPWDSPNVQYF

SEQ ID NO: 174 - protein, 503 aa, synthetic construct
  REGION 1..503: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGQKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGGSGGGGSGGGGSGGGGSGGGGSGSKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 175 - protein, 214 aa, synthetic construct
  REGION 1..214: note = antibody or TCR sequence
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 461 aa, synthetic construct
  REGION 1..461: note = antibody or TCR sequence
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 177 - protein, 477 aa, synthetic construct
  REGION 1..477: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 178 - protein, 214 aa, synthetic construct; identical to SEQ ID NO: 175
  REGION 1..214: note = antibody or TCR sequence

SEQ ID NO: 179 - protein, 487 aa, synthetic construct
  REGION 1..487: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPTREFQDLHWYRKETAKSPEFLFYFGPYGVEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGSTDTQYFGPGTRLTVL

SEQ ID NO: 180 - protein, 487 aa, synthetic construct
  REGION 1..487: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPTKEFQDLHWYRKETAKSPEFLFYFGPYGREKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVL

SEQ ID NO: 181 - protein, 487 aa, synthetic construct
  REGION 1..487: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPSSNFYNLHWYRKETAKSPEFLFYFGPYGVEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFNSETVIDDSGMPEDRFSAKMPNASFSTLKIQPSEPRDSAVYFCASSPGATDTQYFGPGTRLTVL

SEQ ID NO: 182 - protein, 487 aa, synthetic construct
  REGION 1..487: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPNKEFQDLHWYRKETAKSPEFLFYFGPYGTEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGSTDTQYFGPGTRLTVL

SEQ ID NO: 183 - protein, 487 aa, synthetic construct
  REGION 1..487: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGGSGGGGSGGGGSGGGGSGGGGSGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVL

SEQ ID NO: 184 - protein, 454 aa, synthetic construct
  REGION 1..454: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 185 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 186 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 187 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 188 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHLVTEMGQEVTLRCKPISGHNSLFWYRETPMQGLELLIYFQNTAVIDDSGMPEDRFSAKMPNASFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 189 - protein, 472 aa, synthetic construct
  REGION 1..472: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 190 - protein, 454 aa, synthetic construct
  REGION 1..454: note = antibody or TCR sequence
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 191 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 192 - protein, 107 aa, synthetic construct
  REGION 1..107: note = antibody or TCR sequence
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIK

SEQ ID NO: 193 - protein, 122 aa, synthetic construct
  REGION 1..122: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSS

SEQ ID NO: 194 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDTQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 195 - protein, 467 aa, synthetic construct
  REGION 1..467: note = antibody or TCR sequence
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 196 - protein, 120 aa, synthetic construct
  REGION 1..120: note = antibody or TCR sequence
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 197 - protein, 106 aa, synthetic construct
  REGION 1..106: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIK

SEQ ID NO: 198 - protein, 120 aa, synthetic construct
  REGION 1..120: note = antibody or TCR sequence
EVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 199 - protein, 120 aa, synthetic construct
  REGION 1..120: note = antibody or TCR sequence
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSS

SEQ ID NO: 200 - protein, 120 aa, synthetic construct
  REGION 1..120: note = antibody or TCR sequence
EVQLVQSGAEVKKPGASVKVSCKASGYKFTRYVMHWVRQAPGQGLEWMGYINPYNDVTKYAEKFQGRVTLTSDTSTSTAYMELSSLRSEDTAVYYCARGSYYDYEGFVYWGQGTLVTVSS

SEQ ID NO: 201 - protein, 120 aa, synthetic construct
  REGION 1..120: note = antibody or TCR sequence
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSS

SEQ ID NO: 202 - protein, 125 aa, synthetic construct
  REGION 1..125: note = antibody or TCR sequence
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 203 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHLVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 204 - protein, 109 aa, synthetic construct
  REGION 1..109: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 205 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 206 - protein, 454 aa, synthetic construct
  REGION 1..454: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 207 - protein, 125 aa, synthetic construct
  REGION 1..125: note = antibody or TCR sequence
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSS

SEQ ID NO: 208 - protein, 454 aa, synthetic construct; identical to SEQ ID NO: 190
  REGION 1..454: note = antibody or TCR sequence

SEQ ID NO: 209 - protein, 125 aa, synthetic construct
  REGION 1..125: note = antibody or TCR sequence
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGESYISYWAYWGQGTLVTVSS

SEQ ID NO: 210 - protein, 454 aa, synthetic construct
  REGION 1..454: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 211 - protein, 125 aa, synthetic construct
  REGION 1..125: note = antibody or TCR sequence
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSS

SEQ ID NO: 212 - protein, 454 aa, synthetic construct
  REGION 1..454: note = antibody or TCR sequence
IMNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 213 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 214 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETMMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 215 - protein, 470 aa, synthetic construct
  REGION 1..470: note = antibody or TCR sequence
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGGVIQSPRHEVTEMGQEVTLRCKPISGHNSLFWYRETMMRGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 216 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 217 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDLQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 218 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 219 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 220 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 221 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 222 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 223 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 224 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSAGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 225 - protein, 454 aa, synthetic construct; identical to SEQ ID NO: 223
  REGION 1..454: note = antibody or TCR sequence

SEQ ID NO: 226 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 227 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSAGSTDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 228 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSIDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 229 - protein, 467 aa, synthetic construct
  REGION 1..467: note = antibody or TCR sequence
ILNVEQSPQSLHVQEGDSTKFTCSFPVKEFQDIHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 230 - protein, 467 aa, synthetic construct; identical to SEQ ID NO: 222
  REGION 1..467: note = antibody or TCR sequence

SEQ ID NO: 231 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETMMRGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 232 - protein, 467 aa, synthetic construct; identical to SEQ ID NO: 187
  REGION 1..467: note = antibody or TCR sequence

SEQ ID NO: 233 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSAGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 234 - protein, 467 aa, synthetic construct; identical to SEQ ID NO: 218
  REGION 1..467: note = antibody or TCR sequence

SEQ ID NO: 235 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSTGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 236 - protein, 467 aa, synthetic construct; identical to SEQ ID NO: 220
  REGION 1..467: note = antibody or TCR sequence

SEQ ID NO: 237 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 238 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 239 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 240 - protein, 472 aa, synthetic construct; identical to SEQ ID NO: 189
  REGION 1..472: note = antibody or TCR sequence

SEQ ID NO: 241 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGESYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 242 - protein, 472 aa, synthetic construct
  REGION 1..472: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 243 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 244 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNNDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 245 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSAGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 246 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 247 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 248 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSTGAIDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 249 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 250 - protein, 472 aa, synthetic construct; identical to SEQ ID NO: 242
  REGION 1..472: note = antibody or TCR sequence

SEQ ID NO: 251 - protein, 472 aa, synthetic construct; identical to SEQ ID NO: 244
  REGION 1..472: note = antibody or TCR sequence

SEQ ID NO: 252 - protein, 472 aa, synthetic construct; identical to SEQ ID NO: 246
  REGION 1..472: note = antibody or TCR sequence

SEQ ID NO: 253 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNADMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 254 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNDDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 255 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNEDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 256 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNFDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 257 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNHDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 258 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNIDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 259 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 260 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNKDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 261 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNQDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 262 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNRDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 263 - protein, 467 aa, synthetic construct
  REGION 1..467: note = antibody or TCR sequence
ILNVEQSPQSLHVQEGDSTNFTCSFPVKEFQDLHWYRKETAKSPEFLFYFGPYGKEKKKGRISATLNTKEGYSYLYITDSQPEDSATYLCALYNNVDMRFGAGTRLTVKPGGGSGGGGEVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 264 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNESFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 265 - protein, 467 aa, synthetic construct; identical to SEQ ID NO: 218
  REGION 1..467: note = antibody or TCR sequence

SEQ ID NO: 266 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNRSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 267 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNKSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 268 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNQSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 269 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNNSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 270 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNSSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 271 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDRQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 272 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDHQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 273 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDEQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 274 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 275 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDQQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 276 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDNQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 277 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDFQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 278 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDYQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 279 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDIQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 280 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDVQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 281 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDRQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 282 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDHQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 283 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDEQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 284 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDAQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 285 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDQQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 286 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDNQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 287 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDFQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 288 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDYQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 289 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDIQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 290 - protein, 454 aa, synthetic construct
  REGION 1..454: note = antibody or TCR sequence
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGGVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDVQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 291 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNESFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 292 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNRSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 293 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNKSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 294 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNQSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 295 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNNSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 296 - protein, 457 aa, synthetic construct
  REGION 1..457: note = antibody or TCR sequence
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGGGVIQSPRHEVTEMGQEVTLRCKPISGHNSLFWYRETPMQGLELLIYFQNTAVIDDSGMPEDRFSAKMPNSSFSTLKIQPSEPRDSAVYFCASSPGATDKQYFGPGTRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 297 - protein, 454 aa, synthetic construct; identical to SEQ ID NO: 284
  REGION 1..454: note = antibody or TCR sequence

SEQ ID NO: 298 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 299 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 300 - protein, 472 aa, synthetic construct
  REGION 1..472: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 301 - protein, 457 aa, synthetic construct; identical to SEQ ID NO: 239
  REGION 1..457: note = antibody or TCR sequence

SEQ ID NO: 302 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTRYVMHWVRQAPGQGLEWMGYINPYNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 303 - protein, 454 aa, synthetic construct; identical to SEQ ID NO: 247
  REGION 1..454: note = antibody or TCR sequence

SEQ ID NO: 304 - protein, 467 aa, synthetic construct
  REGION 1..467: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 305 - protein, 110 aa, synthetic construct
  REGION 1..110: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNYDMRFGAGTRLTVKP

SEQ ID NO: 306 - protein, 111 aa, synthetic construct
  REGION 1..111: note = antibody or TCR sequence
GVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGATDKQYFGPGTRLTVL

SEQ ID NO: 307 - protein, 111 aa, synthetic construct
  REGION 1..111: note = antibody or TCR sequence
GVIQSPRHEVTEMGQEVTLRCKPISGHNSLFWYRETPMQGLELLIYFQNTAVIDDSGMPEDRFSAKMPNASFSTLKIQPSEPRDSAVYFCASSPGATDKQYFGPGTRLTVL

SEQ ID NO: 308 - protein, 111 aa, synthetic construct
  REGION 1..111: note = antibody or TCR sequence
GVIQSPRHEVTEMGQEVTLRCKPISGHNSLFWYRETPMQGLELLIYFQNTAVIDDSGMPEDRFSAKMPNDSFSTLKIQPSEPRDSAVYFCASSPGSTDAQYFGPGTRLTVL

SEQ ID NO: 309 - protein, 110 aa, synthetic construct
  REGION 1..110: note = antibody or TCR sequence
ILNVEQSPQSLHVQEGDSTKFTCSFPVKEFQDLHWYRKETAKSPEFLFYFGPYGKEKKKGRISATLNTKEGYSYLYITDSQPEDSATYLCALYNNLDMRFGAGTRLTVKP

SEQ ID NO: 310 - protein, 9 aa, synthetic construct
  REGION 1..9: note = target peptide
SLLQHLIGL

SEQ ID NO: 311 - protein, 9 aa, synthetic construct
  REGION 1..9: note = target peptide
SLLMWITQV

SEQ ID NO: 312 - protein, 10 aa, synthetic construct
  REGION 1..10: note = target peptide
STASAITPSV

SEQ ID NO: 313 - protein, 10 aa, Homo sapiens
YLYDSETKNA

SEQ ID NO: 314 - protein, 9 aa, Homo sapiens
HLMDQPLSV

SEQ ID NO: 315 - protein, 9 aa, Homo sapiens
GLLKKINSV

SEQ ID NO: 316 - protein, 9 aa, Homo sapiens
FLVDGSSAL

SEQ ID NO: 317 - protein, 10 aa, Homo sapiens
FLFDGSANLV

SEQ ID NO: 318 - protein, 9 aa, Homo sapiens
FLYKIIDEL

SEQ ID NO: 319 - protein, 11 aa, Homo sapiens
FILDSAETTTL

SEQ ID NO: 320 - protein, 9 aa, Homo sapiens
SVDVSPPKV

SEQ ID NO: 321 - protein, 8 aa, Homo sapiens
VADKIHSV

SEQ ID NO: 322 - protein, 9 aa, Homo sapiens
IVDDLTINL

SEQ ID NO: 323 - protein, 9 aa, Homo sapiens
GLLEELVTV

SEQ ID NO: 324 - protein, 10 aa, Homo sapiens
TLDGAAVNQV

SEQ ID NO: 325 - protein, 10 aa, Homo sapiens
SVLEKEIYSI

SEQ ID NO: 326 - protein, 9 aa, Homo sapiens
LLDPKTIFL

SEQ ID NO: 327 - protein, 9 aa, Homo sapiens
YTFSGDVQL

SEQ ID NO: 328 - protein, 9 aa, Homo sapiens
YLMDDFSSL

SEQ ID NO: 329 - protein, 9 aa, Homo sapiens
KVWSDVTPL

SEQ ID NO: 330 - protein, 11 aa, Homo sapiens
LLWGHPRVALA

SEQ ID NO: 331 - protein, 11 aa, Homo sapiens
KIWEELSVLEV

SEQ ID NO: 332 - protein, 9 aa, Homo sapiens
LLIPFTIFM

SEQ ID NO: 333 - protein, 9 aa, Homo sapiens
FLIENLLAA

SEQ ID NO: 334 - protein, 11 aa, Homo sapiens
LLWGHPRVALA

SEQ ID NO: 335 - protein, 9 aa, Homo sapiens
FLLEREQLL

SEQ ID NO: 336 - protein, 9 aa, Homo sapiens
SLAETIFIV

SEQ ID NO: 337 - protein, 9 aa, Homo sapiens
TLLEGISRA

SEQ ID NO: 338 - protein, 9 aa, Homo sapiens
ILQDGQFLV

SEQ ID NO: 339 - protein, 10 aa, Homo sapiens
VIFEGEPMYL

SEQ ID NO: 340 - protein, 9 aa, Homo sapiens
SLFESLEYL

SEQ ID NO: 341 - protein, 9 aa, Homo sapiens
SLLNQPKAV

SEQ ID NO: 342 - protein, 9 aa, Homo sapiens
GLAEFQENV

SEQ ID NO: 343 - protein, 9 aa, Homo sapiens
KLLAVIHEL

SEQ ID NO: 344 - protein, 9 aa, Homo sapiens
TLHDQVHLL

SEQ ID NO: 345 - protein, 11 aa, Homo sapiens
TLYNPERTITV

SEQ ID NO: 346 - protein, 9 aa, Homo sapiens
KLQEKIQEL

SEQ ID NO: 347 - protein, 10 aa, Homo sapiens
SVLEKEIYSI

SEQ ID NO: 348 - protein, 11 aa, Homo sapiens
RVIDDSLVVGV

SEQ ID NO: 349 - protein, 9 aa, Homo sapiens
VLFGELPAL

SEQ ID NO: 350 - protein, 9 aa, Homo sapiens
GLVDIMVHL

SEQ ID NO: 351 - protein, 9 aa, Homo sapiens
FLNAIETAL

SEQ ID NO: 352 - protein, 9 aa, Homo sapiens
ALLQALMEL

SEQ ID NO: 353 - protein, 9 aa, Homo sapiens
ALSSSQAEV

SEQ ID NO: 354 - protein, 11 aa, Homo sapiens
SLITGQDLLSV

SEQ ID NO: 355 - protein, 9 aa, Homo sapiens
QLIEKNWLL

SEQ ID NO: 356 - protein, 9 aa, Homo sapiens
LLDPKTIFL

SEQ ID NO: 357 - protein, 9 aa, Homo sapiens
RLHDENILL

SEQ ID NO: 358 - protein, 9 aa, Homo sapiens
YTFSGDVQL

SEQ ID NO: 359 - protein, 9 aa, Homo sapiens
GLPSATTTV

SEQ ID NO: 360 - protein, 11 aa, Homo sapiens
GLLPSAESIKL

SEQ ID NO: 361 - protein, 9 aa, Homo sapiens
KTASINQNV

SEQ ID NO: 362 - protein, 9 aa, Homo sapiens
SLLQHLIGL

SEQ ID NO: 363 - protein, 9 aa, Homo sapiens
YLMDDFSSL

SEQ ID NO: 364 - protein, 9 aa, Homo sapiens
LMYPYIYHV

SEQ ID NO: 365 - protein, 9 aa, Homo sapiens
KVWSDVTPL

SEQ ID NO: 366 - protein, 11 aa, Homo sapiens
LLWGHPRVALA

SEQ ID NO: 367 - protein, 9 aa, Homo sapiens
VLDGKVAVV

SEQ ID NO: 368 - protein, 9 aa, Homo sapiens
GLLGKVTSV

SEQ ID NO: 369 - protein, 9 aa, Homo sapiens
KMISAIPTL

SEQ ID NO: 370 - protein, 11 aa, Homo sapiens
GLLETTGLLAT

SEQ ID NO: 371 - protein, 9 aa, Homo sapiens
TLNTLDINL

SEQ ID NO: 372 - protein, 9 aa, Homo sapiens
VIIKGLEEI

SEQ ID NO: 373 - protein, 9 aa, Homo sapiens
YLEDGFAYV

SEQ ID NO: 374 - protein, 11 aa, Homo sapiens
KIWEELSVLEV

SEQ ID NO: 375 - protein, 9 aa, Homo sapiens
LLIPFTIFM

SEQ ID NO: 376 - protein, 10 aa, Homo sapiens
ISLDEVAVSL

SEQ ID NO: 377 - protein, 10 aa, Homo sapiens
KISDFGLATV

SEQ ID NO: 378 - protein, 11 aa, Homo sapiens
KLIGNIHGNEV

SEQ ID NO: 379 - protein, 9 aa, Homo sapiens
ILLSVLHQL

SEQ ID NO: 380 - protein, 9 aa, Homo sapiens
LDSEALLTL

SEQ ID NO: 381 - protein, 13 aa, Homo sapiens
VLQENSSDYQSNL

SEQ ID NO: 382 - protein, 11 aa, Homo sapiens
HLLGEGAFAQV

SEQ ID NO: 383 - protein, 9 aa, Homo sapiens
SLVENIHVL

SEQ ID NO: 384 - protein, 9 aa, Homo sapiens
YTFSGDVQL

SEQ ID NO: 385 - protein, 9 aa, Homo sapiens
SLSEKSPEV

SEQ ID NO: 386 - protein, 10 aa, Homo sapiens
AMFPDTIPRV

SEQ ID NO: 387 - protein, 9 aa, Homo sapiens
FLIENLLAA

SEQ ID NO: 388 - protein, 9 aa, Homo sapiens
FTAEFLEKV

SEQ ID NO: 389 - protein, 9 aa, Homo sapiens
ALYGNVQQV

SEQ ID NO: 390 - protein, 9 aa, Homo sapiens
LFQSRIAGV

SEQ ID NO: 391 - protein, 11 aa, Homo sapiens
ILAEEPIYIRV

SEQ ID NO: 392 - protein, 9 aa, Homo sapiens
FLLEREQLL

SEQ ID NO: 393 - protein, 10 aa, Homo sapiens
LLLPLELSLA

SEQ ID NO: 394 - protein, 9 aa, Homo sapiens
SLAETIFIV

SEQ ID NO: 395 - protein, 11 aa, Homo sapiens
AILNVDEKNQV

SEQ ID NO: 396 - protein, 9 aa, Homo sapiens
RLFEEVLGV

SEQ ID NO: 397 - protein, 9 aa, Homo sapiens
YLDEVAFML

SEQ ID NO: 398 - protein, 11 aa, Homo sapiens
KLIDEDEPLFL

SEQ ID NO: 399 - protein, 9 aa, Homo sapiens
KLFEKSTGL

SEQ ID NO: 400 - protein, 11 aa, Homo sapiens
SLLEVNEASSV

SEQ ID NO: 401 - protein, 10 aa, Homo sapiens
GVYDGREHTV

SEQ ID NO: 402 - protein, 10 aa, Homo sapiens
GLYPVTLVGV

SEQ ID NO: 403 - protein, 9 aa, Homo sapiens
ALLSSVAEA

SEQ ID NO: 404 - protein, 9 aa, Homo sapiens
TLLEGISRA

SEQ ID NO: 405 - protein, 9 aa, Homo sapiens
SLIEESEEL

SEQ ID NO: 406 - protein, 9 aa, Homo sapiens
ALYVQAPTV

SEQ ID NO: 407 - protein, 10 aa, Homo sapiens
KLIYKDLVSV

SEQ ID NO: 408 - protein, 9 aa, Homo sapiens
ILQDGQFLV

SEQ ID NO: 409 - protein, 9 aa, Homo sapiens
SLLDYEVSI

SEQ ID NO: 410 - protein, 9 aa, Homo sapiens
LLGDSSFFL

SEQ ID NO: 411 - protein, 10 aa, Homo sapiens
VIFEGEPMYL

SEQ ID NO: 412 - protein, 9 aa, Homo sapiens
ALSYILPYL

SEQ ID NO: 413 - protein, 9 aa, Homo sapiens
FLFVDPELV

SEQ ID NO: 414 - protein, 11 aa, Homo sapiens
SEWGSPHAAVP

SEQ ID NO: 415 - protein, 9 aa, Homo sapiens
ALSELERVL

SEQ ID NO: 416 - protein, 9 aa, Homo sapiens
SLFESLEYL

SEQ ID NO: 417 - protein, 9 aa, Homo sapiens
KVLEYVIKV

SEQ ID NO: 418 - protein, 10 aa, Homo sapiens
VLLNEILEQV

SEQ ID NO: 419 - protein, 9 aa, Homo sapiens
SLLNQPKAV

SEQ ID NO: 420 - protein, 9 aa, Homo sapiens
KMSELQTYV

SEQ ID NO: 421 - protein, 11 aa, Homo sapiens
ALLEQTGDMSL

SEQ ID NO: 422 - protein, 11 aa, Homo sapiens
VIIKGLEEITV

SEQ ID NO: 423 - protein, 9 aa, Homo sapiens
KQFEGTVEI

SEQ ID NO: 424 - protein, 9 aa, Homo sapiens
KLQEEIPVL

SEQ ID NO: 425 - protein, 9 aa, Homo sapiens
GLAEFQENV

SEQ ID NO: 426 - protein, 9 aa, Homo sapiens
NVAEIVIHI

SEQ ID NO: 427 - protein, 9 aa, Homo sapiens
ALAGIVTNV

SEQ ID NO: 428 - protein, 12 aa, Homo sapiens
NLLIDDKGTIKL

SEQ ID NO: 429 - protein, 10 aa, Homo sapiens
VLMQDSRLYL

SEQ ID NO: 430 - protein, 9 aa, Homo sapiens
KVLEHVVRV

SEQ ID NO: 431 - protein, 9 aa, Homo sapiens
LLWGNLPEI

SEQ ID NO: 432 - protein, 9 aa, Homo sapiens
SLMEKNQSL

SEQ ID NO: 433 - protein, 9 aa, Homo sapiens
KLLAVIHEL

SEQ ID NO: 434 - protein, 10 aa, Homo sapiens
ALGDKFLLRV

SEQ ID NO: 435 - protein, 11 aa, Homo sapiens
FLMKNSDLYGA

SEQ ID NO: 436 - protein, 10 aa, Homo sapiens
KLIDHQGLYL

SEQ ID NO: 437 - protein, 12 aa, Homo sapiens
GPGIFPPPPPQP

SEQ ID NO: 438 - protein, 9 aa, Homo sapiens
ALNESLVEC

SEQ ID NO: 439 - protein, 9 aa, Homo sapiens
GLAALAVHL

SEQ ID NO: 440 - protein, 9 aa, Homo sapiens
LLLEAVWHL

SEQ ID NO: 441 - protein, 9 aa, Homo sapiens
SIIEYLPTL

SEQ ID NO: 442 - protein, 9 aa, Homo sapiens
TLHDQVHLL

SEQ ID NO: 443 - protein, 9 aa, Homo sapiens
SLLMWITQC

SEQ ID NO: 444 - protein, 11 aa, Homo sapiens
FLLDKPQDLSI

SEQ ID NO: 445 - protein, 10 aa, Homo sapiens
YLLDMPLWYL

SEQ ID NO: 446 - protein, 9 aa, Homo sapiens
GLLDCPIFL

SEQ ID NO: 447 - protein, 9 aa, Homo sapiens
VLIEYNFSI

SEQ ID NO: 448 - protein, 11 aa, Homo sapiens
TLYNPERTITV

SEQ ID NO: 449 - protein, 9 aa, Homo sapiens
AVPPPPSSV

SEQ ID NO: 450 - protein, 9 aa, Homo sapiens
KLQEELNKV

SEQ ID NO: 451 - protein, 11 aa, Homo sapiens
KLMDPGSLPPL

SEQ ID NO: 452 - protein, 9 aa, Homo sapiens
ALIVSLPYL

SEQ ID NO: 453 - protein, 9 aa, Homo sapiens
FLLDGSANV

SEQ ID NO: 454 - protein, 10 aa, Homo sapiens
ALDPSGNQLI

SEQ ID NO: 455 - protein, 9 aa, Homo sapiens
ILIKHLVKV

SEQ ID NO: 456 - protein, 9 aa, Homo sapiens
VLLDTILQL

SEQ ID NO: 457 - protein, 9 aa, Homo sapiens
HLIAEIHTA

SEQ ID NO: 458 - protein, 9 aa, Homo sapiens
SMNGGVFAV

SEQ ID NO: 459 - protein, 9 aa, Homo sapiens
MLAEKLLQA

SEQ ID NO: 460 - protein, 9 aa, Homo sapiens
YMLDIFHEV

SEQ ID NO: 461 - protein, 11 aa, Homo sapiens
ALWLPTDSATV

SEQ ID NO: 462 - protein, 9 aa, Homo sapiens
GLASRILDA

SEQ ID NO: 463 - protein, 9 aa, Homo sapiens
ALSVLRLAL

SEQ ID NO: 464 - protein, 9 aa, Homo sapiens
SYVKVLHHL

SEQ ID NO: 465 - protein, 9 aa, Homo sapiens
VYLPKIPSW

SEQ ID NO: 466 - protein, 9 aa, Homo sapiens
NYEDHFPLL

SEQ ID NO: 467 - protein, 9 aa, Homo sapiens
VYIAELEKI

SEQ ID NO: 468 - protein, 12 aa, Homo sapiens
VHFEDTGKTLLF

SEQ ID NO: 469 - protein, 9 aa, Homo sapiens
VLSPFILTL

SEQ ID NO: 470 - protein, 9 aa, Homo sapiens
HLLEGSVGV

SEQ ID NO: 471 - protein, 9 aa, Homo sapiens
ALREEEEGV

SEQ ID NO: 472 - protein, 10 aa, Homo sapiens
KEADPTGHSY

SEQ ID NO: 473 - protein, 9 aa, Homo sapiens
TLDEKVAEL

SEQ ID NO: 474 - protein, 9 aa, Homo sapiens
KIQEILTQV

SEQ ID NO: 475 - DNA, 2082 bp, Homo sapiens
  source 1..2082: mol_type = other DNA
acgcagcggctagggcgcttagctgagccattgtctcgttctttccttccagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 476 - DNA, 2061 bp, Homo sapiens
  source 1..2061: mol_type = other DNA
acgcagcggctagggcgcttagctgagccattgtctcgttctttccttccagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 477 - DNA, 2206 bp, Homo sapiens
  source 1..2206: mol_type = other DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 478 - DNA, 1963 bp, Homo sapiens
  source 1..1963: mol_type = other DNA
acgcagcggctagggcgcttagctgagccattgtctcgttctttccttccagcaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 479 - DNA, 1980 bp, Homo sapiens
  source 1..1980: mol_type = other DNA
agacgcaaaagcccacacttccagtggtgtcagagagtatgagctccaggaggctttgatctcactgccccatgggcttgggccattagggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 480 - DNA, 2242 bp, Homo sapiens
  source 1..2242: mol_type = other DNA
aaaagcaccagtgggtgatcaggcccaggataggaatcatccctaagcagatccttaaatgccaaatataacttcaattttttgtgacggaaaatgtatttgtgcactgtgcctagcatggaaatagagaaaaactgagtctaattgataggcctgattgcatctcgctgaccagaatctctgtacctgagcagcttaggggcaagaaagaacagctgttttcttgaagtgtggaaacattagtaatacttaggaaggggctggccattggagattatgtttgtggtaaaggcctgacctgctgcttgtgaacagccccagtgtggaagaggacacctaaccataaagagaagggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 481 - DNA, 2121 bp, Homo sapiens
  source 1..2121: mol_type = other DNA
acagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 482 - DNA, 2304 bp, Homo sapiens
  source 1..2304: mol_type = other DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 483 - DNA, 2435 bp, Homo sapiens
  source 1..2435: mol_type = other DNA
agagggcctgggaggaagtgggttttgcatacagtccctgttgactctagtgccccctgctggccccagacgcgagttccggcgaggcttcagggtacagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 484 - DNA, 2100 bp, Homo sapiens
  source 1..2100: mol_type = other DNA
acagctcccccgcagccagaagccgggcctgcagcgcctcagcaccgctccgggacaccccacccgcttcccaggcgtgacctgtcaacagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 485 - DNA, 2845 bp, Homo sapiens
  source 1..2845: mol_type = other DNA
agagggcctgggaggaagtgggttttgcatacagtccctgttgactctagtgccccctgctggccccagacgcgagttccggcgaggcttcagggtacagctcccccgcagccagaagccgggcctgcagcgcctcagcaccgctccgggacaccccacccgcttcccaggcgtgacctgtcaacagcaacttcgcggtgtggtgaactctctgaggaaaaacgtaagttcgagccctgattcctccgcttccccgcagggtgaccttgggcttgtgcccccagcaccacccctgtcccgggtccctgttttctctctggaaatgggttgaagaccaaagaaaataatgtgcgccacttgggtcaccccgggccgcctgccccggaaaattggccccagttgaggagttgtggctgtaaggatgccttgaaccgaggcggcggtgctcgtggttggagctctccagggtgggtgcgcatttgtaatgcggtggatgctctgggactcggcccctctgaaggtgctgggggttggggacggcccaggcagtggcgtaggcgtcctaggaaggcgggagcagaggcagaaatgtcgctgcaagaccgtagtcagggtccttgaccacaggggtcacttgtgaccaaccacatggtctgttgttcctcctgccccctggttcagcccaggaaacactggtgctcaggtttggagccagagatttgcactgaaagggcgggattgagtcgccagttgtcagtttcctcagcagtatttgcggaggttttcacaggaggccgttgcttcgtaaatattatacatgtattcttctttttggagcattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 486 - protein, 493 aa, Homo sapiens
MSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 487 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 488 - protein, 509 aa, Homo sapiens; identical to SEQ ID NO: 487

SEQ ID NO: 489 - protein, 509 aa, Homo sapiens; identical to SEQ ID NO: 487

SEQ ID NO: 490 - protein, 509 aa, Homo sapiens; identical to SEQ ID NO: 487

SEQ ID NO: 491 - protein, 509 aa, Homo sapiens; identical to SEQ ID NO: 487

SEQ ID NO: 492 - protein, 509 aa, Homo sapiens; identical to SEQ ID NO: 487

SEQ ID NO: 493 - protein, 493 aa, Homo sapiens; identical to SEQ ID NO: 486

SEQ ID NO: 494 - protein, 493 aa, Homo sapiens; identical to SEQ ID NO: 486

SEQ ID NO: 495 - protein, 493 aa, Homo sapiens; identical to SEQ ID NO: 486

SEQ ID NO: 496 - protein, 509 aa, Homo sapiens; identical to SEQ ID NO: 487